(19) United States
(12) Patent Application Publication
(10) Pub. No.: US 2020/0331904 A1
(43) Pub. Date: Oct. 22, 2020

(54) 3-AMINO-[1,2,4]-TRIAZOLE DERIVATIVES AND THEIR USE FOR CONTROLLING UNDESIRED PLANT GROWTH

(71) Applicants: Bayer CropScience Aktiengesellschaft, Monheim am Rhein (DE); Bayer Aktiengesellschaft, Leverkusen (DE)

(72) Inventors: Estella BUSCATO ARSEQUELL, Frankfurt am Main (DE); Klemens MINN, Hattersheim (DE); Harald JAKOBI, Frankfurt (DE); Hansjoerg DIETRICH, Liederbach am Taunus (DE); Anu Bheemaiah MACHETTIRA, Frankfurt am Main (DE); Elmar GATZWEILER, Bad Nauheim (DE); Dirk SCHMUTZLER, Hattersheim (DE); Christopher Hugh ROSINGER, Hofheim (DE)

(21) Appl. No.: 16/769,075

(22) PCT Filed: Nov. 29, 2018
(86) PCT No.: PCT/EP2018/082940

§ 371 Date: Jun. 2, 2020

(30) Foreign Application Priority Data
Dec. 4, 2017 (EP) 17205161.7

(51) Int. Cl. C07D 417/12; A01N 43/72

(52) U.S. Cl. CPC C07D 417/12; A01N 43/72

(57) ABSTRACT

The invention relates to 3-amino-[1,2,4]-triazole derivatives of the general formula (I) and their agrochemically compatible salts

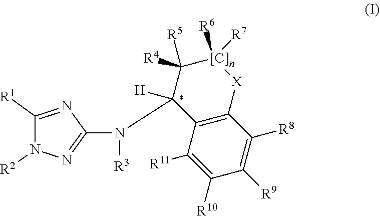

and to the use thereof for controlling unwanted plant growth.

The invention relates to the technical field of crop protection compositions, particularly to that of herbicides for selective control of broad-leaved weeds and weed grasses in crops of useful plants and in the ornamental garden sector and for general control of broad-leaved weeds and weed grasses in areas of the environment where plant growth is disruptive.

More particularly, the invention relates to substituted 3-amino-[1,2,4]-triazole derivatives, to processes for their preparation and to their use for control of harmful plants.

The prior art does not disclose the herbicidal action of such 3-amino-[1,2,4]-triazole derivatives.

The use of the known selective herbicides for control of harmful plants or as plant growth regulators in various crops of useful plants frequently entails an application rate that incurs high costs or results in unwanted damage to the useful plants. Moreover, in many cases, the use of the active compounds is uneconomic owing to comparatively high production costs.

It is therefore desirable to provide alternative chemical active compounds which can be used as herbicides or plant growth regulators and which are associated with certain advantages compared to systems known from the prior art.

It is an object of the present invention to provide alternative active compounds which can be used as herbicides or plant growth regulators, having satisfactory herbicidal action and a broad spectrum of activity against harmful plants and/or having high selectivity in crops of useful plants.

The object is achieved by means of specifically substituted 3-amino-[1,2,4]-triazole derivatives of the formula (I) as claimed in claim 1, which can advantageously be used as herbicides and also as plant growth regulators.

The present invention therefore provides compounds of the formula (I) and their agrochemically compatible salts

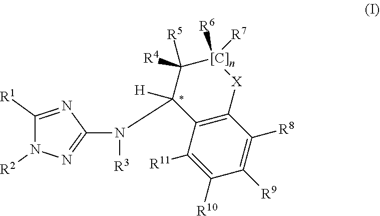

in which
R¹ is selected from the group consisting of
   hydrogen, halogen, hydroxyl, cyano, nitro, amino, C(O)OH, C(O)NH₂;
   (C₁-C₆)-alkyl, (C₁-C₆)-haloalkyl, (C₁-C₆)-alkylcarbonyl, (C₁-C₆)-haloalkylcarbonyl, (C₁-C₆)-alkylcarbonyloxy, (C₁-C₆)-haloalkylcarbonyloxy, (C₁-C₆)-alkylcarbonyl-(C₁-C₄)-alkyl;
   (C₁-C₆)-alkoxy, (C₁-C₄)-alkoxyalkyl, (C₁-C₆)-haloalkoxy, (C₁-C₆)-alkoxycarbonyl, (C₁-C₆)-haloalkoxycarbonyl, (C₁-C₆)-alkoxycarbonyl-(C₁-C₆)-alkyl, (C₁-C₆)-haloalkoxycarbonyl-(C₁-C₆)-alkyl, (C₁-C₆)-alkoxycarbonyl-(C₁-C₆)-haloalkyl, (C₁-C₆)-haloalkoxycarbonyl-(C₁-C₆)-haloalkyl;
   (C₂-C₆)-alkenyl, (C₂-C₆)-haloalkenyl, (C₂-C₆)-alkenylcarbonyl, (C₂-C₆)-haloalkenylcarbonyl, (C₂-C₆)-alkenyloxy, (C₂-C₆)-haloalkenyloxy, (C₂-C₆)-alkenyloxycarbonyl, (C₂-C₆)-haloalkenyloxycarbonyl;
   (C₂-C₆)-alkynyl, (C₂-C₆)-haloalkynyl, (C₂-C₆)-alkynylcarbonyl, (C₂-C₆)-haloalkynylcarbonyl, (C₂-C₆)-alkynyloxy, (C₂-C₆)-haloalkynyloxy, (C₂-C₆)-alkynyloxycarbonyl, (C₂-C₆)-haloalkynyloxycarbonyl;
   tri-(C₁-C₆)-alkylsilyl-(C₂-C₆)-alkynyl, di-(C₁-C₆)-alkylsilyl-(C₂-C₆)-alkynyl, mono-(C₁-C₆)-alkylsilyl-(C₂-C₆)-alkynyl, phenylsilyl-(C₂-C₆)-alkynyl;
   (C₆-C₁₄)-aryl, (C₆-C₁₄)-aryloxy, (C₆-C₁₄)-arylcarbonyl and (C₆-C₁₄)-aryloxycarbonyl, each of which may be substituted in the aryl moiety by halogen, (C₁-C₆)-alkyl and/or (C₁-C₆)-haloalkyl;
   (C₆-C₁₄)-aryl-(C₁-C₆)-alkyl, (C₆-C₁₄)-aryl-(C₁-C₆)-alkoxy, (C₆-C₁₄)-aryl-(C₁-C₆)-alkylcarbonyl, (C₆-C₁₄)-aryl-(C₁-C₆)-alkylcarbonyloxy, (C₆-C₁₄)-aryl-(C₁-C₆)-alkoxycarbonyl, (C₆-C₁₄)-aryl-(C₁-C₆)-alkoxycarbonyloxy;
   mono-((C₁-C₆)-alkyl)amino, mono-((C₁-C₆)-haloalkyl)amino, di-((C₁-C₆)-alkyl)amino, di-((C₁-C₆)-haloalkyl)amino, ((C₁-C₆)-alkyl-(C₁-C₆)-haloalkyl)amino, N—((C₁-C₆)-alkanoyl)amino, N—((C₁-C₆)-haloalkanoyl)amino, aminocarbonyl-(C₁-C₆)-alkyl, di-(C₁-C₆)-alkylaminocarbonyl-(C₁-C₆)-alkyl;
   mono-((C₁-C₆)-alkyl)aminocarbonyl, mono-((C₁-C₆)-haloalkyl)aminocarbonyl, di-((C₁-C₆)-alkyl)aminocarbonyl, di-((C₁-C₆)-haloalkyl)aminocarbonyl, ((C₁-C₆)-alkyl-(C₁-C₆)-haloalkyl)aminocarbonyl, N—((C₁-C₆)-alkanoyl)aminocarbonyl, N—((C₁-C₆)-haloalkanoyl)aminocarbonyl, mono-((C₆-C₁₄)-aryl)aminocarbonyl, di-((C₆-C₁₄)-aryl)aminocarbonyl,
   di-((C₁-C₆)-alkyl)amino, with the proviso that the two (C₁-C₆)-alkyl radicals form a cycle that may optionally be interrupted by NH, O or S;
   (C₁-C₆)-alkoxy-(C₁-C₆)-alkyl, (C₁-C₆)-alkoxy-(C₁-C₆)-alkoxy, (C₁-C₆)-alkoxycarbonyl-(C₁-C₆)-alkoxy;
   (C₃-C₈)-cycloalkyl which may optionally be substituted on the cycloalkyl radical by (C₁-C₆)-alkyl and/or halogen; (C₃-C₈)-cycloalkoxy, (C₃-C₈)-cycloalkyl-(C₁-C₆)-alkyl, (C₃-C₈)-cycloalkyl-(C₁-C₆)-haloalkyl, (C₃-C₈)-cycloalkyl-(C₁-C₆)-alkoxy, (C₃-C₈)-cycloalkyl-(C₁-C₆)-haloalkoxy, (C₃-C₈)-cycloalkylcarbonyl, (C₃-C₈)-cycloalkoxycarbonyl, (C₃-C₈)-cycloalkyl-(C₁-C₆)-alkylcarbonyl, (C₃-C₈)-cycloalkyl-(C₁-C₆)-haloalkylcarbonyl, (C₃-C₈)-cycloalkyl-(C₁-C₆)-alkoxycarbonyl, (C₃-C₈)-cycloalkyl-(C₁-C₆)-haloalkoxycarbonyl, (C₃-C₈)-cycloalkylcarbonyloxy, (C₃-C₈)-cycloalkoxycarbonyloxy, (C₃-C₈)-cycloalkyl-(C₁-C₆)-alkylcarbonyloxy, (C₃-C₈)-cycloalkyl-(C₁-C₆)-halogenalkylcarbonyloxy, (C₃-C₈)-cycloalkyl-(C₁-C₆)-alkoxycarbonyloxy, (C₃-C₈)-cycloalkyl-(C₁-C₆)-haloalkoxycarbonyloxy; (C₁-C₆)-cycloheteroalkyl which may optionally be interrupted once or twice independently of one another by NH, O or S;
   (C₃-C₈)-cycloalkenyl, (C₃-C₈)-cycloalkenyloxy, (C₃-C₈)-cycloalkenyl-(C₁-C₆)-alkyl, (C₃-C₈)-cycloalkenyl-(C₁-C₆)-haloalkyl, (C₃-C₈)-cycloalkenyl-(C₁-C₆)-alkoxy, (C₃-C₈)-cycloalkenyl-(C₁-C₆)-haloalkoxy, (C₃-C₈)-cycloalkenylcarbonyl, (C₃-C₈)-cycloalkenyloxycarbonyl, (C₃-C₈)-cycloalkenyl-(C₁-C₆)-alkylcarbonyl, (C₃-C₈)-cycloalkenyl-(C₁-C₆)-haloalkylcarbonyl, (C₃-C₈)-cycloalkenyl-(C₁-C₆)-alkoxycarbonyl, (C₃-C₈)-cycloalkenyl-(C₁-C₆)-haloalkoxycarbonyl, (C₃-C₈)-cycloalkenylcarbonyloxy, (C₃-C₈)-cycloalkenyloxycarbonyloxy, (C₃-C₈)-cycloalkenyl-(C₁-C₆)-alkylcarbonyloxy, (C₃-C₈)-cycloalkenyl-(C₁-C₆)-haloalkylcarbonyloxy, (C₃-C₈)-cycloalkenyl-(C₁-C₆)-alkoxycarbonyloxy, (C₃-C₈)-cycloalkenyl-(C₁-C₆)-haloalkoxycarbonyloxy;
   hydroxy-(C₁-C₆)-alkyl, hydroxy-(C₁-C₆)-alkoxy, cyano-(C₁-C₆)-alkoxy, cyano-(C₁-C₆)-alkyl;
   (C₁-C₆)-alkylsulfonyl, (C₁-C₆)-alkylthio, (C₁-C₆)-alkylsulfinyl, (C₁-C₆)-haloalkylsulfonyl, (C₁-C₆)-haloalkylthio, (C₁-C₆)-haloalkylsulfinyl, (C₁-C₆)-alkylsulfonyl-(C₁-C₆)-alkyl, (C₁-C₆)-alkylthio-(C₁-C₆)-alkyl, (C₁-C₆)-alkylsulfinyl-(C₁-C₆)-alkyl, (C₁-C₆)-haloalkylsulfonyl-(C₁-C₆)-alkyl, (C₁-C₆)-haloalkylthio-(C₁-C₆)-alkyl, (C₁-C₆)-haloalkylsulfinyl-(C₁-C₆)-alkyl, (C₁-C₆)-alkylsulfonyl-(C₁-C₆)-haloalkyl, (C₁-C₆)-alkylthio-(C₁-C₆)-haloalkyl, (C₁-C₆)-alkylsulfinyl-(C₁-C₆)-haloalkyl, (C₁-C₆)-haloalkylsulfonyl-(C₁-C₆)-haloalkyl, (C₁-C₆)-haloalkylthio-(C₁-C₆)-haloalkyl, (C₁-C₆)-haloalkylsulfinyl-(C₁-C₆)-haloalkyl, (C₁-C₆)-alkylsulfonyloxy, (C₁-C₆)-haloalkylsulfonyloxy, (C₁-C₆)-alkylthiocarbonyl, (C₁-C₆)-haloalkylthiocarbonyl, (C₁-C₆)-alkylthiocarbonyloxy, (C₁-C₆)-haloalkylthiocarbonyloxy, (C₁-C₆)-alkylthio-(C₁-C₆)-alkyl, (C₁-C₆)-alkylthio-(C₁-C₆)-alkoxy, (C₁-C₆)-alkylthio-(C₁-C₆)-alkylcarbonyl, (C₁-C₆)-alkylthio-(C₁-C₆)-alkylcarbonyloxy; (C₄-C₁₄)-arylsulfonyl, (C₆-C₁₄)-arylthio, (C₆-C₁₄)-arylsulfinyl, (C₃-C₈)-cycloalkylthio, (C₃-C₈)-alkenylthio, (C₃-C₈)-cycloalkenylthio, (C₃-C₆)-alkynylthio;
R² is selected from the group consisting of
   (C₁-C₆)-alkyl, (C₁-C₆)-haloalkyl, (C₁-C₆)-alkylcarbonyl, (C₁-C₆)-haloalkylcarbonyl, (C₁-C₆)-alkylcarbonyloxy, (C₁-C₆)-haloalkylcarbonyloxy, (C₁-C₆)-alkylcarbonyl-(C₁-C₄)-alkyl;
   (C₁-C₆)-alkoxy, (C₁-C₆)-haloalkoxy, (C₁-C₆)-alkoxycarbonyl, (C₁-C₆)-haloalkoxycarbonyl, (C₁-C₆)-alkoxycarbonyl-(C₁-C₆)-alkyl, (C₁-C₆)-haloalkoxycarbonyl-(C₁-C₆)-alkyl, (C₁-C₆)-alkoxycarbonyl-(C₁-C₆)-haloalkyl, (C₁-C₆)-haloalkoxycarbonyl-(C₁-C₆)-haloalkyl;
   (C₂-C₆)-alkenyl, (C₂-C₆)-haloalkenyl, (C₂-C₆)-alkenylcarbonyl, (C₂-C₆)-haloalkenylcarbonyl, (C₂-C₆)-alkenyloxy, (C₂-C₆)-haloalkenyloxy, (C₂-C₆)-alkenyloxycarbonyl, (C₂-C₆)-haloalkenyloxycarbonyl;
   (C₂-C₆)-alkynyl, (C₂-C₆)-haloalkynyl, (C₂-C₆)-alkynylcarbonyl, (C₂-C₆)-haloalkynylcarbonyl, (C₂-C₆)-alkynyloxy, (C₂-C₆)-haloalkynyloxy, (C₂-C₆)-alkynyloxycarbonyl, (C₂-C₆)-haloalkynyloxycarbonyl;
   tri-(C₁-C₆)-alkylsilyl-(C₂-C₆)-alkynyl, di-(C₁-C₆)-alkylsilyl-(C₂-C₆)-alkynyl, mono-(C₁-C₆)-alkylsilyl-(C₂-C₆)-alkynyl, phenylsilyl-(C₂-C₆)-alkynyl;
   (C₆-C₁₄)-aryl, (C₆-C₁₄)-aryloxy, (C₆-C₁₄)-arylcarbonyl and (C₆-C₁₄)-aryloxycarbonyl, each of which may be substituted in the aryl moiety by halogen, (C₁-C₆)-alkyl and/or (C₁-C₆)-haloalkyl;
   (C₆-C₁₄)-aryl-(C₁-C₆)-alkyl, (C₆-C₁₄)-aryl-(C₁-C₆)-alkoxy, (C₆-C₁₄)-aryl-(C₁-C₆)-alkylcarbonyl, (C₆-C₁₄)-aryl-(C₁-C₆)-alkylcarbonyloxy, (C₆-C₁₄)-aryl-(C₁-C₆)-alkoxycarbonyl, (C₆-C₁₄)-aryl-(C₁-C₆)-alkoxycarbonyloxy;
   mono-((C₁-C₆)-alkyl)amino, mono-((C₁-C₆)-haloalkyl)amino, di-((C₁-C₆)-alkyl)amino, di-((C₁-C₆)-haloalkyl)amino, ((C₁-C₆)-alkyl-(C₁-C₆)-haloalkyl)amino, N—((C₁-C₆)-alkanoyl)amino, N—((C₁-C₆)-haloalkanoyl)amino, aminocarbonyl-(C₁-C₆)-alkyl, di-(C₁-C₆)-alkylaminocarbonyl-(C₁-C₆)-alkyl;
   mono-((C₁-C₆)-alkyl)aminocarbonyl, mono-((C₁-C₆)-haloalkyl)aminocarbonyl, di-((C₁-C₆)-alkyl)aminocarbonyl, di-((C₁-C₆)-haloalkyl)aminocarbonyl, ((C₁-C₆)-alkyl-(C₁-C₆)-haloalkyl)aminocarbonyl, N—((C₁-C₆)-alkanoyl)aminocarbonyl, N—((C₁-C₆)-haloalkanoyl)aminocarbonyl, mono-((C₆-C₁₄)-aryl)aminocarbonyl, di-((C₆-C₁₄)-aryl)aminocarbonyl,
   (C₁-C₆)-alkoxy-(C₁-C₆)-alkyl, (C₁-C₆)-alkoxy-(C₁-C₆)-alkoxy, (C₁-C₆)-alkoxycarbonyl-(C₁-C₆)-alkoxy;
   (C₃-C₈)-cycloalkyl which may optionally be substituted in the cycloalkyl radical by (C₁-C₆)-alkyl and/or halogen; (C₃-C₈)-cycloalkoxy, (C₃-C₈)-cycloalkyl-(C₁-C₆)-alkyl, (C₃-C₈)-cycloalkyl-(C₁-C₆)-haloalkyl, (C₃-C₈)-cycloalkyl-(C₁-C₆)-alkoxy, (C₃-C₈)-cycloalkyl-(C₁-C₆)-haloalkoxy, (C₃-C₈)-cycloalkylcarbonyl, (C₃-C₈)-cycloalkoxycarbonyl, (C₃-C₈)-cycloalkyl-(C₁-C₆)-alkylcarbonyl, (C₃-C₈)-cycloalkyl-(C₁-C₆)-haloalkylcarbonyl, (C₃-C₈)-cycloalkyl-(C₁-C₆)-alkoxycarbonyl, (C₃-C₈)-cycloalkyl-(C₁-C₆)-haloalkoxycarbonyl, (C₃-C₈)-cycloalkylcarbonyloxy, (C₃-C₈)-cycloalkoxycarbonyloxy, (C₃-C₈)-cycloalkyl-(C₁-C₆)-alkylcarbonyloxy, (C₃-C₈)-cycloalkyl-(C₁-C₆)-haloalkylcarbonyloxy, (C₃-C₈)-cycloalkyl-(C₁-C₆)-alkoxycarbonyloxy, (C₃-C₈)-cycloalkyl-(C₁-C₆)-haloalkoxycarbonyloxy;
   (C₃-C₈)-cycloalkenyl, (C₃-C₈)-cycloalkenyloxy, (C₃-C₈)-cycloalkenyl-(C₁-C₆)-alkyl, (C₃-C₈)-cycloalkenyl-(C₁-C₆)-haloalkyl, (C₃-C₈)-cycloalkenyl-(C₁-C₆)-alkoxy, (C₃-C₈)-cycloalkenyl-(C₁-C₆)-haloalkoxy, (C₃-C₈)-cycloalkenylcarbonyl, (C₃-C₈)-cycloalkenyloxycarbonyl, (C₃-C₈)-cycloalkenyl-(C₁-C₆)-alkylcarbonyl, (C₃-C₈)-cycloalkenyl-(C₁-C₆)-haloalkylcarbonyl, (C₃-C₈)-cycloalkenyl-(C₁-C₆)-alkoxycarbonyl, (C₃-C₈)-cycloalkenyl-(C₁-C₆)-haloalkoxycarbonyl, (C₃-C₈)-cycloalkenylcarbonyloxy, (C₃-C₈)-cycloalkenyloxycarbonyloxy, (C₃-C₈)-cycloalkenyl-(C₁-C₆)-alkylcarbonyloxy, (C₃-C₈)-cycloalkenyl-(C₁-C₆)-haloalkylcarbonyloxy, (C₃-C₈)-cycloalkenyl-(C₁-C₆)-alkoxycarbonyloxy, (C₃-C₈)-cycloalkenyl-(C₁-C₆)-haloalkoxycarbonyloxy;
   hydroxy-(C₁-C₆)-alkyl, hydroxy-(C₁-C₆)-alkoxy, cyano-(C₁-C₆)-alkoxy, cyano-(C₁-C₆)-alkyl;
   (C₁-C₆)-alkylsulfonyl, (C₁-C₆)-alkylthio, (C₁-C₆)-alkylsulfinyl, (C₁-C₆)-haloalkylsulfonyl, (C₁-C₆)-haloalkylthio, (C₁-C₆)-haloalkylsulfinyl, (C₁-C₆)-alkylsulfonyl-(C₁-C₆)-alkyl, (C₁-C₆)-alkylthio-(C₁-C₆)-alkyl, (C₁-C₆)-alkylsulfinyl-(C₁-C₆)-alkyl, (C₁-C₆)-haloalkylsulfonyl-(C₁-C₆)-alkyl, (C₁-C₆)-haloalkylthio-(C₁-C₆)-alkyl, (C₁-C₆)-haloalkylsulfinyl-(C₁-C₆)-alkyl, (C₁-C₆)-alkylsulfonyl-(C₁-C₆)-haloalkyl, (C₁-C₆)-alkylthio-(C₁-C₆)-haloalkyl, (C₁-C₆)-alkylsulfinyl-(C₁-C₆)-haloalkyl, (C₁-C₆)-haloalkylsulfonyl-(C₁-C₆)-haloalkyl, (C₁-C₆)-haloalkylthio-(C₁-C₆)-haloalkyl, (C₁-C₆)-haloalkylsulfinyl-(C₁-C₆)-haloalkyl, (C₁-C₆)-alkylsulfonyloxy, (C₁-C₆)-haloalkylsulfonyloxy, (C₁-C₆)-alkylthiocarbonyl, (C₁-C₆)-haloalkylthiocarbonyl, (C₁-C₆)-alkylthiocarbonyloxy, (C₁-C₆)-haloalkylthiocarbonyloxy, (C₁-C₆)-alkylthio-(C₁-C₆)-alkyl, (C₁-C₆)-alkylthio-(C₁-C₆)-alkoxy, (C₁-C₆)-alkylthio-(C₁-C₆)-alkylcarbonyl, (C₁-C₆)-alkylthio-(C₁-C₆)-alkylcarbonyloxy; (C₄-C₁₄)-arylsulfonyl, (C₆-C₁₄)-arylthio, (C₆-C₁₄)-arylsulfinyl, (C₃-C₈)-cycloalkylthio, (C₃-C₈)-alkenylthio, (C₃-C₈)-cycloalkenylthio, (C₃-C₆)-alkynylthio;
   or
R¹ and R² together with the carbon atom or nitrogen atom to which they are bonded form a saturated five- to seven-membered ring that may be interrupted by the Y group, where Y is selected from C(O), O, S, S(O), S(O)₂, NR^{1a}, C(O)—NR^{1a}, where the saturated five- to seven-membered ring may be substituted by m substituents selected from halogen, acetyl, (C₁-C₆)-alkyl, (C₃-C₆)-cycloalkyl, trifluoromethyl, COOMe, COOEt, CONH₂, nitrile, (C₁-C₆)-alkylsulfonyl, (C₃-C₆)-cycloalkylsulfonyl, phenylsulfonyl or phenyl-(C₁-C₆)-alkylsulfonyl, and where R^{1a} is selected from hydrogen, acetyl, trifluoroacetyl, (C₁-C₆)-alkyl, (C₃-C₆)-cycloalkyl, (C₃-C₆)-cycloalkylcarbonyl, (C₃-C₆)-cycloalkyl-(C₁-C₆)-alkyl, (C₁-C₄)-alkylsulfonyl, phenyl, phenyl-(C₁-C₆)-alkyl, phenylcarbonyl, 2-pyridinyl, 3-pyridinyl, 4-pyridinyl, 2-pyridinylcarbonyl, 3-pyridinylcarbonyl, 4-pyridinylcarbonyl, where the 14 latter radicals may be substituted by halogen, (C₁-C₆)-alkyl, (C₁-C₆)-alkyloxy, nitrile or trifluoromethyl;
R³ is hydrogen, (C₁-C₆)-alkyl, (C₁-C₆)-haloalkyl, (C₁-C₆)-alkylcarbonyl, (C₁-C₆)-haloalkylcarbonyl or (C₁-C₆)-alkylcarbonyloxy;
R⁴ and R⁵ are each independently of one another hydrogen, (C₁-C₆)-alkyl, (C₁-C₆)-haloalkyl, hydroxyl, (C₁-C₆)-alkoxy or (C₁-C₆)-haloalkoxy; or
R⁴ and R⁵ together with the carbon atom to which they are attached form a saturated three- to seven-membered ring which may contain one or more heteroatoms selected from the group consisting of oxygen or sulfur or NH or NR^{1a};
R⁶ and R⁷ are each independently of one another hydrogen, (C₁-C₆)-alkyl, (C₁-C₆)-haloalkyl, (C₁-C₆)-alkoxy, (C₁-C₆)-haloalkoxy, (C₆-C₁₄)-aryl, (C₆-C₁₄)-aryloxy, (C₆-C₁₄)-arylcarbonyl or (C₆-C₁₄)-aryloxycarbonyl;
   or
R⁶ and R⁷ together with the carbon to which they are attached form a (C₃-C₇)-alkylene group which may contain one or more oxygen and/or sulfur atoms, where the (C₃-C₇)-alkylene group may be mono- or polysubstituted by halogen and the respective halogen substituents may be identical or different;
R⁸, R⁹, R¹⁰ and R¹¹ are each independently of one another hydrogen, halogen, cyano, C(O)OH, C(O)NH₂, (C₁-C₆)-alkyl, (C₁-C₆)-alkylcarbonyl, (C₁-C₆)-alkyloxycarbonyl, (C₁-C₆)-alkylaminocarbonyl, (C₁-C₆)-dialkylaminocarbonyl, (C₁-C₆)-haloalkyl, (C₁-C₆)-alkoxy, (C₁-C₆)-haloalkoxy, (C₂-C₆)-alkenyl, (C₂-C₆)-alkynyl, (C₂-C₆)-haloalkynyl, (C₂-C₆)-alkynylcarbonyl, (C₂-C₆)-haloalkynylcarbonyl, (C₂-C₆)-alkynyloxy, (C₂-C₆)-haloalkynyloxy, (C₂-C₆)-alkynyloxycarbonyl, (C₂-C₆)-haloalkynyloxycarbonyl or nitro, where the R⁹ and R¹⁰ radicals may be joined by an —O—CH₂—O— group to form a ring;
X represents a bond, CH₂, O, S, carbonyl, NH, CR¹²R¹³, NR¹⁴, CH₂O or CH₂S, where in the two latter groups the carbon atom is attached to the aromatic moiety and the heteroatom O or S is attached to the partially hydrogenated moiety of the amine; where, when n=0, X cannot be a bond;
R¹² and R¹³ are each independently of one another hydrogen, (C₁-C₆)-alkyl or (C₁-C₆)-haloalkyl;
R¹⁴ is hydrogen, (C₁-C₆)alkyl or (C₁-C₆)haloalkyl;
n is the serial number 0, 1 or 2; and
m is the serial number 0, 1, 2, 3, 4 or 5.

As well as a good profile of efficacy and good crop plant compatibility, the compounds of the formula (I) are notable for their inexpensive preparation, since the substances of the invention can be prepared from inexpensive and readily available precursors by inexpensive processes. It is therefore possible to dispense with the use of intermediates that are costly and difficult to obtain.

There follows a description of preferred, particularly preferred and very particularly preferred definitions of each of the individual substituents. The other substituents of the general formula (I) which are not specified hereinafter have the definition given above. The same also applies to the serial number n, meaning that the serial number n in the embodiments which follow is 0, 1 or 2.

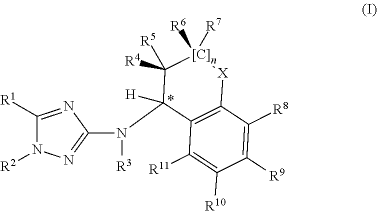

A first embodiment of the present invention encompasses compounds of the general formula (I) in which
R¹ is preferably hydrogen, halogen, cyano, C(═O)NH₂, NO₂, (C₁-C₆)-alkyl, (C₁-C₆)-alkylcarbonyl, (C₁-C₆)-haloalkyl, (C₃-C₆)-cyclopropyl, (C₁-C₆)-alkoxy, (C₁-C₆)-thioalkyl, (C₁-C₆)-alkylthio, (C₂-C₆)-alkynyl, mono-(C₁-C₆)-alkylamino, di-(C₁-C₆)-alkylamino or tri-(C₁-C₆)-alkylsilyl-(C₂-C₆)-alkynyl;
R¹ is particularly preferably hydrogen, cyano, fluorine, chlorine, bromine, iodine, nitro, trimethylsilylethynyl, methyl, ethyl, propyl, isopropyl, butyl, tert-butyl, n-pentyl, n-heptyl, cyclopropyl, cyclobutyl, acetyl, ethynyl, amino, dimethylamino, trifluoromethyl, difluoromethyl, monofluoromethyl, methoxy, ethoxy or methoxymethyl; and
R¹ is very particularly preferably methyl, ethyl, propyl, acetyl or trifluoromethyl.

A second embodiment of the present invention encompasses compounds of the general formula (I) in which
R² is preferably hydrogen, halogen, (C₁-C₆)-alkylphenyl, (C₆-C₁₄)-aryl which may be substituted on the aryl radical by (C₁-C₆)-alkyl, (C₆-C₁₄)-haloalkyl and/or halogen; C₆-aryl-(C₁-C₆)-haloalkyl, (C₁-C₆)-alkyl, (C₁-C₆)-haloalkyl, (C₁-C₆)-alkoxy, (C₁-C₆)-alkoxy-(C₁-C₆)-alkyl, (C₃-C₆)-cycloalkyl which may be substituted on the cycloalkyl radical by (C₁-C₆)-alkyl, (C₆-C₁₄)-haloaryl and/or halogen; 1-(C₁-C₆)-alkylcyclopropyl, 1-((C₁-C₆)-alkyl-C₆-aryl)-cyclopropyl, 1-(monohalophenyl)cyclopropyl, 1-(dihalophenyl)cyclopropyl, mono-(C₁-C₆)-alkylamino, di-(C₁-C₆)-alkylamino, (C₁-C₆)-thioalkyl, (C₁-C₆)-alkylthio, (C₁-C₆)-alkoxy, (C₁-C₄)-alkoxy-(C₁-C₆)-alkyl or amino;
R² is particularly preferably hydrogen, chlorine, phenyl, 2-methylphenyl, 3-trifluoromethylphenyl, methyl, ethyl, isopropyl, butyl, tert-butyl, n-pentyl, n-heptyl, trifluoromethyl, 1-methylcyclopropyl, 1-(p-xylyl)-cyclopropyl, 1-(2,4-dichlorophenyl)cyclopropyl, amino, dimethylamino, trifluoromethyl, difluoromethyl, monofluoromethyl, CHFCH₃, CF(CH₃)₂, CHF(CH₂CH₃), 1-fluorocyclopropyl, cyclopentyl, methoxy, ethoxy, methoxymethyl, ethoxymethyl, thiomethyl, methylthio or methoxy; and
R² is very particularly preferably hydrogen, methyl or ethyl.

A third embodiment of the present invention encompasses compounds of the general formula (I) in which
R¹ and R² preferably form, together with the carbon atom or nitrogen atom to which they are attached, a saturated six- or seven-membered ring as shown below

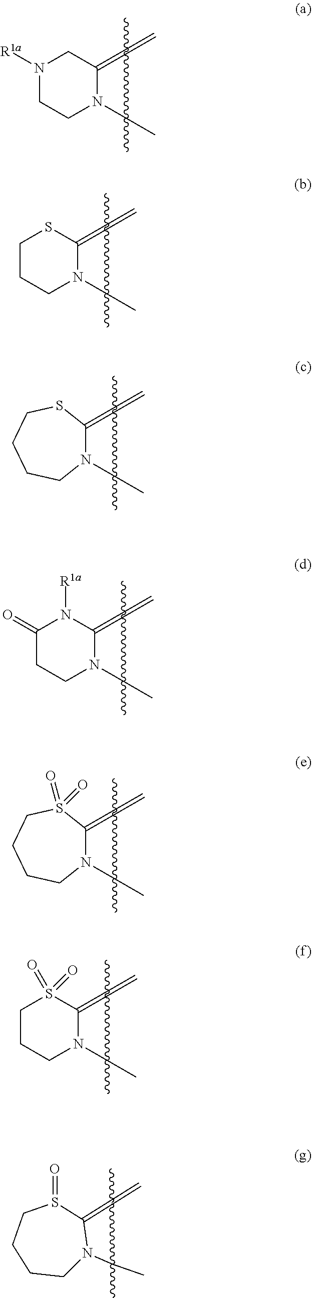

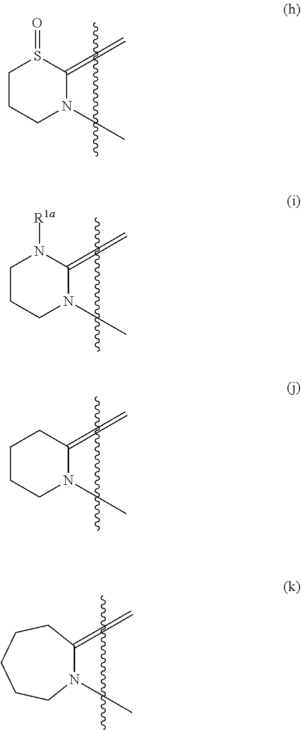

where the saturated six- or seven-membered ring may be substituted by m substituents selected from halogen, acetyl, (C₁-C₆)-alkyl, (C₃-C₆)-cycloalkyl, trifluoromethyl, (C₁-C₆)-alkylsulfonyl, (C₃-C₆)-cycloalkylsulfonyl or phenylsulfonyl,
and where
R^{1a} is preferably selected from hydrogen, acetyl, trifluoroacetyl, (C₁-C₆)-alkyl, (C₃-C₆)-cycloalkyl, (C₃-C₆)-cycloalkylcarbonyl, (C₃-C₆)-cycloalkyl-(C₁-C₆)-alkyl, (C₁-C₄)-alkylsulfonyl, phenyl, phenyl-(C₁-C₆)-alkyl, phenylcarbonyl, 2-pyridinyl, 3-pyridinyl, 4-pyridinyl, where the 11 latter radicals may be substituted by halogen, (C₁-C₆)-alkyl, (C₁-C₆)-alkyloxy, nitrile or trifluoromethyl,
   is further preferably selected from hydrogen, acetyl, (C₁-C₄)-alkyl, (C₁-C₄)-alkylsulfonyl or phenylcarbonyl, where the phenyl may be substituted by a halogen,
   even further preferably hydrogen, (C₁-C₄)-alkyl or acetyl, and most preferably is hydrogen.

Most preferably, R¹ and R² together with the carbon atom or nitrogen atom to which they are attached form a saturated six- or seven-membered ring as shown below

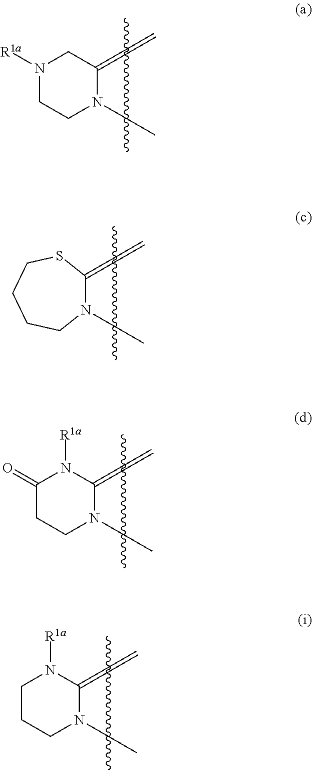

where the saturated six- or seven-membered ring may be substituted by m substituents selected from halogen, acetyl, (C₁-C₆)-alkyl, (C₃-C₆)-cycloalkyl, trifluoromethyl, (C₁-C₆)-alkylsulfonyl, (C₃-C₆)-cycloalkylsulfonyl or phenylsulfonyl, and where
R^{1a} is preferably selected from hydrogen, acetyl, trifluoroacetyl, (C₁-C₆)-alkyl, (C₃-C₆)-cycloalkyl, (C₃-C₆)-cycloalkylcarbonyl, (C₃-C₆)-cycloalkyl-(C₁-C₆)-alkyl, (C₁-C₄)-alkylsulfonyl, phenyl, phenyl-(C₁-C₆)-alkyl, phenylcarbonyl, 2-pyridinyl, 3-pyridinyl, 4-pyridinyl, where the 11 latter radicals may be substituted by halogen, (C₁-C₆)-alkyl, (C₁-C₆)-alkyloxy, nitrile or trifluoromethyl,
   is further preferably selected from hydrogen, (C₁-C₄)-alkyl, acetyl, (C₁-C₄)-alkylsulfonyl or phenylcarbonyl, where the phenyl may be substituted by a halogen,
   even further preferably hydrogen, (C₁-C₄)-alkyl or acetyl, and is most preferably hydrogen.

A fourth embodiment of the present invention encompasses compounds of the general formula (I) in which
R³ is preferably hydrogen.

A fifth embodiment of the present invention encompasses compounds of the general formula (I) in which
R⁴ and R⁵ each independently of one another are preferably hydrogen, (C₁-C₆)-alkyl, hydroxyl, cyclopropyl or (C₁-C₆)-alkoxy;
R⁴ and R⁵ each independently of one another are particularly preferably hydrogen, methyl, ethyl, propyl, cyclopropyl, hydroxyl or methoxy; and
R⁴ and R⁵ each independently of one another are very particularly preferably hydrogen, methyl or ethyl.

In this fifth embodiment, it is especially preferable for at least one of the radicals R⁴ and R⁵ to be hydrogen. It is further preferable for at least one of the radicals R⁴ and R⁵ to be hydrogen and for the other radical R⁴ or R⁵ not to be hydrogen and in particular to be (C₁-C₆)-alkyl. It is very especially preferable for one of the radicals R⁴ and R⁵ to be hydrogen and for the other radical R⁴ or R⁵ to be methyl.

A sixth embodiment of the present invention encompasses compounds of the general formula (I) in which
R⁴ and R⁵ preferably together form a (C₂-C₇)-alkylene group which may contain one or more oxygen and/or sulfur atoms, where the (C₂-C₇)-alkylene group may be mono- or polysubstituted by halogen and the respective halogen substituents may be identical or different;
R⁴ and R⁵ together with the carbon atom to which they are attached particularly preferably form a three- to four-membered ring; and
R⁴ and R⁵ most preferably together form an unsubstituted (C₂-C₃)-alkylene group.

A seventh embodiment of the present invention encompasses compounds of the general formula (I) in which
R⁶ and R⁷ independently of one another are hydrogen, (C₁-C₆)-alkyl or (C₆-C₁₄)-aryl;
R⁶ and R⁷ independently of one another are particularly preferably hydrogen, methyl or phenyl; and
R⁶ and R⁷ are very particularly preferably hydrogen.

An eighth embodiment of the present invention encompasses compounds of the general formula (I) in which
R⁸ is preferably hydrogen, (C₁-C₆)-alkyl or halogen;
R⁸ is particularly preferably hydrogen, methyl or fluorine; and
R⁸ is very particularly preferably hydrogen.

A ninth embodiment of the present invention encompasses compounds of the general formula (I) in which
R⁹ is preferably hydrogen or (C₁-C₆)-alkyl;
R⁹ is particularly preferably hydrogen or methyl; and
R⁹ is very particularly preferably hydrogen.

A tenth embodiment of the present invention encompasses compounds of the general formula (I) in which
R¹⁰ is preferably hydrogen, (C₁-C₆)-alkyl, di-(C₁-C₆)-alkylamino, halogen, (C₂-C₆)-alkenyl, (C₂-C₆)-alkynyl, (C₁-C₆)-alkyl-(C₂-C₆)-alkynyl, (C₁-C₆)-alkoxy-(C₁-C₆)-alkyl-(C₂-C₆)-alkynyl, cyano, (C₁-C₆)-alkoxycarbonyl or aminocarbonyl;
R¹⁰ is particularly preferably hydrogen, methyl, propyl, isopropyl, butyl, tert-butyl, dimethylamino, fluorine, chlorine, bromine, iodine, ethenyl, ethynyl, methylethynyl, ethylethynyl, MeOCH₂C≡C—, cyano, COOMe or CONH₂; and
R¹⁰ is very particularly preferably hydrogen, methyl or fluorine.

An eleventh embodiment of the present invention encompasses compounds of the general formula (I) in which
R¹¹ is preferably hydrogen or (C₁-C₆)-alkyl;
R¹¹ is particularly preferably hydrogen or methyl; and
R¹¹ is very particularly preferably hydrogen.

A twelfth embodiment of the present invention encompasses compounds of the general formula (I) in which
X is preferably CH₂, O or a bond, where, when n=0, X cannot be a bond; and
X is very particularly preferably a bond, where n is 1 or 2.

A thirteenth embodiment of the present invention encompasses compounds of the general formula (I) in which
R¹² is preferably hydrogen.

A fourteenth embodiment of the present invention encompasses compounds of the general formula (I) in which
R¹³ is preferably hydrogen.

A fifteenth embodiment of the present invention encompasses compounds of the general formula (I) in which the serial number
m is preferably 0, 1, 2, 3 or 4, more preferably 0, 1, 2 or 3, and most preferably 0, 1 or 2.

In the context of the present invention, it is possible to combine the individual preferred, particularly preferred, very particularly preferred and most preferred meanings of the substituents R¹ to R¹⁴ and X with one another as desired, where the serial number n is 0, 1 or 2.

This means that the present invention encompasses compounds of the general formula (I) in which, for example, the substituent R¹ has a preferred definition and the substituents R² to R¹⁴ have the general definition or else the substituent R² has a preferred definition, the substituent R³ has a particularly preferred or very particularly preferred definition and the remaining substituents have a general definition.

Four of these combinations of the definitions given above for the substituents R¹ to R¹¹ and X are elucidated by way of example hereinafter and each are disclosed as further embodiments:

combination of the definitions given above for the substituents R¹ to R¹¹ and X in each case as being particularly preferred (twelfth embodiment), combination of the definitions given above for the substituents R¹ to R¹⁴ and X in each case as being very particularly preferred (thirteenth embodiment), and combination of the definition given above for the substituent R¹ as being very particularly preferred with the definitions given above for the substituents R¹ to R¹⁴ and X in each case as being particularly preferred (fourteenth embodiment).

The aforementioned further embodiments that are based on the combinations of the substituents are disclosed explicitly hereinafter for reasons of clarity:

A sixteenth embodiment of the present invention encompasses compounds of the general formula (I) in which
R¹ and R² together with the carbon atom or nitrogen atom to which they are attached a saturated six- or seven-membered ring as shown below

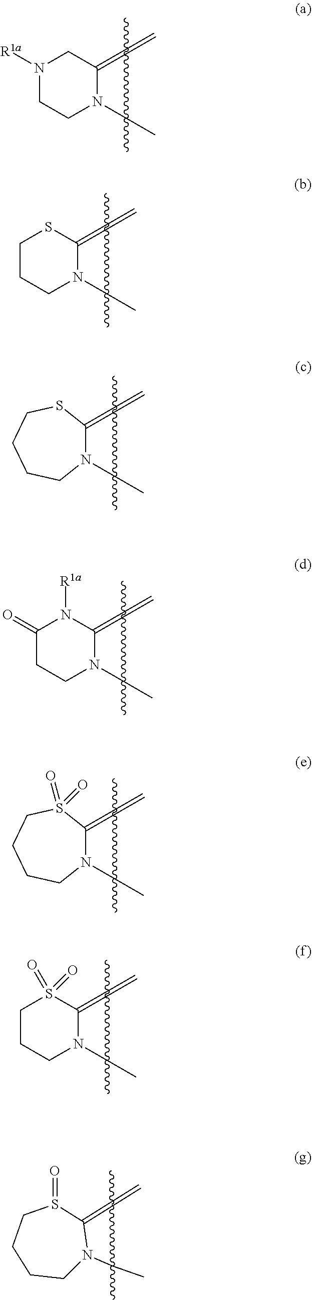

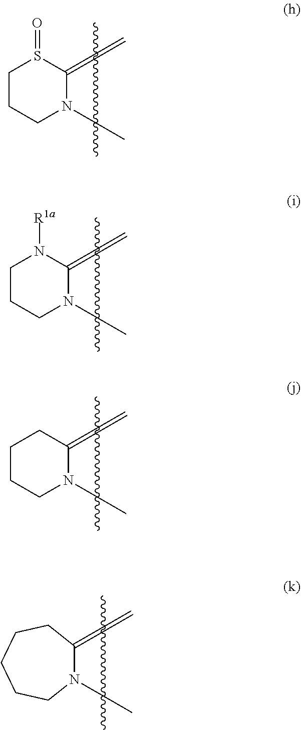

where the saturated six- or seven-membered ring may be substituted by m substituents selected from halogen, acetyl, (C₁-C₆)-alkyl, (C₃-C₆)-cycloalkyl, trifluoromethyl, (C₁-C₆)-alkylsulfonyl, (C₃-C₆)-cycloalkylsulfonyl or phenylsulfonyl,
and where
R^{1a} is hydrogen, acetyl, (C₁-C₄)-alkyl, (C₁-C₄)-alkylsulfonyl or phenylcarbonyl, where the phenyl may be substituted by a halogen;
R³ is hydrogen;
R⁴ and R⁵ each independently of one another are hydrogen, methyl, ethyl, propyl, cyclopropyl, hydroxyl or methoxy;
R⁶ and R⁷ independently of one another are hydrogen, methyl or phenyl;
R⁸ is hydrogen, methyl or amino;
R⁹ is hydrogen or methyl;
R¹⁰ is hydrogen, methyl, propyl, isopropyl, butyl, tert-butyl, dimethylamino, fluorine, chlorine, bromine, iodine, ethenyl, ethynyl, methylethynyl, ethylethynyl, MeOCH₂C≡C—, cyano, COOMe or CONH₂;
R¹¹ is hydrogen or methyl; and
X is a bond, where n is 1 or 2.

A seventeenth embodiment of the present invention encompasses compounds of the general formula (I) in which
R¹ and R² together with the carbon atom or nitrogen atom to which they are attached form a saturated six- or seven-membered ring as shown below

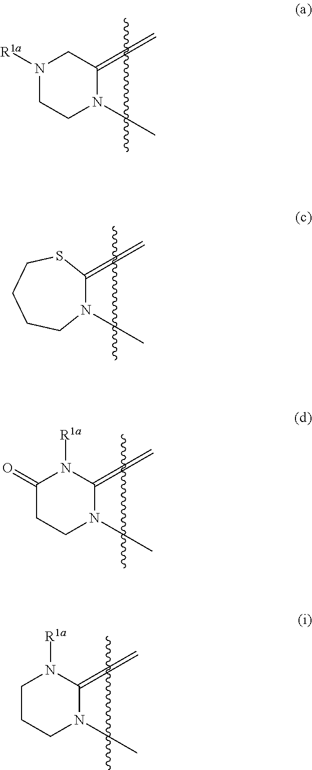

where the saturated six- or seven-membered ring may be substituted by m substituents selected from halogen, acetyl, (C₁-C₆)-alkyl, (C₃-C₆)-cycloalkyl, trifluoromethyl, (C₁-C₆)-alkylsulfonyl, (C₃-C₆)-cycloalkylsulfonyl or phenylsulfonyl,
and where
R^{1a} is hydrogen, acetyl or (C₁-C₄)-alkyl;
R³ is hydrogen;
R⁴ and R⁵ each independently of one another are methyl, ethyl or hydrogen;
R⁶, R⁷, R⁸, R⁹ and R¹¹ are hydrogen;
R¹⁰ is hydrogen, methyl or fluorine; and
X is a bond, where n is 1 or 2.

An eighteenth embodiment of the present invention encompasses compounds of the general formula (I) in which
R¹ is hydrogen, cyano, fluorine, chlorine, bromine, iodine, nitro, trimethylsilylethynyl, methyl, ethyl, propyl, isopropyl, butyl, tert-butyl, n-pentyl, n-heptyl, cyclopropyl, cyclobutyl, acetyl, ethynyl, amino, dimethylamino, trifluoromethyl, difluoromethyl, monofluoromethyl, methoxy, ethoxy or methoxymethyl; R² is hydrogen, chlorine, phenyl, 2-methylphenyl, 3-trifluoromethylphenyl, methyl, ethyl, isopropyl, butyl, tert-butyl, n-pentyl, n-heptyl, trifluoromethyl, 1-methylcyclopropyl, 1-(p-xylyl)cyclopropyl, 1-(2,4-dichlorophenyl)cyclopropyl, amino, dimethylamino, trifluoromethyl, difluoromethyl, monofluoromethyl, CHFCH₃, CF(CH₃)₂, CHF(CH₂CH₃), 1-fluorocyclopropyl, cyclopentyl, methoxy, ethoxy, methoxymethyl, ethoxymethyl, thiomethyl, methylthio or methoxy;
R³ is hydrogen;
R⁴ and R⁵ each independently of one another are hydrogen, methyl, ethyl, propyl, cyclopropyl, hydroxyl or methoxy;
R⁶ and R⁷ independently of one another are hydrogen, methyl or phenyl;
R⁸ is hydrogen, methyl or amino;
R⁹ is hydrogen or methyl;
R¹⁰ is hydrogen, methyl, propyl, isopropyl, butyl, tert-butyl, dimethylamino, fluorine, chlorine, bromine, iodine, ethenyl, ethynyl, methylethynyl, ethylethynyl, MeOCH₂C≡C—, cyano, COOMe or CONH₂;
R¹¹ is hydrogen or methyl; and
X is a bond, where n is 1 or 2.

A nineteenth embodiment of the present invention encompasses compounds of the general formula (I) in which
R¹ is methyl, ethyl, propyl, dimethylamino, diethylamino, 1-pyrrolidino, 1-piperidino, 4-morpholino, acetyl or trifluoromethyl;
R² is methyl or ethyl;
R³ is hydrogen;
R⁴ and R⁵ each independently of one another are hydrogen, methyl or ethyl;
R⁶ and R⁷ are hydrogen;
R⁸ is hydrogen;
R⁹ is hydrogen;
R¹⁰ is hydrogen, methyl or fluorine;
R¹¹ is hydrogen; and
X is a bond, where n is 1 or 2.

In the context of the present invention, the compound of the general formula (I) also includes compounds quaternized on a nitrogen atom by a) protonation, b) alkylation or c) oxidation. In this respect, particular mention should be made of the corresponding N-oxides.

The compounds of the formula (I) are capable of forming salts. Salts may be formed by the action of a base on those compounds of the formula (I) that bear an acidic hydrogen atom. Suitable bases are, for example, organic amines such as trialkylamines, morpholine, piperidine or pyridine, and the hydroxides, carbonates and bicarbonates of ammonium, alkali metals or alkaline earth metals, especially sodium hydroxide, potassium hydroxide, sodium carbonate, potassium carbonate, sodium bicarbonate and potassium bicarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NRR′R″R′″]⁺ in which R to R′″ each independently of one another represent an organic radical, in particular alkyl, aryl, aralkyl or alkylaryl. Also suitable are alkylsulfonium and alkylsulfoxonium salts, such as (C₁-C₄)-trialkylsulfonium and (C₁-C₄)-trialkylsulfoxonium salts.

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts comprise the conjugated base of the acid as the anion.

Suitable substituents present in deprotonated form, such as, for example, sulfonic acids or carboxylic acids, may form inner salts with groups which for their part can be protonated, such as amino groups.

The compounds of the formula (I) and their salts are also referred to for short hereinafter as “compounds (I)” according to the invention or used in accordance with the invention.

In the general formula (I) and in all the other formulae in the present invention, the radicals alkyl, alkoxy, haloalkyl, haloalkoxy, alkylamino, alkylthio, haloalkylthio and the corresponding unsaturated and/or substituted radicals can in each case be straight-chain or branched in the carbon skeleton. Unless stated specifically, preference is given for these radicals to the lower carbon skeletons, for example those having 1 to 6 carbon atoms, in particular 1 to 4 carbon atoms, or in the case of unsaturated groups having 2 to 6 carbon atoms, in particular 2 to 4 carbon atoms. Alkyl radicals, both alone and in the composite definitions such as alkoxy, haloalkyl, etc., are, for example, methyl, ethyl, n-propyl or isopropyl, n-butyl, isobutyl, tert-butyl or 2-butyl, pentyls, hexyls, such as n-hexyl, isohexyl and 1,3-dimethylbutyl, heptyls, such as n-heptyl, 1-methylhexyl and 1,4-dimethylpentyl; alkenyl and alkynyl radicals have the definition of the possible unsaturated radicals corresponding to the alkyl radicals; where at least one double bond or triple bond is present, preferably one double bond or triple bond, respectively. Alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, ethynyl, propargyl, but-2-yn-1-yl, but-3-yn-1-yl and 1-methylbut-3-yn-1-yl.

Cycloalkyl groups are, for example, cyclopropyl, cyclobutyl, cyclopentyl, cyclohexyl, cycloheptyl and cyclooctyl. The cycloalkyl groups can be present in bi- or tricyclic form.

If haloalkyl groups and haloalkyl radicals of haloalkoxy, haloalkylthio, haloalkenyl, haloalkynyl inter alia are stated, the lower carbon skeletons of these radicals having, for example, 1 to 6 carbon atoms or 2 to 6 carbon atoms, in particular 1 to 4 carbon atoms or preferably 2 to 4 carbon atoms, and the corresponding unsaturated and/or substituted radicals are in each case straight-chain or branched in the carbon skeleton. Examples are difluoromethyl, 2,2,2-trifluoroethyl, trifluoroallyl, 1-chloroprop-1-yl-3-yl.

Alkylene groups in these radicals are the lower carbon skeletons, for example those having 1 to 10 carbon atoms, in particular 1 to 6 carbon atoms, or preferably 2 to 4 carbon atoms, and also the corresponding unsaturated and/or substituted radicals in the carbon skeleton which may in each case be straight-chain or branched. Examples are methylene, ethylene, n- and isopropylene and n-, s-, iso-, t-butylene.

Hydroxyalkyl groups in these radicals are the lower carbon skeletons, for example those having 1 to 6 carbon atoms, especially 1 to 4 carbon atoms, and also the corresponding unsaturated and/or substituted radicals in the carbon skeleton which may in each case be straight-chain or branched. Examples of these are 1,2-dihydroxyethyl and 3-hydroxypropyl.

Halogen denotes fluorine, chlorine, bromine or iodine. Haloalkyl, -alkenyl and -alkynyl are alkyl, alkenyl and alkynyl partly or fully substituted by halogen, preferably by fluorine, chlorine or bromine, especially by fluorine and/or chlorine, for example monohaloalkyl, perhaloalkyl, CF₃, CF₂Cl, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same correspondingly applies to haloalkenyl and other halogen-substituted radicals.

Aryl is a monocyclic, bicyclic or polycyclic aromatic system, for example phenyl or naphthyl, preferably phenyl.

Primarily for reasons of higher herbicidal activity, better selectivity and/or better preparability, compounds of the general formula (II) according to the invention or the agrochemical salts or quaternary N derivatives thereof that are of particular interest are those in which individual radicals have one of the preferred definitions already specified or specified below, or especially those in which one or more of the preferred definitions already specified or specified below occur in combination.

The abovementioned general or preferred radical definitions apply both to the end products of the general formula (II) and, correspondingly, to the starting materials and intermediates required in each case for the preparation. These radical definitions can be exchanged for one another, i.e. including between the given preferred ranges.

The present compounds of the general formula (I) have, at the point of attachment to the amino-[1,2,4]-triazole derivatives, a chiral carbon atom which, in the structure shown below, is indicated by the marker (*):

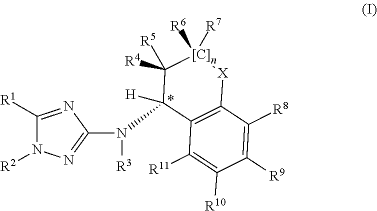

According to the rules of Cahn, Ingold and Prelog (CIP rules), this carbon atom can have either an (R) configuration or an (S) configuration.

The present invention encompasses compounds of the general formula (I) both with (S) and with (R) configuration, meaning that the present invention encompasses the compounds of the general formula (I) in which the carbon atom in question has

(1) an (R) configuration; or
(2) an (S) configuration.

In addition, the scope of the present invention also encompasses

(3) any mixtures of compounds of the general formula (I) having an (R) configuration (compounds of the general formula (I-(R)) with compounds of the general formula (I) having an (S) configuration (compounds of the general formula (I-S)), the present invention also encompassing a racemic mixture of the compounds of the general formula (I) having (R) and (S) configuration.

However, within the context of the present invention, preference is given particularly to compounds of the general formula (I) having (R) configuration with a selectivity of 60 to 100%, preferably 80 to 100%, especially 90 to 100%, very particularly 95 to 100%, where the particular (R) compound is present with an enantioselectivity of in each case more than 50% ee, preferably 60 to 100% ee, especially 80 to 100% ee, very particularly 90 to 100% ee, most preferably 95 to 100% ee, based on the total content of (R) compound in question.

Taking into account the Cahn, Ingold and Prelog rule, at the carbon atom marked by (*) there may also be a situation in which, owing to the priority of the substituents in question, the (S) configuration is preferred at the carbon atom marked by (*). This is the case, for example, when the radicals R⁴ and/or R⁵ correspond to a C₁-C₆-alkoxy radical.

Accordingly, within the context of the present invention, preference is given especially to compounds of the general formula (I) whose spatial arrangement corresponds to that of the compounds of the general formula (I) where R⁴ and R⁵=hydrogen having the (R) configuration, with a selectivity of 60 to 100%, preferably 80 to 100%, especially 90 to 100%, very particularly 95 to 100%, where the respective (R)-analogous compound is present with an enantioselectivity of in each case more than 50% ee, preferably 60 to 100% ee, especially 80 to 100% ee, very particularly 90 to 100% ee, most preferably 95 to 100% ee, based on the total content of (R)-analogous compound in question. Accordingly, the present invention relates especially to compounds of the general formula (I) in which the stereochemical configuration on the carbon atom marked by (*) is present with a stereochemical purity of 60 to 100% (R or R analog), preferably 80 to 100% (R or R analog), especially 90 to 100% (R or R analog), very particularly 95 to 100% (R or R analog).

In particular, the compounds of the general formula (I) according to the invention may have further centers of chirality at the carbon atoms marked by (**) and (***):

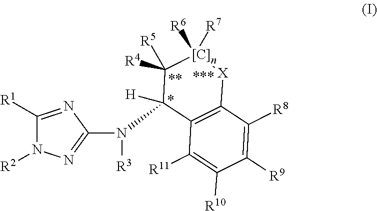

In the context of the present invention, any stereochemical configurations are possible at the carbon atoms marked by (*), (**) and (***):

| Configuration of | Configuration of | Configuration of |
|---|---|---|
| carbon atom (*) | carbon atom (**) | carbon atom (***) |
| R | R | R |
| R | R | S |
| R | S | R |
| S | R | R |
| R | S | S |
| S | R | S |
| S | S | R |
| S | S | S |

In addition, depending on the respective radicals chosen, further stereoelements may be present in the compounds of the general formula (I) according to the invention.

If, for example, one or more alkenyl groups are present, diastereomers (Z and E isomers) may occur.

If, for example, one or more asymmetric carbon atoms are present, enantiomers and diastereomers may occur.

Corresponding stereoisomers can be obtained from the mixtures obtained in the preparation by customary separation methods, for example by chromatographic separation processes. It is likewise possible to selectively prepare stereoisomers by using stereoselective reactions with use of optically active starting materials and/or auxiliaries. The invention thus also relates to all stereoisomers which are encompassed by the general formula (I) but are not shown in their specific stereo form, and to mixtures thereof.

Particular preference is given to compounds of the general formula (I)

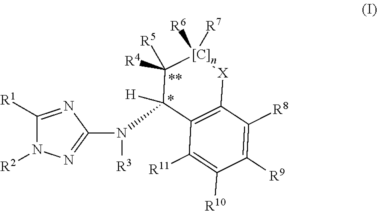

in which the chiral carbon atom marked by (*) has an (R) configuration and the chiral carbon atom marked by (**) has an (S) configuration.

The possible combinations of the various substituents of the general formula (I) should be understood such that the general principles of the construction of chemical compounds have to be observed, i.e. the formula (I) does not encompass any compounds known by the person skilled in the art to be chemically impossible.

The compounds of the formulae (I-a), (I-b), (I-c), (I-d), (I-e), (I-f), (I-g), (I-h) (I-i), (I-j) and (I-k) shown below are preferred compounds of the formula (I) of the present invention.

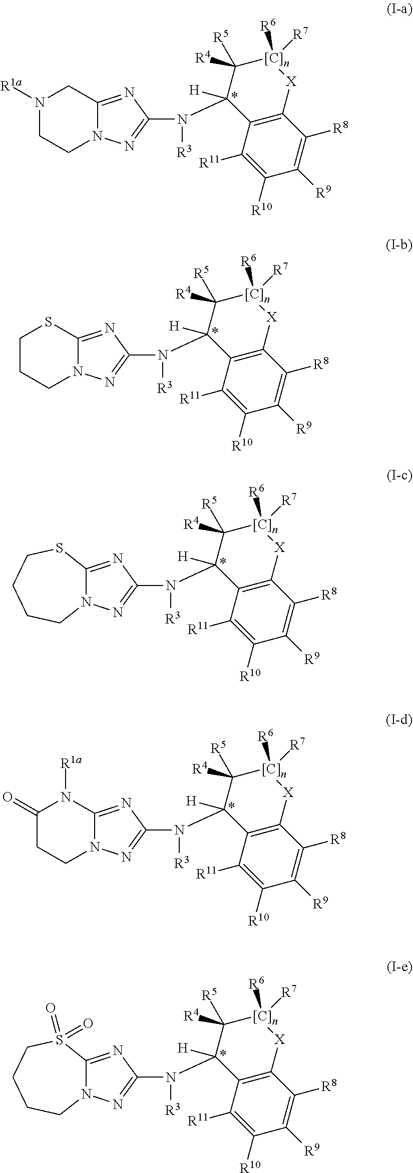

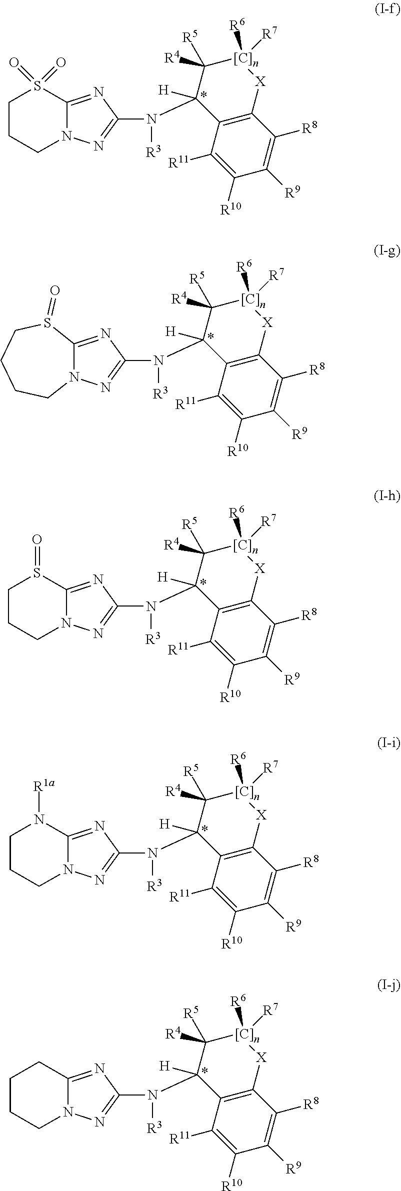

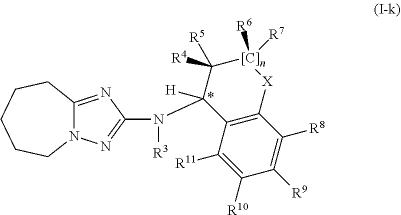

In tables 1 to 11 below, Y represents

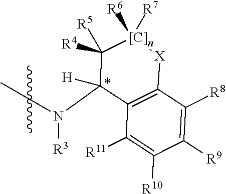

TABLE 1
Examples of the compounds of the general formula (I-a):

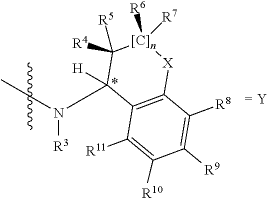

| | |
|---|---|
| | (I-a) |
| 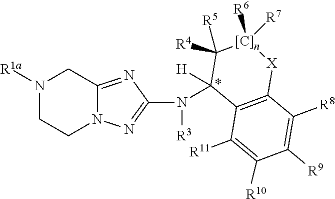 | |

| Example No.: | Y | R^{1a} |
|---|---|---|
| I-001 | (R)-5,7-dimethyl-1,2,3,4-tetrahydronaphthalen-1-yl | [4-(trifluoromethyl)pyridin-3-yl]carbonyl |
| I-002 | 6-methyl-1,2,3,4-tetrahydronaphthalen-1-yl | cyclopropylcarbonyl |
| I-003 | (R)-5,7-dimethyl-1,2,3,4-tetrahydronaphthalen-1-yl | 4-chlorobenzoyl |
| I-004 | 6-methyl-1,2,3,4-tetrahydronaphthalen-1-yl | pyridin-3-ylcarbonyl |
| I-005 | 6-methyl-1,2,3,4-tetrahydronaphthalen-1-yl | 3-(trifluoromethyl)benzoyl |
| I-006 | 6-methyl-1,2,3,4-tetrahydronaphthalen-1-yl | cyclohexylcarbonyl |
| I-007 | 6-methyl-1,2,3,4-tetrahydronaphthalen-1-yl | 4-chlorobenzoyl |
| I-008 | 6-methyl-1,2,3,4-tetrahydronaphthalen-1-yl | 2-chlorobenzoyl |
| I-009 | (R)-5,7-dimethyl-1,2,3,4-tetrahydronaphthalen-1-yl | 3-chlorobenzoyl |
| I-010 | (R)-5,7-dimethyl-1,2,3,4-tetrahydronaphthalen-1-yl | 3-(trifluoromethyl)benzoyl |
| I-011 | 6-methyl-1,2,3,4-tetrahydronaphthalen-1-yl | 3-chlorobenzoyl |
| I-012 | (R)-5,7-dimethyl-1,2,3,4-tetrahydronaphthalen-1-yl | cyclohexylcarbonyl |
| I-013 | 6-methyl-1,2,3,4-tetrahydronaphthalen-1-yl | benzoyl |
| I-014 | (R)-5,7-dimethyl-1,2,3,4-tetrahydronaphthalen-1-yl | 2-chlorobenzoyl |
| I-015 | 6-methyl-1,2,3,4-tetrahydronaphthalen-1-yl | benzylsulfonyl |
| I-016 | (R)-5,7-dimethyl-1,2,3,4-tetrahydronaphthalen-1-yl | benzylsulfonyl |
| I-017 | 6-methyl-1,2,3,4-tetrahydronaphthalen-1-yl | methylsulfonyl |
| I-018 | 6-methyl-1,2,3,4-tetrahydronaphthalen-1-yl | (2-chlorophenyl)sulfonyl |
| I-019 | indan-1-yl | benzoyl |
| I-020 | (R)-7-fluoro-1,2,3,4-tetrahydronaphthalen-1-yl | 3-(trifluoromethyl)benzoyl |
| I-021 | (R)-5-methyl-1,2,3,4-tetrahydronaphthalen-1-yl | cyclopropylcarbonyl |
| I-022 | 1,2,3,4-tetrahydronaphthalen-1-yl | 3-chlorobenzoyl |
| I-023 | indan-1-yl | 3-chlorobenzoyl |
| I-024 | chroman-4-yl | cyclohexylcarbonyl |
| I-025 | (R)-5-fluoro-1,2,3,4-tetrahydronaphthalen-1-yl | [4-(trifluoromethyl)pyridin-3-yl]carbonyl |
| I-026 | (R)-5-methyl-1,2,3,4-tetrahydronaphthalen-1-yl | [4-(trifluoromethyl)pyridin-3-yl]carbonyl |
| I-027 | (R)-5-methyl-1,2,3,4-tetrahydronaphthalen-1-yl | 3-(trifluoromethyl)benzoyl |
| I-028 | 1,2,3,4-tetrahydronaphthalen-1-yl | 4-chlorobenzoyl |
| I-029 | 1,2,3,4-tetrahydronaphthalen-1-yl | 2-chlorobenzoyl |
| I-030 | 1,2,3,4-tetrahydronaphthalen-1-yl | benzoyl |
| I-031 | (R)-7-fluoro-1,2,3,4-tetrahydronaphthalen-1-yl | 2-chlorobenzoyl |
| I-032 | (R)-7-fluoro-1,2,3,4-tetrahydronaphthalen-1-yl | benzoyl |
| I-033 | indan-1-yl | cyclohexylcarbonyl |
| I-034 | (R)-5-methyl-1,2,3,4-tetrahydronaphthalen-1-yl | 3-chlorobenzoyl |
| I-035 | (R)-5,7-dimethyl-1,2,3,4-tetrahydronaphthalen-1-yl | benzoyl |
| I-036 | indan-1-yl | 3-(trifluoromethyl)benzoyl |
| I-037 | (R)-7-fluoro-1,2,3,4-tetrahydronaphthalen-1-yl | cyclopropylcarbonyl |
| I-038 | (R)-7-fluoro-1,2,3,4-tetrahydronaphthalen-1-yl | cyclohexylcarbonyl |
| I-039 | (R)-5,7-dimethyl-1,2,3,4-tetrahydronaphthalen-1-yl | pyridin-3-ylcarbonyl |
| I-040 | (R)-7-fluoro-1,2,3,4-tetrahydronaphthalen-1-yl | 4-chlorobenzoyl |
| I-041 | (R)-5-methyl-1,2,3,4-tetrahydronaphthalen-1-yl | 4-chlorobenzoyl |
| I-042 | chroman-4-yl | [4-(trifluoromethyl)pyridin-3-yl]carbonyl |
| I-043 | 1,2,3,4-tetrahydronaphthalen-1-yl | [4-(trifluoromethyl)pyridin-3-yl]carbonyl |
| I-044 | (R)-7-fluoro-1,2,3,4-tetrahydronaphthalen-1-yl | pyridin-3-ylcarbonyl |
| I-045 | indan-1-yl | acetyl |
| I-046 | indan-1-yl | pyridin-3-ylcarbonyl |
| I-047 | chroman-4-yl | 3-chlorobenzoyl |
| I-048 | indan-1-yl | [4-(trifluoromethyl)pyridin-3-yl]carbonyl |
| I-049 | indan-1-yl | cyclopropylcarbonyl |
| I-050 | (R)-5-methyl-1,2,3,4-tetrahydronaphthalen-1-yl | 2-chlorobenzoyl |
| I-051 | 1,2,3,4-tetrahydronaphthalen-1-yl | 3-(trifluoromethyl)benzoyl |
| I-052 | (R)-5-methyl-1,2,3,4-tetrahydronaphthalen-1-yl | pyridin-3-ylcarbonyl |
| I-053 | (R)-7-fluoro-1,2,3,4-tetrahydronaphthalen-1-yl | acetyl |
| I-054 | indan-1-yl | 2-chlorobenzoyl |
| I-055 | chroman-4-yl | cyclopropylcarbonyl |
| I-056 | chroman-4-yl | 2-chlorobenzoyl |
| I-057 | 1,2,3,4-tetrahydronaphthalen-1-yl | cyclohexylcarbonyl |
| I-058 | chroman-4-yl | benzoyl |
| I-059 | (R)-5-methyl-1,2,3,4-tetrahydronaphthalen-1-yl | benzoyl |
| I-060 | 1,2,3,4-tetrahydronaphthalen-1-yl | cyclopropylcarbonyl |
| I-061 | 1,2,3,4-tetrahydronaphthalen-1-yl | acetyl |
| I-062 | (R)-5,7-dimethyl-1,2,3,4-tetrahydronaphthalen-1-yl | acetyl |
| I-063 | 6-methyl-1,2,3,4-tetrahydronaphthalen-1-yl | [4-(trifluoromethyl)pyridin-3-yl]carbonyl |
| I-064 | chroman-4-yl | pyridin-3-ylcarbonyl |
| I-065 | chroman-4-yl | 3-(trifluoromethyl)benzoyl |
| I-066 | indan-1-yl | 4-chlorobenzoyl |
| I-067 | chroman-4-yl | 4-chlorobenzoyl |
| I-068 | chroman-4-yl | acetyl |
| I-069 | (R)-5-methyl-1,2,3,4-tetrahydronaphthalen-1-yl | acetyl |
| I-070 | (R)-5-methyl-1,2,3,4-tetrahydronaphthalen-1-yl | cyclohexylcarbonyl |
| I-071 | 6-methyl-1,2,3,4-tetrahydronaphthalen-1-yl | acetyl |
| I-072 | (R)-5,7-dimethyl-1,2,3,4-tetrahydronaphthalen-1-yl | cyclopropylcarbonyl |
| I-073 | 1,2,3,4-tetrahydronaphthalen-1-yl | pyridin-3-ylcarbonyl |
| I-074 | (R)-7-fluoro-1,2,3,4-tetrahydronaphthalen-1-yl | [4-(trifluoromethyl)pyridin-3-yl]carbonyl |
| I-075 | (R)-7-fluoro-1,2,3,4-tetrahydronaphthalen-1-yl | 3-chlorobenzoyl |
| I-076 | chroman-4-yl | benzylsulfonyl |
| I-077 | (R)-7-fluoro-1,2,3,4-tetrahydronaphthalen-1-yl | benzylsulfonyl |
| I-078 | 1,2,3,4-tetrahydronaphthalen-1-yl | benzylsulfonyl |
| I-079 | indan-1-yl | benzylsulfonyl |
| I-080 | (R)-5,7-dimethyl-1,2,3,4-tetrahydronaphthalen-1-yl | trifluoromethylsulfonyl |
| I-081 | 6-methyl-1,2,3,4-tetrahydronaphthalen-1-yl | trifluoromethylsulfonyl |
| I-082 | (R)-5-methyl-1,2,3,4-tetrahydronaphthalen-1-yl | cyclopropylsulfonyl |
| I-083 | 6-methyl-1,2,3,4-tetrahydronaphthalen-1-yl | cyclopropylsulfonyl |
| I-084 | chroman-4-yl | cyclopropylsulfonyl |
| I-085 | (R)-5,7-dimethyl-1,2,3,4-tetrahydronaphthalen-1-yl | cyclopropylsulfonyl |
| I-086 | (R)-7-fluoro-1,2,3,4-tetrahydronaphthalen-1-yl | cyclopropylsulfonyl |
| I-087 | 1,2,3,4-tetrahydronaphthalen-1-yl | cyclopropylsulfonyl |
| I-088 | indan-1-yl | cyclopropylsulfonyl |
| I-089 | (R)-5-methyl-1,2,3,4-tetrahydronaphthalen-1-yl | isopropylsulfonyl |
| I-090 | 6-methyl-1,2,3,4-tetrahydronaphthalen-1-yl | isopropylsulfonyl |
| I-091 | chroman-4-yl | isopropylsulfonyl |
| I-092 | (R)-5,7-dimethyl-1,2,3,4-tetrahydronaphthalen-1-yl | isopropylsulfonyl |
| I-093 | (R)-7-fluoro-1,2,3,4-tetrahydronaphthalen-1-yl | isopropylsulfonyl |
| I-094 | 1,2,3,4-tetrahydronaphthalen-1-yl | isopropylsulfonyl |
| I-095 | indan-1-yl | isopropylsulfonyl |
| I-096 | indan-1-yl | methylsulfonyl |
| I-097 | 1,2,3,4-tetrahydronaphthalen-1-yl | methylsulfonyl |
| I-098 | (R)-7-fluoro-1,2,3,4-tetrahydronaphthalen-1-yl | methylsulfonyl |
| I-099 | (R)-5,7-dimethyl-1,2,3,4-tetrahydronaphthalen-1-yl | methylsulfonyl |
| I-100 | (R)-5-methyl-1,2,3,4-tetrahydronaphthalen-1-yl | methylsulfonyl |
| I-101 | (R)-5-methyl-1,2,3,4-tetrahydronaphthalen-1-yl | (2-chlorophenyl)sulfonyl |
| I-102 | chroman-4-yl | (2-chlorophenyl)sulfonyl |
| I-103 | (R)-5,7-dimethyl-1,2,3,4-tetrahydronaphthalen-1-yl | (2-chlorophenyl)sulfonyl |
| I-104 | (R)-7-fluoro-1,2,3,4-tetrahydronaphthalen-1-yl | (2-chlorophenyl)sulfonyl |
| I-105 | 1,2,3,4-tetrahydronaphthalen-1-yl | (2-chlorophenyl)sulfonyl |
| I-106 | indan-1-yl | (2-chlorophenyl)sulfonyl |
| I-107 | 2-methyl-6-chloro-ylindan-1-yl | 4-chlorobenzoyl |
| I-108 | (1R,2S)-2-methyl-6-fluoro-ylindan-1-yl | [4-(trifluoromethyl)pyridin-3-yl]carbonyl |
| I-109 | (1R,2S)-2-methyl-6-fluoro-ylindan-1-yl | 3-(trifluoromethyl)benzoyl |
| I-110 | 2-methyl-6-chloro-ylindan-1-yl | pyridin-3-ylcarbonyl |
| I-111 | (R)-5-fluoro-1,2,3,4-tetrahydronaphthalen-1-yl | 3-chlorobenzoyl |
| I-112 | (1R,2S)-2-methyl-6-fluoro-ylindan-1-yl | pyridin-3-ylcarbonyl |
| I-113 | (R)-5-fluoro-1,2,3,4-tetrahydronaphthalen-1-yl | cyclohexylcarbonyl |
| I-114 | (1R,2S)-2-methyl-6-fluoro-ylindan-1-yl | 3-chlorobenzoyl |
| I-115 | 2-methyl-6-chloro-ylindan-1-yl | [4-(trifluoromethyl)pyridin-3-yl]carbonyl |
| I-116 | 2-methyl-6-chloro-ylindan-1-yl | 3-chlorobenzoyl |
| I-117 | (1R,2S)-2-methyl-6-fluoro-ylindan-1-yl | acetyl |
| I-118 | 2-methyl-6-chloro-ylindan-1-yl | cyclohexylcarbonyl |
| I-119 | 2-methyl-6-chloro-ylindan-1-yl | 2-chlorobenzoyl |
| I-120 | (1R,2S)-2-methyl-6-fluoro-ylindan-1-yl | cyclohexylcarbonyl |
| I-121 | (R)-5-fluoro-1,2,3,4-tetrahydronaphthalen-1-yl | 3-(trifluoromethyl)benzoyl |
| I-122 | (R)-5-fluoro-1,2,3,4-tetrahydronaphthalen-1-yl | 2-chlorobenzoyl |
| I-123 | 2-methyl-6-chloro-ylindan-1-yl | acetyl |
| I-124 | 2-methyl-6-chloro-ylindan-1-yl | 3-(trifluoromethyl)benzoyl |
| I-125 | (1R,2S)-2-methyl-6-fluoro-ylindan-1-yl | cyclopropylcarbonyl |
| I-126 | (1R,2S)-2-methyl-6-fluoro-ylindan-1-yl | 2-chlorobenzoyl |
| I-127 | (R)-5-fluoro-1,2,3,4-tetrahydronaphthalen-1-yl | pyridin-3-ylcarbonyl |
| I-128 | 2-methyl-6-chloro-ylindan-1-yl | cyclopropylcarbonyl |
| I-129 | (R)-5-fluoro-1,2,3,4-tetrahydronaphthalen-1-yl | cyclopropylcarbonyl |
| I-130 | (1R,2S)-2-methyl-6-fluoro-ylindan-1-yl | 4-chlorobenzoyl |
| I-131 | (R)-5-fluoro-1,2,3,4-tetrahydronaphthalen-1-yl | acetyl |
| I-132 | 2-methyl-6-chloro-ylindan-1-yl | benzoyl |
| I-133 | (R)-5-fluoro-1,2,3,4-tetrahydronaphthalen-1-yl | 4-chlorobenzoyl |
| I-134 | (1R,2S)-2-methyl-6-fluoro-ylindan-1-yl | benzoyl |
| I-135 | (R)-5-fluoro-1,2,3,4-tetrahydronaphthalen-1-yl | benzoyl |
| I-136 | 2-methyl-6-chloro-ylindan-1-yl | benzylsulfonyl |
| I-137 | (R)-5-methyl-1,2,3,4-tetrahydronaphthalen-1-yl | benzylsulfonyl |
| I-138 | (1R,2S)-2-methyl-6-fluoro-ylindan-1-yl | benzylsulfonyl |
| I-139 | (R)-5-fluoro-1,2,3,4-tetrahydronaphthalen-1-yl | benzylsulfonyl |
| I-140 | 2-methyl-6-chloro-ylindan-1-yl | cyclopropylsulfonyl |
| I-141 | (1R,2S)-2-methyl-6-fluoro-ylindan-1-yl | cyclopropylsulfonyl |
| I-142 | (R)-5-fluoro-1,2,3,4-tetrahydronaphthalen-1-yl | cyclopropylsulfonyl |
| I-143 | (R)-5-fluoro-1,2,3,4-tetrahydronaphthalen-1-yl | isopropylsulfonyl |
| I-144 | (R)-5-fluoro-1,2,3,4-tetrahydronaphthalen-1-yl | methylsulfonyl |
| I-145 | (1R,2S)-2-methyl-6-fluoro-ylindan-1-yl | methylsulfonyl |
| I-146 | chroman-4-yl | methylsulfonyl |
| I-147 | 2-methyl-6-chloro-ylindan-1-yl | methylsulfonyl |
| I-148 | 2-methyl-6-chloro-ylindan-1-yl | (2-chlorophenyl)sulfonyl |
| I-149 | (1R,2S)-2-methyl-6-fluoro-ylindan-1-yl | (2-chlorophenyl)sulfonyl |
| I-150 | (R)-5-fluoro-1,2,3,4-tetrahydronaphthalen-1-yl | (2-chlorophenyl)sulfonyl |
| I-151 | (1R,2S)-2,6-dimethylindan-1-yl | H |
| I-152 | (1R,2S)-2,6-dimethylindan-1-yl | isopropyl |
| I-153 | (R)-indan-1-yl | H |
| I-154 | (R)-1,2,3,4-tetrahydronaphthalen-1-yl | H |
| I-155 | (R)-chroman-4-yl | H |
| I-156 | (R)-6-methylindan-1-yl | H |
| I-157 | (R)-7-methyl-1,2,3,4-tetrahydronaphthalen-1-yl | H |
| I-158 | (R)-indan-1-yl | acetyl |
| I-159 | (R)-indan-1-yl | benzoyl |
| I-160 | (R)-indan-1-yl | cyclopropylcarbonyl |
| I-161 | (R)-indan-1-yl | 3-(trifluoromethyl)benzoyl |
| I-162 | (R)-indan-1-yl | 4-chlorobenzoyl |
| I-163 | (R)-1,2,3,4-tetrahydronaphthalen-1-yl | acetyl |
| I-164 | (R)-1,2,3,4-tetrahydronaphthalen-1-yl | benzoyl |
| I-165 | (1R,2S)-2,6-dimethylindan-1-yl | acetyl |
| I-166 | (1R,2S)-2,6-dimethylindan-1-yl | benzoyl |
| I-167 | (1R,2S)-2,6-dimethylindan-1-yl | cyclopropylcarbonyl |
| I-168 | (1R,2S)-2,6-dimethylindan-1-yl | 3-(trifluoromethyl)benzoyl |
| I-169 | (1R,2S)-2,6-dimethylindan-1-yl | 2-chlorobenzoyl |
| I-170 | (1R,2S)-2,6-dimethylindan-1-yl | 3-chlorobenzoyl |
| I-171 | (1R,2S)-2,6-dimethylindan-1-yl | 4-chlorobenzoyl |
| I-172 | (1R,2S)-2,6-dimethylindan-1-yl | cyclohexylcarbonyl |
| I-173 | (R)-6-methylindan-1-yl | acetyl |
| I-174 | (R)-6-methylindan-1-yl | benzoyl |
| I-175 | (R)-6-methylindan-1-yl | cyclopropylcarbonyl |
| I-176 | (R)-6-methylindan-1-yl | 3-(trifluoromethyl)benzoyl |
| I-177 | (R)-6-methylindan-1-yl | 2-chlorobenzoyl |
| I-178 | (R)-6-methylindan-1-yl | 3-chlorobenzoyl |
| I-179 | (R)-6-methylindan-1-yl | 4-chlorobenzoyl |
| I-180 | (R)-7-methyl-1,2,3,4-tetrahydronaphthalen-1-yl | acetyl |
| I-181 | (R)-7-methyl-1,2,3,4-tetrahydronaphthalen-1-yl | benzoyl |
| I-182 | (R)-7-methyl-1,2,3,4-tetrahydronaphthalen-1-yl | cyclopropylcarbonyl |
| I-183 | (R)-7-methyl-1,2,3,4-tetrahydronaphthalen-1-yl | 3-(trifluoromethyl)benzoyl |
| I-184 | (R)--7-methyl-1,2,3,4-tetrahydronaphthalen-1-yl | 2-chlorobenzoyl |
| I-185 | (R)-7-methyl-1,2,3,4-tetrahydronaphthalen-1-yl | 3-chlorobenzoyl |
| I-186 | (R)-7-methyl-1,2,3,4-tetrahydronaphthalen-1-yl | 4-chlorobenzoyl |
| I-187 | (R)-7-methyl-1,2,3,4-tetrahydronaphthalen-1-yl | cyclohexylcarbonyl |
| I-188 | 2,6-dimethylchroman-4-yl | acetyl |
| I-189 | 2,6-dimethylchroman-4-yl | benzoyl |
| I-190 | (1R,2S)-2,6-dimethylindan-1-yl | methylsulfonyl |
| I-191 | (1R,2S)-2,6-dimethylindan-1-yl | isopropylsulfonyl |
| I-192 | (1R,2S)-2,6-dimethylindan-1-yl | cyclopropylsulfonyl |
| I-193 | (1R,2S)-2,6-dimethylindan-1-yl | cyclopentylsulfonyl |
| I-194 | (1R,2S)-2,6-dimethylindan-1-yl | benzylsulfonyl |
| I-195 | (1R,2S)-2,6-dimethylindan-1-yl | (2-chlorophenyl)sulfonyl |
| I-196 | (R)-6-methylindan-1-yl | methylsulfonyl |
| I-197 | (R)-6-methylindan-1-yl | isopropylsulfonyl |
| I-198 | (R)-7-methyl-1,2,3,4-tetrahydronaphthalen-1-yl | methylsulfonyl |
| I-199 | (R)-7-methyl-1,2,3,4-tetrahydronaphthalen-1-yl | isopropylsulfonyl |
| I-200 | (R)-7-methyl-1,2,3,4-tetrahydronaphthalen-1-yl | cyclopropylsulfonyl |
| I-201 | (R)-7-methyl-1,2,3,4-tetrahydronaphthalen-1-yl | cyclopentylsulfonyl |
| I-202 | (R)-7-methyl-1,2,3,4-tetrahydronaphthalen-1-yl | benzylsulfonyl |
| I-203 | (R)-7-methyl-1,2,3,4-tetrahydronaphthalen-1-yl | (2-chlorophenyl)sulfonyl |
| I-340 | (1R,2S)-2,6-dimethylindan-1-yl | 2,2,2-trifluoroacetyl |
| I-341 | indan-1-yl | 2,2,2-trifluoroacetyl |
| I-342 | chroman-4-yl | 2,2,2-trifluoroacetyl |
| I-343 | (R)-7-methyl-1,2,3,4-tetrahydronaphthalen-1-yl | ethyl |
| I-344 | (1R,2S)-2,6-dimethylindan-1-yl | propyl |

TABLE 2
Examples of the compounds of the general formula (I-b):

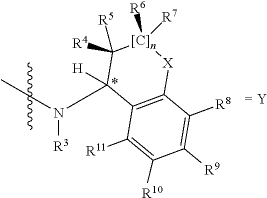

| | |
|---|---|
| | (I-b) |
| 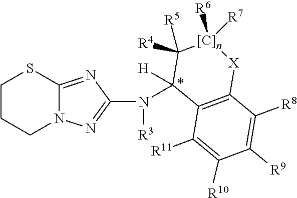 | |

| Example No.: | Y |
|---|---|
| I-204 | chroman-4-yl |
| I-205 | indan-1-yl |
| I-206 | (R)-5-fluoro-1,2,3,4-tetrahydronaphthalen-1-yl |
| I-207 | 7-methyl-1,2,3,4-tetrahydronaphthalen-1-yl |
| I-208 | (R)-7-fluoro-1,2,3,4-tetrahydronaphthalen-1-yl |
| I-209 | 1,2,3,4-tetrahydronaphthalen-1-yl |
| I-210 | 6-methylindan-1-yl |
| I-345 | (R)-indan-1-yl |
| I-347 | (R)-6-methylindan-1-yl |
| I-348 | (1R,2S)-2,6-dimethylindan-1-yl |
| I-349 | (R)-1,2,3,4-tetrahydronaphthalen-1-yl |
| I-350 | 2-methyl-1,2,3,4-tetrahydronaphthalen-1-yl |
| I-351 | 6-chloro-ylindan-1-yl |
| I-376 | (R)-6-methylchroman-4-yl |

TABLE 3
Examples of the compounds of the general formula (I-c):

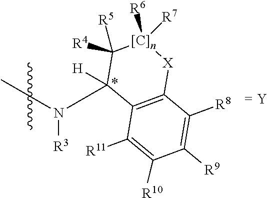

| | |
|---|---|
| | (I-c) |
| 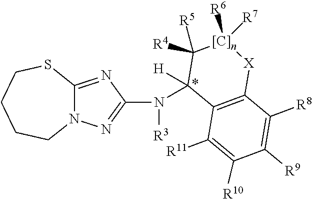 | |

| Example No.: | Y |
|---|---|
| I-211 | chroman-4-yl |
| I-212 | indan-1-yl |
| I-213 | (R)-5-fluoro-1,2,3,4-tetrahydronaphthalen-1-yl |
| I-214 | 7-methyl-1,2,3,4-tetrahydronaphthalen-1-yl |
| I-215 | (R)-7-fluoro-1,2,3,4-tetrahydronaphthalen-1-yl |
| I-216 | 1,2,3,4-tetrahydronaphthalen-1-yl |
| I-248 | 6-methylindan-1-yl |
| I-352 | (R)-indan-1-yl |
| I-353 | (R)-6-methylindan-1-yl |
| I-354 | (1R,2S)-2,6-dimethylindan-1-yl |
| I-355 | (R)-1,2,3,4-tetrahydronaphthalen-1-yl |
| I-356 | 2-methyl-1,2,3,4-tetrahydronaphthalen-1-yl |
| I-357 | 6-chloro-ylindan-1-yl |
| I-377 | (R)-6-methylchroman-4-yl |
| I-402 | (R)-7-methyl-1,2,3,4-tetrahydronaphthalen-1-yl |

TABLE 4
Examples of the compounds of the general formula (I-d):

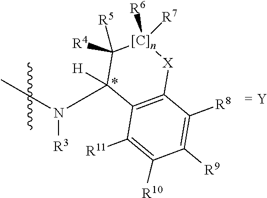

| | |
|---|---|
| | (I-d) |
| 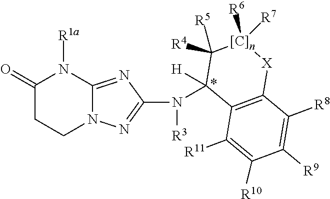 | |

| Example No.: | Y | R^{1a} |
|---|---|---|
| I-217 | indan-1-yl | benzyl |
| I-218 | indan-1-yl | [3-(trifluoromethyl)phenyl]methyl |
| I-219 | indan-1-yl | cyclopropylmethyl |
| I-220 | indan-1-yl | isopropyl |
| I-221 | chroman-4-yl | benzyl |
| I-222 | (R)-7-fluoro-1,2,3,4-tetrahydronaphthalen-1-yl | cyclohexylmethyl |
| I-223 | indan-1-yl | cyclohexylmethyl |
| I-224 | (R)-7-fluoro-1,2,3,4-tetrahydronaphthalen-1-yl | isopropyl |
| I-225 | chroman-4-yl | methyl |
| I-226 | (R)-7-fluoro-1,2,3,4-tetrahydronaphthalen-1-yl | methyl |
| I-227 | 6-methylindan-1-yl | methyl |
| I-228 | 1,2,3,4-tetrahydronaphthalen-1-yl | cyclopropylmethyl |
| I-229 | (R)-7-fluoro-1,2,3,4-tetrahydronaphthalen-1-yl | cyclopropylmethyl |
| I-230 | indan-1-yl | methyl |
| I-231 | 7-methyl-1,2,3,4-tetrahydronaphthalen-1-yl | methyl |
| I-254 | 7-methyl-1,2,3,4-tetrahydronaphthalen-1-yl | cyclopropylmethyl |
| I-255 | chroman-4-yl | cyclohexylmethyl |
| I-256 | 6-methylindan-1-yl | cyclohexylmethyl |
| I-262 | 1,2,3,4-tetrahydronaphthalen-1-yl | isopropyl |
| I-263 | (R)-5-fluoro-1,2,3,4-tetrahydronaphthalen-1-yl | methyl |
| I-264 | chroman-4-yl | isopropyl |
| I-265 | (R)-7-fluoro-1,2,3,4-tetrahydronaphthalen-1-yl | (2-chlorophenyl)methyl |
| I-266 | 1,2,3,4-tetrahydronaphthalen-1-yl | [3-(trifluoromethyl)phenyl]methyl |
| I-267 | 1,2,3,4-tetrahydronaphthalen-1-yl | (2-chlorophenyl)methyl |
| I-268 | (R)-7-fluoro-1,2,3,4-tetrahydronaphthalen-1-yl | [3-(trifluoromethyl)phenyl]methyl |
| I-269 | (R)-5-fluoro-1,2,3,4-tetrahydronaphthalen-1-yl | [3-(trifluoromethyl)phenyl]methyl |
| I-270 | chroman-4-yl | (2-chlorophenyl)methyl |
| I-271 | 1,2,3,4-tetrahydronaphthalen-1-yl | cyclohexylmethyl |
| I-272 | (R)-5-fluoro-1,2,3,4-tetrahydronaphthalen-1-yl | cyclohexylmethyl |
| I-273 | chroman-4-yl | [3-(trifluoromethyl)phenyl]methyl |
| I-274 | (R)-5-fluoro-1,2,3,4-tetrahydronaphthalen-1-yl | benzyl |
| I-275 | (R)-7-fluoro-1,2,3,4-tetrahydronaphthalen-1-yl | benzyl |
| I-276 | (R)-5-fluoro-1,2,3,4-tetrahydronaphthalen-1-yl | cyclopropylmethyl |
| I-277 | chroman-4-yl | cyclopropylmethyl |
| I-278 | 1,2,3,4-tetrahydronaphthalen-1-yl | methyl |
| I-279 | (R)-5-fluoro-1,2,3,4-tetrahydronaphthalen-1-yl | (2-chlorophenyl)methyl |
| I-280 | (R)-5-fluoro-1,2,3,4-tetrahydronaphthalen-1-yl | isopropyl |
| I-281 | 1,2,3,4-tetrahydronaphthalen-1-yl | benzyl |
| I-282 | indan-1-yl | (2-chlorophenyl)methyl |
| I-283 | 6-methylindan-1-yl | isopropyl |
| I-284 | 6-methylindan-1-yl | cyclopropylmethyl |
| I-285 | 6-methylindan-1-yl | benzyl |
| I-286 | indan-1-yl | H |
| I-287 | 1,2,3,4-tetrahydronaphthalen-1-yl | H |
| I-288 | (R)-5-fluoro-1,2,3,4-tetrahydronaphthalen-1-yl | H |
| I-289 | (R)-7-fluoro-1,2,3,4-tetrahydronaphthalen-1-yl | H |
| I-290 | chroman-4-yl | H |
| I-310 | 7-methyl-1,2,3,4-tetrahydronaphthalen-1-yl | [3-(trifluoromethyl)phenyl]methyl |
| I-311 | 7-methyl-1,2,3,4-tetrahydronaphthalen-1-yl | (2-chlorophenyl)methyl |
| I-312 | 6-methylindan-1-yl | (2-chlorophenyl)methyl |
| I-313 | 7-methyl-1,2,3,4-tetrahydronaphthalen-1-yl | cyclohexylmethyl |
| I-314 | 6-methylindan-1-yl | [3-(trifluoromethyl)phenyl]methyl |
| I-315 | 7-methyl-1,2,3,4-tetrahydronaphthalen-1-yl | benzyl |
| I-316 | 7-methyl-1,2,3,4-tetrahydronaphthalen-1-yl | isopropyl |

TABLE 5
Examples of the compounds of the general formula (I-e):

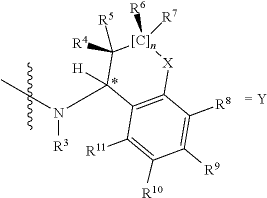

| | |
|---|---|
| | (I-e) |
| 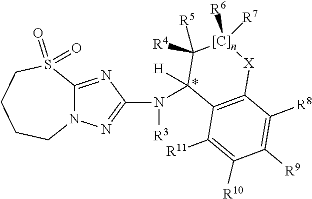 | |

| Example No.: | Y |
|---|---|
| I-232 | 6-methylindan-1-yl |
| I-233 | (R)-7-fluoro-1,2,3,4-tetrahydronaphthalen-1-yl |
| I-234 | 7-methyl-1,2,3,4-tetrahydronaphthalen-1-yl |
| I-235 | (R)-5-fluoro-1,2,3,4-tetrahydronaphthalen-1-yl |
| I-236 | indan-1-yl |
| I-237 | chroman-4-yl |
| I-253 | 1,2,3,4-tetrahydronaphthalen-1-yl |
| I-364 | (R)-indan-1-yl |
| I-365 | (R)-6-methylindan-1-yl |
| I-366 | (1R,2S)-2,6-dimethylindan-1-yl |
| I-367 | 1,2,3,4-tetrahydronaphthalen-1-yl |
| I-368 | 2-methyl-1,2,3,4-tetrahydronaphthalen-1-yl |
| I-375 | 6-chloro-ylindan-1-yl |
| I-381 | (R)-6-methylchroman-4-yl |

TABLE 6
Examples of the compounds of the general formula (I-f):

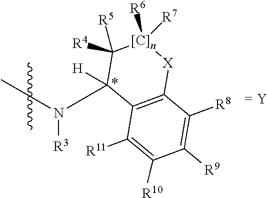

| | |
|---|---|
| | (I-f) |
| 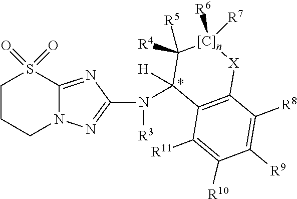 | |

| Example No.: | Y |
|---|---|
| I-238 | 6-methylindan-1-yl |
| I-239 | 7-methyl-1,2,3,4-tetrahydronaphthalen-1-yl |
| I-240 | (R)-5-fluoro-1,2,3,4-tetrahydronaphthalen-1-yl |
| I-241 | indan-1-yl |
| I-242 | chroman-4-yl |
| I-251 | (R)-7-fluoro-1,2,3,4-tetrahydronaphthalen-1-yl |
| I-252 | 1,2,3,4-tetrahydronaphthalen-1-yl |
| I-358 | 2-methyl-1,2,3,4-tetrahydronaphthalen-1-yl |
| I-378 | (R)-6-methylchroman-4-yl |

TABLE 7
Examples of the compounds of the general formula (I-g):

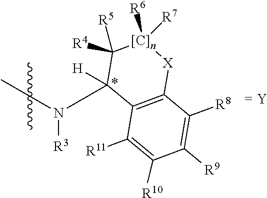

| | |
|---|---|
| | (I-g) |
| 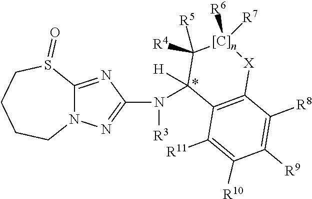 | |

| Example No.: | Y |
|---|---|
| I-243 | (R)-5-fluoro-1,2,3,4-tetrahydronaphthalen-1-yl |
| I-244 | (R)-7-fluoro-1,2,3,4-tetrahydronaphthalen-1-yl |
| I-245 | 6-methylindan-1-yl |
| I-246 | 7-methyl-1,2,3,4-tetrahydronaphthalen-1-yl |
| I-247 | chroman-4-yl |
| I-249 | 1,2,3,4-tetrahydronaphthalen-1-yl |
| I-250 | indan-1-yl |
| I-369 | (R)-indan-1-yl |
| I-370 | (R)-6-methylindan-1-yl |
| I-371 | (1R,2S)-2,6-dimethylindan-1-yl |
| I-372 | (R)-1,2,3,4-tetrahydronaphthalen-1-yl |
| I-373 | 2-methyl-1,2,3,4-tetrahydronaphthalen-1-yl |
| I-374 | 6-chloro-ylindan-1-yl |
| I-380 | (R)-6-methylchroman-4-yl |

TABLE 8
Examples of the compounds of the general formula (I-h):

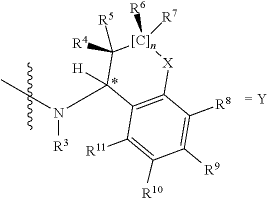

| | |
|---|---|
| | (I-h) |
| 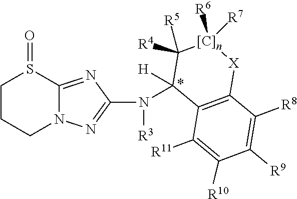 | |

| Example No.: | Y |
|---|---|
| I-346 | (R)-indan-1-yl |
| I-359 | (R)-6-methylindan-1-yl |
| I-360 | (1R,2S)-2,6-dimethylindan-1-yl |
| I-361 | (R)-1,2,3,4-tetrahydronaphthalen-1-yl |
| I-362 | 2-methyl-1,2,3,4-tetrahydronaphthalen-1-yl |
| I-363 | 6-chloro-ylindan-1-yl |
| I-379 | (R)-6-methylchroman-4-yl |

TABLE 9
Examples of the compounds of the general formula (I-i):

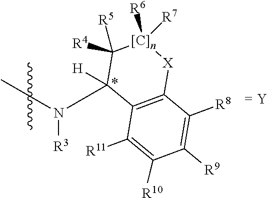

| | |
|---|---|
| | (I-i) |
| 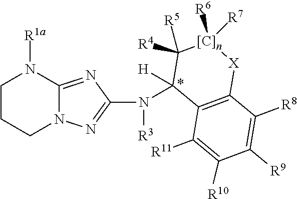 | |

| Example No.: | Y | R^{1a} |
|---|---|---|
| I-257 | (R)-7-fluoro-1,2,3,4-tetrahydronaphthalen-1-yl | cyclopropylcarbonyl |
| I-258 | 1,2,3,4-tetrahydronaphthalen-1-yl | 3-(trifluoromethyl)benzoyl |
| I-259 | 1,2,3,4-tetrahydronaphthalen-1-yl | cyclopropylcarbonyl |
| I-260 | (R)-7-fluoro-1,2,3,4-tetrahydronaphthalen-1-yl | cyclohexylcarbonyl |
| I-261 | 1,2,3,4-tetrahydronaphthalen-1-yl | cyclohexylcarbonyl |
| I-291 | indan-1-yl | cyclopropylcarbonyl |
| I-292 | 6-methylindan-1-yl | 3-(trifluoromethyl)benzoyl |
| I-293 | indan-1-yl | 3-(trifluoromethyl)benzoyl |
| I-294 | (R)-5-fluoro-1,2,3,4-tetrahydronaphthalen-1-yl | cyclopropylcarbonyl |
| I-295 | 6-methylindan-1-yl | cyclopropylcarbonyl |
| I-296 | (R)-7-fluoro-1,2,3,4-tetrahydronaphthalen-1-yl | 3-(trifluoromethyl)benzoyl |
| I-297 | chroman-4-yl | 3-(trifluoromethyl)benzoyl |
| I-298 | chroman-4-yl | cyclopropylcarbonyl |
| I-299 | (R)-5-fluoro-1,2,3,4-tetrahydronaphthalen-1-yl | 3-(trifluoromethyl)benzoyl |
| I-300 | (R)-7-fluoro-1,2,3,4-tetrahydronaphthalen-1-yl | methylsulfonyl |
| I-301 | 6-methylindan-1-yl | methylsulfonyl |
| I-302 | indan-1-yl | methylsulfonyl |
| I-303 | (R)-5-fluoro-1,2,3,4-tetrahydronaphthalen-1-yl | methylsulfonyl |
| I-304 | chroman-4-yl | methylsulfonyl |
| I-305 | 1,2,3,4-tetrahydronaphthalen-1-yl | methylsulfonyl |
| I-306 | chroman-4-yl | cyclohexylcarbonyl |
| I-307 | indan-1-yl | cyclohexylcarbonyl |
| I-308 | 6-methylindan-1-yl | cyclohexylcarbonyl |
| I-309 | (R)-5-fluoro-1,2,3,4-tetrahydronaphthalen-1-yl | cyclohexylcarbonyl |
| I-317 | 7-methyl-1,2,3,4-tetrahydronaphthalen-1-yl | 3-(trifluoromethyl)benzoyl |
| I-318 | 7-methyl-1,2,3,4-tetrahydronaphthalen-1-yl | cyclopropylcarbonyl |
| I-319 | (R)-7-fluoro-1,2,3,4-tetrahydronaphthalen-1-yl | acetyl |
| I-320 | 6-methylindan-1-yl | acetyl |
| I-321 | 7-methyl-1,2,3,4-tetrahydronaphthalen-1-yl | acetyl |
| I-322 | indan-1-yl | acetyl |
| I-323 | (R)-5-fluoro-1,2,3,4-tetrahydronaphthalen-1-yl | acetyl |
| I-324 | chroman-4-yl | acetyl |
| I-325 | 7-methyl-1,2,3,4-tetrahydronaphthalen-1-yl | isopropylsulfonyl |
| I-326 | indan-1-yl | cyclopropylsulfonyl |
| I-327 | 7-methyl-1,2,3,4-tetrahydronaphthalen-1-yl | cyclopropylsulfonyl |
| I-328 | 6-methylindan-1-yl | isopropylsulfonyl |
| I-329 | 7-methyl-1,2,3,4-tetrahydronaphthalen-1-yl | methylsulfonyl |
| I-330 | 7-methyl-1,2,3,4-tetrahydronaphthalen-1-yl | cyclohexylcarbonyl |
| I-331 | 1,2,3,4-tetrahydronaphthalen-1-yl | acetyl |
| I-332 | 1,2,3,4-tetrahydronaphthalen-1-yl | isopropylsulfonyl |
| I-333 | 1,2,3,4-tetrahydronaphthalen-1-yl | cyclopropylsulfonyl |
| I-334 | indan-1-yl | isopropylsulfonyl |
| I-335 | (R)-5-fluoro-1,2,3,4-tetrahydronaphthalen-1-yl | cyclopropylsulfonyl |
| I-336 | 6-methylindan-1-yl | cyclopropylsulfonyl |
| I-337 | chroman-4-yl | isopropylsulfonyl |
| I-338 | chroman-4-yl | cyclopropylsulfonyl |
| I-339 | (R)-5-fluoro-1,2,3,4-tetrahydronaphthalen-1-yl | isopropylsulfonyl |

TABLE 10
Examples of the compounds of the general formula (I-j):

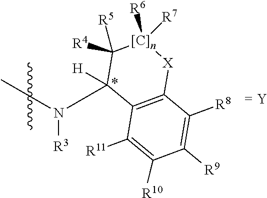

| | |
|---|---|
| | (I-j) |
| 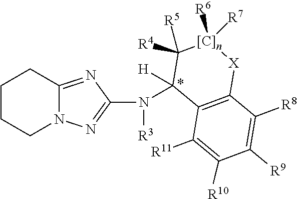 | |

| Example No.: | Y |
|---|---|
| I-382 | 1,2,3,4-tetrahydronaphthalen-1-yl |
| I-383 | 7-methyl-1,2,3,4-tetrahydronaphthalen-1-yl |
| I-384 | indan-1-yl |
| I-385 | 6-methylindan-1-yl |
| I-386 | (1R,2S)-2,6-dimethylindan-1-yl |
| I-387 | (1R,2S)-2-methylindan-1-yl |
| I-388 | 7-fluoro-1,2,3,4-tetrahydronaphthalen-1-yl |
| I-389 | 5-fluoro-1,2,3,4-tetrahydronaphthalen-1-yl |
| I-390 | chroman-4-yl |
| I-391 | 8-methylchroman-4-yl |

TABLE 11
Examples of the compounds of the general formula (I-k):

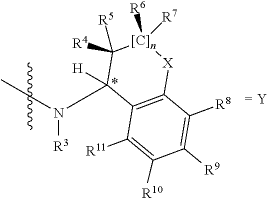

| | |
|---|---|
| | (I-k) |
| 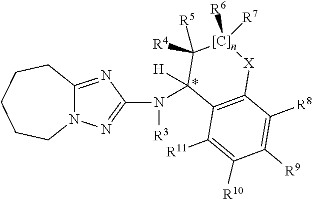 | |

| Example No.: | Y |
|---|---|
| I-392 | 1,2,3,4-tetrahydronaphthalen-1-yl |
| I-393 | 7-methyl-1,2,3,4-tetrahydronaphthalen-1-yl |
| I-394 | indan-1-yl |
| I-395 | 6-methylindan-1-yl |
| I-396 | (1R,2S)-2,6-dimethylindan-1-yl |
| I-397 | (1R,2S)-2-methylindan-1-yl |
| I-398 | 7-fluoro-1,2,3,4-tetrahydronaphthalen-1-yl |
| I-399 | 5-fluoro-1,2,3,4-tetrahydronaphthalen-1-yl |
| I-400 | chroman-4-yl |
| I-401 | 8-methylchroman-4-yl |

The present invention further provides processes for preparing corresponding compounds of the general formula (I) and/or salts thereof and/or agrochemically compatible quaternized nitrogen derivatives thereof

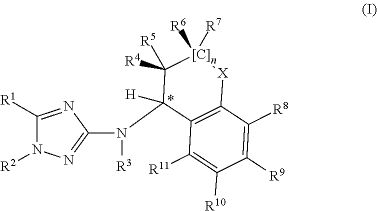

in which the radicals R^{1b} to R¹¹ and also X and the serial number n have the above meanings, and wherein, in a first process,
a compound of the general formula (II)

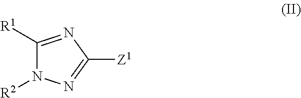

where R¹, R² have the meaning given above and Z represents an exchangeable radical or a leaving group
   is reacted with an amine of the general formula (III)

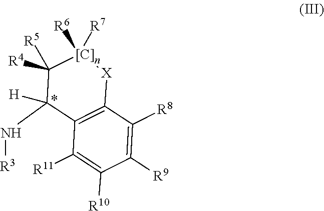

where the radicals R³ to R¹¹ and X and n have the above meaning,
   or with an acid addition salt of the amine of the general formula (II).

The exchangeable radical Z¹ or the leaving group Z¹ represents fluorine, chlorine, bromine, iodine, a (C₁₋₄)-alkylsulfanyl, a (C₁₋₄)-alkylsulfinyl, a (C₁₋₄)-alkylsulfonyl, an unsubstituted or a mono- or poly-fluorine-, -chlorine-, -bromine- or —(C₁₋₄)-alkyl- or —(C₁₋₄)-alkoxy-substituted phenyl-(C₁₋₄)-alkylsulfonyl or a (C₁₋₄)-alkylphenylsulfonyl.

If necessary, a Z¹ radical can be converted into another group of better exchangeability. For example, in the context of a two-stage one-pot method, a (C₁₋₄)-alkylsulfanyl can be converted with an oxidizing agent such as m-chloroperbenzoic acid or Oxone® into (C₁₋₄)-alkylsulfinyl or (C₁₋₄)-alkylsulfonyl or mixtures thereof, and then reacted with an amine of the general formula (III) or an acid addition salt using an auxiliary base, for example triethylamine or potassium carbonate.

The reaction may optionally also be catalyzed by various auxiliaries, for example by the reagents potassium phosphate, copper(I) iodide and N,N-diethyl-2-hydroxybenzamide, or in the manner of the Buchwald-Hartwig coupling by special transition metal catalyst systems and bases. Suitable catalyst systems are, for example, [(2-dicyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate (G3 Brettphos Pd®) and 2-(dicyclohexylphosphino)3,6-dimethoxy-2′,4′,6′-triisopropyl-1,1′-biphenyl (Brettphos®) with t-butoxide or chloro[2-(dicyclohexylphosphino)-3,6-dimethoxy-2′,4′,6′-triisopropylbiphenyl][2-(2-aminoethyl)phenyl]Pd(II) with sodium carbonate or tris(dibenzylideneacetone)dipalladium(0) chloroform complex and (5-diphenylphosphanyl-9,9-dimethylxanthen-4-yl)diphenylphosphane with sodium phenoxide or 2-dicyclohexylphosphino-2′,4′,6′-triisopropylbiphenyl (XPhos) and tris(dibenzylideneacetone)dipalladium(0) with cesium carbonate.

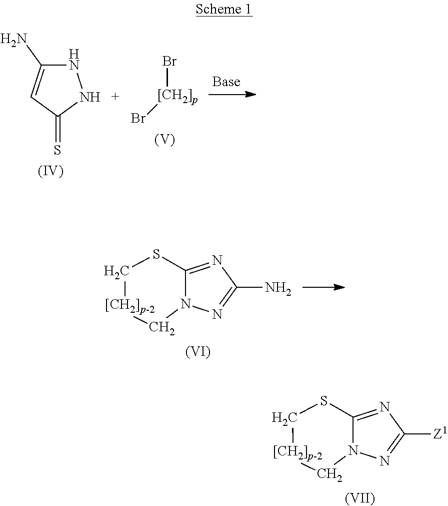

The starting compounds used in the above schemes are either commercially available or preparable by methods known per se. For instance, the substituted cyclic 1,2,4-triazoles of the general formula (I) can be prepared, for example, by the methods described in J. Het. Chem. 40, 821-826 (2003).

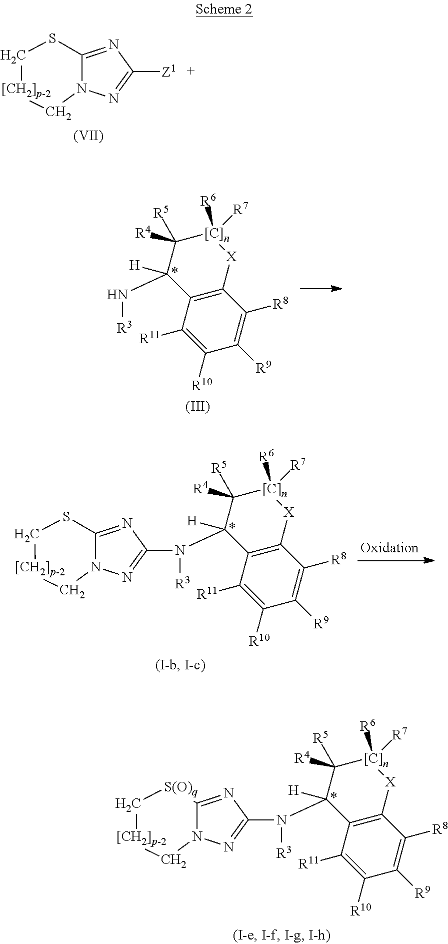

where Z¹ is an exchangeable radical or a leaving group can react with an amine of the general formula (III) or an acid addition salt of the amine of the general formula (III) and the serial number p=2, 3 or 4 and the serial number q=1 or 2.

After the substitution, the sulfur can be successfully oxidized with an oxidizing agent such as m-chloroperbenzoic acid or potassium peroxymonosulfate (Oxone®) (Scheme 2).

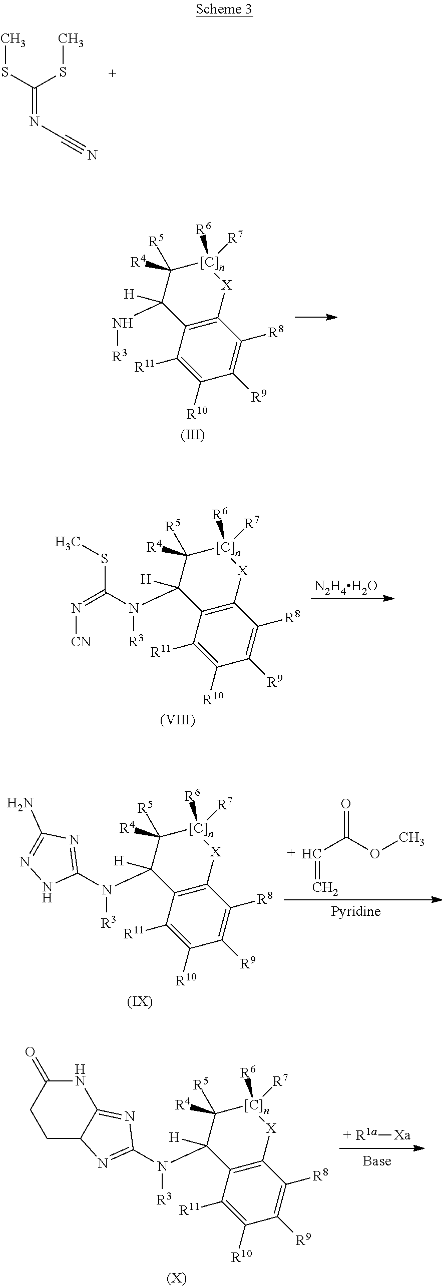

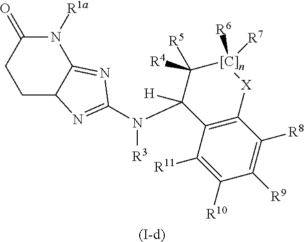

The inventive substituted cyclic 1,2,4-triazoles having the general formula (II) can be prepared as follows (scheme 3). The amines of the general formula (III) or an acid addition salt of the amine of the general formula (III) are reacted with dimethyl N-cyanodithioiminocarbonate, for example by the methods described in J. Med. Chem. 56, 9071-9088 (2013), and cyclized with hydrazine hydrate to give the aminotriazole (IX). The aminotriazole can be converted by a Michael addition with methyl acrylate to give 6,7-dihydro-4H-[1,2,4]triazolo[1,5-a]pyrimidin-5-one. The last stage is effected by analkylation under basic conditions, where Xa=chlorine, bromine, iodine or (C₁₋₄)-alkylsulfonyl or may be a phenyl-(C₁₋₄)-alkylsulfonyl.

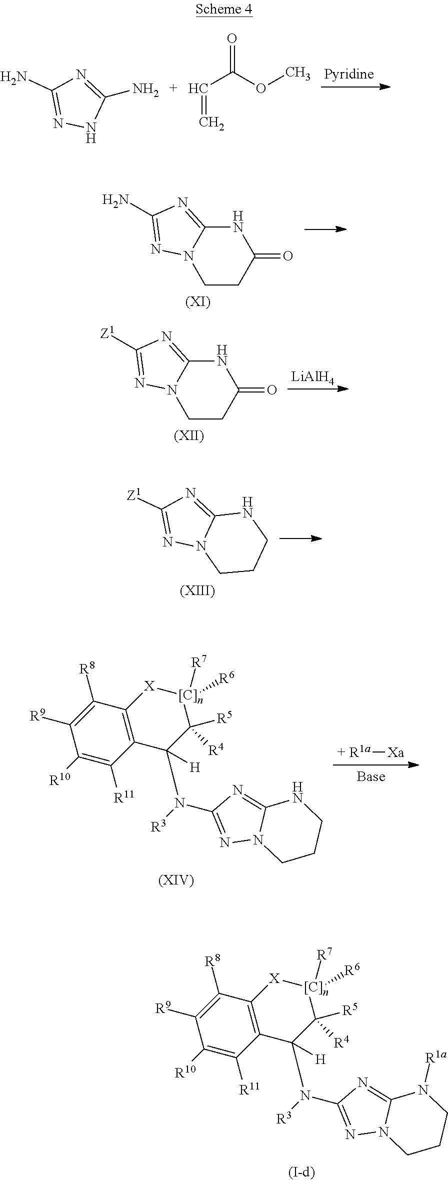

The starting compounds used in scheme 4 above are either commercially available or preparable by known methods. In this way, the inventive substituted cyclic 1,2,4-triazoles having the general formula (II) can be prepared. 1H-1,2,4-Triazole-3,5-diamine is converted by a Michael addition with methyl acrylate to give 2-amino-6,7-dihydro-4H-[1,2,4]triazolo[1,5-a]pyrimidin-5-one (XI). The amino group can be converted to a leaving group Z¹ in a Sandmeyer reaction, for example by the methods described in MedChemComm, 4, 422-431 (2013), where Xa is as defined above.

The exchangeable radical Z¹ or the leaving group Z¹ represents fluorine, chlorine, bromine, iodine, a (C₁₋₄)-alkylsulfanyl, a (C₁₋₄)-alkylsulfinyl, a (C₁₋₄)-alkylsulfonyl, an unsubstituted or a mono- or poly-fluorine-, -chlorine-, -bromine- or —(C₁₋₄)-alkyl- or —(C₁₋₄)-alkoxy-substituted phenyl-(C₁-4)-alkylsulfonyl or a (C₁₋₄)-alkylphenylsulfonyl.

The reaction may optionally also be catalyzed by various auxiliaries, for example by the reagents potassium phosphate, copper(I) iodide and N,N-diethyl-2-hydroxybenzamide, or in the manner of the Buchwald-Hartwig coupling by special transition metal catalyst systems. The latter reaction is effected by an alkylation under basic conditions.

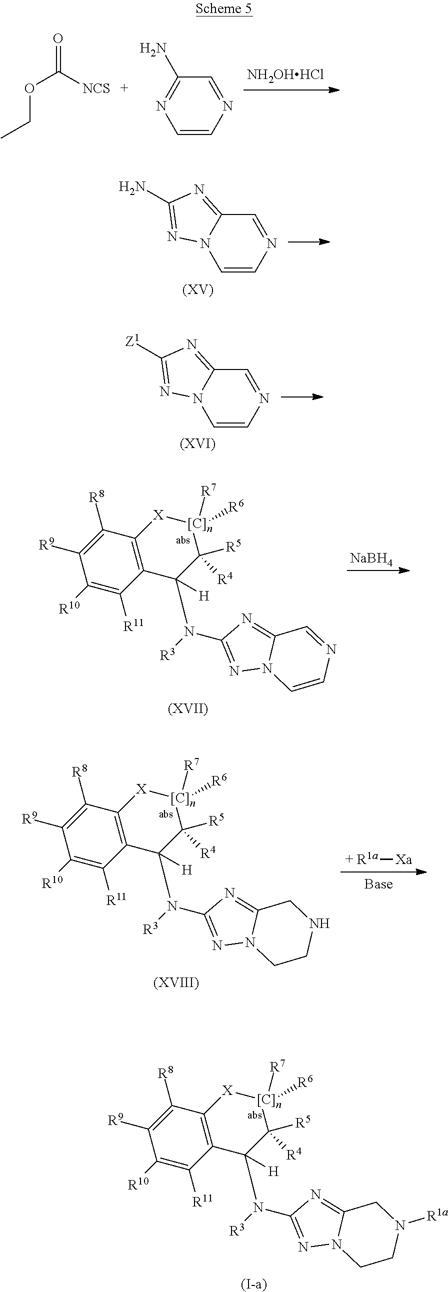

The starting compounds used in scheme 5 above are either commercially available or preparable by known methods. In this way, the inventive substituted cyclic 1,2,4-triazoles having the general formula (I-a) can be prepared. [1,2,4]Triazolo[1,5-a]pyrazine-2-amine is prepared by reaction between 2-aminopyrazine, ethoxycarbonyl isothiocyanate and hydroxylamine hydrochloride under basic conditions (analogously to J. Med. Chem., 2014, vol. 57, pages 3687-3706). Subsequently, the amino group is converted by a Sandmeyer reaction to an exchangeable radical Z¹.

The exchangeable radical Z¹ or the leaving group Z¹ represents fluorine, chlorine, bromine, iodine, a (C₁₋₄)-alkylsulfanyl, a (C₁₋₄)-alkylsulfinyl, a (C₁₋₄)-alkylsulfonyl, an unsubstituted or a mono- or poly-fluorine-, -chlorine-, -bromine- or —(C₁₋₄)-alkyl- or —(C₁₋₄)-alkoxy-substituted phenyl-(C₁₋₄)-alkylsulfonyl or a (C₁₋₄)-alkylphenylsulfonyl.

The reaction may optionally also be catalyzed by various auxiliaries, for example by the reagents potassium phosphate, copper(I) iodide and N,N-diethyl-2-hydroxybenzamide, or in the manner of the Buchwald-Hartwig coupling by special transition metal catalyst systems. In the last step, an alkylation is conducted under basic conditions.

The amines of the general formula (III) or the acid addition salt thereof are commercially available, or the synthesis thereof is described in WO 2004/069814 A1.

Compounds of the general formula (I) can also be prepared by first preparing a compound of the general formula (I) which is then converted to other target molecules in further reaction steps. For example, sulfur atoms in R¹ can be converted to SO or SO₂ by oxidation, or NH can be converted to the corresponding amides by reaction with acid chlorides or anhydrides or to the corresponding sulfonamides by reaction with sulfonyl chlorides

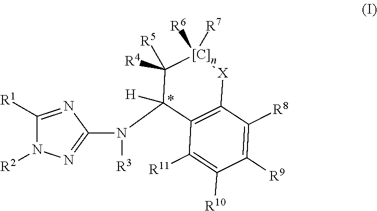

Collections of compounds of the formula (I) and/or salts thereof which can be synthesized by the abovementioned reactions can also be prepared in a parallelized manner, in which case this may be accomplished in a manual, partly automated or fully automated manner. It is possible, for example, to automate the conduct of the reaction, the workup or the purification of the products and/or intermediates. Overall, this is understood to mean a procedure as described, for example, by D. Tiebes in Combinatorial Chemistry—Synthesis, Analysis, Screening (editor: Günther Jung), Wiley, 1999, on pages 1 to 34.

For the parallelized conduct of the reaction and workup, it is possible to use a number of commercially available instruments, for example reaction stations from Radleys, Shirehill, Saffron Walden, Essex, CB11 3AZ, England, or MultiPROBE Automated Workstations from PerkinElmer, Waltham, Mass. 02451, USA. For the parallelized purification of compounds of the general formula (I) and salts thereof or of intermediates which occur in the course of preparation, available apparatuses include chromatography apparatuses, for example from Teledyne ISCO, Inc., 4700 Superior Street, Lincoln, Nebr. 68504, USA.

The apparatuses detailed lead to a modular procedure in which the individual working steps are automated, but manual operations have to be carried out between the working steps. This can be circumvented by using partly or fully integrated automation systems in which the respective automation modules are operated, for example, by robots.

The implementation of single or multiple synthesis steps can be supported by the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Aside from the methods described here, compounds of the general formula (I) and salts thereof can be prepared completely or partially by solid-phase-supported methods. For this purpose, individual intermediates or all intermediates in the synthesis or a synthesis adapted for the corresponding procedure are bound to a synthesis resin. Solid-phase-supported synthesis methods are described adequately in the technical literature, for example Barry A. Bunin in “The Combinatorial Index”, Academic Press, 1998 and Combinatorial Chemistry—Synthesis, Analysis, Screening (editor: Günther Jung), Wiley, 1999. The use of solid-phase-supported synthesis methods permits a number of protocols, which are known from the literature and which in turn may be performed manually or in an automated manner.

Both in the solid and in the liquid phase, the implementation of individual or several synthesis steps may be supported by the use of microwave technology. The specialist literature describes a series of experimental protocols, for example in Microwaves in Organic and Medicinal Chemistry (editors: C. O. Kappe and A. Stadler), Wiley, 2005.

The preparation by the processes described here gives compounds of the formula (I) and salts thereof in the form of substance collections, which are called libraries. The present invention also provides libraries comprising at least two compounds of the formula (I) and salts thereof.

On account of the herbicidal property of the compounds of the general formula (I), the invention also further provides for the use of the compounds of the general formula (I) according to the invention as herbicides for control of harmful plants.

Herbicides are used in agriculturally utilized crops during various cultivation phases. Thus, the application of some products even takes place before or during sowing. Others are applied before the crop plant emerges, i.e. before the seedling breaks through the earth's surface (pre-emergence herbicides). Finally, post-emergence herbicides are used if either already the seed leaves or foliage leaves have been formed by the crop plant.

The compounds of the invention can be employed either pre-emergence or post-emergence, preference being given to pre-emergence use of the compounds of the invention.

The pre-emergence treatment includes both the treatment of the area under cultivation prior to sowing (ppi=pre plant incorporation) and the treatment of the sown areas of cultivation which do not yet sustain any growth.

The application rate of the compounds of the general formula (I) and/or their salts is affected to a certain extent by external conditions such as temperature, humidity, etc. Here, the application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to the pre-emergence and the post-emergence application.

When the inventive compounds of the general formula (I) and/or their salts are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to the pre-emergence and the post-emergence application.

The compounds of the formula (I) according to the invention and their salts, also referred to synonymously and collectively hereinafter as compounds of the formula (I), have excellent herbicidal efficacy against a broad spectrum of economically important monocotyledonous and dicotyledonous harmful plants. The active compounds also have good control over perennial weeds which are difficult to control and produce shoots from rhizomes, root stocks or other perennial organs. It does not matter here whether the substances are applied by the presowing method, the pre-emergence method or the post-emergence method.

Specific examples of some representatives of the monocotyledonous and dicotyledonous weed flora which can be controlled by the compounds of the general formula (I) according to the invention are mentioned hereinafter, without any intention that the enumeration is to impose a restriction to particular species.

On the side of the monocotyledonous weed species, e.g. Agrostis, Alopecurus, Apera, Avena, Brachicaria, Bromus, Dactyloctenium, Digitaria, Echinochloa, Eleocharis, Eleusine, Festuca, Fimbristylis, Ischaemum, Lolium, Monochoria, Panicum, Paspalum, Phalaris, Phleum, Poa, Sagittaria, Scirpus, Setaria, Sphenoclea, and also Cyperus species predominantly from the annual group and on the sides of the perennial species Agropyron, Cynodon, Imperata and Sorghum and also perennial Cyperus species are well controlled.

On the side of the dicotyledonous weed species, the activity spectrum extends, for example, to species such as Galium, Viola, Veronica, Lamium, Stellaria, Amaranthus, Sinapis, Ipomoea, Matricaria, Abutilon and Sida on the annual side, and Convolvulus, Cirsium, Rumex and Artemisia in the case of the perennial weeds. Moreover, herbicidal action is observed in the case of dicotyledonous weeds such as Ambrosia, Anthemis, Carduus, Centaurea, Chenopodium, Cirsium, Convolvulus, Datura, Emex, Galeopsis, Galinsoga, Lepidium, Lindernia, Papaver, Portlaca, Polygonum, Ranunculus, Rorippa, Rotala, Seneceio, Sesbania, Solanum, Sonchus, Taraxacum, Trifolium, Urtica and Xanthium.

If the compounds of the general formula (I) according to the invention are applied to the soil surface before germination, either the emergence of the weed seedlings is prevented completely or the weeds grow until they have reached the cotyledon stage, but then they stop growing and ultimately die completely after three to four weeks have passed.

If the active compounds of the general formula (I) are applied post-emergence to the green parts of the plants, growth likewise stops sharply very rapidly after the treatment, and the weed plants remain at the growth stage at the time of application or die completely after a certain time, such that competition by the weeds, which is harmful to the crop plants, is thus eliminated very early and in a lasting manner.

Although the compounds of the general formula (I) according to the invention have excellent herbicidal activity in respect of monocotyledonous and dicotyledonous weeds, crop plants of economically important crops, for example wheat, barley, rye, rice, corn, sugar beet, cotton, oilseed rape and soybean, are only damaged negligibly, if at all. This is why the present compounds are highly suitable for the selective control of unwanted plant growth in agriculturally useful plants.

In addition, the substances of the general formula (I) according to the invention have excellent growth regulatory properties in crop plants. They engage in the plant's own metabolism in a regulatory fashion and can therefore be employed for the influencing, in a targeted manner, of plant constituents and for facilitating harvesting, such as, for example, by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since this can reduce or completely prevent lodging.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other particular properties relate, for example, to the harvested material with regard to quantity, quality, storability, composition and specific constituents. For instance, there are known transgenic plants with an elevated starch content or altered starch quality, or those with a different fatty acid composition in the harvested material. Other particular properties may be tolerance or resistance to abiotic stressors, for example heat, low temperatures, drought, salinity and ultraviolet radiation.

It is preferable to employ the compounds of the general formula (according to the invention or salts thereof in economically important transgenic crops of useful plants and ornamental plants, for example of cereals such as wheat, barley, rye, oats, sorghum and millet, rice, cassava and corn or else crops of sugar beet, cotton, soybean, oilseed rape, potato, tomato, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants.

Alternatively, novel plants with altered properties can be generated with the aid of recombinant methods (see, for example, EP 0221044, EP 0131624). For example, there have been descriptions in several cases of:
genetic modifications of crop plants for the purpose of modifying the starch synthesized in the plants (for example WO 92/011376, WO 92/014827, WO 91/019806),
transgenic crop plants which are resistant to certain herbicides of the glufosinate type (cf., for example, EP 0242236, EP 0242246) or the glyphosate type (WO 92/000377) or the sulfonylurea type (EP 0257993, U.S. Pat. No. 5,013,659),
transgenic crop plants, for example cotton, which is capable of producing Bacillus thuringiensis toxins (Bt toxins), which make the plants resistant to certain pests (EP 0142924, EP 0193259),
transgenic crop plants having a modified fatty acid composition (WO 91/013972),
genetically modified crop plants with novel constituents or secondary metabolites, for example novel phytoalexins, which bring about an increased disease resistance (EP 0309862, EP 0464461),
genetically modified plants having reduced photorespiration, which have higher yields and higher stress tolerance (EP 0305398),
transgenic crop plants which produce pharmaceutically or diagnostically important proteins (“molecular pharming”),
transgenic crop plants which feature higher yields or better quality,
transgenic crop plants which feature a combination, for example, of the abovementioned novel properties (“gene stacking”).

Numerous molecular biology techniques which can be used to produce novel transgenic plants with modified properties are known in principle; see, for example, I. Potrykus and G. Spangenberg (eds.) Gene Transfer to Plants, Springer Lab Manual (1995), Springer Verlag Berlin, Heidelberg, or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove parts of sequences or add natural or synthetic sequences. To join the DNA fragments with one another, adapters or linkers can be placed onto the fragments, see, for example, Sambrook et al., 1989, Molecular Cloning, A Laboratory Manual, 2nd edition Cold Spring Harbor Laboratory Press, Cold Spring Harbor, N.Y.; or Winnacker “Gene und Klone” [Genes and clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

It is preferable to use the compounds of the general formula (I) according to the invention in transgenic crops which are resistant to growth regulators, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or against herbicides from the group of the sulfonylureas, glyphosate, glufosinate or benzoylisoxazoles and analogous active compounds.

When the active compounds of the general formula (I) according to the invention are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the compounds of the general formula (I) according to the invention as herbicides for control of harmful plants in transgenic crop plants.

The compounds of the general formula (I) can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie [Chemical Technology]”, Volume 7, C. Hanser Verlag Munich, 4th Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London.

The necessary formulation assistants, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H. v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964, Schönfeldt, “Grenzflächenaktive Äthylenoxidaddukte [Interface-active ethylene oxide adducts]”, Wiss. Verlagsgesell., Stuttgart 1976, Winnacker-Küchler, “Chemische Technologie [Chemical Technology]”, Volume 7, C. Hanser Verlag Munich, 4th Ed. 1986.

Based on these formulations, it is also possible to produce combinations with other pesticidally active compounds, such as, for example, insecticides, acaricides, herbicides, fungicides, and also with safeners, fertilizers and/or growth regulators, for example in the form of a finished formulation or as a tank mix.

Wettable powders are preparations which can be dispersed uniformly in water and, as well as the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active compounds are finely ground, for example in customary apparatuses such as hammer mills, blower mills and air-jet mills, and simultaneously or subsequently mixed with the formulation auxiliaries.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents with addition of one or more surfactants of the ionic and/or nonionic type (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusts are obtained by grinding the active compound with finely distributed solid substances, for example talc, natural clays, such as kaolin, bentonite and pyrophillite, or diatomaceous earth.

Suspension concentrates may be water- or oil-based. They can be produced, for example, by wet grinding by means of standard commercial bead mills and optionally the addition of surfactants, as have already been listed above, for example, for the other types of formulation.

Emulsions, e.g. oil-in-water emulsions (EW), can be prepared, for example, by means of stirrers, colloid mills and/or static mixers using aqueous organic solvents and if appropriate surfactants, as have for example already been listed above in connection with the other types of formulation.

Granules can be produced either by spraying the active compound onto adsorptive granular inert material or by applying active compound concentrates to the surface of carriers, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules see, for example, processes in “Spray-Drying Handbook” 3rd Ed. 1979, G. Goodwin Ltd., London; J. E. Browning, “Agglomeration”, Chemical and Engineering 1967, pages 147 ff; “Perry's Chemical Engineer's Handbook”, 5th Ed., McGraw Hill, New York 1973, p. 8-57.

For further details regarding the formulation of crop protection agents, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical formulations contain generally 0.1% to 99% by weight, especially 0.1% to 95% by weight, of active compound of the formula (I).

In wettable powders, the active compound concentration is, for example, about 10% to 90% by weight, the remainder to 100% by weight consisting of customary formulation components. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is present in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary tackifiers, wetting agents, dispersants, emulsifiers, penetrants, preservatives, antifreeze agents and solvents, fillers, carriers and dyes, defoamers, evaporation inhibitors and agents which influence the pH and the viscosity.

The compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of tank mixes.

Active compounds which can be employed in combination with the compounds according to the invention in mixed formulations or in the tank mix are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as are described in, for example, Weed Research 26 (1986) 441-445 or “The Pesticide Manual”, 17th edition, The British Crop Protection Council and the Royal Soc. of Chemistry, 2015 (cf. https://www.bcpc.org/product/the-pesticide-manual-17th-edition) or online (https://www.bcpc.org/product/bcpc-online-pesticide-manual-latest-version) and the literature cited therein. Known herbicides or plant growth regulators which can be combined with the compounds according to the invention are, for example, the following, where said active compounds are designated either with their “common name” in accordance with the International Organization for Standardization (ISO) or with the chemical name or with the code number. They always encompass all of the application forms such as, for example, acids, salts, esters and also all isomeric forms such as stereoisomers and optical isomers, even if they are not explicitly mentioned.

Examples of such herbicidal mixing partners are:

acetochlor, acifluorfen, acifluorfen-sodium, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, -2-ethylhexyl, -isobutyl, -isooctyl, -isopropylammonium, -potassium, -triisopropanolammonium and -trolamine, 2,4-DB, 2,4-DB-butyl, -dimethylammonium, -isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat dibromide, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-5231, i.e. N-[2-chloro-4-fluoro-5-[4-(3-fluoropropyl)-4,5-dihydro-5-oxo-1H-tetrazol-1-yl]phenyl]ethansulfonamide, F-7967, i.e. 3-[7-chloro-5-fluoro-2-(trifluoromethyl)-1H-benzimidazol-4-yl]-1-methyl-6-(trifluoromethyl)pyrimidine-2,4 (1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and also the following compounds:

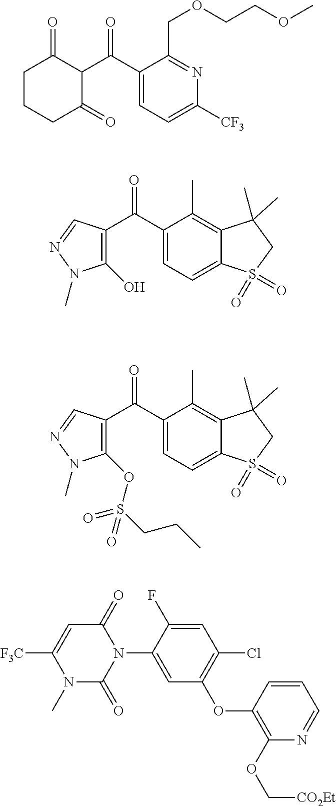 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenoxide mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

The safeners are preferably selected from the group consisting of:
S1) Compounds of the formula (S1)
   where the symbols and indices are defined as follows:

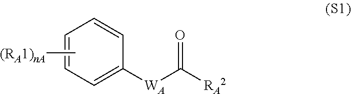

n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴),

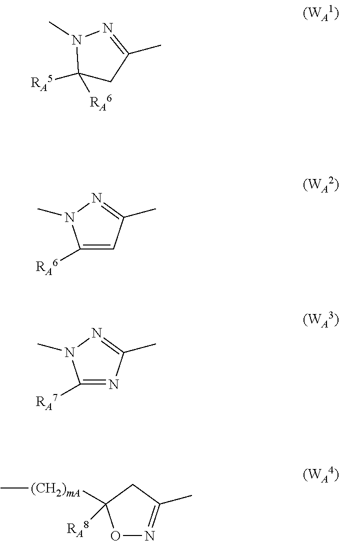

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbon radical, preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₅)-alkyl, cyano or COOR_{A}⁹, where R_{A}⁹ is hydrogen, (C₁-C₅)-alkyl, (C₁-C₅)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;
   preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1°), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1°), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

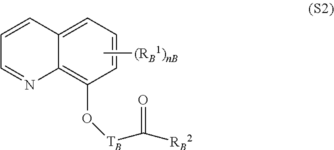

where the symbols and indices have the meanings below:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated
   or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³
R_{B}³ is hydrogen or an unsubstituted or substituted aliphatic hydrocarbon radical, preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
   preferably:
a) compounds of the 8-quinolinoxyacetic acid type (S2^{a}), preferably
   1-methylhexyl (5-chloro-8-quinolinoxy)acetate (“cloquintocet-mexyl”) (S2-1),
   (1,3-dimethylbut-1-yl) (5-chloro-8-quinolinoxy)acetate (S2-2),
   4-allyloxybutyl (5-chloro-8-quinolinoxy)acetate (S2-3),
   1-allyloxyprop-2-yl (5-chloro-8-quinolinoxy)acetate (S2-4),
   ethyl (5-chloro-8-quinolinoxy)acetate (S2-5),
   methyl (5-chloro-8-quinolinoxy)acetate (S2-6),
   allyl (5-chloro-8-quinolinoxy)acetate (S2-7),
   2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Compounds of the formula (S3)

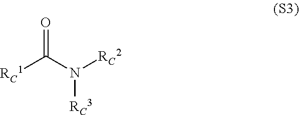

where the symbols and indices are defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
   preferably:
   active compounds of the dichloroacetamide type, which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “diclonon” (dicyclonon) or “BAS145138” or “LAB145138” (S3-9) ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyloxazolidine) (S3-10); and the (R) isomer thereof (S3-11).
S4) N-acylsulfonamides of the formula (S4) and salts thereof,

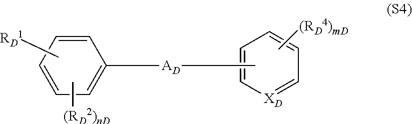

in which the symbols and indices are defined as follows:
A_{D} is SO₂—NR_{D}³—CO or CO—NR_{D}³—SO₂
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven latter radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxyl, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
   among these, preference is given to compounds of the N-acylsulfonamide type, for example of the formula (S4a) below, which are known, for example, from WO-A-97/45016

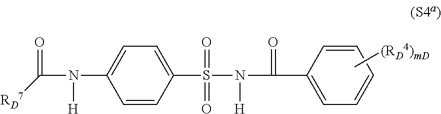

in which
R_{D}⁷ represents (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ represents halogen, (C₁-C₄)-alkyl, (C₁-C₄)-alkoxy, CF₃;
m_{D} represents 1 or 2;
v_{D} represents 0, 1, 2 or 3;
   and also
   acylsulfamoylbenzamides, for example of the formula (S4^{b}) below, which are known, for example, from WO-A-99/16744,

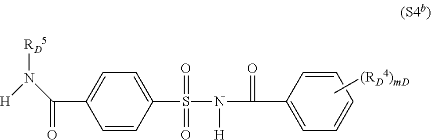

e.g. those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
   and also
   compounds of the N-acylsulfamoylphenylurea type, of the formula (S4°), which are known, for example, from EP-A-365484,

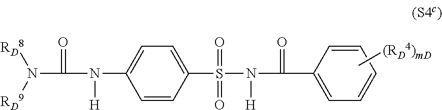

in which
R_{D}⁸ and R_{D}⁹ independently of one another represent hydrogen, (C₁-C₈)-alkyl, (C₃-C₈)-cycloalkyl, (C₃-C₆)-alkenyl, (C₃-C₆)-alkynyl,
R_{D}⁴ represents halogen, (C₁-C₄)-alkyl, (C₁-C₄)-alkoxy, CF₃,
m_{D} represents 1 or 2;
   for example
   1-[4-(N-2-methoxybenzoylsulfamoyl)phenyl]-3-methylurea,
   1-[4-(N-2-methoxybenzoylsulfamoyl)phenyl]-3,3-dimethylurea,
   1-[4-(N-4,5-dimethylbenzoylsulfamoyl)phenyl]-3-methylurea,
      and also
   N-phenylsulfonylterephthalamides of the formula (S4^{d}), which are known, for example, from CN 101838227,

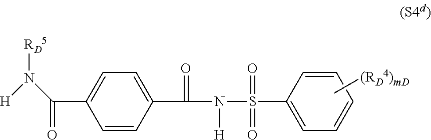

e.g. those in which
R_{D}⁴ represents halogen, (C₁-C₄)-alkyl, (C₁-C₄)-alkoxy, CF₃;
m_{D} represents 1 or 2;
R_{D}⁵ represents hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active compounds from the class of the hydroxyaromatics and the aromatic-aliphatic carboxylic acid derivatives (S5), for example
   ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,

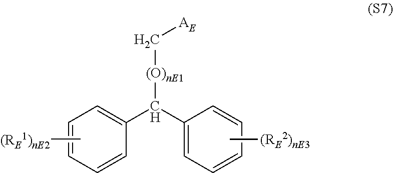

in which the symbols and indices are defined as follows:
R_{E}¹, R_{E}² independently of one another are halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{E} is COOR_{E}³ or COSR_{E}⁴
R_{E}³, R_{E}⁴ independently of one another are hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 or 1
n_{E}², n_{E}³ independently of one another are 0, 1 or 2,
   preferably:
   diphenylmethoxyacetic acid,
   ethyl diphenylmethoxyacetate,
   methyl diphenylmethoxyacetate (CAS reg. no. 41858-19-9) (S7-1).
S8) Compounds of the formula (S8), as described in WO-A-98/27049,

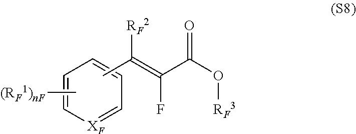

in which
X_{F} represents CH or N,
n_{F} in the case that X_{F}═N is an integer from 0 to 4 and in the case that X_{F}═CH is an integer from 0 to 5,
R_{F}¹ represents halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² represents hydrogen or (C₁-C₄)-alkyl,
R_{F}³ represents hydrogen, (C₁-C₅)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,
   preferably compounds in which
X_{F} represents CH,
n_{F} represents an integer from 0 to 2,
R_{F}¹ represents halogen, (C₁-C₄)-alkyl, (C₁-C₄)-haloalkyl, (C₁-C₄)-alkoxy, (C₁-C₄)-haloalkoxy,
R_{F}² represents hydrogen or (C₁-C₄)-alkyl,
R_{F}³ represents hydrogen, (C₁-C₅)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy,
   or salts thereof.
S9) Active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10^{a}) or (S10^{b})
   as described in WO-A-2007/023719 and WO-A-2007/023764

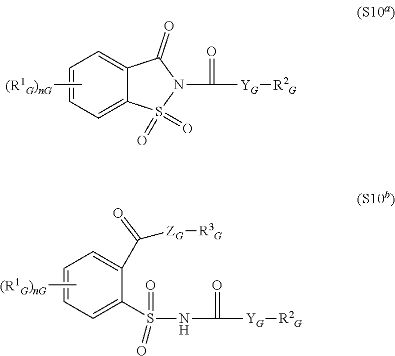

in which
R_{G}¹ represents halogen, (C₁-C₄)-alkyl, methoxy, nitro, cyano, CF₃, OCF₃,
Y_{G}, Z_{G} independently of one another represent O or S,
n_{G} represents an integer from 0 to 4,
R_{G}² represents (C₁-C₁₆)-alkyl, (C₂-C₆)-alkenyl, (C₃-C₆)-cycloalkyl, aryl; benzyl, halobenzyl,
R_{G}³ represents hydrogen or (C₁-C₆)-alkyl.

S11) Active compounds of the oxyimino compound type (S11), which are known as seed-dressing agents, for example
“oxabetrinil” ((Z)-1,3-dioxolan-2-ylmethoxyimino(phenyl)acetonitrile) (S11-1), which is known as a seed-dressing safener for millet/sorghum against metolachlor damage,
“fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
“cyometrinil” or “CGA-43089” ((Z)-cyanomethoxyimino(phenyl)acetonitrile) (S11-3), which is known as a seed-dressing safener for millet/sorghum against metolachlor damage.
S12) Active compounds from the class of the isothiochromanones (S12), for example methyl [(3-oxo-1H-2-benzothiopyran-4 (3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8) (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5) (2-propenyl 1-oxa-4-azaspiro[4.5]decane-4-carbodithioate) (S13-6) from Nitrokemia,
   “disulfoton” (0,0-diethyl S-2-ethylthioethyl phosphorodithioate) (S13-7),
   “dietholate” (0,0-diethyl O-phenyl phosphorothioate) (S13-8),
   “mephenate” (4-chlorophenyl methylcarbamate) (S13-9).
S14) Active compounds which, in addition to herbicidal action against harmful plants, also have safener action on crop plants such as rice, for example
   “dimepiperate” or “MY 93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as a safener for rice against imazosulfuron herbicide damage,
   “cumyluron”=“JC 940” (3-(2-chlorophenylmethyl)-1-(1-methyl-1-phenylethyl)urea, see JP-A-60087254), which is known as safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

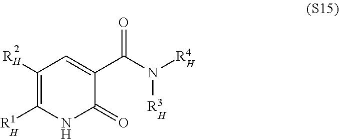

as described in WO-A-2008/131861 and WO-A-2008/131860
in which
R_{H}¹ represents a (C₁-C₆)-haloalkyl radical and
R_{H}² represents hydrogen or halogen and
R_{H}³, R_{H}⁴ independently of one another represent hydrogen, (C₁-C₁₆)-alkyl, (C₂-C₁₆)-alkenyl or (C₂-C₁₆)-alkynyl,
   where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
   where each of the 4 latter radicals is unsubstituted or substituted by one or more radicals from the group consisting of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
R_{H}³ represents (C₁-C₄)-alkoxy, (C₂-C₄)-alkenyloxy, (C₂-C₆)-alkynyloxy or (C₂-C₄)-haloalkoxy and
R_{H}⁴ represents hydrogen or (C₁-C₄)-alkyl or
R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Preferred safeners are: cloquintocet-mexyl, cyprosulfamide, fenchlorazole-ethyl, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

For application, the formulations in the commercial form are diluted if appropriate in a customary manner, for example with water in the case of wettable powders, emulsifiable concentrates, dispersions and water-dispersible granules. Dust-type preparations, granules for soil application or granules for scattering and sprayable solutions are not normally diluted further with other inert substances prior to application.

The required application rate of the compounds of the general formula (varies according to the external conditions such as, inter alia, temperature, humidity and the type of herbicide used. It can vary within wide limits, for example between 0.001 and 10.0 kg/ha or more of active substance, but it is preferably between 0.005 and 5 kg/ha.

The present invention is illustrated in detail by the examples which follow, but these examples do not restrict the invention in any way.

### A. SYNTHESIS EXAMPLES

### N-[(1R)-indan-1-yl]-4-oxo-5,6,7,8-tetrahydro-[1,2,4]triazolo[5,1-b][1,3]thiazepine-2-amine (ex. I-369)

To a solution of 0.050 g (0.18 mmol) of N-[(1R)-indan-1-yl]-5,6,7,8-tetrahydro-[1,2,4]triazolo[5,1-b][1,3]thiazepine-2-amine in 5 ml of acetic acid were added 0.02 ml (0.18 mmol) of hydrogen peroxide and catalytic amounts of sodium tungstate, and the mixture was stirred at room temperature overnight. The reaction mixture was concentrated under reduced pressure without further purification. By column chromatography purification of the resultant crude product (acetonitrile/water gradient), N-[(1R)-indan-1-yl]-4-oxo-5,6,7,8-tetrahydro-[1,2,4]triazolo[5,1-b][1,3]thiazepine-2-amine (0.015 g, 28% of theory) was isolated in solid form.

### N-[(1R)-indan-1-yl]-4,4-dioxo-5,6,7,8-tetrahydro-[1,2,4]triazolo[5,1-b][1,3]thiazepin-2-amine (ex. I-364)

To a solution of 0.050 g (0.18 mmol) of N-[(1R)-indan-1-yl]-5,6,7,8-tetrahydro-[1,2,4]triazolo[5,1-b][1,3]thiazepine-2-amine in 5 ml of acetic acid were added 0.04 ml (0.36 mmol) of hydrogen peroxide and catalytic amounts of sodium tungstate, and the mixture was stirred at room temperature overnight. The reaction mixture was concentrated under reduced pressure without further purification. By column chromatography purification of the resultant crude product (acetonitrile/water gradient), N-[(1R)-indan-1-yl]-4,4-dioxo-5,6,7,8-tetrahydro-[1,2,4]triazolo[5,1-b][1,3]thiazepine-2-amine (0.026 g, 47% of theory) was isolated in solid form.

N-[(1R)-indan-1-yl]-5,6,7,8-tetrahydro-[1,2,4]triazolo[5,1-b][1,3]thiazepine-2-amine (ex. I-352) To a solution under argon of 0.3 g (1.28 mmol) of 2-bromo-5,6,7,8-tetrahydro-[1,2,4]triazolo[5,1-b][1,3]thiazepine and 0.25 g (1.92 mmol) of (1R)-indane-1-amine in 5 ml of dry 1,4-dioxane were added 0.13 g (0.25 mmol) of BrettPhos, 0.16 g (0.12 mmol) of 3G BrettPhos precatalyst and 0.30 g (3.2 mmol) of sodium tert-butoxide. The resulting reaction mixture was then stirred in a microwave apparatus at a temperature of 120° C. for 1.5 h and, after cooling to room temperature, concentrated under reduced pressure. The resulting residue was taken up with dichloromethane, water was added, and the water phase was thoroughly extracted repeatedly with dichloromethane. The combined organic phases were washed with sat. sodium chloride solution, dried over sodium sulfate and concentrated under reduced pressure. By column chromatography purification of the resultant crude product (ethyl acetate/heptane gradient), N-[(1R)-indan-1-yl]-5,6,7,8-tetrahydro-[1,2,4]triazolo[5,1-b][1,3]thiazepine-2-amine (0.19 g, 52% of theory) was isolated in solid form.

### 2-bromo-5,6,7,8-tetrahydro-[1,2,4]triazolo[5,1-b][1,3]thiazepine

8 g (46.9 mmol) of 2-amino-5,6,7,8-tetrahydro-[1,2,4]triazolo[5,1-b][1,3]thiazepine, 11.5 g of copper(II) bromide (51.6 mmol) and 10.7 g (93.9 mmol) of tert-butyl nitrite were heated in 50 ml of acetonitrile at 90° C. for 2 h. The reaction mixture was concentrated under reduced pressure without further purification. The resulting residue was taken up with dichloromethane and washed with 150 ml of 2 N NaOH, and the water phase was thoroughly extracted repeatedly with dichloromethane. The combined organic phases were washed with sat. sodium chloride solution, dried over sodium sulfate and concentrated under reduced pressure. By column chromatography purification of the resultant crude product (ethyl acetate/heptane gradient), 2-bromo-5,6,7,8-tetrahydro-[1,2,4]triazolo[5,1-b][1,3]thiazepine (1.7 g, 15% of theory) was isolated as an oil.

### N-[(1R)-indan-1-yl]-4-oxo-5,6,7,8-tetrahydro-[1,2,4]triazolo[5,1-b][1,3]thiazepine-2-amine (ex. I-369)

To a solution of 0.050 g (0.18 mmol) of N-[(1R)-indan-1-yl]-5,6,7,8-tetrahydro-[1,2,4]triazolo[5,1-b][1,3]thiazepine-2-amine in 5 ml of acetic acid were added 0.02 ml (0.18 mmol) of hydrogen peroxide and catalytic amounts of sodium tungstate, and the mixture was stirred at room temperature overnight. The reaction mixture was concentrated under reduced pressure without further purification. By column chromatography purification of the resultant crude product (acetonitrile/water gradient), N-[(1R)-indan-1-yl]-4-oxo-5,6,7,8-tetrahydro-[1,2,4]triazolo[5,1-b][1,3]thiazepine-2-amine (0.015 g, 28% of theory) was isolated in solid form.

### N-[(1R)-indan-1-yl]-4,4-dioxo-5,6,7,8-tetrahydro-[1,2,4]triazolo[5,1-b][1,3]thiazepin-2-amine (ex. I-364)

To a solution of 0.050 g (0.18 mmol) of N-[(1R)-indan-1-yl]-5,6,7,8-tetrahydro-[1,2,4]triazolo[5,1-b][1,3]thiazepine-2-amine in 5 ml of acetic acid were added 0.04 ml (0.36 mmol) of hydrogen peroxide and catalytic amounts of sodium tungstate, and the mixture was stirred at room temperature overnight. The reaction mixture was concentrated under reduced pressure without further purification. By column chromatography purification of the resultant crude product (acetonitrile/water gradient), N-[(1R)-indan-1-yl]-4,4-dioxo-5,6,7,8-tetrahydro-[1,2,4]triazolo[5,1-b][1,3]thiazepine-2-amine (0.026 g, 47% of theory) was isolated in solid form.

NMR Data of Selected Examples

NMR Peak List Method

The 1H NMR data of selected examples are noted in the form of 1H NMR peak lists. For each signal peak, first the δ value in ppm and then the signal intensity in round brackets are listed. The pairs of 6 value-signal intensity numbers for different signal peaks are listed with separation from one another by semicolons.

The peak list for one example therefore takes the form of:

δ₁ (intensity₁); δ₂ (intensity₂); . . . δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists.

The lists of the 1H NMR peaks are similar to the conventional 1H NMR printouts and thus usually contain all peaks listed in a conventional NMR interpretation.

In addition, like conventional 1H NMR printouts, they may show solvent signals, signals of stereoisomers of the target compounds, which likewise form part of the subject matter of the invention, and/or peaks of impurities.

In the reporting of compound signals in the delta range of solvents and/or water, our lists of 1H NMR peaks show the usual solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of 1H NMR peak lists can be found in the Research Disclosure Database Number 564025.

1H-NMR

I-019: ¹H-NMR(601.6 MHz, d₆-DMSO):

δ=7.5224 (2.0); 7.5068 (5.2); 7.5027 (6.0); 7.4943 (2.3); 7.4790 (0.7); 7.2640 (1.0); 7.2223 (1.2); 7.2105 (1.7); 7.1848 (0.8); 7.1728 (1.6); 7.1607 (0.8); 7.1449 (1.0); 7.1336 (1.3); 7.1205 (0.5); 6.2563 (0.6); 6.2417 (0.6); 5.0460 (0.5); 4.7458 (0.3); 4.7255 (0.4); 4.7198 (0.4); 4.7112 (0.4); 4.6935 (0.3); 4.0784 (0.4); 4.0701 (0.4); 4.0151 (2.2); 3.9084 (0.3); 3.8994 (16.0); 3.3251 (0.7); 3.3056 (49.9); 3.1739 (1.4); 3.1652 (1.4); 2.9319 (0.4); 2.9075 (0.6); 2.8977 (0.5); 2.7802 (0.4); 2.7679 (0.6); 2.7539 (0.6); 2.7426 (0.5); 2.6119 (0.6); 2.5207 (1.1); 2.5180 (1.4); 2.5151 (1.4); 2.5030 (87.2); 2.5001 (119.5); 2.4973 (90.2); 2.4362 (0.7); 2.4126 (0.3); 2.3875 (0.6); 2.3842 (0.7); 1.8281 (0.5); 1.8139 (0.5); −0.0002 (12.0)

I-022: ¹H-NMR(400.0 MHz, d₆-DMSO):

δ=7.5927 (11.1); 7.5756 (6.1); 7.5406 (3.1); 7.5214 (5.8); 7.5005 (3.8); 7.4844 (4.6); 7.4668 (2.2); 7.2961 (2.8); 7.1364 (1.0); 7.1072 (7.5); 7.0692 (4.5); 7.0507 (2.4); 6.2195 (1.9); 6.1996 (2.0); 4.6967 (2.7); 4.6145 (0.6); 4.6055 (0.6); 4.5968 (0.6); 4.5774 (0.6); 4.5391 (0.6); 4.5012 (0.4); 4.4938 (0.3); 4.1490 (0.4); 4.1368 (0.5); 4.0996 (0.6); 4.0796 (0.6); 3.9984 (6.0); 3.8991 (0.4); 3.8879 (0.5); 3.8311 (0.7); 3.7857 (1.2); 3.7762 (1.2); 3.3258 (312.7); 2.9944 (16.0); 2.7674 (0.9); 2.7277 (2.6); 2.7107 (3.2); 2.6966 (3.1); 2.6750 (2.4); 2.6703 (2.7); 2.6467 (0.8); 2.5410 (134.1); 2.5061 (261.4); 2.5017 (334.7); 2.4972 (245.3); 2.3669 (0.6); 2.3284 (2.0); 1.9082 (3.4); 1.8016 (1.0); 1.7788 (1.8); 1.7583 (1.9); 1.7211 (1.2); 1.6918 (1.7); 1.2585 (0.5); 1.2392 (1.6); 0.8525 (0.4); −0.0002 (10.3)

I-023: ¹H-NMR(601.6 MHz, d₆-DMSO):

δ=7.5909 (2.6); 7.5785 (1.4); 7.5766 (1.5); 7.5729 (1.0); 7.5337 (1.0); 7.5210 (1.9); 7.5076 (1.1); 7.4841 (0.8); 7.4751 (0.5); 7.4696 (0.6); 7.2765 (0.6); 7.2651 (0.7); 7.2227 (0.8); 7.2115 (1.3); 7.1853 (0.6); 7.1735 (1.2); 7.1609 (0.6); 7.1451 (0.7); 7.1331 (0.9); 7.1216 (0.4); 6.2605 (0.4); 6.2452 (0.4); 5.0709 (0.3); 5.0511 (0.4); 5.0412 (0.3); 4.0698 (0.3); 4.0093 (1.5); 3.8995 (16.0); 3.3252 (1.1); 3.3065 (45.9); 3.2680 (0.4); 3.1732 (1.3); 3.1656 (1.3); 2.9341 (0.3); 2.9125 (0.4); 2.8988 (0.4); 2.7667 (0.4); 2.7552 (0.4); 2.7410 (0.3); 2.6118 (0.6); 2.5212 (1.4); 2.5181 (1.6); 2.5150 (1.5); 2.5062 (37.5); 2.5032 (82.2); 2.5002 (115.9); 2.4972 (83.8); 2.4942 (39.6); 2.4640 (0.4); 2.4479 (0.4); 2.4393 (0.4); 2.3878 (0.5); 2.3844 (0.7): 1.8311 (0.3); 1.8176 (0.4); 1.8130 (0.4); 0.0053 (0.4); −0.0002 (15.0); −0.0057 (0.6)

I-024: ¹H-NMR(601.6 MHz, d₆-DMSO):

δ=11.9373 (0.5); 7.2634 (1.4); 7.2514 (1.3); 7.1258 (0.9); 7.1143 (1.8); 7.1026 (1.0); 6.8425 (1.2); 6.8302 (2.2); 6.8191 (1.0); 6.7513 (2.2); 6.7379 (2.0); 6.3783 (0.9); 6.3639 (0.6); 4.7401 (1.3); 4.7303 (1.4); 4.7166 (1.1); 4.7072 (0.6); 4.5763 (1.6); 4.2532 (0.6); 4.2398 (1.3); 4.2268 (0.8); 4.2219 (0.8); 4.1900 (0.8); 4.1844 (1.0); 4.1796 (1.0); 4.1730 (1.2); 4.1665 (0.6); 4.1608 (0.7); 4.1554 (0.6); 3.9909 (3.1); 3.9583 (0.6); 3.9543 (0.6); 3.9399 (0.7); 3.8995 (16.0); 3.8776 (0.6); 3.3250 (0.6); 3.3049 (58.3); 3.1736 (0.6); 3.1649 (0.6); 2.7237 (0.6); 2.7128 (0.6); 2.7003 (0.4); 2.6146 (0.5); 2.6116 (0.8); 2.6087 (0.5); 2.5211 (1.5); 2.5180 (1.8); 2.5149 (1.8); 2.5061 (46.2); 2.5031 (100.5); 2.5001 (139.5); 2.4970 (100.1); 2.4940 (46.4); 2.3875 (0.5); 2.3840 (0.8); 2.3813 (0.5); 2.1818 (0.4); 2.0609 (0.4); 2.0554 (0.5); 2.0467 (0.8); 2.0405 (0.8); 2.0326 (1.0); 2.0257 (0.8); 2.0180 (0.7); 2.0026 (0.7); 1.8622 (0.3); 1.8088 (0.4); 1.7868 (0.4); 1.6995 (1.7); 1.6861 (1.6); 1.6439 (1.6); 1.3607 (0.8); 1.3304 (2.6); 1.2860 (0.5); 1.2763 (0.4); 1.2722 (0.4); 1.2587 (0.5); 1.2521 (0.6); 1.2364 (0.9); 1.2122 (0.4); 1.1949 (0.5); 1.1804 (0.7); 1.1749 (0.7); 1.1712 (0.7); 0.0052 (0.5); −0.0002 (19.6); −0.0057 (0.7)

1H-NMR

I-028: ¹H-NMR(400.0 MHz, d₆-DMSO):

δ=7.5541 (16.0); 7.3134 (0.8); 7.2976 (0.9); 7.1187 (1.2); 7.1075 (2.6); 7.0915 (1.2); 7.0704 (1.6); 7.0501 (0.7); 6.2212 (0.8); 6.1987 (0.8); 4.6913 (1.0); 4.6499 (0.4); 3.9977 (2.3); 3.3260 (68.6); 2.9944 (4.5); 2.7444 (0.5); 2.7262 (0.8); 2.7123 (1.0); 2.6965 (1.0); 2.6802 (0.6); 2.6700 (0.7); 2.6656 (0.6); 2.5408 (35.4); 2.5056 (59.1); 2.5014 (75.1); 2.4972 (55.5); 2.3277 (0.4); 1.9067 (1.1); 1.7779 (0.6); 1.7580 (0.6); 1.7377 (0.4); 1.7209 (0.4); 1.6887 (0.5); 1.2404 (0.5); −0.0002 (2.0)

I-029: ¹H-NMR(601.6 MHz, d₆-DMSO):

δ=7.5944 (1.0); 7.5808 (1.4); 7.5619 (0.7); 7.5349 (0.8); 7.5321 (0.9); 7.5221 (1.7); 7.5193 (1.7); 7.5095 (1.5); 7.5064 (1.3); 7.4965 (1.0); 7.4934 (0.9); 7.4795 (1.1); 7.4671 (1.6); 7.4566 (1.1); 7.3276 (0.7); 7.3149 (0.7); 7.2886 (0.5); 7.2770 (0.5); 7.1231 (0.9); 7.1120 (1.7); 7.1000 (1.2); 7.0813 (0.5); 7.0728 (1.0); 7.0579 (1.0); 7.0453 (0.3); 6.2264 (0.6); 6.2113 (0.5); 6.1671 (0.3); 4.9316 (0.3); 4.9034 (0.4); 4.7170 (0.6); 4.7045 (0.5); 4.6902 (0.5); 4.6620 (0.3); 4.6554 (0.4); 4.3281 (0.4); 4.3080 (0.4); 3.9303 (0.4); 3.9198 (0.5); 3.9117 (0.6); 3.8994 (16.0); 3.6861 (0.3); 3.6768 (0.4); 3.6285 (0.5); 3.3252 (1.3); 3.3088 (29.4); 3.2682 (0.4); 3.1693 (2.6); 2.7522 (0.4); 2.7446 (0.5); 2.7401 (0.5); 2.7316 (0.5); 2.7170 (0.4); 2.7097 (0.5); 2.6998 (0.6); 2.6901 (0.5); 2.6815 (0.5); 2.6712 (0.4); 2.6149 (0.4); 2.6120 (0.7); 2.5212 (1.0); 2.5180 (1.3); 2.5149 (1.5); 2.5061 (38.3); 2.5032 (80.3); 2.5002 (109.7); 2.4972 (79.8); 2.4943 (38.0); 2.3842 (0.6); 2.3814 (0.5); 1.9513 (0.3); 1.9464 (0.3); 1.9304 (0.6); 1.9235 (0.8); 1.8964 (0.7); 1.7908 (0.4); 1.7736 (0.5); 1.7570 (0.4); 1.7394 (0.3); 1.7124 (0.4); 1.7011 (0.4); 1.6958 (0.4); 1.6817 (0.4); −0.0002 (12.2)

I-030: ¹H-NMR(601.6 MHz, d₆-DMSO):

δ=7.5278 (0.6); 7.5211 (1.4); 7.5009 (4.9); 7.4927 (1.5); 7.4781 (0.4); 7.3010 (0.6); 7.1273 (0.4); 7.1162 (1.0); 7.1070 (1.7); 7.1041 (1.7); 7.0941 (0.9); 7.0822 (0.4); 7.0659 (1.0); 7.0527 (0.7); 6.1883 (0.4); 6.1714 (0.4); 4.7296 (0.3); 4.6954 (0.6); 4.0046 (1.6); 3.8995 (16.0); 3.3250 (0.7); 3.3069 (43.8); 3.1719 (0.6); 3.1662 (0.6); 2.7444 (0.4); 2.7326 (0.5); 2.7022 (0.4); 2.6925 (0.6); 2.6149 (0.4); 2.6119 (0.6); 2.6088 (0.5); 2.5211 (1.0); 2.5181 (1.2); 2.5150 (1.3); 2.5062 (35.2); 2.5032 (75.6); 2.5002 (104.2); 2.4971 (74.8); 2.4942 (34.7); 2.3876 (0.4); 2.3844 (0.6); 2.3814 (0.4); 1.9341 (0.4); 1.9137 (0.8); 1.8899 (0.4); 1.7764 (0.4); 1.7640 (0.5); 1.6919 (0.4); 1.6845 (0.4); 0.0053 (0.3); −0.0002 (13.0); −0.0056 (0.5)

I-033: ¹H-NMR(601.6 MHz, d₆-DMSO):

δ=7.2714 (2.3); 7.2590 (2.8); 7.2229 (2.1); 7.2116 (3.4); 7.1840 (1.6); 7.1722 (3.1); 7.1602 (1.6); 7.1429 (2.2); 7.1311 (2.7); 7.1190 (1.0); 6.2214 (1.4); 6.2072 (1.4); 5.0709 (0.6); 5.0581 (1.4); 5.0439 (1.5); 5.0311 (0.6); 4.7398 (1.2); 4.5748 (2.4); 3.9893 (5.0); 3.9544 (0.9); 3.9442 (1.0); 3.8994 (16.0); 3.8794 (0.9); 3.3055 (76.4); 3.1736 (0.5); 3.1651 (0.5); 2.9339 (0.7); 2.9184 (0.8); 2.9137 (1.0); 2.9070 (0.9); 2.8965 (0.9); 2.7809 (0.8); 2.7663 (1.7); 2.7535 (1.5); 2.7410 (1.6); 2.7268 (1.5); 2.6146 (0.7); 2.6119 (0.9); 2.6088 (0.7); 2.5208 (1.5); 2.5180 (1.8); 2.5147 (1.9); 2.5059 (54.2); 2.5030 (115.9); 2.5000 (161.2); 2.4969 (116.5); 2.4940 (54.9); 2.4641 (0.8); 2.4582 (0.8); 2.4511 (1.2); 2.4458 (1.4); 2.4384 (1.4); 2.4305 (1.4); 2.4251 (1.2); 2.4180 (0.7); 2.4128 (0.6); 2.3843 (0.9); 2.3811 (0.7); 1.8305 (0.8); 1.8086 (1.0); 1.7970 (0.8); 1.7807 (0.4); 1.7017 (2.6); 1.6681 (1.1); 1.6366 (1.7); 1.3341 (3.6); 1.2813 (0.3); 1.2590 (0.4); 1.2496 (0.4); 1.2364 (0.4); 1.2024 (0.4); 1.1822 (0.7); 1.1682 (0.8); 0.0051 (0.5); −0.0002 (20.1); −0.0059 (0.8)

I-036: ¹H-NMR(601.6 MHz, d₆-DMSO):

δ=7.8895 (2.3); 7.8795 (2.2); 7.8439 (0.7); 7.8296 (0.8); 7.7471 (1.1); 7.7342 (1.6); 7.7209 (0.7); 7.2639 (0.7); 7.2507 (0.3); 7.2244 (1.0); 7.2112 (1.4); 7.1850 (0.7); 7.1732 (1.3); 7.1618 (0.8); 7.1450 (0.8); 7.1326 (1.0); 6.2498 (0.5); 6.2435 (0.4); 5.0556 (0.4); 4.7619 (0.4); 4.7538 (0.4); 4.7475 (0.3); 4.0152 (1.5); 3.8996 (16.0); 3.7877 (0.3); 3.7774 (0.4); 3.3252 (0.9); 3.3061 (66.2); 3.2678 (0.3); 3.1735 (0.8); 3.1654 (0.9); 2.9338 (0.4); 2.9081 (0.5); 2.8944 (0.4); 2.7660 (0.5); 2.7535 (0.5); 2.7406 (0.4); 2.6122 (0.7); 2.5212 (1.0); 2.5182 (1.4); 2.5151 (1.5); 2.5061 (46.4); 2.5033 (99.3); 2.5003 (137.8); 2.4973 (101.4); 2.4944 (49.7); 2.4427 (0.6); 2.3843 (0.8); 1.8124 (0.5); 1.8027 (0.4); −0.0002 (14.3); −0.0056 (0.7)

1H-NMR

I-042: ¹H-NMR(400.0 MHz, d₆-DMSO):

δ=8.9821 (4.7); 8.9715 (2.8); 8.9571 (1.0); 8.8864 (1.5); 7.9255 (2.1); 7.9125 (2.1); 7.8949 (1.1); 7.8819 (1.0); 7.2806 (1.3); 7.2620 (1.5); 7.2407 (0.7); 7.2220 (0.7); 7.1454 (0.7); 7.1422 (0.7); 7.1254 (1.8); 7.1072 (1.6); 7.0911 (0.5); 6.8619 (1.0); 6.8436 (1.8); 6.8246 (1.0); 6.8182 (1.0); 6.7983 (0.4); 6.7641 (2.0); 6.7443 (2.7); 6.7264 (1.0); 6.4815 (0.8); 6.4619 (0.8); 6.4243 (0.6); 6.4033 (0.6); 5.0220 (0.5); 4.9795 (0.7); 4.7679 (0.4); 4.7551 (0.9); 4.7439 (0.7); 4.7347 (0.8); 4.7211 (0.5); 4.7131 (0.4); 4.6983 (0.6); 4.6743 (1.0); 4.6289 (0.5); 4.4693 (0.4); 4.3499 (0.6); 4.2711 (0.5); 4.2512 (1.0); 4.2438 (0.9); 4.2315 (0.9); 4.2226 (1.1); 4.2130 (1.0); 4.2026 (1.2); 4.1876 (1.0); 4.1700 (0.9); 4.1560 (0.6); 4.1371 (0.4); 4.1284 (0.4); 4.1105 (0.8); 4.0974 (1.9); 4.0843 (1.9); 4.0711 (0.8); 4.0331 (0.6); 4.0063 (0.8); 3.9648 (0.4); 3.9017 (16.0); 3.8355 (0.6); 3.7200 (1.7); 3.7068 (2.7); 3.6937 (1.3); 3.3220 (102.8); 3.2672 (0.5); 3.1750 (7.2); 3.1619 (7.0); 2.8133 (0.4); 2.6750 (0.8); 2.6707 (1.1); 2.6663 (0.8); 2.5407 (1.0); 2.5060 (138.0); 2.5016 (175.6); 2.4971 (131.3); 2.3328 (0.8); 2.3284 (1.0); 2.3239 (0.8); 2.0663 (0.6); 2.0544 (0.8); 2.0452 (0.9); 2.0242 (1.3); 2.0153 (1.2); 1.9999 (1.0); 1.9873 (0.9); 1.9770 (0.7); 1.2589 (0.4); 1.2492 (0.6); 1.2357 (1.5); 0.0077 (0.8); −0.0003 (19.1); −0.0084 (0.8)

I-043: ¹H-NMR(601.6 MHz, d₆-DMSO):

δ=8.9774 (2.9); 8.9694 (1.2); 8.9637 (0.6); 8.9551 (0.6); 8.8828 (0.8); 7.9174 (1.2); 7.9084 (1.1); 7.8878 (0.5); 7.8789 (0.6); 7.3309 (0.6); 7.3198 (0.6); 7.2858 (0.4); 7.2729 (0.4); 7.1255 (0.7); 7.1173 (1.3); 7.1078 (0.9); 7.0952 (0.6); 7.0765 (1.0); 7.0649 (0.6); 6.2368 (0.5); 6.2199 (0.4); 4.9797 (0.3); 4.7138 (0.4); 4.7103 (0.4); 4.6563 (0.5); 4.0116 (0.3); 3.8995 (16.0); 3.8163 (0.3); 3.7107 (1.0); 3.7015 (1.4); 3.6923 (0.7); 3.3252 (1.3); 3.3075 (58.4); 3.2840 (0.4); 3.2681 (0.6); 3.1694 (2.6); 2.7445 (0.4); 2.7126 (0.4); 2.7035 (0.4); 2.6915 (0.4); 2.6767 (0.3); 2.6151 (0.4); 2.6121 (0.7); 2.6094 (0.6); 2.5214 (1.0); 2.5184 (1.3); 2.5153 (1.3); 2.5064 (43.9); 2.5034 (97.1); 2.5004 (135.8); 2.4973 (97.7); 2.4943 (45.7); 2.3879 (0.6); 2.3845 (0.8); 2.3817 (0.6); 1.9277 (0.7); 1.9078 (0.6); 1.8885 (0.5); 1.7978 (0.3); 1.7816 (0.4); 1.6975 (0.3); 0.0052 (0.5); −0.0002 (16.2); −0.0058 (0.7)

I-045: ¹H-NMR(601.6 MHz, d₆-DMSO):

δ=7.2761 (0.7); 7.2661 (1.5); 7.2549 (1.0); 7.2234 (1.3); 7.2112 (2.2); 7.1839 (1.0); 7.1722 (1.8); 7.1595 (1.0); 7.1439 (1.0); 7.1304 (1.3); 7.1183 (0.5); 6.2326 (0.6); 6.2150 (1.0); 6.1997 (0.8); 5.0675 (0.6); 5.0553 (0.8); 5.0430 (0.7); 4.6729 (2.2); 4.5825 (3.2); 4.2763 (0.5); 4.0210 (0.8); 4.0129 (1.6); 4.0038 (1.1); 3.9332 (1.9); 3.9240 (3.1); 3.9160 (2.2); 3.8995 (16.0); 3.3251 (0.7); 3.3070 (62.3); 3.1736 (0.9); 3.1648 (0.8); 2.9353 (0.5); 2.9221 (0.5); 2.9165 (0.5); 2.9100 (0.6); 2.8950 (0.6); 2.7825 (0.5); 2.7674 (1.0); 2.7542 (0.9); 2.7419 (0.7); 2.7280 (0.4); 2.6150 (0.4); 2.6119 (0.6); 2.6089 (0.5); 2.5211 (1.0); 2.5179 (1.3); 2.5149 (1.4); 2.5061 (37.8); 2.5031 (81.8); 2.5000 (113.0); 2.4970 (81.5); 2.4940 (37.9); 2.4654 (0.6); 2.4593 (0.5); 2.4523 (0.8); 2.4467 (0.8); 2.4391 (0.9); 2.4315 (0.9); 2.4262 (0.7); 2.4193 (0.5); 2.4134 (0.4); 2.3843 (0.7); 2.1422 (8.0); 2.1054 (4.9); 1.8349 (0.4); 1.8250 (0.6); 1.8110 (0.7); 1.8033 (0.7); 1.7901 (0.5); 0.0053 (0.3); −0.0002 (14.2); −0.0057 (0.6)

I-046: ¹H-NMR(601.6 MHz, d₆-DMSO):

δ=8.6499 (0.8); 8.6415 (1.1); 8.0000 (0.5); 7.9878 (0.8); 7.9749 (0.5); 7.7251 (2.0); 7.7122 (1.8); 7.5630 (0.8); 7.5509 (0.8); 7.5427 (0.7); 7.2890 (0.6); 7.2779 (0.7); 7.2565 (0.5); 7.2436 (0.4); 7.2283 (0.6); 7.2166 (1.2); 7.2038 (0.5); 7.1878 (0.4); 7.1767 (1.0); 7.1650 (0.9); 7.1513 (0.6); 7.1370 (0.9); 7.1245 (0.6); 6.2788 (0.4); 6.2630 (0.4); 5.0790 (0.5); 5.0660 (0.5); 5.0513 (0.3); 4.8131 (1.9); 4.7690 (1.2); 4.1739 (0.7); 4.1641 (0.5); 4.0426 (0.7); 4.0329 (0.6); 4.0178 (1.1); 3.9723 (0.8); 3.9643 (1.1); 3.8996 (16.0); 3.3251 (0.8); 3.3079 (46.0); 3.1731 (0.9); 3.1661 (0.9); 2.9156 (0.4); 2.9053 (0.5); 2.7764 (0.4); 2.7638 (0.4); 2.7503 (0.4); 2.7373 (0.3); 2.6153 (0.4); 2.6120 (0.6); 2.6088 (0.4); 2.5213 (1.0); 2.5183 (1.2); 2.5151 (1.1); 2.5064 (33.7); 2.5034 (76.4); 2.5003 (107.7); 2.4972 (77.0); 2.4942 (35.5); 2.4611 (0.5); 2.4547 (0.5); 2.4474 (0.5); 2.4420 (0.5); 2.3877 (0.4); 2.3845 (0.6); 2.3817 (0.4); 1.8309 (0.4): 1.8233 (0.4); 1.8114 (0.4); 1.7984 (0.4); 0.0052 (0.4); −0.0002 (16.2); −0.0058 (0.6)

1H-NMR

I-047: ¹H-NMR(601.6 MHz, d₆-DMSO):

δ=7.9007 (0.5); 7.8976 (0.4); 7.5884 (2.7); 7.5780 (1.4); 7.5762 (1.5); 7.5460 (0.4); 7.5328 (1.1); 7.5204 (1.6); 7.5064 (1.0); 7.4813 (0.8); 7.4682 (0.5); 7.2630 (0.6); 7.2515 (0.6); 7.1288 (0.6); 7.1150 (1.0); 7.1026 (0.6); 6.8441 (0.7); 6.8320 (1.0); 6.8191 (0.5); 6.7513 (1.1); 6.7378 (1.1); 6.4153 (0.4); 6.4030 (0.5); 4.7295 (0.6); 4.5336 (0.3); 4.2384 (0.5); 4.1874 (0.5); 4.1754 (0.5); 4.1556 (0.3); 4.0786 (0.5); 4.0697 (0.5); 4.0093 (1.4); 3.8997 (16.0); 3.3251 (0.9); 3.3056 (34.4); 3.1737 (2.4); 3.1650 (2.4); 2.6119 (0.6); 2.6088 (0.5); 2.5212 (1.1); 2.5181 (1.6); 2.5149 (1.6); 2.5062 (37.3); 2.5032 (79.8); 2.5002 (109.6); 2.4971 (77.4); 2.4941 (35.6); 2.4760 (0.3); 2.3875 (0.4); 2.3845 (0.6); 2.0403 (0.4); 2.0202 (0.4): 2.0052 (0.5); 1.2366 (0.4); 0.0052 (0.4); −0.0002 (16.6); −0.0057 (0.6)

1-048: ¹H-NMR(400.0 MHz, d₆-DMSO):

δ=8.9835 (2.7); 8.9707 (1.7); 8.9568 (0.7); 8.8859 (0.8); 7.9262 (1.1); 7.9133 (1.2); 7.8971 (0.7); 7.8847 (0.6); 7.6687 (0.3); 7.6501 (0.6); 7.6322 (0.4); 7.6007 (0.3); 7.4203 (0.4); 7.2942 (0.5); 7.2768 (0.6); 7.2379 (0.7); 7.2200 (1.2); 7.2017(0.8); 7.1841 (0.9); 7.1654 (1.1); 7.1461 (0.7); 6.3268 (0.4); 6.3054 (0.4); 5.0819 (0.4); 5.0615 (0.4); 5.0294 (0.4); 5.0122 (0.4); 4.6734 (0.4); 4.4758 (0.3); 4.0218 (0.4); 4.0046 (0.5); 3.9018 (16.0); 3.8311 (0.3); 3.7207 (0.9); 3.7078 (1.3); 3.6947 (0.7); 3.3219 (77.5); 3.2674 (0.4); 3.1684 (2.3); 3.1104 (0.4); 3.0964 (0.3); 2.9134 (0.4); 2.8984 (0.4); 2.8911 (0.4); 2.7849 (0.4); 2.7646 (0.4); 2.6751 (0.6); 2.6705 (0.8); 2.6663 (0.6); 2.6465 (0.5); 2.6314 (0.4); 2.6170 (0.4); 2.5408 (0.6); 2.5102 (56.3); 2.5059 (108.9); 2.5014 (142.1); 2.4969 (107.1); 2.4927 (55.6); 2.4571 (0.8); 2.4465 (0.7); 2.4373 (0.6); 2.4264 (0.5); 2.4176 (0.4); 2.3326 (0.7); 2.3282 (0.9); 2.3237 (0.7); 1.8275 (0.4); 1.8181 (0.5); 1.7972 (0.4); 1.2353 (0.4); 0.0079 (0.6); −0.0002 (17.3); −0.0084 (0.7)

I-049: ¹H-NMR(400.0 MHz, d₆-DMSO):

δ=7.2786 (2.7); 7.2610 (3.4); 7.2285 (2.4); 7.2108 (4.7); 7.1908 (2.1); 7.1754 (3.9); 7.1577 (2.8); 7.1534 (4.0); 7.1338 (3.1); 7.1158 (1.0); 6.2550 (1.9); 6.2340 (1.9); 5.0865 (0.8); 5.0667 (2.2); 5.0462 (2.1); 5.0258 (0.8); 4.9200 (1.2); 4.6035 (1.9); 4.1830 (1.5); 4.0960 (0.4); 4.0829 (0.4); 4.0693 (0.4); 4.0274 (1.7); 3.9916 (0.9); 3.9562 (1.4); 3.9015 (16.0); 3.3206 (131.1); 3.2672 (0.3); 3.1749 (1.0); 3.1618 (1.0); 2.9521 (0.8); 2.9446 (0.9); 2.9302 (1.0); 2.9221 (1.1); 2.9126 (1.5); 2.9052 (1.5); 2.8909 (1.6); 2.8838 (1.4); 2.7948 (1.1); 2.7740 (2.2); 2.7540 (1.8); 2.7348 (1.4); 2.7139 (0.8); 2.6745 (0.8); 2.6701 (1.1); 2.6657 (0.8); 2.5402 (0.8); 2.5054 (141.7); 2.5010 (182.7); 2.4965 (139.0); 2.4617 (2.3); 2.4536 (2.2); 2.4421 (2.0); 2.4308 (2.0); 2.4228 (1.7); 2.4116 (1.0); 2.4037 (0.9); 2.3322 (0.9); 2.3277 (1.2); 2.3233 (0.9); 2.1346 (1.0); 1.8604 (0.5); 1.8391 (1.3); 1.8176 (1.5); 1.8084 (1.4); 1.7974 (0.8); 1.7873 (1.2); 1.7655 (0.4); 0.7924 (12.2); 0.7847 (8.7); 0.7725 (6.5); 0.0076 (0.9); −0.0002 (20.2); −0.0083 (1.0)

I-051: ¹H-NMR(400.0 MHz. d₆-DMSO):

δ=8.3146 (0.3); 7.8803 (7.8); 7.8446 (2.6); 7.8265 (3.5); 7.7523 (3.5); 7.7330 (4.8); 7.7123 (1.9); 7.2978 (2.4); 7.1378 (0.9); 7.1188 (3.6); 7.1081 (6.9); 7.0709 (4.5); 7.0522 (2.2); 6.2208 (1.6); 4.7591 (1.4); 4.7334 (1.4); 4.6992 (2.1); 4.6928 (2.1); 4.6373 (0.6); 4.6277 (0.6); 4.5651 (0.6); 4.1368 (0.6); 4.1202 (0.6); 4.0501 (0.6); 4.0046 (5.6); 3.9032 (0.4); 3.8978 (0.4); 3.7756 (1.2); 3.7657 (1.1); 3.7133 (0.4); 3.3261 (203.2); 2.9945 (16.0); 2.7725 (0.8); 2.7682 (0.8); 2.7289 (2.5); 2.7116 (3.1); 2.6959 (2.8); 2.6752 (2.3); 2.6708 (2.6); 2.6097 (0.4); 2.5833 (0.5); 2.5410 (118.0); 2.5060 (256.1); 2.5016 (326.8); 2.4973 (235.2); 2.3676 (0.5); 2.3282 (1.8); 1.9092 (3.2); 1.8016 (0.9); 1.7809 (1.6); 1.7576 (1.7); 1.6893 (1.6); 1.2979 (0.4); 1.2631 (0.5); 1.2583 (0.6); 1.2403 (2.2); 0.9399 (0.3); 0.8533 (0.6); 0.8340 (0.3); −0.0002 (7.3); −0.0086 (0.3)

1H-NMR

I-054: ¹H-NMR(601.6 MHz, d₆-DMSO):

δ=7.5956 (1.2); 7.5828 (1.6); 7.5769 (0.7); 7.5632 (0.9); 7.5344 (1.0); 7.5232 (2.0); 7.5113 (1.8); 7.5081 (1.6); 7.4984 (1.2); 7.4949 (1.0); 7.4817 (1.2); 7.4692 (1.8); 7.4603 (1.4); 7.4574 (1.4); 7.2886 (0.9); 7.2761 (0.9); 7.2485 (0.6); 7.2354 (0.7); 7.2297 (0.8); 7.2167 (1.6); 7.2026 (0.8); 7.1909 (0.6); 7.1786 (1.5); 7.1675 (1.2); 7.1532 (1.0); 7.1415 (1.0); 7.1248 (0.6); 6.2950 (0.8); 6.2801 (0.8); 6.2329 (0.4); 6.2187 (0.4); 5.0857 (0.3); 5.0725 (0.7); 5.0596 (0.7); 5.0247 (0.4); 5.0125 (0.4); 4.9375 (0.4); 4.9091 (0.6); 4.7291 (0.6); 4.7021 (0.4); 4.3369 (0.5); 4.3086 (0.5); 4.0877 (0.4); 4.0796 (0.8); 4.0709 (0.8); 4.0619 (0.5); 3.9154 (0.7); 3.8994 (16.0); 3.6923 (0.4); 3.6828 (0.5); 3.6342 (0.6); 3.3251 (1.2); 3.3078 (68.6); 3.2680 (0.4); 3.1738 (2.6); 3.1651 (2.6); 2.9317 (0.4); 2.9258 (0.5); 2.9193 (0.6); 2.9144 (0.5); 2.9047 (0.5); 2.8998 (0.6); 2.7763 (0.5); 2.7626 (0.5); 2.7510 (0.7); 2.7372 (0.5); 2.6120 (0.6); 2.5210 (1.1); 2.5181 (1.3); 2.5150 (1.6); 2.5062 (39.8); 2.5033 (83.2); 2.5003 (113.1); 2.4972 (82.0); 2.4943 (38.9); 2.4722 (0.6); 2.4648 (0.6); 2.4595 (0.6); 2.4520 (0.6); 2.4445 (0.6); 2.4390 (0.6); 2.4314 (0.5); 2.4264 (0.5); 2.3874 (0.6); 2.3843 (0.6); 1.8409 (0.5); 1.8269 (0.6); 1.8210 (0.6); 1.8123 (0.4); 1.8070 (0.7); 1.7929 (0.4); −0.0002 (12.2)

I-055: ¹H-NMR(601.6 MHz, d₆-DMSO):

δ=7.2695 (2.4); 7.2563 (2.4); 7.1288 (1.7); 7.1149 (3.1); 7.1030 (1.7); 6.8461 (2.1); 6.8336 (3.7); 6.8214 (1.8); 6.7518 (4.1); 6.7385 (3.8); 6.3852 (1.3); 4.9219 (1.0); 4.9112 (0.8); 4.7372 (1.7); 4.7233 (1.7); 4.7152 (0.9); 4.6025 (1.5); 4.2560 (1.0); 4.2422 (2.0); 4.2290 (1.5); 4.1922 (2.2); 4.1872 (2.6); 4.1819 (2.8); 4.1752 (3.0); 4.1633 (1.5); 4.1581 (1.2); 4.0333 (1.4); 4.0005 (0.5); 3.9573 (1.1); 3.8994 (16.0); 3.3248 (1.0); 3.3082 (65.7); 3.3064 (72.7); 3.1736 (0.5); 3.1653 (0.5); 2.6128 (1.0); 2.5394 (0.4); 2.5179 (2.4); 2.5151 (2.8); 2.5031 (128.1); 2.5002 (175.8); 2.4974 (131.8); 2.4760 (0.7); 2.3844 (1.1); 2.1395 (0.7); 2.0927 (0.6); 2.0678 (0.7); 2.0593 (0.8); 2.0511 (1.3); 2.0446 (1.5); 2.0364 (1.7); 2.0308 (1.3); 2.0226 (1.3); 2.0060 (1.5); 1.2495 (0.4); 1.2358 (0.7); 0.7862 (10.0); 0.7739 (5.0); 0.7602 (0.7); 0.0050 (0.6); −0.0002 (19.5)

I-057: ¹H-NMR(400.0 MHz, d₆-DMSO):

δ=7.3139 (1.1); 7.2975 (1.2); 7.1361 (0.3); 7.1184 (1.4); 7.1066 (3.2); 7.1011 (2.0); 7.0953 (1.5); 7.0903 (1.6); 7.0709 (2.0); 7.0653 (1.5); 7.0488 (0.9); 6.1783 (0.8); 6.1562 (0.8); 4.7353 (0.9); 4.6974 (0.8); 4.6877 (0.8); 4.5688 (1.8); 3.9826 (3.5); 3.9409 (0.8); 3.9016 (16.0); 3.8739 (0.6); 3.3199 (81.1); 2.7702 (0.3); 2.7449 (0.9); 2.7282 (1.4); 2.7150 (1.6); 2.6971 (1.6); 2.6839 (0.9); 2.6702 (1.0); 2.5054 (82.1); 2.5010 (108.5); 2.4966 (85.0); 2.3322 (0.5); 2.3276 (0.7); 2.3235 (0.6); 1.9290 (1.0); 1.9159 (1.5); 1.9066 (1.6); 1.8936 (0.9); 1.7998 (0.4); 1.7758 (0.8); 1.7541 (0.8); 1.6895 (2.7); 1.3323 (2.7); 1.3103 (1.8); 1.2583 (0.4); 1.2347 (0.4); 1.1730 (0.6); −0.0002 (12.0); −0.0083 (0.5)

I-058: ¹H-NMR(400.0 MHz, d₆-DMSO):

δ=7.9551 (1.0); 7.9375 (1.1); 7.9341 (0.9); 7.6247 (0.6); 7.6063 (0.4); 7.5473 (0.4); 7.5406 (0.6); 7.5268 (1.6); 7.5042 (15.2); 7.4728 (0.7); 7.2669 (1.3); 7.2488 (1.4); 7.1365 (0.9); 7.1332 (1.0); 7.1157 (1.8); 7.0981 (1.1); 7.0947 (1.1); 6.8506 (1.2); 6.8323 (2.1); 6.8137 (1.0); 6.7545 (2.4); 6.7345 (2.1); 6.4351 (1.1); 6.4137 (1.1); 4.7205 (1.3); 4.6724 (0.5); 4.6632 (0.5); 4.6553 (0.4); 4.6458 (0.4); 4.5505 (0.4); 4.5344 (0.5); 4.5180 (0.4); 4.2593 (0.5); 4.2397 (1.0); 4.2328 (1.0); 4.2196 (1.0); 4.2131 (0.9); 4.1834 (1.1); 4.1731 (1.1); 4.1616 (0.6); 4.1564 (0.7); 4.1097 (0.4); 4.0968 (0.8); 4.0838 (0.9); 4.0704 (0.4); 4.0174 (3.1); 3.9016 (16.0); 3.8179 (0.4); 3.7907 (0.4); 3.3212 (102.5); 3.2673 (0.5); 3.1749 (3.4); 3.1620 (3.3); 2.7897 (0.4); 2.6747 (0.7); 2.6702 (0.9); 2.6657 (0.7); 2.5404 (0.8); 2.5099 (57.7); 2.5057 (110.8); 2.5012 (143.9); 2.4966 (108.3); 2.4925 (56.3); 2.3324 (0.6); 2.3281 (0.9); 2.3234 (0.7); 2.0093 (1.3); 1.2487 (0.3); 1.2354 (1.0); 0.0078 (0.6); −0.0003 (16.5); −0.0086 (0.7)

I-060: ¹H-NMR(601.6 MHz, d₆-DMSO):

δ=7.3564 (0.3); 7.3430 (0.4); 7.3174 (1.0); 7.3080 (1.0); 7.1288 (0.7); 7.1177 (1.3); 7.1087 (2.3); 7.1059 (2.3); 7.0946 (1.3); 7.0856 (0.6); 7.0658 (1.5); 7.0540 (0.9); 6.1502 (0.5); 4.9162 (0.5); 4.6945 (0.7); 4.5973 (0.7); 4.1807 (0.6); 4.0341 (0.5); 4.0186 (0.7); 3.9468 (0.5); 3.8995 (16.0); 3.3251 (1.6); 3.3083 (79.4); 3.2678 (0.4); 3.1694 (1.6); 2.7645 (0.4); 2.7465 (0.6); 2.7351 (0.8); 2.7058 (0.6); 2.6952 (1.0); 2.6675 (0.5); 2.6119 (0.8); 2.5182 (1.8); 2.5152 (2.0); 2.5032 (105.0); 2.5003 (145.0); 2.4974 (108.2); 2.3843 (0.8); 2.1335 (0.5); 2.1073 (0.4); 1.9165 (1.2); 1.8960 (0.6); 1.8579 (0.5); 1.7779 (0.6); 1.7634 (0.6); 1.6904 (0.6); 1.6793 (0.5); 1.0154 (0.4); 1.0023 (0.8); 0.7858 (4.7); 0.7731 (2.5); −0.0002 (13.3)

1H-NMR

I-061: ¹H-NMR(601.6MHz,d₆-DMSO):

δ=7.3182 (1.1); 7.3078 (2.3); 7.2962 (1.5); 7.1275 (0.8); 7.1167 (2.3); 7.1054 (4.3); 7.0933 (2.0); 7.0812 (0.8); 7.0657 (2.9); 7.0542 (1.7); 6.1666 (1.0); 6.1508 (1.3); 6.1440 (1.7); 6.1282 (1.5); 4.6963 (1.4); 4.6678 (4.0); 4.5765 (5.6); 4.2698 (0.3); 4.0089 (1.4); 4.0009 (2.8); 3.9919 (2.0); 3.9283 (3.1); 3.9192 (4.9); 3.9113 (3.1); 3.8994 (16.0); 3.8896 (1.9); 3.8812 (1.8); 3.3058 (99.4); 3.1730 (0.4); 2.7734 (0.5); 2.7627 (0.6); 2.7449 (1.0); 2.7343 (1.4); 2.7052 (1.0); 2.6950 (1.8); 2.6938 (1.8); 2.6842 (1.0); 2.6659 (0.8); 2.6125 (0.8); 2.6092 (0.7); 2.5209 (1.8); 2.5179 (2.3); 2.5147 (2.5); 2.5029 (111.7); 2.4999 (155.7); 2.4970 (118.8); 2.3841 (1.0); 2.1388 (11.9); 2.1023 (7.9); 1.9274 (1.3); 1.9135 (2.3); 1.8973 (1.0); 1.7968 (0.4); 1.7686 (1.2); 1.7559 (1.0); 1.7419 (0.5); 1.7148 (0.6); 1.7046 (1.0); 1.6864 (1.1); 1.2371 (0.6); −0.0002 (16.3)

I-064: ¹H-NMR(400.0 MHz, d₆-DMSO):

δ=8.6481 (1.8); 8.6395 (2.3); 8.0070 (1.0); 7.9875 (2.1); 7.9681 (1.2); 7.7273 (3.6); 7.7077 (3.2); 7.5692 (1.8); 7.5568 (1.9); 7.5520 (1.8); 7.5382 (1.6); 7.2828 (1.2); 7.2641 (1.4); 7.2482 (0.9); 7.2292 (0.8); 7.1375 (0.7); 7.1190 (1.5); 7.1098 (1.2); 7.1043 (1.2); 6.8574 (0.9); 6.8399 (2.0); 6.8224 (1.6); 6.8042 (0.5); 6.7598 (1.8); 6.7462 (1.4); 6.7405 (1.7); 6.7272 (1.1); 6.4565 (1.0); 6.4348 (1.1); 6.4059 (0.8); 6.3838 (0.7); 4.8150 (4.4); 4.7738 (3.2); 4.7592 (1.0); 4.7368 (0.9); 4.7226 (0.7); 4.7086 (0.6); 4.6978 (0.5); 4.6879 (0.5); 4.2716 (0.4); 4.2517 (1.0); 4.2439 (1.0); 4.2321 (1.0); 4.2242 (1.2); 4.1933 (1.5); 4.1834 (2.0); 4.1722 (2.3); 4.1584 (1.8); 4.0978 (0.6); 4.0844 (0.7); 4.0705 (0.4); 4.0428 (1.8); 4.0210 (2.3); 4.0084 (1.9); 3.9754 (2.0); 3.9634 (2.1); 3.9017 (16.0); 3.3229 (144.7); 3.2674 (0.6); 3.1751 (3.3); 3.1624 (3.2); 2.6752 (0.7); 2.6707 (1.0); 2.6661 (0.7); 2.5408 (0.9); 2.5238 (3.6); 2.5105 (65.8); 2.5061 (128.8); 2.5016 (168.8); 2.4971 (126.2); 2.4927 (64.8); 2.3328 (0.8); 2.3284 (1.0); 2.3239 (0.8); 2.3194 (0.4); 2.0665 (0.6); 2.0553 (0.8); 2.0241 (1.4); 2.0151 (1.4); 2.0000 (1.2); 1.2496 (0.3); 1.2356 (0.8); 0.0080 (0.7); −0.0002 (19.4); −0.0084 (0.8)

I-065: ¹H-NMR(601.6 MHz, d₆-DMSO):

δ=8.1025 (0.3); 7.8896 (2.0); 7.8790 (3.5); 7.8381 (0.9); 7.8270 (0.9); 7.7465 (1.2); 7.7334 (1.8); 7.7201 (0.8); 7.2632 (0.7); 7.2530 (0.7); 7.1275 (0.8); 7.1134 (1.3); 7.1024 (0.8); 6.8441 (0.9); 6.8318 (1.3); 6.8202 (0.6); 6.7504 (1.5); 6.7371 (1.4); 6.4084 (0.6); 4.7920 (0.3); 4.7603 (0.5); 4.7303 (0.6); 4.2403 (0.7); 4.1782 (0.8); 4.1748 (0.8); 4.1617 (0.6); 4.0795 (0.5); 4.0715 (0.4); 4.0165 (1.7); 3.8996 (16.0); 3.7894 (0.4); 3.3254 (1.5); 3.3079 (101.6); 3.2682 (0.7); 3.1738 (1.6); 3.1652 (1.6); 2.6154 (0.5); 2.6122 (0.7); 2.6092 (0.6); 2.5215 (1.4); 2.5184 (1.7); 2.5152 (1.8); 2.5065 (46.9); 2.5035 (101.9); 2.5005 (141.3); 2.4974 (101.5); 2.4944 (47.1); 2.3877 (0.5); 2.3846 (0.8); 2.3815 (0.6); 2.0560 (0.4); 2.0306 (0.6); 2.0229 (0.6); 1.9992 (0.7); 1.8625 (0.5); 1.2353 (0.3); 0.0052 (0.3); −0.0002 (15.7); −0.0059 (0.6)

I-066: ¹H-NMR(601.6 MHz, d₆-DMSO):

δ=7.6684 (0.4); 7.6340 (0.3); 7.5953 (0.4); 7.5678 (1.1); 7.5542 (13.7); 7.5415 (0.7); 7.5210 (0.3); 7.2751 (0.8); 7.2625 (1.0); 7.2232 (1.1); 7.2111 (1.8); 7.1846 (0.9); 7.1735 (1.6); 7.1616 (0.8); 7.1436 (1.0); 7.1323 (1.3); 7.1194 (0.5); 6.2595 (0.6); 6.2574 (0.6); 6.2436 (0.6); 5.0526 (0.5); 5.0478 (0.5); 4.7133 (0.4); 4.0799 (0.6); 4.0714 (0.6); 4.0089 (2.1); 3.8996 (16.0); 3.7918 (0.4); 3.7780 (0.4); 3.7681 (0.3); 3.5745 (0.3); 3.3251 (1.5); 3.3090 (111.0); 3.1737 (2.5); 3.1652 (2.4); 2.9344 (0.4); 2.9193 (0.4); 2.9092 (0.5); 2.8964 (0.5); 2.7805 (0.4); 2.7669 (0.7); 2.7537 (0.5); 2.7412 (0.5); 2.6152 (0.6); 2.6119 (0.8); 2.5212 (1.1); 2.5183 (1.4); 2.5152 (1.4); 2.5062 (54.4); 2.5034 (116.1); 2.5004 (160.8); 2.4975 (117.8); 2.4946 (57.3); 2.4460 (0.7); 2.4171 (0.5); 2.3878 (0.8); 2.3848 (1.0); 1.8293 (0.4); 1.8127 (0.5); 1.7953 (0.4); 0.0051 (0.4): −0.0002 (12.8)

I-067: ¹H-NMR(601.6 MHz. d₆-DMSO):

δ=7.9467 (0.3); 7.9324 (0.4); 7.5745 (0.6); 7.5521 (16.0); 7.5356 (0.4); 7.2631 (1.0); 7.2499 (1.0); 7.1258 (0.9); 7.1141 (1.6); 7.1002 (0.9); 6.8429 (1.0); 6.8310 (1.8); 6.8188 (0.9); 6.7501 (2.0); 6.7367 (1.8); 6.4150 (0.9); 6.4031 (0.8); 4.7224 (1.0); 4.6749 (0.3); 4.6681 (0.3); 4.6490 (0.3); 4.2381 (0.9); 4.2230 (0.7); 4.1737 (0.8); 4.1591 (0.6); 4.0884 (0.4); 4.0799 (0.8); 4.0706 (0.8); 4.0627 (0.4); 4.0095 (2.5); 3.8995 (14.4); 3.3250 (1.3); 3.3074 (74.8); 3.2682 (0.6); 3.1738 (3.6); 3.1651 (3.5); 2.6120 (0.7); 2.5210 (1.2); 2.5183 (1.5); 2.5152 (1.7); 2.5032 (89.2); 2.5003 (122.3); 2.4974 (90.1); 2.3844 (0.7); 2.0451 (0.6); 2.0345 (0.7); 2.0157 (0.6); 2.0025 (0.8); 1.8624 (0.4); 1.2360 (0.5); −0.0002 (12.3)

1H-NMR

I-068: ¹H-NMR(400.0 MHz, d₆-DMSO):

δ=7.2649 (1.2); 7.2460 (1.3); 7.1347 (0.9); 7.1152 (1.8); 7.0969 (1.1); 6.8503 (1.2); 6.8317 (2.0); 6.8132 (1.0); 6.7549 (2.4); 6.7345 (2.2); 6.4180 (0.7); 6.3943 (1.6); 6.3714 (1.0); 4.7479 (0.5); 4.7349 (1.0); 4.7149 (1.0); 4.7005 (0.5); 4.6756 (2.8); 4.5836 (4.0); 4.2683 (0.4); 4.2600 (0.4); 4.2403 (1.0); 4.2338 (0.9); 4.2205 (0.9); 4.1989 (0.8); 4.1896 (1.1); 4.1834 (1.1); 4.1735 (1.1); 4.1624 (0.4); 4.1557 (0.6); 4.1466 (0.4); 4.0959 (0.3); 4.0833 (0.3); 4.0280 (0.8); 4.0149 (1.8); 4.0023 (1.3); 3.9351 (1.9); 3.9213 (3.1); 3.9117 (2.9); 3.9015 (16.0); 3.3213 (95.3); 3.2672 (0.5); 3.1748 (1.5); 3.1619 (1.5); 2.6748 (0.4); 2.6701 (0.6); 2.6657 (0.4); 2.5402 (0.4); 2.5234 (2.1); 2.5100 (38.6); 2.5057 (76.0); 2.5011 (99.8); 2.4966 (75.2); 2.4922 (38.9); 2.3325 (0.5); 2.3280 (0.6); 2.3236 (0.5); 2.1409 (8.9); 2.1031 (5.9); 2.0754 (0.3); 2.0630 (0.4); 2.0503 (0.7); 2.0391 (0.9); 2.0295 (1.1); 2.0183 (1.3); 2.0092 (1.2); 1.9957 (0.9); 1.9792 (0.6); 1.9683 (0.4); 0.0080 (0.4); −0.0002 (12.4); −0.0084 (0.5)

I-073: ¹H-NMR(601.6 MHz, d₆-DMSO):

δ=8.6463 (1.4); 8.6393 (1.9); 7.9992 (0.9); 7.9860 (1.5); 7.7224 (2.7); 7.7094 (2.6); 7.5615 (1.4); 7.5528 (1.5); 7.5502 (1.5); 7.5410 (1.2); 7.3304 (0.8); 7.3178 (0.9); 7.2929 (0.6); 7.2814 (0.7); 7.1208 (1.2); 7.1120 (2.2); 7.0991 (1.7); 7.0850 (0.7); 7.0713 (1.2); 7.0589 (1.4); 6.2088 (0.7); 6.1938 (0.8); 6.1569 (0.5); 6.1420 (0.6); 4.8075 (3.2); 4.7635 (2.1); 4.7127 (0.6); 4.6809 (0.4); 4.6679 (0.5); 4.1692 (1.2); 4.1610 (0.8); 4.0302 (1.3); 4.0054 (1.6); 3.9678 (1.5); 3.9599 (1.7); 3.8996 (16.0); 3.3344 (0.6); 3.3253 (1.7); 3.3090 (117.1); 3.2679 (0.4); 3.1694 (0.7); 2.7513 (0.6); 2.7334 (0.7); 2.7128 (0.6); 2.7002 (0.8); 2.6737 (0.6); 2.6654 (0.4); 2.6124 (0.9); 2.6091 (0.8); 2.5215 (1.3); 2.5185 (1.7); 2.5153 (1.6); 2.5063 (52.1); 2.5034 (113.2); 2.5004 (158.0); 2.4974 (115.3); 2.4945 (55.1); 2.3877 (0.7); 2.3846 (0.9); 1.9439 (0.5); 1.9269 (0.9); 1.8979 (1.0); 1.8583 (0.4); 1.7907 (0.6); 1.7751 (0.8); 1.7595 (0.7); 1.7458 (0.4); 1.7299 (0.4); 1.7088 (0.6); 1.6963 (0.6); 1.6890 (0.6); 1.6605 (0.4); −0.0002 (15.1)

I-076: ¹H-NMR(601.6 MHz, d₆-DMSO):

δ=7.4139 (1.6); 7.4097 (2.0); 7.4040 (2.8); 7.3979 (3.2); 7.3908 (0.7); 7.3817 (1.4); 7.3760 (7.5); 7.3715 (5.2); 7.3684 (2.3); 7.3652 (3.0); 7.2679 (1.6); 7.2564 (1.6); 7.1311 (0.9); 7.1285 (0.9); 7.1173 (1.6); 7.1058 (1.0); 7.1027 (1.0); 6.8496 (1.2); 6.8478 (1.3); 6.8356 (2.0); 6.8250 (1.1); 6.8231 (1.0); 6.7539 (2.3); 6.7402 (2.0); 6.7385 (2.0); 6.4168 (1.7); 6.4025 (1.7); 4.7342 (0.5); 4.7261 (1.0); 4.7170 (0.8); 4.7106 (1.0); 4.7019 (0.5); 4.6232 (9.2); 4.3574 (6.5); 4.2625 (0.4); 4.2580 (0.6); 4.2490 (0.6); 4.2443 (1.2); 4.2396 (0.9); 4.2306 (0.8); 4.2258 (0.8); 4.1971 (0.7); 4.1914 (0.8); 4.1861 (0.8); 4.1797 (1.0); 4.1732 (0.5); 4.1679 (0.5); 4.1618 (0.4); 3.8992 (16.0); 3.8684 (1.4); 3.8600 (2.7); 3.8510 (1.7); 3.6377 (2.0); 3.6284 (3.1); 3.6195 (1.7); 3.3083 (94.7); 3.1737 (1.2); 3.1650 (1.2); 2.6148 (0.4); 2.6121 (0.7); 2.6089 (0.5); 2.5212 (1.2); 2.5182 (1.5); 2.5151 (1.5); 2.5062 (39.5); 2.5032 (87.2); 2.5002 (122.5); 2.4972 (88.7); 2.4942 (42.0); 2.3878 (0.5); 2.3843 (0.8); 2.0612 (0.4); 2.0518 (0.7); 2.0463 (0.8); 2.0378 (0.9); 2.0295 (0.7); 2.0242 (0.4); 2.0177 (0.4); 2.0118 (0.6); 2.0057 (0.8); 2.0015 (0.9); 1.9903 (0.6); 1.9835 (0.4); 1.9783 (0.4); 1.9731 (0.3); −0.0002 (13.1); −0.0059 (0.6)

I-078: ¹H-NMR(601.6 MHz, d₆-DMSO):

δ=7.4141 (1.2); 7.4096 (1.4); 7.4041 (1.9); 7.3981 (2.4); 7.3919 (0.6); 7.3821 (1.1); 7.3764 (5.4); 7.3717 (3.9); 7.3655 (2.6); 7.3181 (1.1); 7.3064 (1.1); 7.1316 (0.4); 7.1226 (1.0); 7.1195 (1.1); 7.1117 (2.1); 7.1089 (1.9); 7.1012 (1.0); 7.0981 (1.1); 7.0889 (0.4); 7.0702 (1.3); 7.0670 (1.2); 7.0555 (0.7); 6.1949 (1.5); 6.1796 (1.6); 4.7030 (0.4); 4.6911 (0.6); 4.6814 (0.6); 4.6677 (0.4); 4.6211 (7.1); 4.3509 (5.3); 3.8992 (16.0); 3.8558 (1.2); 3.8473 (2.3); 3.8385 (1.3); 3.6343 (1.6); 3.6252 (2.4); 3.6163 (1.3); 3.3251 (0.8); 3.3073 (84.6); 2.7478 (0.5); 2.7383 (0.6); 2.7273 (0.4); 2.7074 (0.4); 2.6968 (0.8); 2.6878 (0.4); 2.6695 (0.4); 2.6148 (0.4); 2.6120 (0.6); 2.5211 (1.0); 2.5181 (1.2); 2.5150 (1.2); 2.5062 (34.3); 2.5031 (75.9); 2.5001 (106.4); 2.4970 (76.0); 2.4940 (35.2); 2.3872 (0.4); 2.3843 (0.6); 2.3810 (0.5); 1.9454 (0.3); 1.9327 (0.5); 1.9187 (1.0); 1.9125 (0.6); 1.9027 (0.4); 1.8882 (0.3); 1.7769 (0.7); 1.7610 (0.6); 1.7491 (0.4); 1.7105 (0.4); 1.6954 (0.4); 1.6821 (0.4); 1.6787 (0.4); 0.0053 (0.3); −0.0002 (13.2); −0.0057 (0.5)

1H-NMR

I-079: ¹H-NMR(601.6 MHz, d₆-DMSO):

δ=7.4176 (1.5); 7.4129 (1.7); 7.4076 (2.4); 7.4015 (2.9); 7.3848 (1.3); 7.3790 (6.2); 7.3743 (4.6); 7.3683 (2.9); 7.2764 (1.5); 7.2651 (1.8); 7.2280 (1.3); 7.2160 (2.0); 7.1904 (1.0); 7.1779 (1.8); 7.1663 (1.0); 7.1515 (1.2); 7.1399 (1.6); 7.1283 (0.5); 5.0584 (0.8); 5.0459 (1.5); 5.0330 (0.7); 4.6260 (8.5); 4.3637 (5.2); 3.8993 (16.0); 3.8709 (1.3); 3.8625 (2.5); 3.8538 (1.5); 3.6661 (0.4); 3.6403 (2.3); 3.6314 (3.3); 3.6225 (2.2); 3.5220 (3.8); 3.3998 (0.7); 3.3778 (0.4); 3.3713 (0.4); 3.3251 (0.7); 3.1695 (2.6); 2.9429 (0.4); 2.9382 (0.4); 2.9296 (0.5); 2.9240 (0.5); 2.9170 (0.7); 2.9126 (0.6); 2.9033 (0.6); 2.8979 (0.6); 2.8904 (0.6); 2.7862 (0.5); 2.7718 (1.0); 2.7589(0.8); 2.7464 (0.7); 2.7317 (0.8); 2.6151 (0.6); 2.6123 (0.7); 2.6094 (0.6); 2.5584 (0.4); 2.5490 (1.0); 2.5397 (0.9); 2.5214 (1.4); 2.5185 (1.6); 2.5153 (1.6); 2.5065 (45.3); 2.5035 (98.9); 2.5005 (137.8); 2.4975 (99.8); 2.4945 (47.5); 2.4708 (0.7); 2.4655 (0.6); 2.4576 (0.7); 2.4529 (0.9); 2.4449 (0.9); 2.4370 (0.8); 2.4318 (0.8); 2.4248 (0.5); 2.4191 (0.4); 2.3849 (0.8); 2.3820 (0.6); 1.8498 (0.4); 1.8358 (0.9); 1.8295 (0.4); 1.8219 (0.8); 1.8153 (0.9); 1.8077 (0.4); 1.8015 (0.8); −0.0002 (8.8)

I-084: ¹H-NMR(400.0 MHz, d₆-DMSO):

δ=8.3177 (0.3); 7.2681 (3.0); 7.2497 (3.3); 7.1385 (5.0); 7.1351 (5.1); 7.1185 (5.6); 7.1004 (3.1); 7.0969 (2.8); 6.8497 (2.2); 6.8314 (3.8); 6.8128 (1.8); 6.7777 (0.6); 6.7580 (4.2); 6.7378 (3.8); 6.4639 (2.1); 6.4419 (2.1); 4.7574 (0.8); 4.7439 (1.8); 4.7328 (1.4); 4.7223 (1.8); 4.7090 (0.8); 4.4571 (9.7); 4.2726 (0.6); 4.2649 (0.8); 4.2448 (2.0); 4.2376 (1.6); 4.2251 (1.6); 4.2174 (1.4); 4.2023 (1.4); 4.1933 (1.7); 4.1867 (1.7); 4.1773 (1.8); 4.1665 (0.7); 4.1594 (0.9); 4.1506 (0.6); 4.1011 (0.4); 4.0883 (0.5); 4.0750 (0.4); 4.0574 (2.5); 4.0441 (4.8); 4.0308 (3.0); 3.9021 (16.0); 3.7881 (3.6); 3.7743 (5.7); 3.7609 (2.9); 3.3231 (93.2); 3.1745 (1.7); 3.1614 (1.6); 2.8051 (0.6); 2.7878 (1.4); 2.7799 (1.1); 2.7735 (2.4); 2.7674 (1.1); 2.7571 (1.6); 2.7417 (0.6); 2.6752 (1.1); 2.6708 (1.5); 2.6662 (1.1); 2.5061 (199.3); 2.5018 (255.5); 2.4975 (190.0); 2.3327 (1.1); 2.3285 (1.5); 2.3241 (1.2); 2.0817 (0.5); 2.0703 (0.6); 2.0571 (1.1); 2.0460 (1.4); 2.0362 (1.6); 2.0253 (2.0); 2.0157 (1.9); 2.0102 (1.8); 2.0014 (1.8); 1.9873 (1.0); 1.9752 (0.7); 1.9674 (0.5); 1.9599 (0.4); 1.8905 (0.5); 1.8778 (0.9); 1.8700 (1.0); 1.8579 (1.6); 1.8493 (0.7); 1.8452 (1.0); 1.8374 (1.0); 1.8247 (0.5); 1.2368 (0.5); 1.0138 (15.1); 1.0021 (7.2); 0.9942 (6.6); 0.9047 (0.7); 0.8973 (0.8); 0.8847 (1.9); 0.5708 (0.7); 0.5592 (2.3); 0.5541 (3.6); 0.5467 (3.3); 0.5420 (3.5); 0.5341 (1.4); 0.5251 (0.6); 0.5125 (1.4); 0.5048 (3.1); 0.4999 (2.3); 0.4922 (1.6); 0.4846 (3.1); 0.4791 (1.9); 0.4718 (0.7); 0.4670 (0.6); −0.0001 (6.4)

I-087: ¹H-NMR(400.0 MHz, d₆-DMSO):

δ=7.3441 (0.3); 7.3130 (0.8); 7.2939 (1.9); 7.2848 (2.1); 7.1582 (2.2); 7.1205 (1.0); 7.1078 (1.7); 7.0949 (1.0); 7.0902 (1.0); 7.0722 (1.3); 7.0668 (1.1); 7.0496 (1.3); 7.0323 (2.0); 6.7784 (0.9); 4.7062 (0.4); 4.6952 (0.4); 4.4471 (1.6); 4.0335 (1.1); 3.9019 (16.0); 3.7847 (1.0); 3.7706 (1.5); 3.7576 (0.8); 3.3237 (37.0); 3.1670 (1.2); 2.7860 (0.5); 2.7713 (0.8); 2.7555 (0.6); 2.7302 (0.5); 2.7158 (0.6); 2.6979 (0.6); 2.6805 (0.5); 2.6754 (0.7); 2.6710 (0.9); 2.6666 (0.7); 2.5064 (107.2); 2.5020 (137.6); 2.4975 (103.4); 2.3332 (0.7); 2.3288 (0.9); 2.3242 (0.7); 1.9353 (0.5); 1.9219 (0.7); 1.9111 (0.7); 1.8945 (0.6); 1.8818 (0.8); 1.8743 (0.8); 1.8624 (1.1); 1.8493 (0.7); 1.8416 (0.6); 1.8292 (0.3); 1.7768 (0.4); 1.7530 (0.4); 1.7106 (0.3); 1.6932 (0.3); 1.4256 (0.3); 1.0125 (4.4); 0.9979 (2.3); 0.9037(1.1); 0.8968 (1.2); 0.8845 (3.3); 0.5733 (0.5); 0.5607 (1.5); 0.5564 (2.3); 0.5486 (2.2); 0.5444 (2.3); 0.5366 (0.9); 0.5278 (0.4); 0.5155 (1.0); 0.5076 (2.1); 0.5032 (1.5); 0.4951 (1.1); 0.4875 (2.1); 0.4827 (1.2); 0.4748 (0.5); 0.4704 (0.5); 0.0080 (0.4); −0.0002 (12.1); −0.0084 (0.5)

I-088: ¹H-NMR(400.0 MHz, d₆-DMSO):

δ=7.2749 (1.2); 7.2573 (1.6); 7.2319 (0.9); 7.2137 (2.0); 7.1936 (0.9); 7.1779 (1.6); 7.1602 (1.0); 7.1520 (1.2); 7.1334 (1.3); 7.1154 (0.5); 5.0785 (0.6); 5.0596 (1.2); 5.0402 (0.6); 4.4609 (4.2); 4.1073 (0.4); 4.0579 (1.5); 4.0455 (2.6); 4.0325 (1.7); 3.9749 (0.4); 3.9635 (0.4); 3.9016 (16.0); 3.7915 (1.8); 3.7778 (2.8); 3.7641 (1.5); 3.1684 (0.9); 2.9485 (0.3); 2.9343 (0.4); 2.9261 (0.4); 2.9164 (0.5); 2.9086 (0.6); 2.8907 (0.8); 2.7947 (0.8); 2.7765 (2.0); 2.7593 (1.3); 2.7385(0.6); 2.7315 (0.5); 2.6751 (0.4); 2.6705 (0.6); 2.6660 (0.4); 2.5237 (1.6); 2.5059 (85.8); 2.5015 (110.3); 2.4970 (82.5); 2.4645 (1.3); 2.4559 (1.2); 2.4448 (1.1); 2.4332 (1.0); 2.4252 (0.9); 2.4142 (0.6); 2.4059 (0.5); 2.3324 (0.6); 2.3282 (0.8); 2.3236 (0.6); 1.8429 (0.7); 1.8338 (0.4); 1.8219 (0.7); 1.8118 (0.7); 1.8006 (0.4); 1.7911 (0.7); 1.0159 (7.3); 1.0038 (3.4); 0.9968 (3.3); −0.0002 (9.3); −0.0083 (0.4)

1H-NMR

I-091: ¹H-NMR(400.0 MHz, d₆-DMSO):

δ=7.2681 (2.0); 7.2495 (2.2); 7.1353 (1.0); 7.1169 (2.1); 7.0990 (1.2); 6.8528 (1.4); 6.8340 (2.5); 6.8159 (1.2); 6.7570 (2.7); 6.7366 (2.4); 6.4543 (1.8); 6.4324 (1.9); 4.7481 (0.6); 4.7337 (1.2); 4.7241 (1.1); 4.7132 (1.2); 4.7002 (0.6); 4.4760 (7.3); 4.2680 (0.4); 4.2609 (0.6); 4.2411 (1.4); 4.2333 (1.1); 4.2214 (1.1); 4.2136 (1.0); 4.1995 (0.9); 4.1906 (1.2); 4.1849 (1.2); 4.1750 (1.2); 4.1629 (0.5); 4.1576 (0.6); 4.1494 (0.4); 3.9958 (1.8); 3.9827 (3.6); 3.9698 (2.3); 3.9017 (9.5); 3.8168 (2.4); 3.8034 (3.7); 3.7908 (1.8); 3.5716 (0.5); 3.5544 (1.2); 3.5374 (1.6); 3.5205 (1.2); 3.5038 (0.5); 3.3193 (37.1); 3.1746 (0.8); 3.1616 (0.8); 2.6702 (0.7); 2.5013 (125.0); 2.3285 (0.8); 2.0766 (0.3); 2.0676 (0.4); 2.0534 (0.7); 2.0421 (1.0); 2.0317 (1.2); 2.0230 (1.4); 2.0107 (1.6); 2.0012 (1.3); 1.9754 (0.5); 1.7163 (1.1); 1.7040 (1.1); 1.3383 (0.3); 1.2599 (16.0); 1.2430 (15.8); −0.0002 (7.4)

I-094: ¹H-NMR(400.0 MHz, d₆-DMSO):

δ=7.3174 (1.3); 7.3014 (1.6); 7.2961 (1.6); 7.1380 (0.4); 7.1242 (1.2); 7.1201 (1.5); 7.1152 (1.5); 7.1089 (3.7); 7.1026 (2.2); 7.0974 (1.6); 7.0926 (1.7); 7.0726 (2.1); 7.0665 (1.6); 7.0506 (1.0); 6.2324 (1.8); 6.2094 (1.8); 4.7166 (0.4); 4.6988 (0.8); 4.6870 (0.9); 4.6660 (0.5); 4.4683 (7.1); 3.9831 (1.5); 3.9700 (3.2); 3.9566 (2.0); 3.9013 (12.3); 3.8135 (2.1); 3.8002 (3.3); 3.7869 (1.6); 3.5694 (0.4); 3.5522 (1.2); 3.5352 (1.6); 3.5184 (1.2); 3.5015 (0.5); 3.3183 (31.9); 3.1751 (0.6); 3.1628 (0.6); 2.7476 (0.7); 2.7298 (1.1); 2.7158 (1.1); 2.6988 (1.2); 2.6835 (0.6); 2.6705 (0.8); 2.6610 (0.5); 2.5056 (81.3); 2.5013 (103.0); 2.4971 (77.8); 2.3280 (0.7); 2.3240 (0.5); 1.9333 (1.0); 1.9203 (1.6); 1.9097 (1.6); 1.8948 (0.9); 1.8763 (0.5); 1.8576 (0.4); 1.8046 (0.4); 1.7770 (1.0); 1.7572 (0.9); 1.7347 (0.6); 1.7260 (0.5); 1.7040 (0.8); 1.6937 (0.8); 1.6697 (0.5); 1.2606 (16.0); 1.2437 (15.8); −0.0002 (6.8)

I-095: ¹H-NMR(400.0 MHz, d₆-DMSO):

δ=7.6779 (0.4); 7.6511 (0.4); 7.2736 (0.8); 7.2563 (1.0); 7.2303 (0.7); 7.2127 (1.2); 7.1930 (0.6); 7.1773 (1.0); 7.1549 (0.9); 7.1359 (0.8); 5.0510 (0.6); 4.4787 (2.8); 4.4316 (0.7); 3.9959 (0.8); 3.9826 (1.5); 3.9698 (1.0); 3.9169 (0.5); 3.9018 (16.0); 3.8208 (1.1); 3.8072 (1.7); 3.7941 (1.0); 3.7833 (0.6); 3.7690 (0.6); 3.5856 (0.4); 3.5735 (0.6); 3.5564 (1.0); 3.5474 (0.7); 3.5395 (1.4); 3.5222 (1.4); 3.5040 (1.4); 3.4066 (16.3); 3.1685 (1.7); 2.9122 (0.3); 2.9051 (0.4); 2.8906 (0.6); 2.8836 (0.3); 2.7758 (0.4); 2.7320 (0.3); 2.6711 (0.7); 2.5240 (1.6); 2.5063 (107.2); 2.5020 (137.8); 2.4977 (104.6); 2.4561 (1.3); 2.4440 (1.0); 2.4336 (1.0); 2.4255 (0.8); 2.4133 (0.6); 2.4065 (0.6); 2.3838 (0.4); 2.3690 (0.3); 2.3330 (0.8); 2.3286 (1.0); 2.3241 (0.8); 1.8811 (0.4); 1.8368 (0.4); 1.8160 (0.5); 1.8058 (0.4); 1.7849 (0.4); 1.4260 (0.4); 1.3655 (0.7); 1.3487 (1.0); 1.3322 (0.4); 1.2612 (7.8); 1.2442 (9.9); 1.2267 (2.8); 1.1912 (0.5); 1.1786 (0.7); 1.1630 (0.6); 1.1494 (0.5); 1.1321 (0.5); 1.0623 (0.4); 1.0455 (0.4); −0.0001 (7.8)

I-096: ¹H-NMR(400.0 MHz, d₆-DMSO):

δ=7.2748 (1.3); 7.2573 (1.8); 7.2309 (1.0); 7.2138 (2.2); 7.1938 (1.0); 7.1777 (1.8); 7.1547 (1.5); 7.1343 (1.4); 7.1174 (0.5); 5.0754 (0.6); 5.0566 (1.3); 5.0378 (0.7); 4.4061 (4.2); 4.0558 (1.4); 4.0439 (2.5); 4.0315 (1.7); 3.9636 (0.5); 3.9018 (10.0); 3.8803 (0.7); 3.8691 (0.7); 3.7166 (2.2); 3.7031 (3.2); 3.6897 (1.9); 3.1685 (0.8); 3.0767 (16.0); 2.9527 (0.4); 2.9459 (0.4); 2.9314 (0.4); 2.9237 (0.4); 2.9141 (0.7); 2.9064 (0.7); 2.8914 (0.8); 2.8850 (0.6); 2.7977 (0.5); 2.7771 (1.0); 2.7569 (0.8); 2.7382 (0.6); 2.7173 (0.4); 2.6753 (0.5); 2.6707 (0.6); 2.6664 (0.5); 2.5061 (89.7); 2.5018 (114.2); 2.4974 (86.1); 2.4653 (1.3); 2.4569 (1.1); 2.4454 (1.0); 2.4341 (1.0); 2.4263 (0.8); 2.4150 (0.5); 2.4068 (0.4); 2.3284 (0.7); 1.8400 (0.8); 1.8306 (0.4); 1.8192 (0.8); 1.8098 (0.7); 1.7978 (0.4); 1.7882 (0.7); 1.2359 (0.4); −0.0001 (8.4)

I-097: ¹H-NMR(400.0 MHz, d₆-DMSO):

δ=7.3175 (1.1); 7.3012 (1.3); 7.2961 (1.3); 7.1384 (0.3); 7.1251 (1.0); 7.1205 (1.2); 7.1154 (1.2); 7.1092 (3.0); 7.1033 (1.8); 7.0974 (1.3); 7.0924 (1.5); 7.0731 (1.8); 7.0671 (1.4); 7.0555 (0.8); 7.0508 (0.8): 6.2463 (1.2); 6.2232 (1.2); 4.7036 (0.6); 4.6929 (0.8); 4.6699 (0.4); 4.3964 (5.6); 4.0411 (1.4); 4.0284 (2.5); 4.0153 (1.5); 3.9017 (12.7); 3.7095 (1.8); 3.6956 (2.8); 3.6823 (1.5); 3.3202 (35.2); 3.0968 (0.4); 3.0727 (16.0); 3.0283 (0.3); 2.7482 (0.6); 2.7298 (0.9); 2.7164 (0.9); 2.6996 (1.0); 2.6852 (0.5); 2.6747 (0.6); 2.6703 (0.8); 2.6656 (0.6); 2.6607 (0.4); 2.5233 (1.6); 2.5097 (41.5); 2.5056 (81.4); 2.5011 (105.8); 2.4966 (78.9); 2.3323 (0.5); 2.3278 (0.7); 2.3235 (0.6); 1.9363 (0.9); 1.9222 (1.4); 1.9111 (1.3); 1.8966 (0.7); 1.8789 (0.4); 1.8574 (0.4); 1.8058 (0.3); 1.7778 (0.8); 1.7551 (0.7); 1.7351 (0.6); 1.7297 (0.4); 1.7112 (0.6); 1.6952 (0.6); 1.6726 (0.4); −0.0002 (6.7)

1H-NMR

I-105: ¹H-NMR(400.0MHz, d₆-DMSO):

δ=8.1083 (1.4); 8.0898 (1.3); 7.7486 (0.4); 7.7367 (2.9); 7.7353 (3.0); 7.7288 (2.2); 7.7235 (1.6); 7.7202 (1.3); 7.6250 (0.7); 7.6172 (0.7); 7.6111 (0.6); 7.6047 (1.0); 7.5984 (0.7); 7.5905 (0.5); 7.5835 (0.5); 7.3016 (0.8); 7.2856 (1.0); 7.2801 (1.0); 7.1222 (0.8); 7.1177 (1.0); 7.1137 (1.0); 7.1069 (2.3); 7.1004 (1.3); 7.0960 (1.0); 7.0905 (1.0); 7.0775 (0.4); 7.0686 (1.2); 7.0624 (1.0); 7.0467 (0.6); 6.2468 (1.0); 6.2239 (1.0); 4.6714 (0.6); 4.5054 (4.0); 3.9019 (16.0); 3.8575 (1.6); 3.8454 (1.7); 3.3203 (35.7); 2.7415 (0.4); 2.7243 (0.6); 2.7109 (0.7); 2.6930 (0.8); 2.6754 (0.7); 2.6703 (0.7); 2.6662 (0.6); 2.5058 (70.2); 2.5014 (89.5); 2.4969 (66.5); 2.3325 (0.4); 2.3279 (0.5); 2.3237 (0.4); 1.9088 (0.9); 1.9005 (1.0); 1.8869 (0.6); 1.7628 (0.6); 1.7445 (0.6); 1.7205 (0.5); 1.7054 (0.4); 1.6842 (0.4); 1.6821 (0.4); −0.0002 (6.3)

I-106: ¹H-NMR(400.0 MHz, d₆-DMSO):

δ=8.1129 (1.9); 8.0935 (1.8); 7.7506 (0.4); 7.7380 (4.2); 7.7309 (3.0); 7.7264 (2.3); 7.7233 (1.9); 7.7027 (0.3); 7.6275 (1.0); 7.6193 (1.0); 7.6140 (0.8); 7.6071 (1.3); 7.6005 (0.9); 7.5933 (0.7); 7.5861 (0.7); 7.2572 (1.3); 7.2392 (1.8); 7.2267 (1.0); 7.2092 (2.1); 7.1910 (0.9); 7.1757 (1.7); 7.1539 (1.8); 7.1350 (1.3); 7.1170 (0.4); 5.0532 (0.7); 5.0344 (1.4); 5.0153 (0.7); 4.5209 (5.1); 3.9020 (16.0); 3.8636 (3.6); 3.8515 (3.7); 3.1686 (1.0); 2.9461 (0.4); 2.9381 (0.4); 2.9248 (0.4); 2.9161 (0.4); 2.9075 (0.6); 2.8991 (0.6); 2.8852 (0.6); 2.8778 (0.6); 2.7896 (0.4); 2.7687 (1.0); 2.7485 (0.8); 2.7296 (0.6); 2.7088 (0.3); 2.6749 (0.5); 2.6706 (0.8); 2.6665 (0.6); 2.5060 (102.7); 2.5017 (132.8); 2.4974 (100.5); 2.4625 (0.9); 2.4509 (1.0); 2.4426 (0.9); 2.4315 (0.9); 2.4202 (0.9); 2.4122 (0.8); 2.4011 (0.5); 2.3930 (0.4); 2.3283 (0.8); 1.8281 (0.8); 1.8185 (0.4); 1.8071 (0.8); 1.7971 (0.8); 1.7860 (0.4); 1.7761 (0.7); −0.0001 (7.8 )

I-150: ¹H-NMR(400.0 MHz, d₆-DMSO):

δ=8.0734 (3.0); 8.0545 (3.8); 7.7066 (3.2); 7.7028 (6.0); 7.6936 (8.6); 7.6920 (7.9); 7.6827 (0.9); 7.6100 (0.4); 7.5989 (1.8); 7.5899 (1.8); 7.5861 (1.8); 7.5788 (2.3); 7.5678 (2.1); 7.5574 (1.3); 7.3730 (3.9); 7.3516 (10.7); 7.3270 (11.9); 7.3106 (1.8); 7.3055 (4.3); 6.6022 (2.4); 6.5812 (2.5); 4.6352 (0.4); 4.6192 (1.5); 4.6000 (1.9); 4.5810 (1.4); 4.5639 (0.4); 4.4421 (9.7); 3.9021 (16.0); 3.8023 (3.4); 3.7949 (4.5); 3.7800 (5.1); 3.7762 (5.2); 3.3225 (139.5); 2.6752 (0.8); 2.6708 (1.2); 2.6663 (0.9); 2.5239 (4.1); 2.5106 (74.5); 2.5063 (145.5); 2.5018 (189.4); 2.4972 (142.1); 2.4932 (74.7); 2.3329 (0.8); 2.3285 (1.1); 2.3240 (0.8); 1.3511 (9.9); 1.3338 (9.9); 0.0080 (0.4); −0.0002 (12.2); −0.0084 (0.6)

I-151: ¹H-NMR(599.8 MHz, CDCl3):

δ=7.2623 (12.6); 7.0953 (14.4); 7.0605 (6.9); 7.0480 (9.7); 6.9935 (9.0); 6.9810 (6.2); 5.2973 (2.7); 4.7937 (3.8); 4.7786 (7.5); 4.7635 (4.1); 4.2471 (5.9); 4.2310 (5.6); 4.0188 (8.3); 4.0104 (15.5); 4.0019 (8.8); 3.7596 (1.0); 3.7320 (26.0); 3.7051 (1.0); 3.3146 (0.3); 3.3063 (0.6); 3.2977 (0.3); 3.0180 (4.0); 3.0054 (4.7); 2.9926 (5.4); 2.9776 (15.6); 2.9681 (13.6); 2.9569 (7.1); 2.9451 (4.6); 2.9343 (1.9); 2.4926 (3.7); 2.4772 (4.3); 2.4674 (3.5); 2.4518 (3.8); 2.3913 (0.3); 2.2881 (49.3); 2.2529 (0.6); 2.2408 (1.8); 2.2287 (3.6); 2.2160 (4.5); 2.2034 (3.4); 2.1909 (1.5); 2.1799 (0.6); 2.1699 (0.6); 1.7155 (13.4); 1.3263 (0.4); 1.3150 (0.5); 1.2854 (25.9); 1.2746 (25.5); 1.2500 (0.8); 1.2368 (0.6); 1.1458 (49.8); 1.1352 (50.0); 1.0396 (0.3); 1.0289 (0.3); −0.0001 (11.6)

I-152: ¹H-NMR(400.0 MHz, CDCl3):

δ=7.2598 (72.1); 7.0975 (2.2); 7.0662 (1.3); 7.0474 (2.0); 6.9958 (1.8); 6.9787 (0.9); 5.2978 (3.8); 4.8064 (0.7); 4.7838 (1.2); 4.7615 (0.8); 4.1899 (0.9); 4.1661 (0.9); 4.0307 (1.5); 4.0170 (2.8); 4.0034 (1.7); 3.7442 (4.8); 3.0302 (0.8); 3.0117(1.0); 2.9921 (2.4); 2.9818 (2.5); 2.9764 (3.6); 2.9680(1.5); 2.9633 (1.6); 2.9601 (1.8); 2.9435 (1.1); 2.5039 (0.7); 2.4807 (0.7); 2.4654 (0.6); 2.4435 (0.7); 2.2892 (9.9); 2.2341 (0.6); 2.2159 (0.8); 2.1987(0.6); 1.5736 (4.4); 1.2900 (8.3); 1.2732 (8.1); 1.1524 (16.0); 1.1360 (15.8); 0.0079 (1.0); −0.0002 (26.7); −0.0085 (1.1)

1H-NMR

I-153: ¹H-NMR(400.0 MHz, CDCl3):

δ=7.5192 (0.6); 7.3979 (2.4); 7.3809 (3.0); 7.2602 (112.3); 7.2434 (4.7); 7.2399 (5.8); 7.2372 (4.5); 7.2277 (5.2); 7.2249 (4.2); 7.2159 (3.1); 7.2111 (2.6); 7.2069 (2.3); 7.1986 (2.2); 7.1922 (1.5); 7.1824 (1.0); 7.1761 (0.6); 6.9962 (0.6); 5.2977 (10.6); 5.2550 (0.8); 5.2361 (1.8); 5.2158 (1.8); 5.1965 (0.8); 4.3103 (1.4); 4.2880 (1.3); 4.0298 (16.0); 4.0126 (3.8); 3.9993 (6.6); 3.9849 (4.1); 3.3208 (6.2); 3.3068 (8.5); 3.2932 (5.8); 3.0219 (0.8); 3.0130 (0.8); 3.0005 (0.8); 2.9913 (0.9); 2.9823 (1.4); 2.9733 (1.6); 2.9608 (1.5); 2.9519 (1.5); 2.8957 (1.1); 2.8747 (2.3); 2.8549 (1.8); 2.8354 (1.2); 2.8154 (0.8); 2.7229 (1.1); 2.7139 (1.1); 2.7050 (1.3); 2.6960 (1.4); 2.6913 (1.4); 2.6855 (1.1); 2.6822 (1.3); 2.6764 (1.1); 2.6734 (1.4); 2.6643 (1.4); 2.6538 (1.0); 2.6447 (0.9); 1.9009 (1.0); 1.8797 (2.2); 1.8693 (1.1); 1.8610 (2.2); 1.8585 (1.3); 1.8505 (1.3); 1.8481 (2.0); 1.8396 (1.1); 1.8293 (2.0); 1.8268 (1.1); 1.8079 (0.9); 1.6369 (4.7); 0.0079 (1.5); −0.0002 (41.1); −0.0085 (1.5)

I-154: ¹H-NMR(400.0 MHz, CDCl3):

δ=7.5187 (0.7); 7.4546 (2.1); 7.4392 (2.3); 7.4317 (2.3); 7.2598 (126.5); 7.1838 (0.6); 7.1711 (2.7); 7.1664 (4.4); 7.1574 (8.1); 7.1487 (5.5); 7.1431 (3.0); 7.1308 (0.7); 7.1029 (2.7); 7.0947 (2.2); 7.0804 (1.5); 6.9958 (0.7); 4.8784 (0.8); 4.8642 (1.3); 4.8561 (1.1); 4.8438 (1.4); 4.8294 (0.8); 4.3036 (1.7); 4.2818 (1.6); 4.0262 (16.0); 4.0044 (3.7); 3.9911 (6.4); 3.9769 (3.9); 3.3168 (5.8); 3.3028 (8.6); 3.2892 (5.3); 2.8535 (0.7); 2.8276 (1.1); 2.8119 (2.1); 2.7965 (1.3); 2.7881 (1.2); 2.7712 (1.8); 2.7547 (1.3); 2.7287 (0.6); 2.1272 (0.5); 2.1220 (0.5); 2.1127 (0.7); 2.1031 (1.2); 2.0916 (1.3); 2.0808 (1.2); 2.0710 (1.1); 2.0591 (0.9); 1.9709 (0.6); 1.9639 (0.8); 1.9506 (1.4); 1.9442 (1.3); 1.9285 (1.6); 1.9171 (2.0); 1.9115 (1.4); 1.9031 (1.4); 1.8960 (1.8); 1.8854 (0.8); 1.8814 (0.8); 1.8765 (1.1); 1.8684 (1.0); 1.8584 (0.8); 1.8519 (1.1); 1.8444 (1.1); 1.8283 (1.0); 1.8082 (0.7); 1.6273 (2.7); 0.0079 (1.5); −0.0002 (47.6); −0.0085 (1.4)

I-155: ¹H-NMR(400.0 MHz, d₆-DMSO):

δ=8.3146 (0.4); 7.2662 (4.7); 7.2475 (5.1); 7.1282 (2.7); 7.1108 (5.0); 7.0935 (3.0); 6.8470 (3.5); 6.8291 (6.0); 6.8113 (2.8); 6.7674 (0.6); 6.7505 (7.1); 6.7306 (6.3); 6.3341 (0.3); 6.2876 (0.5); 6.2582 (2.8); 6.2362 (2.7); 4.7420 (1.4); 4.7283 (2.9); 4.7165 (2.3); 4.7069 (2.9); 4.6936 (1.5); 4.5500 (0.3); 4.5341 (0.5); 4.2670 (1.2); 4.2586 (1.6); 4.2390 (3.5); 4.2308 (3.0); 4.2195 (2.9); 4.2109 (2.6); 4.1930 (2.5); 4.1838 (3.0); 4.1777 (3.0); 4.1679 (3.1); 4.1564 (1.4); 4.1497 (1.7); 4.1413 (1.2); 4.1118 (0.5); 4.0987 (0.9); 4.0876 (1.2); 4.0767 (1.2); 4.0140 (0.5); 3.9014 (16.0); 3.8506 (4.2); 3.8371 (7.8); 3.8195 (13.7); 3.7784 (0.6); 3.6897 (0.6); 3.6655 (0.3); 3.6423 (0.3); 3.5844 (0.4); 3.4549 (0.4); 3.3232 (70.7); 3.1739 (3.9); 3.1613 (4.0); 3.1469 (3.9); 3.1336 (6.6); 3.1206 (3.7); 3.0504 (0.4); 2.6699 (1.5); 2.5401 (1.2); 2.5051 (216.7); 2.5010 (280.7); 2.4970 (207.0); 2.3279 (1.7); 2.0763 (0.6); 2.0651 (0.9); 2.0554 (1.1); 2.0424 (2.0); 2.0329 (2.6); 2.0217 (3.2); 2.0138 (3.4); 2.0004 (4.0); 1.9917 (3.0); 1.9767 (1.7); 1.9653 (1.1); 1.9571 (0.8); 1.9502 (0.7); 1.9429 (0.4); 1.2355 (0.5); −0.0007 (2.2)

I-156: ¹H-NMR(400.0 MHz, CDCl3):

δ=7.2600 (83.9); 7.1988 (3.7); 7.1361 (2.0); 7.1172 (3.1); 7.0495 (2.4); 7.0305 (1.6); 5.2979 (6.0); 5.2159 (0.6); 5.1975 (1.3); 5.1764 (1.4); 5.1578 (0.6); 4.2817 (1.2); 4.2603 (1.1); 4.0348 (11.0); 4.0156 (2.8); 4.0021 (4.9); 3.9880 (3.0); 3.3229 (4.1); 3.3090 (6.0); 3.2954 (3.9); 2.9590 (0.5); 2.9458 (0.5); 2.9371 (0.6); 2.9284 (0.9); 2.9202 (1.0); 2.9076 (0.9); 2.8988 (0.9); 2.8414 (0.6); 2.8212 (1.3); 2.8017 (1.1); 2.7822 (0.7); 2.7626 (0.5); 2.7099 (0.8); 2.7013 (0.7); 2.6921 (0.9); 2.6832 (1.0); 2.6786 (1.0); 2.6726 (0.8); 2.6698 (0.9); 2.6605 (1.0); 2.6519 (1.0); 2.6413 (0.6); 2.6325 (0.6); 2.3263 (16.0); 1.8830 (0.6); 1.8621 (1.5); 1.8517 (0.7); 1.8432 (1.5); 1.8308 (1.4); 1.8218 (0.8); 1.8117 (1.4); 1.7903 (0.6); 1.6231 (3.1); 0.0079 (1.2); −0.0002 (30.2); −0.0084 (1.3)

I-157: ¹H-NMR(400.0 MHz, CDCl3):

δ=7.3390 (2.6); 7.3194 (3.0); 7.2603 (85.3); 6.9962 (0.6); 6.9852 (2.2); 6.9602 (1.5); 6.9153 (3.0); 5.2976 (8.9); 4.8384 (0.6); 4.8239 (1.0); 4.8034(1.0); 4.7903 (0.5); 4.2774 (1.3); 4.2569 (1.1); 4.0226 (11.2); 4.0010 (2.6); 3.9877 (4.5); 3.9734 (2.6); 3.3129 (4.1); 3.2990 (6.0); 3.2854 (3.7); 2.8074 (0.6); 2.7810 (0.8); 2.7655 (1.6); 2.7507 (0.9); 2.7430 (0.9); 2.7257 (1.2); 2.7088 (0.9); 2.2935 (16.0); 2.2769 (2.2); 2.2315 (1.1); 2.0863 (0.6); 2.0773 (1.0); 2.0654 (1.0); 2.0543 (0.8); 2.0424 (0.7); 2.0335 (0.6); 1.9505 (0.6); 1.9366 (1.0); 1.9311 (1.1); 1.9237 (0.9); 1.9166 (1.1); 1.9050 (1.1); 1.8975 (1.3); 1.8903 (1.1); 1.8834 (1.1); 1.8780 (0.9); 1.8673 (0.6); 1.8604 (0.6); 1.8554 (0.8); 1.8420 (0.8); 1.8341 (0.8); 1.8270 (0.8); 1.8192 (0.9); 1.8078 (0.8); 1.7998 (0.7); 1.7918 (0.6); 1.7840 (0.6); 1.6404 (5.5); 0.0080 (1.0); −0.0002 (31.1); −0.0084 (0.9)

1H-NMR

I-158: ¹H-NMR(400.0 MHz, CDCl3):

δ=7.3884 (2.1); 7.3707 (2.5); 7.2619 (66.1); 7.2445 (4.4); 7.2369 (4.1); 7.2202 (2.4); 7.2152 (2.3); 7.2046 (1.7); 7.1894 (0.8); 5.2378 (0.6); 5.2182 (1.6); 5.1993 (1.6); 5.1800 (0.6); 4.7757 (3.8); 4.6449 (7.3); 4.4727 (0.8); 4.4508 (0.8); 4.1122 (1.0); 4.0986 (2.7); 4.0862 (4.1); 4.0736 (3.3); 4.0438 (3.0); 4.0312 (2.6); 4.0189 (1.0); 3.9337 (1.4); 3.9200 (1.9); 3.9073 (1.0); 3.0220 (0.6); 3.0093 (0.6); 3.0004 (0.7); 2.9914 (1.0); 2.9820 (1.2); 2.9691 (1.0); 2.9603 (1.2); 2.9545 (3.8); 2.9040 (0.8); 2.8829 (4.4); 2.8815 (4.2); 2.8633 (1.4); 2.8443 (0.9); 2.8241 (0.6); 2.7149 (0.6); 2.7060 (0.6); 2.6958 (1.0); 2.6838 (1.2); 2.6742 (1.0); 2.6643 (1.2); 2.6551 (1.0); 2.6459 (0.6); 2.6366 (0.5); 2.2305 (7.3); 2.1854 (16.0); 1.8847 (1.1); 1.8662 (1.1); 1.8532 (1.2); 1.8459 (0.9); 1.8342 (1.0); 1.6434 (1.7); 0.0080 (0.8); −0.0002 (25.2); −0.0085 (0.7)

I-159: ¹H-NMR(400.0 MHz, CDCl3):

δ=7.4946 (0.8); 7.4909 (0.7); 7.4846 (1.1); 7.4777 (2.2); 7.4737 (3.9); 7.4652 (16.0); 7.4596 (2.9); 7.4556 (2.7); 7.4519 (1.9); 7.3802 (1.1); 7.3626 (1.3); 7.2604 (55.1); 7.2457 (2.5); 7.2425 (3.2); 7.2379 (2.4); 7.2307 (2.8); 7.2277 (2.4); 7.2157 (1.5); 7.2141 (1.6); 7.2084 (1.4); 7.1982 (1.1); 7.1924 (0.8); 7.1829 (0.6); 5.2066 (0.7); 5.1879 (0.7); 4.1209 (1.5); 3.4840 (6.2); 2.9821 (0.7); 2.9730 (0.8); 2.9606 (0.7); 2.9533 (1.7); 2.8944 (0.5); 2.8829 (1.1); 2.8814 (1.1); 2.8742 (1.2); 2.8540 (0.9); 2.8346 (0.6); 2.6854 (0.5); 2.6762 (0.5); 2.6539 (0.6); 2.6449 (0.5); 1.8766 (0.8); 1.8580 (0.8); 1.8449 (0.8); 1.8262 (0.7); 1.6209 (0.6); 0.0079 (0.6); −0.0002 (19.9); −0.0085 (0.7)

I-160: ¹H-NMR(400.0 MHz. CDCl3):

δ=8.0165 (0.5); 7.5209 (0.8); 7.3970 (5.6); 7.3799 (6.7); 7.2621 (148.6); 7.2491 (9.2); 7.2457 (12.6); 7.2417 (10.2); 7.2339 (12.9); 7.2307 (10.5); 7.2223 (7.0); 7.2172 (5.6); 7.2123 (5.4); 7.2049 (5.0); 7.1984 (3.4); 7.1892 (2.4); 7.1827 (1.4); 6.9979 (0.8); 5.2453 (1.3); 5.2263 (3.7); 5.2070 (3.7); 5.1885 (1.4); 4.8534 (3.9); 4.4660 (1.5); 4.1081 (7.9); 4.0531 (3.0); 3.4838 (11.5); 3.0299 (1.7); 3.0207 (1.9); 3.0083 (1.9); 2.9989 (2.1); 2.9902(3.4); 2.9810 (3.6); 2.9685 (3.5); 2.9595 (3.7); 2.9546 (6.0); 2.9042 (2.6); 2.8830 (10.0); 2.8817 (8.6); 2.8637 (4.4); 2.8441 (3.0); 2.8238 (1.9); 2.7204 (2.3); 2.7112 (2.2); 2.7024 (2.8); 2.7009 (2.8); 2.6932 (2.9); 2.6888 (3.1); 2.6828 (2.5); 2.6795 (2.8); 2.6736 (2.4); 2.6707 (3.3); 2.6615 (3.1); 2.6511 (2.2); 2.6419 (1.8); 1.9088 (1.9); 1.8877 (4.2); 1.8770 (2.1); 1.8690 (4.2); 1.8560 (4.1); 1.8478 (2.1); 1.8373 (4.0); 1.8160 (1.9); 1.7438 (1.6); 1.6653 (1.8); 1.0818 (2.3); 1.0713 (7.8); 1.0640 (9.2); 1.0533 (8.5); 1.0441 (3.1); 0.9134 (0.5); 0.8957 (4.4); 0.8863 (16.0); 0.8790 (12.0); 0.8665 (15.2); 0.8591 (12.0); 0.8486 (3.5); 0.0080 (1.5); −0.0002 (55.4); −0.0085 (1.6)

I-161: ¹H-NMR(400.0 MHz, CDCl3):

δ=7.7741 (1.5); 7.7593 (5.1); 7.7572 (4.8); 7.6732 (1.3); 7.6542 (2.8); 7.6300 (1.6); 7.6103 (1.8); 7.5909 (0.6); 7.3752 (1.3); 7.3576 (1.6); 7.2666 (0.6); 7.2610 (52.2); 7.2568 (0.9); 7.2560 (0.8); 7.2552 (0.7); 7.2544 (0.6); 7.2536 (0.6); 7.2463 (3.1); 7.2432 (4.0); 7.2384 (2.9); 7.2369 (2.8); 7.2315 (3.3); 7.2285 (2.8); 7.2147 (2.2); 7.2092 (1.4); 7.1976 (1.2); 7.1924 (0.9); 7.1825 (0.7); 5.2021 (0.8); 5.1845 (0.8); 4.4170 (0.5); 4.1368 (1.8); 3.4828 (16.0); 3.0129 (0.5); 3.0004 (0.5); 2.9913 (0.6); 2.9826 (0.9); 2.9732 (0.9); 2.9610 (0.9); 2.9519 (0.9); 2.8948 (0.7); 2.8747 (1.4); 2.8546 (1.1); 2.8351 (0.8); 2.6808 (0.6); 2.6705 (0.7); 2.6587 (0.6); 2.6495 (0.7); 2.6397 (0.6); 1.8757 (0.9); 1.8571 (0.9); 1.8440 (0.9); 1.8254 (0.8); 0.0079 (0.6); −0.0002 (20.6); −0.0085 (0.6)

I-162: ¹H-NMR(400.0 MHz, CDCl3):

δ=7.5185 (1.1); 7.4622 (1.8); 7.4552 (1.0); 7.4467 (1.8); 7.4399 (16.0); 7.4334 (19.5); 7.4267 (1.6); 7.4182 (1.2); 7.4135 (1.7); 7.4114 (2.0); 7.3781 (2.0); 7.3602 (2.4); 7.2597 (201.4); 7.2487 (4.2); 7.2455 (5.2); 7.2411 (3.7); 7.2394 (3.5); 7.2336 (4.4); 7.2308 (3.4); 7.2171 (2.8); 7.2098 (2.4); 7.2000 (1.6); 7.1943 (1.1); 7.1847 (0.9); 7.1780 (0.6); 6.9956 (1.1); 5.2043 (1.0); 5.1869 (1.1); 4.7637 (0.9); 4.3568 (0.7); 4.1250 (2.9); 3.0254 (0.6); 3.0164 (0.7); 3.0041 (0.7); 2.9948 (0.7); 2.9858 (1.2); 2.9765 (1.3); 2.9645 (1.2); 2.9551 (1.2); 2.8971 (0.9); 2.8770 (2.0); 2.8570 (1.6); 2.8371 (1.1); 2.8172 (0.7); 2.7032 (0.6); 2.6938 (0.6); 2.6838 (0.9); 2.6718 (1.0); 2.6653 (0.8); 2.6620 (0.8); 2.6526 (1.0); 2.6439 (0.9); 2.6339 (0.6); 2.6247 (0.6); 1.8991 (0.7); 1.8780 (1.4); 1.8673 (0.8); 1.8595 (1.4); 1.8463 (1.4); 1.8383 (0.7); 1.8277 (1.3); 1.8065 (0.6); 1.5849 (1.4); 0.0080 (2.2); −0.0002 (76.0); −0.0085 (2.1)

1H-NMR

I-163: ¹H-NMR(400.0 MHz, CDCl3):

δ=7.4330 (2.0); 7.4178 (2.1); 7.4118 (2.2); 7.2626 (51.4); 7.1722 (2.8); 7.1637 (4.5); 7.1567 (3.5); 7.1491 (2.8); 7.1354 (0.5); 7.1084 (1.9); 7.1025 (2.2); 7.0873 (1.4); 4.8632 (0.6); 4.8461 (1.3); 4.8303 (1.4); 4.8155 (0.7); 4.7641 (3.3); 4.6364 (7.2); 4.4941 (0.9); 4.4721 (1.0); 4.4402 (0.5); 4.0828 (3.5); 4.0796 (3.5); 4.0664 (3.8); 4.0303 (2.8); 4.0172 (2.9); 4.0048 (1.1); 3.9265 (1.3); 3.9125 (1.8); 3.8998 (1.0); 3.4825 (4.8); 2.9541 (1.1); 2.8825 (1.0); 2.8811 (1.0); 2.8551 (0.7); 2.8284 (1.1); 2.8130 (2.2); 2.7961 (1.6); 2.7756 (1.7); 2.7594 (1.1); 2.7332 (0.6); 2.2265 (7.2); 2.1822 (16.0); 2.1162 (0.6); 2.1016 (0.7); 2.0923 (1.3); 2.0812 (1.4); 2.0700 (1.3); 2.0601 (1.2); 2.0486 (0.9); 1.9678 (0.5); 1.9491 (1.2); 1.9427 (1.2); 1.9305 (1.2); 1.9237 (1.3); 1.9160 (1.5); 1.9093 (1.3); 1.8944 (1.5); 1.8750 (1.1); 1.8685 (0.9); 1.8603 (1.0); 1.8529 (1.3); 1.8452 (1.1); 1.8342 (1.0); 1.8104 (0.7); 1.6795 (1.3); 0.0079 (0.5); −0.0002 (19.9); −0.0085 (0.6)

I-164: ¹H-NMR(400.0 MHz, CDCl3):

δ=7.4921 (0.8); 7.4887 (0.7); 7.4822 (1.0); 7.4754 (2.1); 7.4715 (3.8); 7.4631 (16.0); 7.4536 (2.4); 7.4497 (1.6); 7.4213 (1.0); 7.4052 (1.0); 7.3997 (1.0); 7.2613 (29.6); 7.1721 (1.3); 7.1673 (1.5); 7.1640 (1.3); 7.1567 (3.8); 7.1496 (2.0); 7.1459 (1.2); 7.1406 (1.3); 7.1022 (1.5); 7.0959 (1.3); 7.0842 (0.7); 7.0801 (0.9); 4.8336 (0.8); 4.8170 (0.8); 4.8023 (0.6); 4.4172 (0.6); 4.3988 (0.5); 4.1069 (1.5); 3.4793 (11.1); 2.8201 (0.6); 2.8052 (1.1); 2.7851 (0.7); 2.7669 (0.9); 2.7506 (0.7); 2.0801 (0.5); 2.0697 (0.6); 2.0582 (0.5); 1.9478 (0.6); 1.9415 (0.6); 1.9336 (0.6); 1.9265 (0.7); 1.9155 (0.7); 1.9077 (0.6); 1.9000 (0.7); 1.8930 (0.6); 1.8860 (0.6); 1.8432 (0.6); 1.8352 (0.6); 1.8234 (0.5); −0.0002 (11.5)

I-165: ¹H-NMR(400.0 MHz, CDCl3):

δ=7.2616 (36.9); 7.0879 (3.4); 7.0622 (2.1); 7.0133 (1.9); 6.9975 (1.2); 4.7909 (2.6); 4.7703 (1.7); 4.7483 (0.9); 4.6619 (5.0); 4.3688 (0.7); 4.3444 (1.0); 4.1049 (1.2); 4.0932 (3.0); 4.0802 (3.2); 4.0419 (2.0); 4.0291 (2.1); 4.0162 (0.8); 3.9388 (0.9); 3.9250 (1.2); 3.9124 (0.6); 3.4840 (1.4); 3.0502 (0.7); 3.0312 (0.8); 3.0120 (0.8); 2.9928 (0.9); 2.9544 (0.6); 2.8828 (0.5); 2.5163 (0.9); 2.4934 (1.0); 2.4782 (0.8); 2.4546 (1.0); 2.2965 (16.0); 2.2563 (0.6); 2.2333 (5.9); 2.1940 (11.0); 1.6614 (1.0); 1.2923 (10.2); 1.2755 (10.0); −0.0002 (14.1)

I-166: ¹H-NMR(400.0 MHz, CDCl3):

δ=7.5186 (0.6); 7.4955 (1.5); 7.4865 (2.4); 7.4820 (3.4); 7.4724 (16.0); 7.4696 (15.0); 7.4640 (2.2); 7.4598 (2.4); 7.4554 (1.8); 7.4516 (1.3); 7.4369 (0.6); 7.2598 (91.4); 7.2100 (0.5); 7.0857 (3.0); 7.0765 (2.4); 7.0570 (3.5); 7.0102 (2.6); 6.9955 (1.2); 6.9912 (1.5); 4.7824 (1.2); 4.7596 (1.8); 4.7376 (1.0); 4.2816 (0.9); 4.2600 (0.8); 4.1151 (2.1); 3.4857 (3.4); 3.0426 (1.2); 3.0233 (1.3); 3.0041 (1.4); 2.9850 (1.5); 2.5079 (0.9); 2.4855 (1.1); 2.4701 (0.8); 2.4477 (1.0); 2.2947 (15.8); 2.2685 (0.5); 2.2471 (0.9); 2.2291 (1.1); 2.2117 (0.8); 1.5978 (1.2); 1.2850 (9.3); 1.2683 (9.1); 0.0080 (0.9); −0.0002 (35.3); −0.0085 (1.0

I-167: ¹H-NMR(400.0 MHz, CDCl3):

δ=7.2605 (75.5); 7.0988 (3.3); 7.0814 (1.8); 7.0623 (3.0); 7.0142 (2.3); 6.9965 (1.6); 4.8880 (1.0); 4.8022 (1.0); 4.7794 (1.7); 4.7580 (1.0); 4.3182 (0.8); 4.2955 (0.8); 4.1223 (2.3); 4.0595 (0.8); 3.4869 (1.2); 3.0503 (1.0); 3.0311 (1.1); 3.0117 (1.2); 2.9927 (1.2); 2.5182 (0.9); 2.4954 (1.0); 2.4805 (0.8); 2.4569 (1.0); 2.2993 (16.0); 2.2802 (0.5); 2.2601 (0.8); 2.2409 (1.0); 2.2234 (0.7); 1.6281 (1.2); 1.2976 (13.6); 1.2808 (13.3); 1.0889 (0.7); 1.0784 (2.4); 1.0711 (2.7); 1.0678 (2.6); 1.0602 (2.5); 1.0510 (1.0); 0.8997 (1.1); 0.8905 (4.1); 0.8831 (3.2); 0.8707 (3.9); 0.8632 (3.2); 0.8528 (0.9); 0.0080 (0.8); −0.0002 (29.0); −0.0085 (0.9)

I-168: ¹H-NMR(400.0 MHz, CDCl3):

δ=7.7731 (3.9); 7.7713 (4.2); 7.7691 (4.3); 7.7627 (2.6); 7.7572 (2.3); 7.6825 (1.6); 7.6633 (3.2); 7.6323 (1.9); 7.6236 (0.5); 7.6126 (2.3); 7.5927 (0.8); 7.2603 (63.8); 7.0770 (5.1); 7.0577 (3.7); 7.0115 (2.8); 6.9922 (1.5); 4.7765 (1.1); 4.7552 (1.7); 4.7337 (1.1); 4.3159 (0.8); 4.2947 (0.8); 4.1347 (2.0); 3.4835 (7.0); 3.0428 (1.2); 3.0237 (1.3); 3.0044 (1.4); 2.9853 (1.5); 2.5083 (1.0); 2.4856 (1.1); 2.4700 (0.8); 2.4474 (1.1); 2.2929 (16.0); 2.2688 (0.5); 2.2489 (0.9); 2.2297 (1.1); 2.2113 (0.8); 1.6263 (0.6); 1.2963 (0.6); 1.2825 (9.1); 1.2658 (8.8); 0.0080 (0.6); −0.0002 (24.6); −0.0085 (0.7)

1H-NMR

I-169: ¹H-NMR(400.0 MHz, CDCl3):

δ=7.4791 (0.8); 7.4745 (1.1); 7.4623 (1.1); 7.4593 (1.3); 7.4566 (1.7); 7.4385 (0.8); 7.4334 (0.8); 7.4204 (1.9); 7.4163 (2.0); 7.4101 (2.0); 7.4024 (1.8); 7.3981 (1.5); 7.3925 (2.4); 7.3859 (3.1); 7.3805 (2.1); 7.3747 (1.4); 7.3667 (3.3); 7.3634 (2.4); 7.3579 (2.9); 7.3421 (2.4); 7.2681 (0.5); 7.2673 (0.6); 7.2664 (0.8); 7.2656 (1.0); 7.2608 (66.2); 7.2551 (1.0); 7.2527 (0.6); 7.1043 (2.0); 7.0813 (1.3); 7.0633 (4.2); 7.0531 (2.6); 7.0096 (2.3); 6.9967 (1.4); 6.9903 (1.2); 5.0444 (0.8); 5.0310 (0.8); 4.9552 (0.9); 4.9472 (0.8); 4.7792 (1.0); 4.7649 (0.8); 4.7576 (0.8); 4.7434 (1.2); 4.7222 (0.7); 4.5226 (1.2); 4.4283 (1.2); 4.3828 (0.7); 4.3508 (0.9); 4.3275 (1.0); 4.2769 (1.1); 4.2546 (1.1); 4.1862 (1.1); 4.1744 (1.9); 4.1603 (1.4); 4.1442 (0.9); 4.0993 (0.6); 3.9714 (0.5); 3.9567 (0.5); 3.7579 (0.7); 3.7172 (0.8); 3.4815 (16.0); 3.0511 (0.7); 3.0384 (0.9); 3.0327 (0.8); 3.0191 (1.0); 3.0129 (0.9); 2.9997 (1.1); 2.9940 (1.0); 2.9810 (1.0); 2.9539 (1.4); 2.8826 (1.2); 2.8812 (1.2); 2.5191 (0.5); 2.4983 (1.2); 2.4784 (1.1); 2.4599 (1.1); 2.4391 (0.7); 2.3051 (8.8); 2.2897 (11.7); 2.2614 (0.9); 2.2417 (1.2); 2.2239 (1.2); 2.2046 (0.9); 1.6398 (0.6); 1.3010 (6.0); 1.2842 (6.4); 1.2728 (9.1); 1.2659 (1.6); 1.2560 (8.7); 0.0080 (0.7); −0.0002 (26.2); −0.0085 (0.9)

I-170: ¹H-NMR(400.0 MHz, CDCl3):

δ=7.4888 (0.6); 7.4835 (2.6); 7.4809 (5.8); 7.4770 (5.4); 7.4720 (4.3); 7.4676 (3.0); 7.4642 (3.3); 7.4590 (1.5); 7.4250 (2.1); 7.4171 (0.5); 7.4061 (3.5); 7.3979 (0.9); 7.3849 (2.2); 7.3631 (3.0); 7.3595 (5.3); 7.3558 (2.8); 7.3442 (1.6); 7.3406 (2.7); 7.3370 (1.3); 7.2607 (57.2); 7.0787 (4.2); 7.0581 (3.6); 7.0132 (2.6); 7.0115 (2.7); 6.9926 (1.5); 4.7792 (1.1); 4.7574 (1.8); 4.7355 (1.2); 4.3223 (0.8); 4.3003 (0.8); 4.1149 (2.1); 3.4831 (10.5); 3.0435 (1.2); 3.0244 (1.3); 3.0050 (1.4); 2.9858 (1.5); 2.9540 (1.0); 2.8828 (0.9); 2.8814 (0.9); 2.5088 (0.9); 2.4864 (1.1); 2.4709 (0.8); 2.4483 (1.0); 2.2948 (16.0); 2.2695 (0.6); 2.2487 (0.9); 2.2303 (1.2); 2.2126 (0.8); 1.6356 (0.5); 1.2842 (9.7); 1.2674 (9.4); 0.0079 (0.6); −0.0002 (21.9); −0.0085 (0.7)

I-171: ¹H-NMR(400.0 MHz, CDCl3):

δ=7.4642 (0.8); 7.4616 (0.7); 7.4562 (0.5); 7.4491 (0.9); 7.4413 (16.0); 7.4384 (18.5); 7.4308 (0.8); 7.4238 (0.6); 7.4184 (0.8); 7.4158 (0.9); 7.2609 (46.1); 7.0775 (4.3); 7.0574 (3.0); 7.0127 (2.2); 7.0109 (2.3); 6.9938 (1.2); 6.9919 (1.2); 4.7767 (1.2); 4.7552 (1.8); 4.7336 (1.0); 4.3243 (0.8); 4.3019 (0.8); 4.1100 (2.2); 3.4822 (9.7); 3.0422 (1.0); 3.0231 (1.1); 3.0037 (1.2); 2.9846 (1.3); 2.9542 (0.8); 2.8828 (0.7); 2.8813 (0.7); 2.5084 (0.8); 2.4855 (1.0); 2.4697 (0.7); 2.4478 (0.9); 2.2932 (13.8); 2.2472 (0.8); 2.2280 (1.0); 2.2107 (0.7); 1.2823 (9.2); 1.2655 (9.0); −0.0002 (18.4); −0.0085 (0.5)

I-172: ¹H-NMR(400.0 MHz, CDCl3):

δ=7.5191 (0.6); 7.2603 (103.5); 7.2106 (0.5); 7.0938 (3.4); 7.0822 (1.7); 7.0631 (2.7); 7.0150 (2.2); 6.9962 (1.8); 4.7965 (1.1); 4.7754 (2.1); 4.7528 (1.4); 4.7261 (1.5); 4.2806 (1.2); 4.2563 (1.1); 4.0870 (1.4); 4.0343 (1.2); 3.4887 (2.4); 3.0508 (0.8); 3.0319 (0.9); 3.0123 (1.0); 2.9934 (1.0); 2.5167 (1.3); 2.4942 (1.4); 2.4789 (1.0); 2.4558 (1.0); 2.2973 (16.0); 2.2586 (0.7); 2.2396 (0.9); 2.2222 (0.6); 1.8224 (1.3); 1.7770 (1.2); 1.7444 (1.7); 1.5920 (1.9); 1.2955 (13.4); 1.2787 (13.8); 0.0079 (1.1); −0.0002 (38.8); −0.0085 (1.1)

I-173: ¹H-NMR(400.0 MHz, CDCl3):

δ=7.2625 (38.9); 7.1882 (3.5); 7.1420 (1.4); 7.1230 (2.2); 7.0562 (2.0); 7.0374 (1.3); 5.1779 (1.3); 5.1587 (1.3); 4.7786 (2.7); 4.6478 (5.5); 4.4566 (0.7); 4.4344 (0.7); 4.4174 (0.5); 4.1142 (0.7); 4.1002 (2.1); 4.0876 (3.2); 4.0749 (2.6); 4.0456 (2.3); 4.0332 (2.0); 3.9352 (1.0); 3.9215 (1.5); 3.9085 (0.8); 3.4840 (3.6); 2.9546 (1.5); 2.9369 (0.7); 2.9281 (0.8); 2.9072 (0.8); 2.8828 (1.0); 2.8815 (1.0); 2.8486 (0.5); 2.8287 (1.2); 2.8085 (0.9); 2.7895 (0.6); 2.6824 (0.7); 2.6706 (0.9); 2.6612 (0.8); 2.6511 (0.9); 2.6420 (0.8); 2.3289 (16.0); 2.2315 (5.6); 2.1863 (11.9); 1.8660 (0.8); 1.8472 (0.9); 1.8349 (0.9); 1.8273 (0.7); 1.8156 (0.7); 1.6689 (1.0); −0.0002 (14.2); −0.0085 (0.5)

I-174: ¹H-NMR(400.0 MHz, CDCl3):

δ=7.4941 (0.8); 7.4907 (0.7); 7.4842 (1.1); 7.4774 (2.2); 7.4735 (3.8); 7.4651 (16.0); 7.4557 (2.4); 7.4516 (1.6); 7.2606 (34.2); 7.1795 (2.2); 7.1360 (1.5); 7.1169 (2.4); 7.0541 (1.7); 7.0523 (1.8); 7.0332 (1.1); 5.1662 (0.7); 5.1475 (0.7); 4.3725 (0.5); 4.3530 (0.5); 4.1199 (1.5); 3.4806 (7.4); 2.9276 (0.6); 2.9187 (0.6); 2.9059 (0.6); 2.8974 (0.6); 2.8199 (0.9); 2.8000 (0.7); 2.7808 (0.5); 2.6721 (0.5); 2.6626 (0.5); 2.6593 (0.5); 2.6406 (0.6); 2.6317 (0.5); 2.3248 (11.5); 1.8578 (0.8); 1.8389 (0.8); 1.8263 (0.8); 1.8074 (0.7); −0.0002 (13.1)

1H-NMR

I-175: ¹H-NMR(400.0 MHz, CDCl3):

δ=7.2615 (43.3); 7.1956 (3.3); 7.1412 (1.9); 7.1221 (2.9); 7.0560 (2.2); 7.0370 (1.4); 5.1867 (1.0); 5.1680 (1.0); 4.8635 (1.1); 4.4088 (0.5); 4.1123 (2.3); 4.0598 (0.8); 3.4846 (4.7); 2.9544 (3.0); 2.9458 (0.5); 2.9371 (0.8); 2.9282 (0.8); 2.9154 (0.8); 2.9068 (0.8); 2.8831 (2.2); 2.8817 (2.2); 2.8503 (0.6); 2.8301 (1.2); 2.8102 (1.0); 2.7912 (0.7); 2.7072 (0.7); 2.6984 (0.6); 2.6894 (0.8); 2.6805 (0.8); 2.6758 (0.9); 2.6699 (0.7); 2.6668 (0.8); 2.6609 (0.7); 2.6577 (0.9); 2.6488 (0.9); 2.6385 (0.6); 2.6294 (0.5); 2.3306 (16.0); 1.8914 (0.5); 1.8704 (1.2); 1.8599 (0.6); 1.8516 (1.2); 1.8390 (1.1); 1.8304 (0.6); 1.8201 (1.2); 1.7988 (0.6); 1.0830 (0.7); 1.0724 (2.2); 1.0651 (2.7); 1.0545 (2.4); 1.0452 (0.9); 0.8963 (1.2); 0.8869 (4.5); 0.8796 (3.4); 0.8671 (4.2); 0.8597 (3.4); 0.8492 (1.0); −0.0002 (16.5)

I-176: ¹H-NMR(400.0 MHz, CDCl3):

δ=7.7740 (1.8); 7.7671 (2.0); 7.7606 (5.0); 7.7585 (5.1); 7.7568 (4.7); 7.6749 (1.5); 7.6560 (3.2); 7.6304 (1.8); 7.6107 (2.1); 7.5907 (0.7); 7.2607 (55.7); 7.1745 (3.0); 7.1377 (2.1); 7.1186 (3.3); 7.0546 (2.5); 7.0355 (1.6); 5.1618 (0.9); 5.1438 (1.0); 4.3728 (0.7); 4.3574 (0.7); 4.1417 (2.0); 3.4834 (10.6); 2.9594 (0.5); 2.9543 (1.6); 2.9477 (0.5); 2.9379 (0.5); 2.9295 (0.8); 2.9204 (0.9); 2.9080 (0.9); 2.8993 (0.8); 2.8827 (1.2); 2.8813 (1.2); 2.8407 (0.6); 2.8210 (1.3); 2.8011 (1.0); 2.7817 (0.7); 2.6685 (0.7); 2.6589 (0.8); 2.6465 (0.6); 2.6374 (0.8); 2.6282 (0.7); 2.3247 (16.0); 1.8585 (1.1); 1.8477 (0.5); 1.8397 (1.1); 1.8270 (1.0); 1.8188 (0.5); 1.8082 (1.0); 0.0079 (0.6); −0.0002 (22.0); −0.0085 (0.6)

I-177: ¹H-NMR(400.0 MHz, CDCl3):

δ=7.4785 (0.7); 7.4740 (1.0); 7.4616 (1.0); 7.4588 (1.2); 7.4558 (1.6); 7.4424 (0.8); 7.4377 (0.9); 7.4337 (0.8); 7.4281 (0.9); 7.4240 (1.4); 7.4189 (2.0); 7.4164 (2.1); 7.4105 (1.8); 7.4028 (2.0); 7.3978 (1.4); 7.3916 (2.8); 7.3858 (2.6); 7.3802 (1.8); 7.3733 (1.5); 7.3666 (2.3); 7.3602 (2.1); 7.3552 (2.4); 7.3525 (2.3); 7.3375 (2.8); 7.3198 (0.5); 7.2612 (55.7); 7.1977 (1.8); 7.1667 (1.8); 7.1430 (1.0); 7.1329 (1.4); 7.1238 (1.7); 7.1139 (2.1); 7.0518 (1.7); 7.0328 (1.0); 5.1863 (0.6); 5.1682 (0.8); 5.1518 (0.9); 5.1337 (0.8); 5.0280 (0.7); 5.0142 (0.8); 4.9594 (0.8); 4.9474 (0.7); 4.5154 (1.2); 4.4233 (1.4); 4.3814 (0.9); 4.3371 (1.0); 4.3206 (0.8); 4.2421 (0.5); 4.1909 (1.0); 4.1798 (1.7); 4.1660 (1.3); 4.1516 (0.7); 4.1101 (0.5); 3.7695 (0.6); 3.7532 (0.6); 3.7272 (0.5); 3.7144 (0.7); 3.4815 (16.0); 2.9540 (1.4); 2.9408 (0.7); 2.9315 (0.8); 2.9157 (0.9); 2.9032 (0.6); 2.8938 (0.5); 2.8825 (0.8); 2.8811 (0.8); 2.8319 (0.9); 2.8128 (1.2); 2.7935 (0.9); 2.7742 (0.6); 2.6772 (0.7); 2.6683 (0.7); 2.6589 (0.9); 2.6498 (0.9); 2.6399 (0.7); 2.6305 (0.6); 2.3353 (7.9); 2.3199 (10.0); 1.8759 (0.8); 1.8565 (1.0); 1.8445 (0.8); 1.8358 (0.8); 1.8250 (1.0); 1.8042 (0.7); 1.6460 (0.5); 0.0079 (0.6); −0.0002 (21.2); −0.0085 (0.7)

I-178: ¹H-NMR(400.0 MHz, CDCl3):

δ=7.4875 (0.8); 7.4823 (1.8); 7.4791 (1.9); 7.4743 (3.4); 7.4692 (6.0); 7.4656 (7.2); 7.4630 (4.1); 7.4577 (1.1); 7.4228 (1.8); 7.4095 (0.6); 7.4039 (3.0); 7.3904 (0.7); 7.3825 (1.9); 7.3554 (2.8); 7.3519 (4.6); 7.3483 (2.7); 7.3365 (1.6); 7.3329 (2.6); 7.3294 (1.4); 7.2605 (50.9); 7.1781 (3.2); 7.1380 (2.1); 7.1190 (3.3); 7.0546 (2.5); 7.0355 (1.6); 5.1644 (1.0); 5.1464 (1.0); 4.7581 (0.5); 4.3738 (0.7); 4.3567 (0.7); 4.1229 (2.2); 3.4836 (9.8); 2.9604 (0.5); 2.9476 (0.5); 2.9388 (0.6); 2.9304 (0.8); 2.9213 (0.9); 2.9089 (0.9); 2.8999 (0.9); 2.8421 (0.6); 2.8218 (1.3); 2.8018 (1.0); 2.7826 (0.7); 2.6708 (0.7); 2.6618 (0.8); 2.6489 (0.7); 2.6395 (0.9); 2.6302 (0.7); 2.3260 (16.0); 1.8597 (1.1); 1.8490 (0.6); 1.8410 (1.2); 1.8282 (1.1); 1.8200 (0.6); 1.8095 (1.0); 0.0080 (0.6); −0.0002 (18.7); −0.0085 (0.6)

I-179: ¹H-NMR(400.0 MHz, CDCl3):

δ=7.4606 (1.2); 7.4537 (0.7); 7.4451 (1.2); 7.4383 (10.7); 7.4316 (12.7); 7.4249 (1.1); 7.4163 (0.8); 7.4116 (1.1); 7.4095 (1.4); 7.2607 (35.2); 7.1755 (2.7); 7.1371 (1.8); 7.1180 (2.7); 7.0537 (2.0); 7.0347 (1.3); 5.1528 (0.7); 4.7598 (0.6); 4.1180 (2.1); 3.4823 (16.0); 2.9286 (0.7); 2.9196 (0.7); 2.9072 (0.7); 2.8984 (0.7); 2.8210 (1.1); 2.8011 (0.8); 2.7818 (0.6); 2.6699 (0.6); 2.6609 (0.6); 2.6570 (0.6); 2.6509 (0.5); 2.6477 (0.6); 2.6384 (0.7); 2.6294 (0.6); 2.3247 (13.4); 2.0028 (0.6); 1.8562 (1.0); 1.8374 (1.0); 1.8247 (0.9); 1.8059 (0.9); −0.0002 (13.0)

1H-NMR

I-180: ¹H-NMR(400.0 MHz, CDCl3):

δ=7.2623 (36.5); 7.2402 (3.8); 6.9930 (6.8); 4.8018 (0.9); 4.7867 (1.2); 4.7731 (3.1); 4.6490 (5.0); 4.4492 (0.7); 4.4273 (1.0); 4.0810 (2.5); 4.0708 (2.5); 4.0381 (2.1); 4.0256 (2.0); 3.9300 (0.9); 3.9164 (1.3); 3.9033 (0.7); 3.4833 (5.1); 2.9545 (2.0); 2.8827 (1.8); 2.8814 (1.7); 2.7797 (0.8); 2.7645 (1.5); 2.7486 (0.9); 2.7252 (1.2); 2.7090 (0.8); 2.2794 (16.0); 2.2289 (5.1); 2.1855 (11.1); 2.0836 (0.6); 2.0745 (1.0); 2.0629 (1.0); 2.0518 (0.9); 2.0425 (0.8); 2.0308 (0.7); 1.9370 (0.6); 1.9284 (0.8); 1.9220 (0.9); 1.9155 (0.9); 1.9036 (1.0); 1.8946 (1.0); 1.8885 (1.0); 1.8810 (0.9); 1.8749 (1.0); 1.8643 (0.6); 1.8530 (0.8); 1.8452 (0.7); 1.8384 (0.6); 1.8304 (0.8); 1.8215 (0.8); 1.8097 (0.8); 1.7871 (0.5); 1.6780 (0.7); −0.0002 (14.1)

I-181: ¹H-NMR(400.0 MHz, CDCl3):

δ=7.4922 (0.9); 7.4894 (0.7); 7.4822 (1.1); 7.4723 (3.6); 7.4638 (16.0); 7.4546 (2.3); 7.4501 (1.6); 7.2605 (31.9); 7.2296 (2.4); 6.9962 (0.5); 6.9893 (9.0); 6.9866 (8.3); 4.7747 (1.1); 4.3811 (0.6); 4.3623 (0.6); 4.1148 (1.6); 3.4807 (5.4); 2.9534 (0.6); 2.7726 (0.6); 2.7575 (1.1); 2.7423 (0.7); 2.7350 (0.6); 2.7180 (0.9); 2.7010 (0.7); 2.2739 (12.7); 2.0634 (0.5); 2.0529 (0.6); 2.0410 (0.5); 1.9297 (0.6); 1.9237 (0.6); 1.9156 (0.6); 1.9086 (0.7); 1.8975 (0.8); 1.8825 (0.7); 1.8645 (0.6); 1.8203 (0.6); 1.8121 (0.6); 1.8004 (0.6); −0.0002 (12.1)

I-182: ¹H-NMR(400.0 MHz, CDCl3):

δ=7.2610 (47.0); 7.2490 (3.5); 6.9955 (9.4); 6.9929 (8.8); 4.8758 (0.9); 4.8285 (0.8); 4.8151 (1.2); 4.7956 (1.2); 4.7813 (0.6); 4.3985 (0.7); 4.3782 (0.7); 4.1110 (2.1); 4.0551 (0.8); 3.4861 (3.8); 2.9551 (0.6); 2.7832 (0.7); 2.7679 (1.4); 2.7526 (0.8); 2.7448 (0.7); 2.7276 (1.0); 2.7108 (0.8); 2.2818 (16.0); 2.0817 (0.8); 2.0702 (0.8); 2.0588 (0.7); 2.0499 (0.7); 2.0379 (0.5); 1.9417 (0.7); 1.9351 (0.7); 1.9269 (0.7); 1.9200 (0.8); 1.9088 (0.8); 1.8997 (0.8); 1.8932 (0.8); 1.8862 (0.7); 1.8798 (0.7); 1.8693 (0.5); 1.8577 (0.6); 1.8489 (0.6); 1.8401 (0.5); 1.8333 (0.7); 1.8255 (0.8); 1.8137 (0.7); 1.7980 (0.6); 1.7901 (0.7); 1.7773 (0.6); 1.7638 (0.5); 1.7560 (0.5); 1.6323 (1.0); 1.0822 (0.6); 1.0717 (2.2); 1.0644 (2.5); 1.0537 (2.4); 1.0445 (0.9); 0.8947 (1.2); 0.8855 (3.8); 0.8781 (3.0); 0.8656 (3.7); 0.8582 (3.0); 0.8478 (0.9); −0.0002 (18.1); −0.0085 (0.6)

I-183: ¹H-NMR(400.0 MHz, CDCl3):

δ=7.7722 (1.7); 7.7590 (4.6); 7.7569 (4.7); 7.6738 (1.4); 7.6545 (3.0); 7.6289 (1.8); 7.6090 (2.0); 7.5893 (0.7); 7.2604 (49.2); 7.2207 (3.0); 7.2110 (0.6); 6.9911 (11.2); 6.9884 (10.7); 4.7730 (1.3); 4.3862 (0.7); 4.3673 (0.7); 4.1354 (2.0); 3.4831 (9.1); 2.9542 (0.7); 2.8827 (0.6); 2.8813 (0.6); 2.7734 (0.8); 2.7581 (1.4); 2.7430 (0.9); 2.7362 (0.8); 2.7192 (1.2); 2.7017 (0.8); 2.2737 (16.0); 2.0597 (0.7); 2.0502 (0.8); 2.0380 (0.6); 2.0272 (0.6); 1.9282 (0.8); 1.9223 (0.8); 1.9137 (0.8); 1.9070 (0.9); 1.8959 (0.9); 1.8810 (0.9); 1.8649 (0.7); 1.8428 (0.6); 1.8354 (0.6); 1.8273 (0.6); 1.8203 (0.8); 1.8122 (0.8); 1.8009 (0.7); 1.7853 (0.5); 0.0080 (0.5); −0.0002 (19.2); −0.0085 (0.5)

I-184: ¹H-NMR(400.0 MHz, CDCl3):

δ=7.4773 (0.7); 7.4728 (1.1); 7.4603 (1.0); 7.4576 (1.3); 7.4546 (1.7); 7.4416 (0.9); 7.4371 (1.2); 7.4323 (0.7); 7.4268 (1.0); 7.4235 (1.7); 7.4209 (2.0); 7.4187 (2.3); 7.4149 (1.8); 7.4091 (1.7); 7.4012 (2.0); 7.3965 (1.4); 7.3901 (2.9); 7.3840 (2.9); 7.3789 (1.8); 7.3718 (1.6); 7.3651 (2.2); 7.3574 (2.1); 7.3532 (2.9); 7.3499 (2.8); 7.3367 (3.5); 7.3326 (2.7); 7.3191 (0.7); 7.2608 (57.9); 7.2507 (2.0); 7.2212 (1.7); 6.9965 (5.9); 6.9939 (5.8); 6.9867 (7.5); 6.9841 (7.0); 5.0124 (1.1); 4.9507 (1.1); 4.8139 (0.5); 4.7942 (0.9); 4.7799 (0.9); 4.7607 (0.7); 4.5547 (0.5); 4.5119 (1.4); 4.4235 (2.0); 4.4045 (0.8); 4.3807 (1.0); 4.3530 (1.3); 4.3341 (1.0); 4.2159 (0.5); 4.2050 (0.5); 4.1717 (2.2); 4.1583 (1.3); 4.1430 (0.7); 4.1188 (0.5); 4.1051 (0.6); 3.7647 (0.6); 3.7485 (0.6); 3.7218 (0.5); 3.7086 (0.8); 3.4817 (16.0); 2.7687 (1.2); 2.7527 (1.4); 2.7292 (1.2); 2.7117 (1.2); 2.6960 (0.7); 2.2862 (9.0); 2.2698 (10.0); 2.0840 (0.6); 2.0751 (0.7); 2.0631 (0.7); 2.0515 (0.9); 2.0401 (0.8); 2.0286 (0.6); 2.0165 (0.5); 1.9438 (0.7); 1.9373 (0.8); 1.9232 (1.1); 1.9168 (0.9); 1.9097 (1.0); 1.9038 (1.1); 1.8970 (0.9); 1.8903 (1.2); 1.8850 (1.0); 1.8759 (0.9); 1.8695 (0.9); 1.8553 (0.7); 1.8351 (0.8); 1.8279 (0.9); 1.8198 (0.8); 1.8129 (1.0); 1.8057 (0.9); 1.7932 (0.8); 1.7786 (0.6); 1.6370 (0.6); 0.0080 (0.6); −0.0002 (22.5); −0.0085 (0.6)

1H-NMR

I-185: ¹H-NMR(400.0 MHz, CDCl3):

δ=7.4859 (0.6); 7.4808 (1.4); 7.4775 (1.4); 7.4729 (2.5); 7.4678 (4.4); 7.4641 (5.4); 7.4614 (2.9); 7.4562 (0.7); 7.4216 (1.4); 7.4027 (2.2); 7.3893 (0.5); 7.3814(1.4); 7.3545 (2.0); 7.3510 (3.4); 7.3474 (2.0); 7.3357 (1.2); 7.3321(2.0); 7.3286 (1.0); 7.2610 (35.1); 7.2255 (2.5); 6.9914 (8.7); 6.9888(8.6); 4.7742 (1.1); 4.3911 (0.6); 4.3726 (0.6); 4.1165 (1.7); 3.4830 (16.0); 2.7741 (0.6); 2.7591 (1.2); 2.7438 (0.7); 2.7366 (0.7); 2.7191(0.9); 2.7023 (0.6); 2.2752 (12.9); 2.0614 (0.6); 2.0514 (0.6); 2.0395(0.5); 1.9303 (0.6); 1.9242 (0.6); 1.9166 (0.6); 1.9095 (0.7); 1.8982 (0.8); 1.8828 (0.7); 1.8645 (0.6); 1.8442 (0.5); 1.8214 (0.6); 1.8134 (0.6): 1.8015 (0.6); −0.0002 (13.7)

I-186: ¹H-NMR(400.0 MHz, CDCl3):

δ=7.4597 (1.3); 7.4528 (0.7); 7.4442 (1.4); 7.4374 (12.2); 7.4310 (14.4); 7.4242 (1.2); 7.4156 (0.9); 7.4109 (1.3); 7.4087 (1.5); 7.2611 (42.3); 7.2229 (3.1); 6.9908 (10.8); 6.9880 (10.1); 4.8033 (0.6); 4.7708 (1.4); 4.7575 (1.1); 4.3899 (0.8); 4.3697 (0.8); 4.1136 (2.3); 3.4826 (16.0); 2.7734 (0.7); 2.7584 (1.4); 2.7431 (0.8); 2.7362(0.8); 2.7198 (1.1); 2.7023 (0.8); 2.2743(15.7); 2.0608(0.7); 2.0500(0.8); 2.0390 (0.6); 2.0289 (0.6); 1.9278 (0.8); 1.9211 (0.7); 1.9132 (0.8); 1.9065 (0.8); 1.8954 (0.9); 1.8862 (0.7); 1.8796 (0.9); 1.8728 (0.8); 1.8649 (0.7); 1.8544 (0.5); 1.8436 (0.6); 1.8355 (0.6); 1.8281 (0.6); 1.8207 (0.8); 1.8124 (0.8); 1.8012 (0.7); 1.7857 (0.5); −0.0002 (16.4)

I-187: ¹H-NMR(400.0 MHz, CDCl3):

δ=7.2618 (45.9); 7.2440 (3.5); 6.9952 (8.4); 6.9930 (7.9); 4.8082 (0.9); 4.7891 (1.1); 4.7746 (0.8); 4.7081 (1.4); 4.3868 (1.0); 4.3660 (1.0); 4.0758 (1.4); 4.0251 (1.2); 3.4849 (11.8); 2.7817 (0.7); 2.7668 (1.4); 2.7521 (0.8); 2.7432(0.7); 2.7265 (1.0); 2.7092 (0.8); 2.2801 (16.0); 2.0771 (0.8); 2.0655 (0.9); 2.0541 (0.8); 2.0452 (0.7); 2.0419 (0.7); 2.0334 (0.6); 1.9239 (0.7); 1.9099 (0.8); 1.8967 (0.9); 1.8903 (0.8); 1.8833 (0.7); 1.8767 (0.9); 1.8544 (1.2); 1.8462 (1.4); 1.8308 (1.9); 1.8228 (2.0); 1.8159 (1.8); 1.8121 (1.8); 1.7879 (0.9); 1.7690 (1.4); 1.7286 (1.9); 1.5494 (0.8); 1.2791 (2.0); −0.0002 (18.3); −0.0085 (0.6)

I-188: ¹H-NMR(400.0 MHz, CDCl3):

δ=7.5198 (0.7); 7.2609 (116.2); 7.2412 (2.4); 7.2111 (0.6); 7.0846 (1.1); 6.9969 (0.8); 6.9876 (0.6); 6.9629 (1.1); 6.9417 (1.3); 6.7694 (0.8); 6.7485 (0.7); 6.7201 (1.9); 6.6995 (1.6); 5.0117 (0.7); 4.9962 (0.7); 4.7867 (1.6); 4.6764 (0.6); 4.6499 (3.9); 4.6399 (1.9); 4.3058 (1.0); 4.3019 (1.1); 4.2900 (1.0); 4.2862 (1.0); 4.2775 (1.0); 4.2736 (1.0); 4.2618 (1.0); 4.2579 (0.9); 4.0950 (1.9); 4.0862 (2.8); 4.0746 (2.3); 4.0367 (2.0); 4.0245 (1.8); 3.9387 (0.9); 3.9250 (1.2); 3.4887 (5.2); 2.9549 (2.1); 2.8838 (1.8); 2.8824 (1.8); 2.5285 (0.6); 2.5119 (0.6); 2.4959 (0.7); 2.2833 (0.5); 2.2461 (7.8); 2.2403 (16.0); 2.2156 (0.8); 2.1927 (8.7); 2.1847 (4.3); 1.6331 (0.9); 1.6048 (2.3); 1.6005 (2.0); 1.5898 (2.1); 1.5767 (1.7); 1.5725 (2.1); 1.5439 (0.8); 1.4168 (3.5); 1.4007 (13.1); 1.3849 (9.8); 0.0080 (1.2); −0.0002 (45.2); −0.0085 (1.3)

I-189: ¹H-NMR(400.0 MHz, CDCl3):

δ=7.5188 (0.8); 7.4994 (1.0); 7.4894 (1.2); 7.4819 (2.2); 7.4781 (3.8); 7.4693 (16.0); 7.4658 (10.1); 7.4599 (3.0); 7.4559 (2.6); 7.2600 (112.8); 7.2335 (1.7); 7.2100 (0.6); 7.0740 (0.8); 7.0067 (0.6); 7.0014 (0.5); 6.9959 (0.7); 6.9862 (0.7); 6.9806 (0.6); 6.9618 (1.0); 6.9565 (1.0); 6.9412 (1.2); 6.9358 (1.2); 6.7632 (1.4); 6.7424 (1.2); 6.7144 (3.1); 6.6937 (2.6); 4.7849 (0.6); 4.6650 (0.5); 4.2912 (0.7); 4.2761 (1.2); 4.2673 (1.0); 4.2514 (1.1); 4.2324 (0.6); 4.2124 (0.5); 4.2033 (0.5); 4.1159 (1.9); 3.4879 (2.0); 2.5301 (0.6); 2.5186 (0.6); 2.5151 (0.6); 2.5011 (0.7); 2.4861 (0.7); 2.2362 (12.7); 1.6232 (0.6); 1.5946 (1.4); 1.5622 (1.4); 1.5341 (0.6); 1.4101 (3.6); 1.3939 (10.5); 1.3780 (7.3); 0.0080 (1.2); −0.0002 (42.4); −0.0085 (1.3)

I-190: ¹H-NMR(400.0 MHz, CDCl3):

δ=7.2607 (18.0); 7.0829 (2.1); 7.0621 (1.3); 7.0172 (1.0); 7.0154 (1.0); 6.9966 (0.6); 4.7646 (0.7); 4.5052 (4.4); 4.1557 (0.9); 4.1426 (1.7); 4.1282 (1.2); 3.8162 (1.5); 3.8015 (1.8); 3.7888 (1.1); 3.4850 (1.1); 3.0310 (0.5); 3.0116 (0.6); 2.9925 (0.6); 2.9539 (1.2); 2.9475 (16.0); 2.8828 (0.8); 2.8815 (0.8); 2.2992 (6.4); 1.2891 (5.9); 1.2723 (5.8); −0.0002 (6.6)

I-191: ¹H-NMR(4(K).0 MHz, CDCl3):

δ=7.2600 (39.5); 7.0944 (1.8); 7.0794 (0.9); 7.0603 (1.7); 7.0138 (1.2); 6.9960 (0.8); 4.7653 (0.8); 4.7435 (0.5); 4.5742 (5.0); 4.3065 (0.6); 4.2828 (0.5); 4.1251 (1.1); 4.1120 (2.3); 4.0982 (1.5); 3.8637 (1.8); 3.8491 (2.2); 3.8362 (1.2); 3.2981 (1.0); 3.2810 (1.4); 3.2639 (1.1); 3.0483 (0.6); 3.0292 (0.6); 3.0098 (0.7); 2.9906 (0.7); 2.4921 (0.6); 2.3019 (8.0); 2.2357 (0.6); 1.5921 (1.0); 1.4100 (16.0); 1.3929 (15.8); 1.2915 (7.2); 1.2747 (7.1); -0.0002 (13.8)

1H-NMR

I-192: ¹H-NMR(400.0 MHz, d₆-DMSO):

δ=7.0532 (2.3); 7.0344 (3.2); 6.9595 (2.7); 6.9393 (2.3); 6.9274 (4.3); 6.2833 (1.0); 6.2622 (1.0); 4.6172 (1.1); 4.5955 (2.1); 4.5732 (1.1); 4.4547 (4.5); 4.0362 (2.9); 3.9015 (11.4); 3.7920 (2.6); 3.7784 (4.1); 3.7655 (2.1); 3.3426 (278.0); 3.2674 (1.4); 3.1690 (0.9); 2.9640 (1.0); 2.9444 (1.1); 2.9269 (1.2); 2.9081 (1.2); 2.8125 (0.4); 2.7949 (1.0); 2.7808 (1.7); 2.7650 (1.2); 2.7487 (0.4); 2.6722 (0.6); 2.5068 (91.3); 2.5029 (115.0); 2.4991 (87.9); 2.4145 (1.2); 2.3899 (1.6); 2.3766 (1.1); 2.3536 (1.4); 2.3297 (0.8); 2.3028 (0.6); 2.2827 (0.9); 2.2644 (1.1); 2.2453 (1.1); 2.2201 (16.0); 1.2523 (0.4); 1.2360 (0.4); 1.1940 (0.4): 1.1672 (8.8); 1.1507 (8.6); 1.0161 (10.4); 1.0047 (5.7); 0.9975 (5.2); −0.0002 (11.3)

I-193: ¹H-NMR(400.0 MHz, CDCl3):

δ=7.2602 (86.5); 7.0900 (3.5); 7.0799 (1.9); 7.0608 (3.3); 7.0158 (2.4); 7.0139 (2.4); 6.9962 (1.8); 4.7884 (0.9); 4.7662 (1.7); 4.7441 (1.0); 4.5583 (9.6); 4.3069 (1.2); 4.2832 (1.1); 4.1221 (2.2); 4.1089 (4.5); 4.0950 (2.8); 3.8540 (3.1); 3.8421 (3.6); 3.8389 (4.0); 3.8257 (2.0); 3.5477 (1.7); 3.5276 (2.6); 3.5074 (1.8); 3.4873 (1.4); 3.0482 (1.2); 3.0290 (1.3); 3.0097 (1.4); 2.9908 (1.4); 2.9551 (1.3); 2.8837 (1.1); 2.8823 (1.1); 2.5146 (0.9); 2.4924 (1.1); 2.4764 (0.8); 2.4549 (1.0); 2.3006 (16.0); 2.2743 (0.6); 2.2519 (0.9); 2.2348 (1.2); 2.2176 (0.9); 2.0510 (1.6); 2.0366 (4.4); 2.0310 (3.2); 2.0173 (5.0); 1.9976 (2.2); 1.8576 (1.0); 1.8456 (1.5); 1.8350 (1.7); 1.8189 (1.4); 1.7079 (0.6); 1.6800 (1.8); 1.6707 (1.2); 1.6670 (1.3); 1.6629 (1.9); 1.6515 (1.7); 1.6439 (0.9); 1.6391 (1.0); 1.6327 (0.8); 1.5932 (1.5); 1.2906 (15.0); 1.2738 (14.6); 0.0079 (0.8); −0.0002 (32.8); −0.0085 (1.0)

I-194: ¹H-NMR(400.0 MHz, CDCl3):

δ=7.4161 (0.6); 7.4116 (1.0); 7.4071 (5.1); 7.4017 (5.6); 7.3992 (3.6); 7.3937 (3.8); 7.3893 (5.8); 7.3874 (5.5); 7.3845 (2.5); 7.3829 (3.0); 7.3799 (2.4); 7.3776 (2.5); 7.3737 (5.7); 7.3687 (4.4); 7.3633 (1.5); 7.3602 (2.0); 7.3572 (1.7); 7.3520 (1.0); 7.3476 (1.0); 7.2592 (68.3); 7.0765 (5.2); 7.0581 (3.2); 7.0132 (2.3); 7.0114 (2.3); 6.9950 (1.4); 6.9922 (1.3); 4.7601 (0.9); 4.7379 (1.7); 4.7159 (1.0); 4.3467 (16.0); 4.3209 (6.8); 4.2785 (1.5); 4.2551 (1.2); 3.8081 (1.9); 3.7940 (4.4); 3.7806 (2.5); 3.5483 (2.0); 3.5447 (1.9); 3.5356 (3.3); 3.5303 (3.2); 3.5220 (1.4); 3.5167 (1.5); 3.0410 (1.1); 3.0220 (1.2); 3.0026 (1.3); 2.9836 (1.4); 2.5075 (0.9); 2.4859 (1.0); 2.4695 (0.7); 2.4472 (0.9); 2.2977 (15.2); 2.2608 (0.5); 2.2414 (0.8); 2.2211 (1.1); 2.2040 (0.8); 1.5847 (2.0); 1.2833 (14.0); 1.2665 (13.6); 0.0080 (0.7); −0.0002 (25.4); −0.0085 (0.7)

I-195: ¹H-NMR(400.0 MHz, CDCl3):

δ=8.1600 (2.2); 8.1574 (4.0); 8.1549 (2.2); 8.1401 (2.5); 8.1386 (2.8); 8.1367 (2.6); 8.1352 (2.4); 7.5730 (0.5); 7.5640 (7.4); 7.5615 (7.8); 7.5547 (5.1); 7.5530 (5.1); 7.5516 (5.1); 7.5480 (4.2); 7.5281 (0.6); 7.4741 (2.6); 7.4646 (2.3); 7.4616 (1.8); 7.4543 (2.6); 7.4523 (1.9); 7.4460 (2.1); 7.4408 (1.7); 7.4325 (1.7); 7.2602 (53.1); 7.0719 (5.4); 7.0516 (3.3); 7.0084 (2.4); 7.0066 (2.4); 6.9895 (1.3); 6.9876 (1.3); 4.7681 (0.8); 4.7460 (1.6); 4.7241 (1.0); 4.5318 (11.2); 4.3058 (1.1); 4.2825 (1.0); 4.0887 (2.1); 4.0756 (4.3); 4.0617 (3.0); 3.9062 (3.8); 3.8921 (5.0); 3.8792 (2.6); 3.4842 (10.3); 3.0382 (1.2); 3.0190 (1.3); 2.9996 (1.4); 2.9805 (1.4); 2.5038 (0.9); 2.4816 (1.1); 2.4656 (0.8); 2.4430 (1.0); 2.2945 (16.0); 2.2608 (0.6); 2.2439 (0.8); 2.2415 (0.9); 2.2213 (1.2); 2.2042 (0.9); 1.2763 (14.9); 1.2595 (14.5); −0.0002 (19.9); −0.0085 (0.6)

I-196: ¹H-NMR(400.0 MHz, CDCl3):

δ=7.2607 (20.8); 7.1843 (1.2); 7.1830 (1.2); 7.1418 (0.7); 7.1228 (1.1); 7.0592 (0.8); 7.0573 (0.8); 7.0554 (0.7); 7.0382 (0.5); 4.4959 (3.9); 4.1628 (0.9); 4.1496 (1.5); 4.1355 (1.1); 3.8158 (1.4); 3.8017 (1.8); 3.7885 (1.2); 3.4859 (3.2); 2.9548 (0.5); 2.9436 (16.0); 2.3300 (5.6); 1.8645 (0.6); 1.8459 (0.6); 1.8330 (0.5); 1.8143 (0.5); −0.0002 (7.7)

I-197: ¹H-NMR(400.0 MHz, CDCl3):

δ=7.2606 (29.8); 7.1891 (1.7); 7.1402 (1.0); 7.1211 (1.5);7.0575 (I.I); 7.0557 (I.I); 7.0365 (0.7); 5.1736 (0.5); 5.1541 (0.6); 4.5592 (5.0); 4.1305 (I.I); 4.1175 (2.2); 4.1035 (1.5); 3.8580 (1.8); 3.8441 (2.5); 3.8311 (1.5); 3.4865 (1.2); 3.2911 (1.0); 3.2740 (1.4); 3.2569 (1.1); 2.9541 (0.8); 2.8820 (0.5); 2.8271 (0.6); 2.8072 (0.5); 2.6691 (0.5); 2.6512 (0.5); 2.6496 (0.5); 2.6422 (0.5); 2.3309 (7.7); 1.8659 (0.7); 1.8472 (0.7); 1.8344 (0.7); 1.8157 (0.7); 1.4049 (16.0); 1.3878 (15.9);-0.0002 (11.0)

I-198: ¹H-NMR(400.0 MHz, CDCl3):

δ=7.2607 (20.0); 7.2352 (1.5); 6.9943 (4.4); 6.9914 (4.2); 4.4904 (4.1); 4.1531 (0.9); 4.1399 (1.6); 4.1258 (1.1); 3.8112 (1.5); 3.7970 (1.8); 3.7838 (1.2); 3.4856 (2.5); 2.9420 (16.0); 2.7663 (0.6); 2.2804 (6.4); −0.0002 (7.6)

1H-NMR

I-199: ¹H-NMR(400.0 MHz, CDCl3):

δ=7.2603 (32.7); 7.2432 (1.9); 6.9935 (5.7); 6.9906 (5.4); 4.7815 (0.5); 4.5564 (5.3); 4.3963 (0.6); 4.3755 (0.6); 4.1242 (1.1); 4.1112 (2.2); 4.0973 (1.4); 3.8535 (1.8); 3.8397 (2.5); 3.8266 (1.5); 3.4870 (0.9); 3.2893 (1.0); 3.2722 (1.4); 3.2551 (I.I); 2.7657 (0.7); 2.7245 (0.6); 2.2817 (8.6); 1.9031 (0.5); 1.8955 (0.5); 1.8882 (0.5); 1.6039 (0.6); 1.4041 (16.0); 1.3870 (15.8);-0.0002 (11.9)

I-200: ¹H-NMR(400.0 MHz, CDCl3):

δ=7.2607 (47.2); 7.2364 (3.6); 6.9944 (10.9); 6.9916 (10.2); 4.8050 (0.8); 4.7856 (0.9); 4.5448 (10.6); 4.4138 (1.0); 4.3932 (0.9); 4.1536 (2.2); 4.1405 (3.9); 4.1263 (2.7); 3.8496 (3.4); 3.8355 (4.6); 3.8223 (2.9); 3.4855 (2.9); 2.7813 (0.7); 2.7662 (1.3); 2.7511 (0.8); 2.7427 (0.7); 2.7260 (1.0); 2.7091 (0.8); 2.3676 (0.6); 2.3555 (1.4); 2.3478 (1.4); 2.3434 (0.8); 2.3357 (2.8); 2.3279 (0.8); 2.3236 (1.4); 2.3159 (1.5); 2.3038 (0.7); 2.2803 (16.0); 2.0742 (0.8); 2.0626 (0.8); 2.0516 (0.7); 2.0423 (0.7); 2.0304 (0.6); 1.9299 (0.8); 1.9241 (0.8); 1.9153 (0.7); 1.9090 (1.0); 1.9026 (0.5); 1.8946 (0.9); 1.8820 (0.8); 1.8755 (1.0); 1.8622 (0.5); 1.8581 (0.5); 1.8531 (0.6); 1.8457 (0.7); 1.8376 (0.6); 1.8309 (0.8); 1.8223 (0.8); 1.8107 (0.6); 1.8065 (0.6); 1.8031 (0.6); 1.6209 (0.7); 1.2758 (0.6); 1.2733 (0.8); 1.2616 (3.2); 1.2562 (2.9); 1.2536 (2.0); 1.2497 (2.9); 1.2445 (3.5); 1.2330 (1.1); 1.2301 (0.8); 1.0829 (1.0); 1.0801 (1.0); 1.0716 (1.7); 1.0689 (2.9); 1.0632 (3.7); 1.0606 (1.4); 1.0517 (2.5); 1.0491 (3.3); 1.0437 (2.9); 1.0321 (0.8); 1.0294 (0.8); −0.0002 (17.8); −0.0085 (0.5)

I-201: ¹H-NMR(400.0 MHz, CDCl3):

δ=7.2602 (70.7); 7.2544 (0.5); 7.2415 (3.6); 6.9934 (11.0); 6.9905 (10.1); 4.8017 (0.9); 4.7821 (1.0); 4.5412 (10.0); 4.3961 (1.1); 4.3751 (1.0); 4.1205 (2.2); 4.1075 (3.9); 4.0934 (2.6); 3.8439 (3.3); 3.8300 (4.6); 3.8169 (2.7); 3.5408 (1.6); 3.5207 (2.4); 3.5003 (1.7); 3.4873 (1.8); 2.7804 (0.7); 2.7654 (1.4); 2.7506 (0.8); 2.7415 (0.7); 2.7252 (1.1); 2.7081 (0.8); 2.2811 (16.0); 2.0751 (0.8); 2.0633 (1.0); 2.0494 (1.3); 2.0441 (2.1); 2.0304 (4.8); 2.0249 (3.2); 2.0110 (4.8); 1.9943 (1.9); 1.9916 (2.2); 1.9461 (0.5); 1.9317 (0.8); 1.9263 (0.8); 1.9170 (0.7); 1.9100 (1.0); 1.9001 (0.9); 1.8943 (1.0); 1.8851 (1.0); 1.8741 (1.1); 1.8583 (1.2); 1.8536 (1.6); 1.8433 (1.8); 1.8312 (2.3); 1.8154 (1.8); 1.7879 (0.8); 1.7047 (0.5); 1.6769 (1.7); 1.6677 (1.2); 1.6639 (1.3); 1.6598 (1.8); 1.6484 (1.6); 1.6408 (0.9); 1.6359 (1.0); 1.6296 (0.8); 1.6167 (0.6); 1.6022 (1.2); 0.0080 (0.8); −0.0002 (26.4); −0.0085 (0.8)

I-202: ¹H-NMR(400.0 MHz, CDCl3):

δ=7.4144 (0.6); 7.4104 (1.1); 7.4045 (4.4); 7.3998 (5.0); 7.3973 (3.1); 7.3920 (4.2); 7.3865 (5.4); 7.3845 (4.8); 7.3794 (2.3); 7.3740 (2.5); 7.3696 (5.6); 7.3648 (4.5); 7.3591 (1.6); 7.3568 (2.0); 7.3525 (1.6); 7.3483 (0.9); 7.3439 (1.0); 7.2597 (67.7); 7.2247 (3.6); 6.9923 (10.5); 6.9895 (9.8); 4.7720 (0.8); 4.7536 (0.9); 4.3414 (15.4); 4.3085 (9.4); 3.8160 (2.0); 3.8029 (3.8); 3.7889 (2.5); 3.5388 (3.0); 3.5250 (4.4); 3.5117 (2.6); 3.4872 (6.3); 2.7772 (0.7); 2.7619 (1.3); 2.7469 (0.7); 2.7377 (0.7); 2.7214 (1.1); 2.7043 (0.8); 2.2795 (16.0); 2.0610 (0.8); 2.0498 (0.8); 2.0388 (0.8); 2.0294 (0.7); 2.0042 (2.0); 1.9136 (0.9); 1.9070 (0.9); 1.8960 (1.0); 1.8810 (1.1); 1.8736 (0.9); 1.8657 (1.1); 1.8589 (0.8); 1.8459 (0.7); 1.8318 (0.6); 1.8244 (0.8); 1.8162 (0.8); 1.8000 (0.6); 1.5927 (0.7); 0.0080 (0.7); −0.0002 (25.0); −0.0085 (0.7)

I-203: ¹H-NMR(400.0 MHz, CDCl3):

δ=8.1558 (2.1); 8.1532 (3.7); 8.1507 (2.1); 8.1360 (2.4); 8.1344 (2.8); 8.1326 (2.5); 8.1310 (2.3); 7.5617 (6.7); 7.5591 (7.7); 7.5522 (5.0); 7.5504 (5.5); 7.5496 (5.4); 7.5460 (4.2); 7.5260 (0.6); 7.4706 (2.5); 7.4609 (2.3); 7.4585 (1.7); 7.4508 (2.6); 7.4487 (1.9); 7.4422 (2.0); 7.4376 (1.8); 7.4289 (1.6); 7.2830 (0.5); 7.2711 (0.6); 7.2654 (1.3); 7.2647 (1.3); 7.2598 (72.3); 7.2563 (1.3); 7.2554 (1.2); 7.2547 (1.4); 7.2541 (1.4); 7.2499 (0.6); 7.2236 (3.8); 6.9957 (0.6); 6.9860 (11.0); 6.9831 (10.4); 4.7834 (0.9); 4.7633 (1.0); 4.5158 (10.6); 4.3612 (1.1); 4.3405 (1.0); 4.0977 (2.0); 4.0848 (4.0); 4.0708 (2.8); 3.9035 (3.5); 3.8894 (4.6); 3.8765 (2.5); 3.4866 (2.6); 2.7982 (0.5); 2.7713 (0.8); 2.7558 (1.4); 2.7408 (0.8); 2.7324 (0.8); 2.7165 (1.2); 2.6992 (0.8); 2.2737 (16.0); 2.0572 (0.8); 2.0456 (0.8); 2.0348 (0.8); 2.0250 (0.7); 2.0133 (0.6); 1.9170 (0.9); 1.9116 (0.9); 1.9029 (0.8); 1.8963 (1.1); 1.8818 (1.0); 1.8693 (0.9); 1.8628 (1.1); 1.8518 (0.6); 1.8453 (0.6); 1.8402 (0.7); 1.8330 (0.7); 1.8250 (0.7); 1.8185 (0.8); 1.8099 (0.8); 1.7983 (0.7); 1.7909 (0.6); 1.7824 (0.5); 1.6019 (0.7); 0.0080 (0.7); −0.0002 (27.8); −0.0060 (0.5); −0.0085 (0.8)

1H-NMR

I-204: ¹H-NMR(400.0 MHz, d₆-DMSO):

δ=7.2569 (4.2); 7.2389 (4.5); 7.1334 (2.1); 7.1298 (2.1); 7.1123 (4.3); 7.0950 (2.7); 7.0913 (2.5); 6.8534 (3.0); 6.8510 (3.2); 6.8346 (5.2); 6.8326 (5.3); 6.8162 (2.6); 6.8137 (2.5); 6.7503 (5.9); 6.7298 (5.4); 6.3340 (4.5); 6.3122 (4.6); 4.7057 (1.3); 4.6923 (2.7); 4.6810 (2.0); 4.6707 (2.6); 4.6572 (1.3); 4.2586 (1.0); 4.2504 (1.3); 4.2305 (3.2); 4.2219 (2.3); 4.2109 (2.6); 4.2025 (2.4); 4.1927 (2.2); 4.1837 (2.7); 4.1781 (2.5); 4.1675 (2.7); 4.1571 (1.0); 4.1493 (1.2); 4.1405 (0.9); 4.0136 (5.4); 3.9990 (9.4); 3.9842 (5.5); 3.9019 (16.0); 3.3233 (169.5); 3.2669 (0.9); 3.2283 (5.8); 3.2147 (5.9); 3.2093 (4.4); 3.2009 (6.1); 3.1743 (1.0); 3.1621 (1.0); 2.6749 (1.0); 2.6704 (1.3); 2.6660 (1.0); 2.5404 (1.2); 2.5057 (187.5); 2.5014 (238.6); 2.4970 (173.1); 2.3323 (1.1); 2.3280 (1.4); 2.3240 (1.1); 2.2869 (1.6); 2.2718 (3.9); 2.2585 (5.3); 2.2454 (3.7); 2.2306 (1.5); 2.0717 (0.5); 2.0624 (0.7); 2.0502 (0.9); 2.0372 (1.6); 2.0283 (1.9); 2.0246 (2.0); 2.0165 (2.5); 2.0045 (2.9); 1.9953 (2.8); 1.9885 (2.5); 1.9804 (2.6); 1.9727 (1.6); 1 9653 (1.6); 1.9537 (1.0); 1.9452 (0.8): 1.9388 (0.7); 1.9304 (0.5); −0.0002 (1.6)

I-205: ¹H-NMR(400.0 MHz, d₆-DMSO):

δ=8.3150 (0.3); 7.2650 (6.0); 7.2476 (8.3); 7.2235 (4.6); 7.2058 (9.6); 7.1876 (4.2); 7.1728 (8.2); 7.1546 (9.7); 7.1356 (6.3); 7.1179 (2.0); 6.1817 (7.0); 6.1592 (7.3); 5.0362 (2.1); 5.0161 (5.6); 4.9959 (5.5); 4.9756 (2.0); 4.0121 (9.1); 3.9975 (16.0); 3.9827 (9.3); 3.9020 (5.3); 3.3231 (243.0); 3.2675 (0.5); 3.2604 (0.4); 3.2295 (9.8); 3.2159 (9.8); 3.2098 (7.0); 3.2021 (10.3); 3.1746 (0.4); 2.9409 (1.8); 2.9334 (2.0); 2.9195 (2.1); 2.9116 (2.2); 2.9019 (3.2); 2.8940 (3.3); 2.8801 (3.3); 2.8723 (3.1); 2.7856 (2.3); 2.7650 (4.8); 2.7449 (3.8); 2.7258 (2.9); 2.7055 (1.7); 2.6747 (1.5); 2.6702 (2.0); 2.5405 (1.5); 2.5055 (280.8); 2.5013 (354.4); 2.4970 (255.2); 2.4650 (2.4); 2.4568 (2.2); 2.4458 (3.3); 2.4375 (3.5); 2.4263 (3.6); 2.4150 (3.7); 2.4070 (3.2); 2.3959 (1.8); 2.3879 (1.5); 2.3280 (2.1); 2.2879 (2.7); 2.2732 (6.5); 2.2596 (8.9); 2.2463 (6.0); 2.2313 (2.5); 1.8376 (1.6); 1.8160 (4.2); 1.8064 (1.8); 1.7951 (4.2); 1.7850 (4.0); 1.7733 (1.8); 1.7642 (3.8); 1.7426 (1.4); 1.2357 (0.4); 0.0000 (2.0)

I-207: ¹H-NMR(400.0 MHz, d₆-DMSO):

δ=7.1154 (3.8); 6.9580 (0.4); 6.9383 (8.9); 6.9153 (0.4); 6.0515 (1.9); 6.0286 (2.0); 4.6299 (0.5); 4.6193 (0.7); 4.5993 (1.1); 4.5783 (0.5); 4.0024 (2.4); 3.9877 (4.2); 3.9728 (2.5); 3.9017 (2.3); 3.3232 (74.1); 3.2246 (2.6); 3.2112 (2.6); 3.2059 (1.9); 3.1973 (2.8); 2.7076 (0.3); 2.6826 (0.8); 2.6696 (1.5); 2.6657 (1.6); 2.6518 (1.2); 2.6338 (1.3); 2.6204 (0.7); 2.5924 (0.4); 2.5055 (81.0); 2.5011 (106.0); 2.4968 (77.2); 2.3320 (0.5); 2.3278 (0.6); 2.3236 (0.5); 2.2834 (0.7); 2.2682 (1.7); 2.2554 (2.4); 2.2418 (1.6); 2.2269 (0.8); 2.2111 (16.0); 1.8926(I.I); 1.8821 (1.8); 1.8716 (1.7); 1.8585 (1.0); 1.8385 (0.5); 1.7528 (0.4); 1.7257 (1.0); 1.7027 (1.0); 1.6847 (0.8); 1.6729 (0.9); 1.6521 (0.8); 1.6327 (0.5); −0.0002 (0.8)

I-209: ¹H-NMR(400.0 MHz, d₆-DMSO):

δ=8.3149 (0.4); 7.3052 (4.8); 7.2902 (5.5); 7.2834 (5.9); 7.2721 (0.6); 7.2144 (1.0); 7.1333 (1.3); 7.1206 (5.3); 7.1155 (9.1); 7.1067 (16.0); 7.0982 (10.9); 7.0921 (6.9); 7.0793 (2.1); 7.0653 (7.1); 7.0581 (5.5); 7.0435 (3.6); 6.9590 (1.0); 6.1107 (6.2); 6.0878 (6.4); 4.6712 (1.6); 4.6610 (2.5); 4.6409 (3.6); 4.6190 (1.7); 4.0007 (8.2); 3.9860 (14.7); 3.9712 (8.6); 3.9018 (7.7); 3.3245 (289.0); 3.2671 (0.4); 3.2236 (8.9); 3.2100 (9.1); 3.1963 (9.6); 3.1687 (0.7); 2.7799 (0.7); 2.7609 (1.1); 2.7380 (2.6); 2.7204 (3.9); 2.7069 (4.4); 2.6901 (4.8); 2.6751 (3.6); 2.6705 (3.0); 2.6660 (2.4); 2.6474 (1.3); 2.5405 (0.7); 2.5055 (262.0); 2.5012 (350.1); 2.4968 (260.8); 2.3322 (1.5); 2.3277 (2.0); 2.2830 (2.4); 2.2678 (5.9); 2.2547 (8.2); 2.2410 (5.7); 2.2263 (2.4); 1.9529 (0.8); 1.9062 (5.6); 1.8967 (6.0); 1.8857 (3.8); 1.8668 (1.8); 1.8458 (0.7); 1.7817 (1.4); 1.7548 (3.5); 1.7362 (3.6); 1.7304 (3.3); 1.7127 (2.8); 1.7043 (3.0); 1.6829 (2.6); 1.6641 (1.8); 1.6405 (0.6); −0.0002 (2.0)

I-210; ¹H-NMR(400.0 MHz, d₆-DMSO):

δ=7.0972 (2.0); 7.0784 (2.9); 7.0576 (4.0); 6.9909 (2.5); 6.9723 (1.8); 6.1376 (1.9); 6.1153 (2.0); 4.9988 (0.6); 4.9789 (1.5); 4.9585 (1.5); 4.9390 (0.6); 4.0109 (2.2); 3.9962 (4.1); 3.9820 (2.4); 3.9022 (1.2); 3.8993 (1.0); 3.3251 (72.1); 3.2277 (2.6); 3.2169 (2.8); 3.2031 (2.8); 2.8697 (0.6); 2.8470 (0.6); 2.8322 (0.9); 2.8127 (0.9); 2.7212 (0.6); 2.7012 (1.2); 2.6802 (1.1); 2.6628 (1.1); 2.6418 (0.5); 2.5029 (101.6); 2.4989 (97.0); 2.4447 (0.5); 2.4227 (1.0); 2.4142 (0.9); 2.4034 (1.0); 2.3834 (0.4); 2.3296 (0.6); 2.3258 (0.6); 2.2534 (16.0); 1.8106 (0.3); 1.7887 (1.0); 1.7673 (1.0); 1.7588 (1.0); 1.7367 (0.9); 0.0002 (0.4); −0.0027 (0.4)

1H-NMR

I-211: ¹H-NMR(400.0 MHz, d₆-DMSO):

δ=7.2640 (3.1); 7.2449 (3.4); 7.1355 (1.6); 7.1317 (1.6); 7.1142 (3.2); 7.0970 (2.0); 7.0933 (1.9); 6.8543 (2.3); 6.8517 (2.4); 6.8355 (3.9); 6.8333 (4.0); 6.8172 (1.9); 6.8145 (1.9); 6.7518 (4.4); 6.7498 (4.3); 6.7315 (4.0); 6.7294 (3.8); 6.3254 (3.7); 6.3037 (3.8); 4.6969 (0.9); 4.6835 (2.0); 4.6728 (1.5); 4.6625 (2.0); 4.6494 (1.0); 4.2564 (0.7); 4.2483 (1.0); 4.2352 (0.8); 4.2283 (2.4); 4.2202 (1.9); 4.2084 (2.0); 4.2000 (1.8); 4.1919 (1.7); 4.1825 (2.0); 4.1769 (2.0); 4.1665 (2.3); 4.1562 (5.3); 4.1440 (4.1); 4.1313 (4.8); 3.9018 (16.0); 3.3218 (148.8); 2.8940 (4.0); 2.8877 (3.2); 2.8808 (3.9); 2.8727 (3.3); 2.8669 (4.4); 2.6747 (0.8); 2.6701 (1.1); 2.6658 (0.8); 2.5233 (3.5); 2.5096 (80.6); 2.5057 (155.8); 2.5012 (200.8); 2.4967 (149.9); 2.3324 (1.0); 2.3279 (1.3); 2.3236 (1.0); 2.0585 (3.9); 2.0531 (3.4); 2.0447 (3.3); 2.0308 (2.8); 2.0190 (2.0); 2.0068 (1.8); 1.9959 (1.8); 1.9884 (1.9); 1.9804 (2.0); 1.9736 (1.3); 1.9656 (1.2); 1.9537 (0.8); 1.9458 (0.7); 1.9389 (0.6); 1.9305 (0.4); 1.7841 (1.5); 1.7733 (2.7); 1.7613 (3.4); 1.7489 (2.6); −0.0002 (8.7)

I-213: ¹H-NMR(400.0 MHz. CDCl3):

δ=7.2623 (29.1); 7.2386 (7.9); 7.1354 (2.0); 7.1158 (4.2); 7.1003 (4.5); 7.0814 (2.2); 6.9103 (3.6); 6.8878 (5.9); 6.8668 (3.3); 5.2968 (16.0); 4.8646 (1.4); 4.8528 (2.8); 4.8323 (3.4); 4.8169 (2.0); 4.2825 (4.1); 4.2493 (10.8); 4.2372 (8.2); 4.2243 (10.0); 2.8527 (8.4); 2.8396 (8.4); 2.8254 (9.7); 2.8025 (2.0); 2.7856 (1.2); 2.7738 (2.2); 2.7578 (3.9); 2.7442 (2.3); 2.7192 (2.1); 2.7038 (3.8); 2.6884 (2.4); 2.6605 (1.6); 2.6450 (1.0); 2.2097 (2.3); 2.1952 (5.1); 2.1819 (7.6); 2.1742 (5.4); 2.1685 (5.7); 2.1540 (2.9); 2.0975 (1.4); 2.0746 (2.3); 2.0646 (3.2); 2.0425 (3.4); 2.0365 (2.4); 1.9461 (0.8); 1.9193 (3.0); 1.9025 (5.2); 1.8964 (4.6); 1.8799 (7.6); 1.8653 (9.4); 1.8534 (10.8); 1.8283 (5.4); 1.6900 (1.0); 1.2754 (0.6); 1.2576 (1.4); 1.2397 (0.6); −0.0002 (9.8)

I-215:1 ¹H-NMR(400.0 MHz. CDCl3):

δ=7.2612 (59.4); 7.1884 (5.4); 7.1820 (5.7); 7.1631 (5.8); 7.1571 (5.9); 7.0475 (4.1); 7.0271 (6.1); 7.0121 (5.5); 6.8782 (3.4); 6.8714 (3.3); 6.8573 (6.3); 6.8506 (6.1); 6.8362 (3.3); 6.8296 (3.0); 5.2976 (11.1); 4.8270 (3.7); 4.8071 (4.1); 4.2582 (11.6); 4.2456 (11.1); 4.2337 (16.0); 4.2144 (5.9); 2.8612 (10.8); 2.8480 (10.8); 2.8339 (12.4); 2.7956 (2.0); 2.7534 (4.9); 2.7369 (4.9); 2.7210 (5.3); 2.7071 (3.7); 2.6806 (1.8); 2.1994 (6.4); 2.1867 (9.6); 2.1732 (7.5); 2.1584 (4.3); 2.1411 (2.8); 2.1284 (2.6); 2.1174 (3.5); 2.1068 (4.5); 2.0856 (2.9); 2.0431 (1.9); 1.9219 (1.1); 1.8742 (9.1); 1.8616 (12.9); 1.8495 (10.4); 1.8354 (11.5); 1.8186 (8.7); 1.7935 (6.2); 1.6244 (2.6); 1.2758 (0.8); 12579 (2.0); −0.0002 (21.5)

I-216: ¹H-NMR(400.0 MHz, d₆-DMSO):

δ=7.8443 (0.7); 7.5893 (3.8); 7.3122 (3.0); 7.2967 (3.4); 7.2901 (3.6); 7.2788 (0.4); 7.1359 (0.7); 7.1228 (3.2); 7.1175 (5.2); 7.1086 (9.8); 7.1003 (6.3); 7.0938 (4.1); 7.0805 (1.3); 7.0673 (4.4); 7.0605 (3.4); 7.0453 (2.2); 6.1013 (4.0); 6.0783 (4.2); 4.6556 (1.6); 4.6367 (2.2); 4.6158 (1.0); 4.1435 (5.8); 4.1311 (4.6); 4.1187 (6.0); 4.0754 (2.8); 4.0312 (16.0); 3.9018 (6.4); 3.3253 (273.7); 3.2671 (0.7); 3.1684 (0.6); 2.8909 (5.2); 2.8846 (4.0); 2.8775 (5.0); 2.8697 (4.1); 2.8638 (5.5); 2.7820 (0.5); 2.7644 (0.7); 2.7402 (1.6); 2.7227 (2.4); 2.7083 (2.6); 2.6917 (2.9); 2.6757 (2.2); 2.6708 (1.9); 2.6662 (1.6); 2.6492 (0.8); 2.5406 (0.6); 2.5059 (183.6); 2.5016 (238.2); 2.4972 (173.0); 2.3325 (1.1); 2.3283 (1.4); 2.3241 (1.1); 2.0563 (4.5); 1.9549 (0.5); 1.9182 (2.2); 1.9073 (3.6); 1.8977 (3.7); 1.8854 (2.2); 1.8668 (1.1); 1.8455 (0.5); 1.7819 (2.8); 1.7715 (4.0); 1.7561 (6.0); 1.7352 (3.8); 1.7134 (1.9); 1.7027 (2.0); 1.6820 (1.6); 1.6623 (1.1); 1.6390 (0.4); 1.3228 (0.5); 1.2354 (0.3); −0.0002 (3.2)

I-217: ¹H-NMR(400.0 MHz, d₆-DMSO):

δ=7.6503 (0.4); 7.3360 (0.5); 7.3155 (8.4); 7.3046 (13.5); 7.2929 (1.8); 7.2776 (2.0); 7.2665 (1.5); 7.2583 (2.8); 7.2453 (1.5); 7.2375 (0.7); 7.2333 (0.8); 7.2237 (0.5); 7.2151 (1.0); 7.1972 (2.1); 7.1795 (1.0); 7.1633 (1.7); 7.1455 (0.9); 7.1264 (1.2); 7.1097 (1.4); 7.0923 (0.5); 6.3615 (1.1); 6.3398 (1.2); 5.0203 (0.4); 5.0009 (1.1); 4.9815 (1.1); 4.9486 (4.1); 4.9451 (4.3); 4.9203 (0.7); 4.9063 (0.3); 4.1067 (2.2); 4.0885 (4.9); 4.0704 (2.4); 4.0140 (0.4); 3.9019 (16.0); 3.3249 (56.9); 3.1684 (1.5); 3.0540 (2.4); 3.0359 (5.0); 3.0177 (2.3); 2.9939 (0.4); 2.9422 (0.4); 2.9338 (0.4); 2.9211 (0.4); 2.9121 (0.4); 2.9029 (0.6); 2.8949 (0.7); 2.8814 (0.6); 2.8735 (0.6); 2.7838 (0.4); 2.7629 (0.9); 2.7429 (0.8); 2.7243 (0.6); 2.7028 (0.3); 2.6747 (0.5); 2.6703 (0.6); 2.6658 (0.5); 2.6462 (0.4); 2.6312 (0.4); 2.6168 (0.4); 2.5235 (1.9); 2.5101 (42.8); 2.5058 (87.0); 2.5013 (114.9); 2.4967 (85.2); 2.4924 (43.7); 2.4682 (0.8); 2.4597 (0.6); 2.4487 (0.8); 2.4403 (0.8); 2.4291 (0.8); 2.4178 (0.8); 2.4096 (0.7); 2.3986 (0.4); 2.3906 (0.4); 2.3325 (0.5); 2.3280 (0.7); 2.3236 (0.5); 1.8279 (0.8); 1.8183 (0.4); 1.8075 (0.8); 1.7969 (0.8); 1.7862 (0.4); 1.7765 (0.8); −0.0001 (2.3)

I-218: ¹H-NMR(400.0 MHz, d₆-DMSO):

δ=7.7007 (2.8); 7.6322 (1.6); 7.6163 (3.0); 7.5785 (2.1); 7.5593 (1.8); 7.5402 (0.7); 7.2677 (1.6); 7.2494 (2.1); 7.2161 (1.1); 7.1983 (2.5); 7.1801 (1.2); 7.1638 (2.0); 7.1459 (1.0); 7.1252 (1.3); 7.1062 (1.6); 7.0883 (0.6); 6.3748 (1.6); 6.3532 (1.6); 5.0244 (6.8); 5.0113 (1.6); 4.9905 (1.3); 4.9709 (0.5); 4.1112 (2.5); 4.0930 (5.7); 4.0750 (2.7); 3.9018 (16.0); 3.3239 (61.7); 3.1686 (1.4); 3.0695 (2.7);

1H-NMR

3.0515 (5.7); 3.0333 (2.5); 2.9424 (0.4); 2.9344 (0.5); 2.9207 (0.5); 2.9124 (0.5); 2.9031 (0.7); 2.8951 (0.8); 2.8815 (0.8); 2.8738 (0.7); 2.7821 (0.5); 2.7613 (1.1); 2.7413 (0.9); 2.7218 (0.7); 2.7012 (0.4); 2.6750 (0.6); 2.6705 (0.8); 2.6661 (0.6); 2.5239 (2.4); 2.5103 (54.8); 2.5060 (108.3); 2.5015 (140.2); 2.4969 (102.4); 2.4926 (51.6); 2.4656 (0.8); 2.4572 (0.7); 2.4462 (0.9); 2.4379 (0.9); 2.4348 (0.8); 2.4266 (0.9); 2.4151 (0.9); 2.4071 (0.8); 2.3961 (0.5); 2.3876 (0.4); 2.3328 (0.6); 2.3282 (0.8); 2.3237 (0.6); 1.8484 (0.4); 1.8270 (1.0); 1.8175 (0.4); 1.8066 (1.0); 1.7961 (0.9); 1.7851 (0.4); 1.7756 (0.9); −- 0.0002 (3.0)

I-219: ¹H-NMR(400.0 MHz, d₆-DMSO):

δ=7.3118 (2.1); 7.2941 (2.6); 7.2260 (1.5); 7.2090 (3.1); 7.1895 (1.4); 7.1747 (2.6); 7.1540 (3.0); 7.1360 (2.1); 7.1181 (0.7); 6.3538 (2.3); 6.3321 (2.4); 5.0420 (0.6); 5.0219 (1.8); 5.0017 (1.8); 4.9813 (0.6); 4.0553 (3.5); 4.0371 (7.7); 4.0189 (3.7); 3.9017 (16.0); 3.6354 (5.6); 3.6178 (5.7); 3.5919 (0.4); 3.3234 (91.2); 3.1683 (1.0); 2.9787 (3.8); 2.9606 (8.1); 2.9424 (4.0); 2.9321 (0.9); 2.9236 (0.8); 2.9143 (1.1); 2.9060 (1.1); 2.8921 (1.1); 2.8843 (1.0); 2.7978 (0.7); 2.7771 (1.5); 2.7570 (1.2); 2.7380 (1.0); 2.7166 (0.5); 2.6744 (0.6); 2.6703 (0.8); 2.6657 (0.6); 2.5232 (2.6); 2.5056 (100.3); 2.5012 (129.1); 2.4968 (94.5); 2.4682 (1.4); 2.4599 (1.3); 2.4486 (1.2); 2.4372 (1.2); 2.4291 (1.1); 2.4180 (0.6); 2.4100 (0.5); 2.3322 (0.6); 2.3280 (0.8); 2.3233 (0.6); 1.8677 (0.5); 1.8461 (1.4); 1.8370 (0.6); 1.8254 (1.4); 1.8152 (1.3); 1.8035 (0.6); 1.7943 (1.2); 1.7726 (0.4); 1.2157 (0.4); 1.2045 (0.8); 1.1970 (0.8); 1.1853 (1.4); 1.1732 (0.9); 1.1665 (0.9); 1. 1547 (0.5); 1. 1484 (0.4); 0.4521 (1.0); 0.4405 (2.9); 0.4362 (3.6); 0.4267 (2.2); 0.4205 (3.0); 0.4161 (3.4); 0.4069 (1.6); 0.3903 (0.4); 0.3845 (0.4); 0.3714 (0.5); 0.3479 (1.6); 0.3355 (4.2); 0.3266 (3.7); 0.3228 (3.7); 0.3108 (1.1); −0.0003 (1.2)

I-220: ¹H-NMR(400.0 MHz, d₆-DMSO):

δ=7.3169 (2.6); 7.2994 (3.2); 7.2269 (1.8); 7.2094 (3.8); 7.1903 (1.7); 7.1748 (3.2); 7.1562 (3.7); 7.1374 (2.5); 7.1196 (0.9); 6.2958 (3.2); 6.2742 (3.3); 5.0420 (0.8); 5.0221 (2.3); 5.0018 (2.3); 4.9824 (0.8); 4.8776 (0.7); 4.8604 (1.8); 4.8432 (2.6); 4.8257 (1.9); 4.8083 (0.7); 4.0313 (4.2); 4.0132 (9.2); 3.9950 (4.5); 3.9017 (8.4); 3.3217 (72.6); 3.1748 (0.6); 3.1615 (0.6); 2.9575 (0.7); 2.9499 (0.8); 2.9351 (0.9); 2.9271 (1.1); 2.9178 (5.8); 2.9103 (2.0); 2.8997 (10.2); 2.8817 (4.5); 2.7991 (0.9); 2.7783 (1.9); 2.7583 (1.5); 2.7389 (1.2); 2.7182 (0.7); 2.6700 (0.8); 2.5052 (104.6); 2.5011 (134.2); 2.4970 (101.0); 2.4681 (1.8); 2.4598 (1.6); 2.4484 (1.6); 2.4374 (1.6); 2.4289 (1.3); 2.4178 (0.8); 2.4099 (0.7); 2.3279 (0.8); 1.8773 (0.6); 1.8559 (1.6); 1.8462 (0.7); 1.8351 (1.6); 1.8250 (1.6); 1.8137 (0.7); 1.8043 (1.5); 1.7828 (0.5); 1.4002 (15.6); 1.3961 (16.0): 1.3830 (15.6); 1.3789 (15.7); 1.2356 (0.4); −0.0002 (2.4)

I-221: ¹H-NMR(400.0 MHz, d₆-DMSO):

δ=7.3148 (9.2); 7.3039 (16.0); 7.2759 (0.5); 7.2656 (2.8); 7.2547 (1.6); 7.2441 (3.3); 7.2324 (0.8); 7.1237 (0.9); 7.1196 (0.9); 7.1023 (1.8); 7.0852 (1.1); 7.0811 (1.1); 6.8239 (1.3); 6.8055 (2.2); 6.7892 (1.0); 6.7869 (1.1); 6.7392 (2.4); 6.7189 (2.2); 6.4920 (1.5); 6.4704 (1.5); 4.9435 (7.5); 4.7040 (0.5); 4.6906 (1.0); 4.6786 (0.8); 4.6698 (1.0); 4.6561 (0.4); 4.2556 (0.4); 4.2476 (0.5); 4.2278 (1.2); 4.2198 (1.0); 4.2086 (1.0); 4.1995 (0.9); 4.1898 (0.8); 4.1798 (1.1); 4.1739 (1.0); 4.1643 (1.0); 4.1518 (0.4); 4.1463 (0.5); 4.1373 (0.4); 4.1112 (2.6); 4.0931 (5.8); 4.0750 (2.8); 3.3274 (57.0); 3.0544 (2.8); 3.0363 (5.9); 3.0181 (2.6); 2.9940 (4.2); 2.6740 (0.4); 2.6703 (0.6); 2.5404 (42.7); 2.5094 (36.6); 2.5055 (71.9); 2.5010 (93.2); 2.4966 (67.3); 2.3277 (0.5); 2.3233 (0.4); 2.0628 (0.3); 2.0503 (0.6); 2.0402 (0.8); 2.0288 (1.0); 2.0208 (1.1); 2.0085 (1.4); 1.9992 (1.1); 1.9841 (0.6); 1.9735 (0.4); 1.2396 (0.3); −0.0002 (2.0)

I-223: ¹H-NMR(400.0 MHz, d₆-DMSO):

δ=7.3181 (2.2); 7.3005 (2.9); 7.2219 (1.5); 7.2044 (3.4); 7.1862 (1.5); 7.1702 (2.8); 7.1458 (2.3); 7.1266 (2.4); 7.1092 (1.0); 6.3858 (2.6); 6.3642 (2.8); 5.0191 (0.6); 4.9994 (1.9); 4.9794 (1.9); 4.9595 (0.7); 4.0526 (3.3); 4.0344 (7.4); 4.0163 (3.6); 3.9018 (16.0); 3.6073 (4.8); 3.5887 (5.0); 3.3219 (99.0); 3.1685 (0.7); 2.9701 (3.6); 2.9519 (7.8); 2.9337 (4.1); 2.9158 (1.2); 2.9079 (1.4); 2.8938 (1.2); 2.8866 (1.1); 2.7927 (0.7); 2.7724 (1.6); 2.7523 (1.2); 2.7330 (1.0); 2.7122 (0.6); 2.6704 (1.0); 2.5056 (138.4); 2.5013 (173.7); 2.4970 (128.8); 2.4595 (1.7); 2.4511 (1.7); 2.4400 (1.6); 2.4287 (1.6); 2.4204 (1.3); 2.4092 (0.8); 2.4011 (0.7); 2.3280 (1.2); 1.8695 (0.5); 1.8480 (1.4); 1.8381 (0.8); 1.8274 (1.7); 1.8168 (1.8); 1.8057 (1.3); 1.7963 (2.1); 1.7869 (1.2); 1.7759 (1.2); 1.6558 (2.6); 1.6097 (3.2); 1.5860 (3.7); 1.1284 (4.4); 0.9294 (1.8); 0.9026 (1.6); −0.0002 (2.6)

I-225: ¹H-NMR(400.0 MHz, d₆-DMSO):

δ=7.2855 (1.5); 7.2670 (1.5); 7.1379 (0.7); 7.1339 (0.7); 7.1165 (1.5); 7.0993 (0.9); 7.0955 (0.9); 6.8558 (1.0): 6.8533 (1.1): 6.8349 (1.8); 6.8185 (0.9); 6.8161 (0.8); 6.7545 (2.0); 6.7341 (1.8); 6.4730

1H-NMR

(1.5); 6.4512 (1.6); 4.7200 (0.4); 4.7059 (0.9); 4.696 (0.7); 4.6845 (0.9); 4.6707 (0.4); 4.2640 (0.5); 4.2520 (0.4); 4.2440 (1.1); 4.2364 (0.8); 4.2246 (0.9); 4.2160 (0.8); 4.2051 (0.7); 4.1958 (0.9); 4.1890 (0.8); 4.1797 (0.9); 4.1683 (0.3); 4.1619 (0.4); 4.0388 (2.2); 4.0206 (4.9); 4.0024 (2.4); 3.9017 (4.7); 3.3222 (41.8); 3.1811 (16.0); 3.1622 (0.7); 2.9506 (2.3); 2.9324 (4.6); 2.9142 (2.1); 2.6705 (0.4); 2.5057 (55.6); 2.5013 (71.1); 2.4969 (52.9); 2.3283 (0.4); 2.3239 (0.3); 2.0647 (0.6); 2.0539 (0.7); 2.0439 (0.8); 2.0352 (1.0); 2.0215 (1.1); 2.0121 (0.9); 1.9970 (0.5); 1.9860 (0.3); −0.0003 (1.0)

I-227: ¹H-NMR(400.0 MHz, d₆-DMSO):

δ=7.0922 (3.1); 7.0830 (2.6); 6.9950 (1.8); 6.9758 (1.3); 6.2921 (1.8); 6.2700 (1.9); 5.0116 (0.4); 4.9911 (1.2); 4.9708 (1.2); 4.9507 (0.4); 4.0334 (2.3); 4.0152 (5.1); 3.9970 (2.5); 3.9018 (4.6); 3.3197 (38.2); 3.1843 (16.0); 3.1751 (1.2); 3.1615 (0.7); 2.9504 (2.4); 2.9323 (4.9); 2.9140 (2.2); 2.8839 (0.4); 2.8769 (0.4); 2.8634 (0.4); 2.8554 (0.5); 2.8447 (0.6); 2.8385 (0.7); 2.8239 (0.7); 2.8168 (0.6); 2.7335 (0.4); 2.7124 (0.9); 2.6929 (0.8); 2.6744 (0.9); 2.6530 (0.4); 2.5053 (56.4); 2.5009 (72.4); 2.4966 (54.1); 2.4692 (0.7); 2.4576 (0.8); 2.4499 (0.8); 2.4383 (0.8); 2.4268 (0.8); 2.4192 (0.7); 2.4079 (0.4); 2.4007 (0.3); 2.3276 (0.4); 2.3235 (0.3); 2.2547 (11.5); 1.8100 (0.9); 1.8010 (0.4); 1.7888 (0.9); 1.7793 (0.9): 1.7669 (0.4); 1.7580 (0.8); −0.0002 (1.5)

I-228: ¹H-NMR(400.0 MHz, d₆-DMSO):

δ=7.3405 (2.2); 7.3248 (2.5); 7.3184 (2.5); 7.1354 (0.6); 7.1222 (2.4); 7.1168 (3.7); 7.1079 (7.0); 7.0998 (4.4); 7.0929 (2.9); 7.0796 (1.1); 7.0676 (3.2); 7.0607 (2.4); 7.0499 (1.3); 7.0456 (1.5); 6.2803 (3.4); 6.2575 (3.5); 4.6851 (0.7); 4.6666 (1.3); 4.6553 (1.5); 4.6337 (0.7); 4.0434 (4.4); 4.0252 (9.7); 4.0070 (4.6); 3.9018 (16.0); 3.6708 (0.6); 3.6530 (0.7); 3.6361 (3.6); 3.6257 (3.8); 3.6187 (3.9); 3.6082 (3.5); 3.5915 (0.7); 3.5735 (0.6); 3.3213 (108.8); 3.1747 (0.9); 3.1620 (0.9); 2.9728 (4.3); 2.9546 (8.6); 2.9363 (3.8); 2.7859 (0.4); 2.7710 (0.5); 2.7445 (1.2); 2.7265 (1.6); 2.7128 (2.0); 2.6964 (2.1); 2.6804 (1.2); 2.6749 (1.1); 2.6702 (1.3); 2.6663 (1.0); 2.6545 (0.7); 2.6398 (0.3); 2.5232 (3.4); 2.5099 (64.8); 2.5057 (123.6); 2.5013 (157.1); 2.4968 (113.3); 2.4927 (56.1); 2.3325 (0.7); 2.3279 (0.9); 2.3235 (0.7); 1.9844 (0.3); 1.9791 (0.4); 1.9650 (0.8); 1.9576 (1.0); 1.9457 (1.4); 1.9319 (2.4); 1.9206 (2.0); 1.9051 (1.2); 1.8857 (0.7); 1.8182 (0.6); 1.8129 (0.6); 1.7901 (1.5); 1.7711 (1.4); 1.7661 (1.4); 1.7472 (1.0); 1.7324 (0.7); 1.7136 (1.0); 1.6986 (1.0); 1.6937 (1.1); 1.6744 (0.7); 1.2192 (0.4); 1.2119 (0.5); 1.2010 (1.1); 1.1934 (1.0); 1.1820 (1.7); 1.1698 (1.1); 1.1629 (1.1); 1.1509 (0.6); 0.4515 (1.2); 0.4406 (3.4); 0.4361 (4.2); 0.4268 (2.5); 0.4206 (3.5); 0.4160 (3.9); 0.4070 (1.8); 0.3893 (0.4); 0.3847 (0.4); 0.3707 (0.6); 0.3470 (2.0); 0.3348 (4.9); 0.3259 (4.4); 0.3230 (4.1); 0.3109 (1.2); −0.0002 (2.2)

I-230: ¹H-NMR(400.0 MHz, d₆-DMSO):

δ=7.3054 (1.3); 7.2876 (1.7); 7.2282 (1.0); 7.2110 (2.0); 7.1926 (0.9); 7.1776 (1.7); 7.1576 (1.9); 7.1400 (1.3); 7.1219 (0.4); 6.3370 (1.5); 6.3150 (1.6); 5.0462 (0.4); 5.0261 (1.2); 5.0058 (1.2); 4.9860 (0.4); 4.0351 (2.3); 4.0169 (5.0); 3.9987 (2.4); 3.9019 (4.6); 3.3237 (50.4); 3.1840 (16.0); 2.9511 (2.7); 2.9329 (5.0); 2.9145 (2.6); 2.9035 (0.8); 2.8894 (0.8); 2.8817 (0.7); 2.7981 (0.5); 2.7770 (1.0); 2.7568 (0.8); 2.7384 (0.6); 2.7174 (0.4); 2.6749 (0.3); 2.6706 (0.4); 2.6662 (0.3); 2.5057 (57.5); 2.5014 (72.9); 2.4970 (53.8); 2.4720 (1.0); 2.4638 (0.9); 2.4524 (0.8); 2.4410 (0.8); 2.4331 (0.7); 2.4219 (0.4); 2.4139 (0.3); 2.3282 (0.4); 1.8355 (0.9); 1.8264 (0.4); 1.8147 (0.9); 1.8046 (0.8); 1.7929 (0.4); 1.7838 (0.8); −0.0001 (1.3)

I-231: ¹H-NMR(400.0 MHz, d₆-DMSO):

δ=7.1387 (3.0); 6.9620 (0.4); 6.9422 (6.8); 6.2051 (1.8); 6.1821 (1.8); 4.6432 (0.4); 4.6254 (0.7); 4.6126 (0.8); 4.5911 (0.4); 4.0252 (2.3); 4.0070 (5.0); 3.9888 (2.4); 3.9018 (6.8); 3.3215 (61.5); 3.1813 (16.0); 3.1616 (0.5); 2.9456 (2.3); 2.9275 (4.8); 2.9092 (2.1); 2.6901 (0.6); 2.6704 (1.3); 2.6593(1.1); 2.6417 (1.1); 2.6279 (0.6); 2.5992 (0.3); 2.5055 (57.7); 2.5011 (75.2); 2.4967 (56.6); 2.3318 (0.3); 2.3278 (0.4); 2.3239 (0.4); 2.2141 (12.1); 1.9447 (0.4); 1.9281 (0.6); 1.9148 (1.0); 1.9022 (1.4); 1.8903 (0.7); 1.8732 (0.6); 1.8556 (0.4); 1.7529 (0.8); 1.7336 (0.7); 1.7294 (0.7); 1.7099 (0.6); 1.7001 (0.4); 1.6858 (0.6); 1.6581 (0.6); 1.6436 (0.4); 1.6382 (0.3); −0.0002 (0.8)

I-232: ¹H-NMR(400.0 MHz, d₆-DMSO):

δ=7.1197 (2.3); 7.1007 (3.2); 7.0659 (4.1); 7.0144 (2.5); 6.9953 (1.8); 6.7486 (2.4); 6.7266 (2.5); 5.0499 (0.6); 5.0296 (1.6); 5.0096 (1.6); 4.9895 (0.6); 4.3079 (2.8); 4.2959 (2.3); 4.2833 (2.9); 3.9021 (3.5); 3.6604 (2.5); 3.6532 (2.0); 3.6460 (2.4); 3.6388 (2.0); 3.6320 (2.5); 3.3217 (55.1); 3.1745 (0.7); 3.1616 (0.7); 2.8985 (0.5); 2.8915 (0.6); 2.8766 (0.6); 2.8694 (0.6); 2.8599 (0.8); 2.8523 (0.9); 2.8382 (0.9); 2.8308 (0.8); 2.7531 (0.6); 2.7326 (1.2); 2.7133 (1.0); 2.6936 (0.7); 2.6705 (1.0); 2.5054 (91.9); 2.5012 (118.4); 2.4968 (86.3); 2.4723 (1.4); 2.4643 (1.2); 2.4528 (1.1); 2.4413 (1.1); 2.4334 (1.0);

1H-NMR

2.4221 (0.6); 2.4147 (0.6); 2.3278 (0.7); 2.3236 (0.5); 2.2608 (16.0); 2.1394 (2.2); 2.1261 (1.7); 1.9352 (1.0); 1.9245 (1.7); 1.9125 (2.2); 1.9011 (1.5); 1.8373 (0.5); 1.8156 (1.2); 1.8064 (0.5); 1.7946 (1.2); 1.7847 (1.2); 1.7731 (0.5); 1.7638 (1.1); 1.7420 (0.4); −0.0002 (1.1)

I-234: ¹H-NMR(400.0 MHz, d₆-DMSO):

δ=7.1158 (3.6); 6.9815 (0.4); 6.9620 (8.2); 6.9399 (0.4); 6.6806 (2.0); 6.6580 (2.2); 4.6638 (0.8); 4.6513 (1.0); 4.6296 (0.5); 4.3023 (2.4); 4.2908 (2.2); 4.2785 (2.6); 3.9018 (16.0); 3.6590 (2.2); 3.6452 (2.3); 3.6310 (2.3); 3.3218 (80.9); 2.7269 (0.4); 2.7015 (0.7); 2.6822 (1.2); 2.6707 (1.8); 2.6542 (1.3); 2.6402 (0.7); 2.6126 (0.4); 2.5057 (89.6); 2.5016 (111.0); 2.4979 (86.2); 2.3279 (0.7); 2.3187 (0.6); 2.2192 (13.9); 2.1378 (2.1); 1.9045 (3.4); 1.7803 (0.4); 1.7764 (0.4); 1.7547 (0.9); 1.7353 (0.9); 1.7289 (0.9): 1.7124 (1.0); 1.6982 (0.8); 1.6891 (0.7); 1.6729 (0.7); 1.6518 (0.5); −0.0001 (5.5)

I-236: ¹H-NMR(400.0 MHz, d₆-DMSO):

δ=7.2773 (2.4); 7.2597 (3.5); 7.2460 (1.9); 7.2282 (3.9); 7.2113 (1.8); 7.1960 (3.4); 7.1749 (3.8); 7.1565 (2.5); 7.1388 (0.8); 6.7863 (3.3); 6.7644 (3.4); 5.0840 (0.8); 5.0646 (2.3); 5.0444 (2.3); 5.0245 (0.8); 4.3094 (3.9); 4.2972 (3.2); 4.2846 (4.0); 3.9017 (16.0); 3.6611 (3.4); 3.6545 (2.7); 3.6469 (3.4); 3.6392 (2.8); 3.6326 (3.6); 3.3220 (111.2); 3.1747 (0.5); 3.1621 (0.5); 2.9631 (0.7); 2.9555 (0.8); 2.9417 (0.8); 2.9334 (0.9); 2.9237 (1.2); 2.9160 (1.4); 2.9021 (1.3); 2.8942 (1.3); 2.8181 (0.9); 2.7972 (1.9); 2.7769 (1.5); 2.7578 (1.2); 2.7369 (0.7); 2.6749 (0.6); 2.6704 (0.9); 2.6659 (0.6); 2.5403 (0.4); 2.5235 (3.1); 2.5099 (60.5); 2.5058 (116.8); 2.5013 (150.4); 2.4968 (113.0); 2.4666 (2.1); 2.4552 (1.8); 2.4470 (1.6); 2.4360 (0.9); 2.4280 (0.8); 2.3323 (0.7); 2.3281 (0.9); 2.3238 (0.7); 2.1399 (3.1); 2.1268 (2.4); 1.9378 (1.4); 1.9269 (2.4); 1.9146 (3.0); 1.9026 (2.1); 1.8629 (0.7); 1.8413 (1.8); 1.8317 (0.8); 1.8208 (1.8); 1.8102 (1.7); 1.7991 (0.8); 1.7898 (1.6); 1.7681 (0.6); 0.0080 (0.3); −0.0002 (9.5); −0.0082 (0.5)

I-237: ¹H-NMR(400.0 MHz, d₆-DMSO):

δ=7.2618 (1.8); 7.2430 (1.9); 7.1578 (0.8); 7.1542 (0.9); 7.1368 (1.8); 7.1193 (1.1); 7.1158 (1.1); 6.9162 (2.1); 6.8946 (2.2); 6.8729 (1.2); 6.8705 (1.4); 6.8521 (2.2); 6.8358 (1.0); 6.8333 (1.1); 6.7736 (2.4); 6.7718 (2.4); 6.7532 (2.2); 4.7532 (0.5); 4.7396 (1.1); 4.7276 (0.9); 4.7186 (1.1); 4.7053 (0.5); 4.3122 (2.4); 4.3001 (2.0); 4.2875 (2.4); 4.2652 (0.3); 4.2573 (0.4); 4.2374 (1.2); 4.2294 (1.2); 4.2184 (1.9); 4.2094 (2.2); 4.1950 (1.2); 4.1773 (0.4); 3.9018 (16.0); 3.6652 (2.1); 3.6591 (1.7); 3.6511 (2.1); 3.6433 (1.7); 3.6370 (2.2); 3.3225 (88.5); 3.1745 (1.3); 3.1617 (1.2); 2.6703 (0.6); 2.6659 (0.5); 2.5406 (0.5); 2.5057 (83.8); 2.5012 (107.4); 2.4968 (80.4); 2.3324 (0.5); 2.3279 (0.6); 2.3234 (0.5); 2.1380 (1.9); 2.1252 (1.5); 2.1152 (1.0); 2.1029 (0.6); 2.0909 (0.5); 2.0782 (0.7); 2.0666 (0.9); 2.0568 (0.8); 2.0467 (0.8); 2.0356 (0.5); 2.0233 (0.5); 2.0092 (1.0); 2.0002 (1.0); 1.9948 (0.7); 1.9858 (0.6); 1.9740 (0.6); 1.9661 (0.4); 1.9594 (0.4); 1.9510 (0.3); 1.9366 (0.9); 1.9249 (1.5); 1.9129 (1.8); 1.9024 (1.3); −0.0002 (3.8)

I-238: ¹H-NMR(400.0 MHz, d₆-DMSO):

δ=7.1219 (2.3); 7.1030 (3.2); 7.0644 (4.1); 7.0164 (2.5); 6.9975 (1.7); 6.8310 (2.4); 6.8091 (2.5); 5.0709 (0.6); 5.0512 (1.6); 5.0311 (1.6); 5.0107 (0.6); 4.2074 (2.4); 4.1929 (4.4); 4.1783 (2.5); 3.9020 (2.3); 3.8244 (2.6); 3.8102 (2.4); 3.7955 (2.8); 3.3222 (76.5); 2.9043 (0.5); 2.8968 (0.5); 2.8830 (0.6); 2.8750 (0.6); 2.8657 (0.8); 2.8583 (0.9); 2.8436 (0.9); 2.8369 (0.8); 2.7574 (0.6); 2.7364 (1.2); 2.7162 (1.0); 2.6982 (0.8); 2.6752 (0.8); 2.6706 (0.8); 2.5645 (0.8); 2.5503 (1.9); 2.5364 (2.7); 2.5054 (86.0); 2.5011 (111.4); 2.4968 (82.0); 2.4688 (1.2); 2.4607 (1.2); 2.4493 (1.1); 2.4380(I.I); 2.4300 (0.9); 2.4186 (0.6); 2.4110 (0.5); 2.3322 (0.5); 2.3277 (0.6); 2.3238 (0.5); 2.2593 (16.0); 1.8445 (0.4); 1.8230 (1.2); 1.8136 (0.5); 1.8023 (1.2); 1.7922 (1.2); 1.7804 (0.5); 1.7713 (1.1); 1.7497 (0.4); −0.0001 (0.9)

I-239: ¹H-NMR(400.0 MHz, d₆-DMSO):

δ=7.1009 (3.9); 6.9838 (0.5); 6.9639 (9.0); 6.9401 (0.4); 6.7817 (2.3); 6.7591 (2.4); 4.7050 (0.5); 4.6879 (0.8); 4.6749 (1.1); 4.6541 (0.6); 4.2000 (2.3); 4.1856 (4.1); 4.1711 (2.3); 3.9019 (16.0); 3.8196 (2.4); 3.8053 (2.3); 3.7907 (2.6); 3.3239 (104.7); 3.2672 (0.4); 2.7299 (0.4); 2.7028 (0.8); 2.6840 (1.2); 2.6711 (1.8); 2.6559 (1.4); 2.6419 (0.7); 2.6139 (0.4); 2.5462 (1.8); 2.5405 (2.2); 2.5325 (2.6); 2.5234 (3.2); 2.5096 (45.7); 2.5057 (90.7); 2.5013 (118.5); 2.4969 (86.0); 2.3324 (0.5); 2.3280 (0.7); 2.3235 (0.5); 2.2171 (16.0); 1.9331 (0.7); 1.9194 (1.0); 1.9021 (1.6); 1.8767 (0.9); 1.8586 (0.5); 1.7798 (0.4); 1.7571 (1.0); 1.7377 (1.0); 1.7316 (1.0); 1.7145 (0.9); 1.7016 (0.8); 1.6879 (0.6); 1.6800 (0.7); 1.6638 (0.4); 1.6580 (0.4); −0.0002 (0.8)

1H-NMR

I-241: ¹H-NMR(400.0 MHz, d₆-DMSO):

δ=7.2678 (5.3); 7.2494 (11.4); 7.2309 (8.5); 7.2131 (3.7); 7.1973 (7.0); 7.1798 (4.0); 7.1720 (5.5); 7.1530 (5.4); 7.1350 (1.8); 6.8659 (7.2); 6.8440 (7.4); 5.1102 (1.8); 5.0903 (4.9); 5.0699 (4.8); 5.0502 (1.8); 4.2083 (7.0); 4.1938 (12.7); 4.1791 (7.2); 4.0976 (0.8); 4.0848 (0.8); 3.9019 (16.0); 3.8242 (7.6); 3.8096 (6.9); 3.7951 (8.0); 3.3219 (201.5); 3.2671 (1.4); 3.1745 (3.8); 3.1617 (3.8); 2.9692 (1.4); 2.9611 (1.7); 2.9477 (1.7); 2.9395 (1.8); 2.9300 (2.5); 2.9219 (2.8); 2.9083 (2.7); 2.9003 (2.6); 2.8211 (1.9); 2.8006 (4.1); 2.7807 (3.2); 2.7613 (2.5); 2.7407 (1.4); 2.6745 (1.4); 2.6701 (1.8); 2.6655 (1.4); 2.5512 (5.5); 2.5371 (7.8); 2.5230 (9.8); 2.5054 (247.3); 2.5011 (322.8); 2.4968 (236.2); 2.4745 (4.2); 2.4630 (3.4); 2.4518 (3.2); 2.4434 (2.8); 2.4325 (1.6); 2.4242 (1.3); 2.3320 (1.4); 2.3279 (1.8); 2.3235 (1.4); 1.8708 (1.2); 1.8492 (3.5); 1.8395 (1.4); 1.8287 (3.4); 1.8182 (3.3); 1.8072 (1.4); 1.7977 (3.1); 1.7762 (1.0); 1.2351 (0.4); −0.0002 (2.4)

I-242: ¹H-NMR(400.0 MHz, d₆-DMSO):

δ=7.2480 (2.4); 7.2288 (2.6); 7.1591 (1.2); 7.1557 (1.2); 7.1386 (2.4); 7.1205 (1.6); 7.1171 (1.4); 7.0084 (2.8); 6.9867 (2.9); 6.8690 (1.8); 6.8507 (3.1); 6.8318 (1.4); 6.7755 (3.4); 6.7554 (3.1); 4.7822 (0.7); 4.7691 (1.5); 4.7577 (1.1); 4.7478 (1.5); 4.7344 (0.7); 4.2674 (0.3); 4.2595 (0.5); 4.2398 (1.7); 4.2314 (1.9); 4.2203 (2.7); 4.2110 (5.7); 4.1964 (5.7); 4.1816 (3.3); 3.9016 (16.0); 3.8238 (3.0); 3.8094 (2.8); 3.7949 (3.1); 3.3249 (141.4); 3.2669 (0.4); 2.6699 (0.7); 2.5504 (2.2); 2.5369 (3.1); 2.5052 (102.5); 2.5011 (130.8); 2.4969 (95.9); 2.3277 (0.8); 2.3237 (0.6); 2.1031 (0.6); 2.0918 (0.6); 2.0793 (0.9); 2.0678 (1.2); 2.0569 (1.0); 2.0483 (1.0); 2.0371 (0.6); 2.0186 (0.6); 2.0045 (1.3); 1.9952 (1.3); 1.9899 (1.0); 1.9806 (0.8); 1.9690 (0.7): 1.9617 (0.6); 1.9545 (0.5); −0.0004 (0.8)

I-245: ¹H-NMR(400.0 MHz, d₆-DMSO):

δ=7.1134 (2.2); 7.0944 (3.1); 7.0619 (3.7); 7.0073 (2.4); 6.9882 (1.7); 6.5489 (1.8); 6.5269 (1.8); 5.0390 (0.6); 5.0194 (1.5); 4.9990 (1.5); 4.9795 (0.6); 4.2622 (1.8); 4.2440 (3.3); 3.9016 (7.6); 3.5561 (0.8); 3.5475 (0.9); 3.5197 (1.0); 3.5114 (1.0); 3.3317 (123.6); 3.1750 (0.5); 3.1621 (0.4); 3.0322 (0.9); 3.0006 (1.4); 2.9666 (0.9); 2.8881 (0.5); 2.8659 (0.6); 2.8547 (0.8); 2.8492 (0.9); 2.8336 (0.8); 2.8282 (0.8); 2.7442 (0.6); 2.7231 (1.2); 2.7034 (1.0); 2.6841 (0.8); 2.6754 (0.6); 2.6707 (0.8); 2.6661 (0.9); 2.5545 (0.5); 2.5240 (3.4); 2.5063 (89.7); 2.5019 (113.9); 2.4976 (83.5); 2.4729 (1.1); 2.4616 (1.3); 2.4536 (1.1); 2.4422 (1.1); 2.4309 (1.1); 2.4229 (0.9); 2.4119 (0.6); 2.4038 (0.4); 2.3328 (0.5); 2.3286 (0.7); 2.3243 (0.5); 2.2561 (16.0); 2.0609 (1.6); 2.0308 (1.5); 2.0217 (1.6); 2.0081 (0.5); 1.8354 (0.4); 1.8140 (1.1); 1.8045 (0.5); 1.7924 (1.4); 1.7829 (1.4); 1.7694 (0.8); 1.7618 (1.5); 1.7392 (0.8); 1.7318 (0.6): 1.7181 (0.4): -0.0001 (5.3)

I-246: ¹H-NMR(400.0 MHz, d₆-DMSO):

δ=7.1166 (3.9); 6.9744 (0.5); 6.9542 (9.0); 6.9305 (0.5); 6.4798 (2.2); 6.4571 (2.3); 4.6709 (0.5); 4.6543 (0.9); 4.6418 (1.1); 4.6199 (0.5); 4.2514 (2.3); 4.2364 (3.0); 3.9016 (14.4); 3.5505 (0.8); 3.5474 (0.8); 3.5425 (0.8); 3.5148 (0.9); 3.5070 (1.0); 3.3221 (87.5); 3.2671 (0.4); 3.1747 (0.8); 3.1619 (0.8); 3.0361 (0.8); 3.0042 (1.3); 2.9696 (0.8); 2.7221 (0.4); 2.6967 (0.8); 2.6752 (1.6); 2.6703 (1.6); 2.6658 (1.7); 2.6474 (1.4); 2.6341 (0.7); 2.6057 (0.4); 2.5499 (0.4); 2.5100 (49.2); 2.5058 (95.1); 2.5014 (123.6); 2.4969 (93.2); 2.3325 (0.6); 2.3281 (0.8); 2.3237 (0.6); 2.2135 (16.0); 2.0552 (1.7); 2.0184 (1.7); 1.9261 (0.7); 1.9144 (1.0); 1.8943 (1.7); 1.8707 (0.8); 1.7896 (0.4); 1.7751 (0.8); 1.7512 (1.5); 1.7283 (1.6); 1.7095 (1.0); 1.6887 (0.9); 1.6646 (0.8); 1.6452 (0.6); −0.0002 (7.9);-0.0081 (0.5)

I-247: ¹H-NMR(400.0 MHz, d₆-DMSO):

δ=7.2645 (1.8); 7.2453 (2.0); 7.1503 (1.1); 7.1466 (1.1); 7.1290 (2.2); 7.1118 (1.4); 7.1080 (1.4); 6.8652 (1.6); 6.8623 (1.7); 6.8464 (2.8); 6.8439 (2.9); 6.8280 (1.4); 6.8252 (1.4); 6.7671 (3.1); 6.7653 (3.0); 6.7467 (2.9); 6.7312 (2.7); 6.7094 (2.8); 4.7440 (0.6); 4.7304 (1.3); 4.7199 (1.0); 4.7093 (1.3); 4.6963 (0.6); 4.2603 (3.0); 4.2465 (3.7); 4.2385 (3.5); 4.2255 (1.3); 4.2187 (1.2); 4.2119 (2.0); 4.2041 (1.7); 4.1981 (1.6); 4.1846 (1.1); 4.1701 (0.5); 4.0978 (0.6); 4.0847 (0.6); 3.9017 (16.0); 3.5558 (0.8); 3.5473 (1.0); 3.5346 (0.3); 3.5186 (1.0); 3.5108 (1.0); 3.3229 (130.1); 3.2670 (0.6); 3.1748 (2.8); 3.1617 (2.7); 3.0436 (1.0); 3.0117 (1.6); 2.9799 (1.0); 2.6749 (0.5); 2.6704 (0.7); 2.6660 (0.5); 2.5403 (0.4); 2.5235 (3.1); 2.5102 (50.3); 2.5058 (98.5); 2.5013 (127.5); 2.4968 (94.7); 2.4924 (48.8); 2.3325 (0.6); 2.3281 (0.8); 2.3236 (0.7); 2.0902 (0.4); 2.0562 (2.6); 2.0264 (2.4); 2.0185 (2.5); 2.0028 (1.7); 1.9953 (1.2); 1.9883 (0.9); 1.9796 (0.6); 1.9680 (0.6); 1.9607 (0.5); 1.9534 (0.4); 1.7892 (0.4); 1.7791 (0.5); 1.7675 (0.6); 1.7600 (0.7); 1.7530 (0.6); 1.7416 (0.7); 1.7158 (0.4); 1.4258 (0.4); −0.0002 (2.8)

1H-NMR

I-248: ¹H-NMR(400.0 MHz, d₆-DMSO):

δ=7.0985 (2.2); 7.0796 (3.2); 7.0549 (3.9); 6.9926 (2.4); 6.9738 (1.7); 6.1306 (2.2); 6.1084 (2.3); 4.9959 (0.6); 4.9760 (1.6); 4.9558 (1.6); 4.9355 (0.6); 4.1547 (3.0); 4.1426 (2.4); 4.1298 (3.1); 3.9015 (12.0); 3.3226 (112.1); 2.8903 (2.7); 2.8837 (2.1); 2.8767 (3.0); 2.8690 (2.6); 2.8632 (3.1); 2.8474 (0.7); 2.8380 (0.9); 2.8303 (0.9); 2.8159 (0.9); 2.8088 (0.8); 2.7237 (0.6); 2.7031 (1.2); 2.6826 (1.0); 2.6751 (0.7); 2.6699 (1.0); 2.6656 (1.2); 2.6438 (0.4); 2.5099 (47.1); 2.5057 (91.3); 2.5013 (118.1); 2.4968 (87.7); 2.4583 (0.9); 2.4505 (0.8); 2.4394 (1.1); 2.4317 (1.1); 2.4201 (1.2); 2.4085 (1.2); 2.4011 (1.1); 2.3898 (0.6); 2.3818 (0.5); 2.3326 (0.6); 2.3282 (0.8); 2.3235 (0.6); 2.2519 (16.0); 2.0614 (2.3); 2.0488 (1.8); 2.0337 (0.8); 1.8069 (0.5); 1.7852 (2.2); 1.7745 (2.1); 1.7639 (3.2); 1.7549 (2.9); 1.7337 (1.9): 1.7118 (0.5); −0.0002 (2.0)

I-249: ¹H-NMR(400.0 MHz, d₆-DMSO):

δ=7.3059 (1.4); 7.2880 (1.6); 7.1498 (0.4); 7.1363 (1.4); 7.1318 (1.6); 7.1274 (1.7); 7.1207 (4.3); 7.1144 (2.3); 7.1095 (1.6); 7.1042 (2.0); 7.0909 (0.7); 7.0822 (2.2); 7.0761 (1.8); 7.0602 (1.1); 6.5334 (2.3); 6.5105 (2.4); 4.7137 (0.5); 4.6951 (0.8); 4.6830 (1.0); 4.6619 (0.5); 4.2478 (2.2); 4.2332 (3.0); 4.2249 (2.1); 3.9018 (16.0); 3.5477 (0.8); 3.5407 (0.8); 3.5349 (0.7); 3.5126 (0.9); 3.5048 (0.9); 3.3217 (113.4); 3.1747 (1.0); 3.1616 (1.0); 3.0425 (0.9); 3.0109 (1.4); 2.9790 (0.8); 2.7783 (0.3); 2.7528 (0.7); 2.7340 (1.1); 2.7209 (1.3); 2.7038 (1.4); 2.6895 (0.7); 2.6751 (0.7); 2.6702 (0.8); 2.6654 (0.8); 2.5475 (0.4); 2.5056 (86.4); 2.5012 (111 .7); 2.4968 (83.3); 2.3323 (0.6); 2.3280 (0.7); 2.3240 (0.6); 2.0545 (1.5); 2.0167 (1.6); 1.9480 (0.7); 1.9359 (1.0); 1.9241 (1.7); 1.9145 (1.6); 1.8969 (0.8); 1.8801 (0.5); 1.7802 (1.3); 1.7585 (1.5); 1.7361 (1.2): 1.7187 (1.1); 1.6990 (0.9); 1.6753 (0.5); −0.0002 (6.6)

I-250: ¹H-NMR(400.0 MHz, d₆-DMSO):

δ=7.2723 (3.3); 7.2547 (4.5); 7.2396 (2.9); 7.2219 (6.1); 7.2042 (2.6); 7.1874 (5.0); 7.1669 (4.8); 7.1459 (3.8); 7.1282 (1.2); 6.5904 (4.2); 6.5685 (4.3); 5.0740 (1.0); 5.0543 (2.9); 5.0345 (2.9); 5.0145 (1.0); 4.2988 (0.6); 4.2628 (3.8); 4.2439 (7.1); 4.2080 (0.4); 3.9017 (16.0); 3.5549 (1.7); 3.5472 (1.9); 3.5191 (2.0); 3.5109 (2.1); 3.3231 (171.9); 3.1680 (0.6); 3.0356 (1.9); 3.0040 (3.2); 2.9716 (2.0); 2.9596 (1.2); 2.9513 (1.2); 2.9379 (1.2); 2.9294 (1.3); 2.9202 (1.8); 2.9122 (1.9); 2.8983 (1.9); 2.8907 (1.8); 2.8079 (1.4); 2.7871 (3.0); 2.7671 (2.4); 2.7478 (1.8); 2.7273 (1.1); 2.6744 (0.9); 2.6704 (1.3); 2.6663 (1.0); 2.5580 (0.8); 2.5536 (0.8); 2.5234 (6.0); 2.5056 (172.8); 2.5014 (220.5); 2.4971 (167.4); 2.4749 (4.1); 2.4663 (3.7); 2.4550 (2.9); 2.4437 (2.6); 2.4354 (2.2); 2.4244 (1.3); 2.4162 (1.1); 2.3323 (1.1); 2.3281 (1.4); 2.3239 (1.1); 2.0602 (3.5); 2.0304 (3.3); 2.0206 (3.6); 1.8611 (0.9); 1.8396 (2.5); 1.8299 (1.1); 1.8188 (2.5); 1.8086 (2.5); 1.7970 (1.6); 1.7876 (2.8); 1.7658 (1.9); 1.7334 (1.4); 1.7215 (0.8); 1.7003 (0.6); 1.2349 (0.4); 0.0080 (0.4); −0.0001 (12.8)

I-252: ¹H-NMR(400.0 MHz, d₆-DMSO):

δ=8.3145 (0.4); 7.2873 (5.6); 7.2706 (6.8); 7.2661 (6.7); 7.2505 (0.4); 7.1595 (1.6); 7.1457 (5.1); 7.1417 (5.8); 7.1296 (12.4); 7.1271 (12.2); 7.1153 (5.6); 7.1104 (6.6); 7.0920 (9.2); 7.0870 (6.8); 7.0704 (4.0); 6.8292 (8.4); 6.8063 (8.7); 4.7482 (1.7); 4.7305 (2.9); 4.7171 (3.9); 4.6960 (1.8); 4.1980 (8.1); 4.1837 (14.6); 4.1690 (8.4); 3.9020 (16.0); 3.8183 (8.8); 3.8041 (8.2); 3.7893 (9.3); 3.3235 (389.3); 3.1744 (0.9); 3.1617 (0.9); 2.8009 (0.8); 2.7833 (1.2); 2.7587 (2.8); 2.7401 (4.5); 2.7285 (4.9); 2.7127 (5.1); 2.6980 (2.6); 2.6702 (3.5); 2.5453 (6.6); 2.5403 (6.2); 2.5317 (9.4); 2.5233 (11.8); 2.5056 (301.6); 2.5012 (391.6); 2.4968 (287.0); 2.3320 (1.6); 2.3278 (2.2); 2.3235 (1.7); 1.9774 (0.9); 1.9565 (2.4); 1.9436 (3.8); 1.9286 (6.1); 1.9028 (3.3); 1.8850 (2.0); 1.8640 (0.8); 1.8135 (1.5); 1.7866 (3.8); 1.7676 (3.8); 1.7624 (3.7); 1.7440 (3.2); 1.7320 (3.2); 1.7184 (2.4); 1.7107 (2.7); 1.6895 (1.6); 1.6707 (0.6); 1.2355 (0.4); −0.0002 (3.2)

I-253: ¹H-NMR(400.0 MHz, d₆-DMSO):

δ=7.3011 (0.9); 7.2893 (1.0); 7.2867 (1.0); 7.1484 (0.3); 7.1395 (0.9); 7.1368 (1.0); 7.1285 (2.1); 7.1249 (2.1); 7.1164 (0.8); 7.1133 (0.9); 7.1044 (0.4); 7.0850 (1.1); 7.0817 (1.0); 7.0708 (0.7); 6.7080 (1.4); 6.6930 (1.5); 4.7035 (0.5); 4.6943 (0.5); 4.2953 (1.6); 4.2871 (1.2); 4.2783 (1.5); 3.8994 (16.0); 3.6520 (1.4); 3.6478 (0.9); 3.6424 (1.3); 3.6373 (0.9); 3.6328 (1.5); 3.3249 (0.4); 3.3044 (18.2); 2.7566 (0.4); 2.7439 (0.6); 2.7351 (0.4); 2.7181 (0.4); 2.7070 (0.7); 2.6970 (0.4); 2.5210 (0.6); 2.5179 (0.8); 2.5148 (0.8); 2.5060 (18.9); 2.5030 (41.8); 2.4999 (58.6); 2.4969 (42.5); 2.4939 (19.7); 2.3840 (0.3); 2.1359 (1.1); 2.1267 (0.8); 2.1212 (0.5); 1.9606 (0.3); 1.9519 (0.4); 1.9477 (0.4); 1.9315 (0.9); 1.9207 (1.1); 1.9121 (1.4); 1.9032 (1.1); 1.7837 (0.5); 1.7804 (0.4); 1.7684 (0.4); 1.7644 (0.4); 1.7525 (0.4); 1.7241 (0.4); 1.7158 (0.4); 1.7016 (0.4); −0.0002 (10.2); −0.0058 (0.4)

1H-NMR

I-254: ¹H-NMR(400.0 MHz, d₆-DMSO):

δ=7.1437 (4.0); 6.9603 (0.5); 6.9400 (9.0); 6.9167 (0.4); 6.2125 (2.2); 6.1898 (2.3); 4.6450 (0.5); 4.6277 (0.9); 4.6148 (1.0); 4.5934 (0.5); 4.0457 (3.0); 4.0275 (6.5); 4.0093 (3.1); 3.9017 (8.9); 3.6544 (0.4); 3.6375 (2.8); 3.6315 (2.9); 3.6198 (2.9); 3.6139 (2.9); 3.5968 (0.4); 3.5792 (0.4); 3.3216 (62.8); 3.1736 (0.3); 2.9734 (3.2); 2.9553 (6.6); 2.9370 (2.9); 2.7162 (0.4); 2.6895 (0.8); 2.6704 (1.8); 2.6577 (1.3); 2.6402 (1.4); 2.6271 (0.7); 2.5983 (0.4); 2.5234 (2.0); 2.5058 (87.3); 2.5014 (112.3); 2.4970 (84.3); 2.3452 (0.4); 2.3324 (0.6); 2.3283 (0.7); 2.3235 (0.6); 2.3182 (0.6); 2.2121 (16.0); 1.9451 (0.6); 1.9268 (0.8); 1.9143 (1.3); 1.9056 (1.8); 1.8953 (1.1); 1.8787 (0.8); 1.8608 (0.6); 1.7898 (0.4); 1.7616 (1.1); 1.7425 (1.0); 1.7376 (1.0); 1.7191 (0.7); 1.6998 (0.5); 1.6840 (0.8); 1.6616 (0.8); 1.6387 (0.5); 1.2196 (0.4); 1.2071 (0.8); 1.1995 (0.8); 1.1883 (1.2); 1.1764 (0.8); 1.1696 (0.9); 1.1575 (0.5); 1.1507 (0.3); 0.4557 (0.8); 0.4400 (3.2); 0.4305 (2.0); 0.4244 (2.6); 0.4200 (3.0); 0.4108 (1.4); 0.3751 (0.4); 0.3523 (1.3); 0.3399 (3.7); 0.3310 (3.4); 0.3275 (3.3); 0.3158 (1.0); −0.0002 (2.2)

I-255: ¹H-NMR(400.0 MHz, d₆-DMSO):

δ=7.2964 (1.9); 7.2775 (2.1); 7.1300 (0.9); 7.1260 (1.0); 7.1084 (1.9); 7.0913 (1.2); 7.0876 (1.2); 6.8394 (1.5); 6.8209 (2.4); 6.8046 (1.1); 6.8022 (1.2); 6.7434 (2.6); 6.7247 (2.4); 6.7229 (2.4); 6.5102 (2.3); 6.4885 (2.4); 4.7052 (0.5); 4.6918 (1.2); 4.6794 (0.9); 4.6707 (1.2); 4.6566 (0.6); 4.5344 (0.3); 4.2750 (0.4); 4.2671 (0.6); 4.2554 (0.5); 4.2473 (1.3); 4.2393 (1.1); 4.2277 (1.0); 4.2192 (0.9); 4.2008 (0.8); 4.1918 (1.1); 4.1857 (1.1); 4.1756 (1.1); 4.1646 (0.5); 4.1579 (0.6); 4.1495 (0.4); 4.0578 (2.6); 4.0396 (5.8); 4.0215 (2.8); 3.9019 (16.0); 3.6218 (0.3); 3.6029 (2.7); 3.5846 (2.9); 3.5700 (0.4); 3.3223 (96.3); 3.1733 (0.5); 3.1633 (0.5); 2.9708 (2.6); 2.9526 (5.3); 2.9344 (2.4); 2.7896 (0.3); 2.6748 (0.5); 2.6704 (0.7); 2.6660 (0.5); 2.5233 (1.9); 2.5057 (98.3); 2.5013 (130.3); 2.4969 (99.7); 2.3324 (0.6); 2.3281 (0.8); 2.3238 (0.7); 2.0766 (0.4); 2.0638 (0.7); 2.0536 (0.9); 2.0423 (1.2); 2.0302 (1.3); 2.0222 (1.6); 2.0138 (1.2); 1.9987 (0.7); 1.9876 (0.5); 1.9795 (0.3); 1.8026 (0.6); 1.7921 (0.6); 1.7835 (0.8); 1.7752 (0.7); 1.7640 (0.6); 1.7557 (0.6); 1.6568 (2.0); 1.6087 (2.3); 1.5849 (2.9); 1.1754 (0.3); 1.1277 (3.4); 1.1107 (2.0); 0.9572 (0.7); 0.9290 (1.5); 0.9007 (1.2); −0.0002 (1.8)

I-256: ¹H-NMR(400.0 MHz, d₆-DMSO):

δ=7.1025 (4.3); 7.0764 (3.3); 6.9887 (2.5); 6.9698 (1.8); 6.8367 (0.4); 6.3308 (2.5); 6.3093 (2.7); 4.9860 (0.6); 4.9663 (1.7); 4.9462 (1.7); 4.9259 (0.6); 4.0517 (2.9); 4.0334 (6.4); 4.0154 (3.2); 3.9019 (12.4); 3.8126 (0.7); 3.6105 (4.1); 3.5918 (4.2); 3.4995 (0.6); 3.3215 (96.3); 3.1750 (1.4); 3.1619 (1.4); 2.9703 (3.2); 2.9522 (6.6); 2.9340 (3.0); 2.8838 (0.6); 2.8691 (0.6); 2.8616 (0.7); 2.8516 (0.9); 2.8447 (1.0); 2.8299 (0.9); 2.8230 (0.9); 2.7294 (0.6); 2.7091 (1.2); 2.6886 (1.0); 2.6705 (1.7); 2.6500 (0.5); 2.5058 (121.7); 2.5015 (152.7); 2.4972 (113.6); 2.4661 (1.3); 2.4578 (1.1); 2.4465 (1.3); 2.4382 (1.3); 2.4271 (1.3); 2.4155 (1.3); 2.4079 (1.2); 2.3969 (0.8); 2.3890 (0.6); 2.3283 (1.0); 2.2496 (16.0); 1.8458 (0.6); 1.8239 (1.5); 1.8140 (1.1); 1.8028 (1.9); 1.7929 (2.1); 1.7817 (1.4); 1.7722 (1.8); 1.7505 (0.7); 1.6584 (2.3); 1.6178 (2.7); 1.5906 (3.3); 1.2646 (1.1); 1.2473 (1.2); 1.2360 (0.7); 1.1317 (4.1); 1.1153 (2.5); 0.9359 (1.6); 0.9067 (1.4); 0.8548 (0.9); 0.8387 (0.8); 0.0000 (3.2)

I-258: ¹H-NMR(400.0 MHz, d₆-DMSO):

δ=7.8158 (1.7); 7.7954 (1.2); 7.7786 (0.7); 7.6189 (0.5); 7.5990 (0.8); 7.5804 (0.3); 7.3413 (0.4); 7.3277 (0.5); 7.3182 (0.5); 7.1126 (0.7); 7.1012 (1.4); 7.0906 (1.1); 7.0787 (1.1); 7.0710 (0.8); 7.0537 (1.2); 7.0448 (1.0); 7.0398 (1.0); 7.0217 (0.6); 7.0021 (0.9); 6.9818 (0.5); 5.8925 (0.8); 5.8699 (0.8); 4.0080 (0.4); 3.9611 (0.6); 3.9471 (1.0); 3.9336 (0.5); 3.9281 (0.5); 3.9017 (16.0); 3.8721 (0.3); 3.8509 (0.4); 3.7668 (0.6); 3.7520 (1.2); 3.7367 (0.6); 3.3204 (14.8); 3.1861 (0.7); 3.1740 (0.6); 2.7133 (0.3); 2.6744 (0.6); 2.6699 (0.7); 2.6291 (0.5); 2.6152 (0.5); 2.6019 (0.5); 2.5052 (64.0); 2.5010 (82.4); 2.4968 (61.5); 2.3274 (0.7); 2.3234 (0.7); 2.3054 (0.6); 2.2960 (0.6); 2.2805 (0.4); 1.9723 (0.5); 1.9580 (0.7); 1.9439 (0.6); 1.9087 (0.4); 1.8958 (0.5); 1.8849 (0.5); 1.8727 (0.3); 1.7768 (0.4); 1.7533 (0.5); 1.7350 (0.4); 1.6491 (0.3); 1.6223 (0.4); 1.6159 (0.3); 1.5176 (0.5); 1.4987 (0.6); 1.4787 (0.4); −0.0003 (1.2 )

1H-NMR

I-259: ¹H-NMR(400.0 MHz, d₆-DMSO):

δ=7.3431 (1.1); 7.3278 (1.0); 7.3205 (1.1); 7.1098 (1.6); 7.1009 (2.7); 7.0922 (2.0); 7.0873 (1.3); 7.0753 (0.4); 7.0688 (0.3); 7.0589 (1.3); 7.0507 (1.0); 7.0370 (0.7); 6.2175 (1.3); 6.1950 (1.4); 4.6664 (0.6); 4.6554 (0.6); 3.9431 (1.4); 3.9276 (3.0); 3.9122 (1.6); 3.9020 (16.0); 3.8736 (0.8); 3.8596 (1.3); 3.8461 (0.9); 3.8359 (1.4); 3.8218 (0.8); 3.8027 (0.4); 3.5783 (0.5); 3.5706 (0.6); 3.5599 (1.0); 3.5468 (0.6); 3.5393 (0.5); 3.3199 (25.2); 3.1748 (0.5); 3.1620 (0.5); 2.7392 (0.5); 2.7203 (0.7); 2.7074 (0.9); 2.6927 (0.9); 2.6754 (0.7); 2.6704 (0.7); 2.5056 (67.7); 2.5014 (85.5); 2.4973 (63.4); 2.3281 (0.5); 2.1005 (0.4); 2.0857 (1.3); 2.0711 (1.9); 2.0568 (1.4); 2.0416 (0.6); 1.9440 (0.6); 1.9312 (1.1); 1.9197 (1.0); 1.9039 (0.6); 1.8836 (0.4); 1.8094 (0.7); 1.7855 (0.6); 1.7661 (0.3); 1.7125 (0.4); 1.6936 (0.5); 1.6727 (0.3); 0.9053 (0.4); 0.8929 (1.4); 0.8860 (2.4); 0.8811 (2.1); 0.8745 (2.2); 0.8589 (1.2); 0.8524 (2.3); 0.8450 (1.4); 0.8325 (2.1); 0.8255 (1.2); 0.8130 (0.5); −0.0002 (1.5)

I-261: ¹H-NMR(400.0 MHz, d₆-DMSO):

δ=7.3621 (0.9); 7.3455 (0.9); 7.3398 (0.8); 7.1301 (0.7); 7.1248 (1.2); 7.1159 (2.0); 7.1076 (1.4); 7.1013 (0.9); 7.0883 (0.4); 7.0768 (1.0); 7.0699 (0.7); 7.0547 (0.4); 6.1692 (0.9); 6.1466 (1.0); 4.7237 (0.4); 4.7124 (0.4); 3.9113 (1.3); 3.9019 (16.0); 3.8968 (2.7); 3.8811 (1.3); 3.8538 (0.7); 3.8463 (0.5); 3.8323 (0.8); 3.8200 (1.5); 3.8055 (1.4); 3.7931 (0.6); 3.3210 (27.7); 3.1747 (0.3); 2.7376 (0.5); 2.7218 (0.5); 2.7038 (0.6); 2.6750 (0.4); 2.6704 (0.5); 2.6661 (0.5); 2.5236 (1.2); 2.5059 (58.8); 2.5015 (76.3); 2.4970 (55.7); 2.3324 (0.4); 2.3281 (0.5); 2.3237 (0.4); 2.0748 (0.4); 2.0602 (1.0); 2.0459 (1.4); 2.0317 (1.0); 2.0182 (0.4); 1.9533 (0.5); 1.9383 (0.7); 1.9299 (0.8); 1.8205 (1.0); 1.8022 (1.3); 1.7794 (0.5); 1.6995 (1.0); 1.6917 (1.1); 1.6701 (1.1); 1.6140 (0.5); 1.5905 (0.5); 1.3355 (0.6); 1.3288 (0.5); 1.3070 (0.8); 1.2994 (0.7); 1.2769 (0.8); 1.2581 (0.7); 1.2492 (0.7); 1.2360 (1.0); 1.2058 (0.8); 1.1836 (0.9); −0.0002 (1.4)

I-262: ¹H-NMR(400.0 MHz, d₆-DMSO):

δ=7.3455 (1.0); 7.3307 (1.1); 7.3235 (1.2); 7.1225 (1.2); 7.1176 (2.0); 7.1087 (3.5); 7.1003 (2.5); 7.0943 (1.5); 7.0818 (0.5); 7.0681 (1.5); 7.0604 (1.1); 7.0513 (0.6); 7.0454 (0.7); 6.2218 (1.6); 6.1991 (1.6); 4.8625 (0.4); 4.8455 (1.0); 4.8282 (1.4); 4.8108 (1.0); 4.7938 (0.4); 4.6818 (0.3); 4.6628 (0.6); 4.6501 (0.7); 4.6296 (0.4); 4.0187 (2.2); 4.0006 (5.0); 3.9825 (2.4); 3.9019 (16.0); 3.3217 (66.1); 2.9107 (2.5); 2.8926 (5.3); 2.8744 (2.3); 2.7467 (0.6); 2.7300 (0.8); 2.7144 (0.9); 2.6966 (1.0); 2.6804 (0.6); 2.6745 (0.5); 2.6702 (0.7); 2.6546 (0.4); 2.5100 (32.6); 2.5056 (65.4); 2.5011 (85.8); 2.4966 (62.2); 2.4922 (30.5); 2.3320 (0.3); 2.3278 (0.5); 2.3234 (0.4); 1.9638 (0.4); 1.9446 (0.7); 1.9332 (1.3); 1.9209 (1.2); 1.9069 (0.6); 1.8875 (0.4); 1.8317 (0.3); 1.8031 (0.8); 1.7839 (0.6); 1.7792 (0.7); 1.7602 (0.4); 1.7124 (0.5); 1.6971 (0.5); 1.6731 (0.3); 1.3997 (7.3); 1.3924 (7.6); 1.3824 (7.4); 1.3751 (7.2); −0.0002 (2.2)

I-264: ¹H-NMR(400.0 MHz, d₆-DMSO):

δ=7.2989 (2.8); 7.2804 (3.1); 7.1348 (1.4); 7.1316 (1.4); 7.1145 (2.9); 7.0966 (1.8); 6.8534 (2.0); 6.8518 (2.0); 6.8347 (3.5); 6.8164 (1.7); 6.7522 (4.0); 6.7319 (3.6); 6.4323 (3.2); 6.4108 (3.3); 4.8667 (0.7); 4.8497 (1.8); 4.8325 (2.4); 4.8152 (1.8); 4.7982 (0.7); 4.7153 (0.8); 4.7019 (1.8); 4.6914 (1.3); 4.6810 (1.8); 4.6675 (0.8); 4.2776 (0.6); 4.2686 (0.8); 4.2579 (0.8); 4.2496 (1.9); 4.2403 (1.6); 4.2312 (1.5); 4.2215 (1.3); 4.2045 (1.2); 4.1909 (1.8); 4.1790 (1.6); 4.1615 (0.9); 4.1522 (0.6); 4.0349 (4.0); 4.0168 (8.6); 3.9987 (4.2); 3.9018 (6.4); 3.3213 (75.6); 3.1728 (1.0); 3.1640 (0.9); 2.9169 (4.4); 2.8988 (9.1); 2.8807 (4.1); 2.6706 (0.8); 2.5050 (115.0); 2.5012 (139.7); 2.4973 (104.7); 2.3279 (0.8); 2.0918 (0.4); 2.0794 (0.4); 2.0667 (1.0); 2.0532 (2.0); 2.0377 (3.2); 2.0274 (2.5); 2.0139 (1.0); 2.0037 (0.5); 1.9922 (0.3); 1.9889 (0.3); 1.3998 (15.9); 1.3963 (15.7); 1.3826 (16.0); 1.3791 (15.4); 1.2355 (0.3); −0.0002 (2.1)

1H-NMR

I-266: ¹H-NMR(400.0 MHz, d₆-DMSO):

δ=7.7076 (4.9); 7.6350 (3.0); 7.6164 (5.9); 7.5780 (3.4); 7.5590 (3.0); 7.5404 (1.3); 7.3628 (0.4); 7.3440 (0.5); 7.3027 (2.6); 7.2858 (2.9); 7.2833 (2.9); 7.1270 (0.8); 7.1127 (2.2); 7.1092 (2.4); 7.0948 (4.0); 7.0909 (4.5); 7.0699 (3.0); 7.0573 (4.1); 7.0401 (1.9); 6.3045 (3.2); 6.2817 (3.4); 5.0616 (0.8); 5.0222 (6.1); 5.0143 (6.1); 4.9747 (0.7); 4.6816 (0.7); 4.6636 (1.3); 4.6513 (1.5); 4.6299 (0.7); 4.0971 (4.0); 4.0790 (8.6); 4.0609 (4.1); 3.9019 (16.0); 3.3256 (273.4); 3.2672 (0.5); 3.1746 (0.8); 3.1627 (0.7); 3.0619 (4.1); 3.0439 (8.6); 3.0258 (3.9); 3.0117 (0.4); 2.9461 (0.4); 2.7593 (0.5); 2.7318 (1.1); 2.7132 (1.7); 2.7012 (2.0); 2.6853 (2.1); 2.6747 (1.9); 2.6707 (2.3); 2.6434 (0.5); 2.5058 (171.2); 2.5015 (221.5); 2.4972 (168.6); 2.3939 (0.4); 2.3326 (1.1); 2.3283 (1.5); 2.3240 (1.2); 2.0576 (0.3); 2.0425 (0.4); 1.9553 (0.4); 1.9271 (1.7); 1.9131 (2.7); 1.9019 (2.7); 1.8877 (1.5); 1.8697 (0.8); 1.8494 (0.4); 1.7980 (0.7); 1.7741 (1.6); 1.7548 (1.4); 1.7322 (1.0); 1.7164 (0.9); 1.6971 (1.1); 1.6923 (1.0); 1.6814 (1.2); 1.6593 (0.8); 1.6359 (0.4); 1.2355 (0.6); 0.8525 (0.3); −0.0003 (1.3)

I-267: ¹H-NMR(400.0 MHz, d₆-DMSO):

δ=7.4868 (0.4); 7.4777 (2.8); 7.4711 (2.0); 7.4672 (2.4); 7.4601 (2.1); 7.4547 (3.7); 7.4468 (0.6); 7.4355 (0.4); 7.3369 (0.3); 7.3338 (0.3); 7.3260 (0.5); 7.3196 (1.0); 7.3057 (4.6); 7.3013 (3.6); 7.2950 (5.5); 7.2875 (5.8); 7.2829 (7.7); 7.2684 (3.5); 7.2640 (3.3); 7.1990 (2.8); 7.1933 (1.7); 7.1865 (2.2); 7.1756 (1.9); 7.1136 (0.8); 7.0995 (2.4); 7.0957 (2.5); 7.0821 (4.9); 7.0766 (5.1); 7.0622 (2.6); 7.0579 (3.1); 7.0429 (4.4); 7.0245 (2.0); 6.2986 (3.5); 6.2759 (3.6); 4.9785 (11.7); 4.6370 (0.7); 4.6189 (1.3); 4.6066 (1.6); 4.5855 (0.8); 4.4646 (0.4); 4.4502 (0.4); 4.1727 (4.2); 4.1546 (9.4); 4.1365 (4.4); 4.0134 (0.5); 3.9018 (16.0); 3.6098 (2.4); 3.3239 (204.1); 3.1747 (1.0); 3.1618 (1.0); 3.1314 (4.5); 3.1134 (9.6); 3.0952 (4.2); 2.7546 (0.5); 2.7382 (1.0); 2.7205 (0.7); 2.7121 (1.2); 2.6928 (2.1); 2.6838 (2.1); 2.6701 (3.4); 2.6268 (0.6); 2.5233 (3.6); 2.5098 (77.9); 2.5056 (153.0); 2.5012 (198.0); 2.4967 (145.4); 2.4925 (73.8); 2.3323 (0.9); 2.3279 (1.2); 2.3234 (0.9); 1.9418 (0.4); 1.9200 (1.0); 1.9068 (1.5); 1.8902 (2.6); 1.8648 (1.4); 1.8456 (0.8); 1.7679 (0.6); 1.7634 (0.6); 1.7407 (1.6); 1.7215 (1.6); 1.7161 (1.5); 1.6981 (1.4); 1.6841 (1.4); 1.6694 (1.0); 1.6603 (1.2); 1.6463 (0.7); −0.0002 (1.6)

I-270: ¹H-NMR(400.0 MHz, d₆-DMSO):

δ=7.4870 (0.4); 7.4781 (2.5); 7.4717 (1.9); 7.4684 (2.0); 7.4602 (1.9); 7.4551 (3.3); 7.4475 (0.5); 7.3256 (0.5); 7.3196 (0.8); 7.3040 (4.0); 7.2941 (4.9); 7.2862 (4.0); 7.2834 (4.3); 7.2815 (4.2); 7.2682 (0.9); 7.2627 (0.5); 7.2379 (2.8); 7.2190 (3.0); 7.1979 (2.7); 7.1843 (2.0); 7.1747 (1.7); 7.1124 (1.4); 7.1087 (1.4); 7.0913 (2.8); 7.0739 (1.8); 7.0702 (1.7); 6.8131 (2.0); 6.8108 (2.1); 6.7923 (3.5); 6.7759 (1.7); 6.7735 (1.7); 6.7259 (3.8); 6.7056 (3.6); 6.4955 (3.3); 6.4739 (3.4); 4.9803 (11.0); 4.6693 (0.8); 4.6555 (1.7); 4.6443 (1.3); 4.6346 (1.7); 4.6215 (0.8); 4.2278 (0.5); 4.2195 (0.7); 4.1993 (2.1); 4.1891 (5.0); 4.1708 (10.8); 4.1600 (2.7); 4.1525 (5.3); 4.1379 (0.7); 4.1316 (0.8); 4.1227 (0.5); 3.9018 (16.0); 3.3252 (166.1); 3.1685 (0.6); 3.1373 (4.2); 3.1192 (8.8); 3.1011 (3.9); 2.6750 (0.6); 2.6704 (0.9); 2.6660 (0.7); 2.5057 (120.5); 2.5013 (153.6); 2.4969 (113.5); 2.3324 (0.7); 2.3281 (0.9); 2.3236 (0.7); 2.0572 (0.5); 2.0455 (0.6); 2.0327 (1.0); 2.0225 (1.3); 2.0114 (1.4); 2.0005 (1.2); 1.9916 (1.3); 1.9839 (1.2); 1.9781 (1.5); 1.9696 (1.6); 1.9634 (1.1); 1.9547 (1.0); 1.9434 (0.7); 1.9354 (0.6); 1.9285 (0.5); 1.1800 (0.4); −0.0002 (1.3)

I-271: ¹H-NMR(400.0 MHz, d₆-DMSO):

δ=7.3393 (2.2); 7.3229 (2.5); 7.3178 (2.4); 7.1309 (0.6); 7.1169 (2.0); 7.1129 (2.3); 7.1073 (2.3); 7.1013 (5.5); 7.0955 (3.2); 7.0894 (2.4); 7.0847 (2.6); 7.0712 (1.0); 7.0624 (3.0); 7.0567 (2.4); 7.0409 (1.5); 6.3081 (3.0); 6.2853 (3.1); 4.6681 (0.7); 4.6492 (1.2); 4.6381 (1.4); 4.6166 (0.7); 4.0402 (3.7); 4.0220 (8.1); 4.0039 (3.9); 3.9018 (16.0); 3.6038 (4.7); 3.5852 (4.9); 3.3225 (107.0); 3.1671 (0.3); 2.9644 (3.3); 2.9465 (6.4); 2.9282 (2.9); 2.7821 (0.3); 2.7679 (0.5); 2.7405 (1.0); 2.7228 (1.6); 2.7088 (1.8); 2.6913 (1.9); 2.6752 (1.6); 2.6707 (1.4); 2.6664 (1.1); 2.6501 (0.6); 2.5058 (132.2); 2.5015 (166.4); 2.4972 (123.3); 2.3283 (1.0); 2.3238 (0.8); 1.9460 (1.5); 1.9312 (2.6); 1.9197 (2.4); 1.9050 (1.3); 1.8866 (0.8); 1.8671 (0.4); 1.8109 (1.2); 1.7880 (2.3); 1.7652 (2.1); 1.7462 (1.4); 1.7291 (1.0); 1.7097 (1.1); 1.6674 (2.9); 1.6575 (3.0); 1.6095 (3.3); 1.5845 (3.9); 1.2363 (0.4); 1.1299 (4.6); 0.9565 (1.0); 0.9288 (2.0); 0.9030 (1.7); 0.8743 (0.7); −0.0002 (2.9)

1H-NMR

I-273: ¹H-NMR(400.0 MHz, d₆-DMSO):

δ=8.3149 (0.3); 7.7053 (5.0); 7.6375 (2.9); 7.6167 (5.6); 7.5788 (3.5); 7.5595 (3.0); 7.5405 (1.1); 7.2539 (3.0); 7.2348 (3.1); 7.1261 (1.4); 7.1222 (1.5); 7.1048 (2.8); 7.0875 (1.8); 7.0837 (1.8); 7.0561 (0.4); 7.0418 (0.3); 7.0222 (0.4); 6.8261 (2.0); 6.8234 (2.2); 6.8073 (3.5); 6.8050 (3.6); 6.7889 (1.8); 6.7862 (1.8); 6.7423 (4.0); 6.7400 (3.9); 6.7218 (3.7); 6.7196 (3.4); 6.5115 (3.2); 6.4898 (3.3); 5.0629 (0.7); 5.0234 (6.3); 5.0172 (6.4); 4.9778 (0.6); 4.7129 (0.8); 4.6991 (1.7); 4.6871 (1.3); 4.6776 (1.7); 4.6642 (0.8); 4.5505 (0.4); 4.5346 (0.6); 4.5183 (0.5); 4.4867 (0.3); 4.2559 (0.6); 4.2475 (0.9); 4.2357 (0.8); 4.2278 (2.0); 4.2198 (1.7); 4.2083 (1.6); 4.1998 (1.4); 4.1878 (1.3); 4.1782 (1.7); 4.1725 (1.7); 4.1624 (1.7); 4.1513 (0.8); 4.1443 (1.0); 4.1358 (0.8); 4.1142 (4.2); 4.0960 (9.3); 4.0779 (4.5); 4.0636 (0.7); 4.0573 (0.4); 4.0458 (0.6); 3.9850 (0.4); 3.9019 (16.0); 3.3235 (73.8); 3.2674 (0.9); 3.1686 (4.6); 3.0690 (4.3); 3.0510 (9.3); 3.0328 (4.1); 2.8064 (0.4); 2.7901 (0.5); 2.7742 (0.4); 2.7020 (0.4); 2.6750 (0.9); 2.6704 (1.3); 2.6660 (0.9); 2.5237 (3.8); 2.5102 (84.0); 2.5059 (168.7); 2.5014 (220.6); 2.4969 (161.9); 2.4925 (81.6); 2.3373 (0.5); 2.3326 (1.0); 2.3281 (1.3); 2.3236 (1.0); 2.0722 (0.4); 2.0605 (0.5); 2.0472 (1.0); 2.0369 (1.4); 2.0253 (1.7); 2.0144 (2.0); 2.0065 (2.3); 1.9979 (1.9); 1.9831 (1.1); 1.9718 (0.7); 1.9634 (0.5); 1.9572 (0.5); 1.9486 (0.3); 1.2747 (0.4); 1.2590 (0.7); 1.2437 (0.6); 1.2351 (0.4): 1.1789 (0.7); 1.1608 (1.3); 1.1430 (0.6); −0.0002 (5.4)

I-277: ¹H-NMR(400.0 MHz, d₆-DMSO):

δ=7.2935 (4.9); 7.2762 (5.3); 7.1350 (2.5); 7.1313 (2.4); 7.1138 (5.0); 7.0965 (3.2); 7.0928 (2.9); 6.8524 (3.7); 6.8498 (3.6); 6.8336 (6.2); 6.8315 (6.0); 6.8152 (3.1); 6.8126 (2.9); 6.7516 (7.1); 6.7313 (6.4); 6.4864 (5.6); 6.4647 (5.7); 4.7215 (1.5); 4.7079 (3.1); 4.6948 (2.3); 4.6867 (3.0); 4.6729 (1.4); 4.2749 (1.1); 4.2667 (1.5); 4.2549 (1.4); 4.2470 (3.6); 4.2388 (3.0); 4.2277 (2.9); 4.2190 (2.5); 4.2055 (2.3); 4.1960 (2.9); 4.1900 (3.0); 4.1803 (3.0); 4.1687 (1.2); 4.1621 (1.5); 4.1535 (1.0); 4.0598 (7.2); 4.0415 (16.0); 4.0233 (7.7); 3.9019 (14.4); 3.6741 (1.1); 3.6565 (1.3); 3.6395 (5.7); 3.6262 (7.3); 3.6225 (7.2); 3.6091 (5.8); 3.5923 (1.3); 3.5744 (1.1); 3.3250 (290.4); 3.1689 (0.9); 2.9789 (7.8); 2.9608 (16.0); 2.9425 (6.9); 2.6751 (1.2); 2.6707 (1.5); 2.6661 (1.2); 2.5060 (212.7); 2.5016 (264.0); 2.4971 (190.8); 2.3327 (1.2); 2.3284 (1.5); 2.3238 (1.1); 2.1018 (0.4); 2.0924 (0.8); 2.0807 (0.9); 2.0675 (1.8); 2.0577 (2.5); 2.0455 (3.2); 2.0345 (3.7); 2.0269 (4.0); 2.0184 (3.3); 2.0031 (1.8); 1.9923 (1.1); 1.9842 (0.8); 1.9771 (0.7); 1.9686 (0.4); 1.2333 (0.4); 1.2186 (0.6); 1.2122 (0.9); 1.1995 (1.9); 1.1933 (1.8); 1.1815 (3.1); 1.1696 (1.9); 1.1633 (2.0); 1.1506 (1.0); 0.4529 (2.0); 0.4378 (7.2); 0.4286 (4.4); 0.4223 (6.0); 0.4177 (6.6); 0.4089 (3.1); 0.3908 (0.6); 0.3857 (0.6); 0.3719 (0.8); 0.3474 (3.3); 0.3356 (8.1); 0.3264 (7.7); 0.3121 (2.1); −0.0001 (2.7)

I-278: ¹H-NMR(400.0 MHz, d₆-DMSO):

δ=7.3366 (1.2); 7.3209 (1.4); 7.3147 (1.4); 7.1254 (1.2); 7.1200 (1.9); 7.1110 (3.6); 7.1031 (2.3); 7.0958 (1.6); 7.0827 (0.6); 7.0707 (1.7); 7.0640 (1.3); 7.0485 (0.9); 6.2653 (1.6); 6.2424 (1.6); 4.6853 (0.4); 4.6662 (0.7); 4.6546 (0.9); 4.6327 (0.4); 4.0240 (2.3); 4.0059 (5.0); 3.9877 (2.5); 3.9018 (6.6); 3.3246 (64.2); 3.1803 (16.0); 2.9457 (2.3); 2.9276 (4.7); 2.9093 (2.1); 2.7470 (0.7); 2.7282 (0.9); 2.7142 (1.1); 2.6987 (1.2); 2.6842 (0.6); 2.6711 (0.7); 2.6577 (0.4); 2.5060 (56.7); 2.5018 (71.2); 2.4976 (53.1); 2.3285 (0.4); 2.3242 (0.3); 1.9636 (0.4); 1.9571 (0.5); 1.9432 (0.8); 1.9281 (1.3); 1.9016 (0.7); 1.8827 (0.4); 1.8118 (0.3); 1.7838 (0.8); 1.7645 (0.8); 1.7594 (0.8); 1.7411 (0.5); 1.7320 (0.4); 1.7148 (0.6); 1.6932 (0.6); 1.6759 (0.4); −0.0001 (0.8)

I-281: ¹H-NMR(400.0 MHz, d₆-DMSO):

δ=7.3346 (0.4); 7.3147 (11.0); 7.3039 (16.0); 7.2945 (2.7); 7.2754 (0.5); 7.2654 (1.2); 7.2557 (1.5); 7.2438 (1.6); 7.2357 (0.6); 7.2319 (0.8); 7.1257 (0.5); 7.1113 (1.4); 7.1075 (1.5); 7.0946 (3.1); 7.0891 (3.5); 7.0758 (1.4); 7.0715 (1.7); 7.0569 (2.5); 7.0383(I.I); 6.2879 (2.0); 6.2652 (2.1); 4.9416 (7.5); 4.6720 (0.5); 4.6534 (0.8); 4.6411 (0.9); 4.6199 (0.5); 4.0944 (2.7); 4.0762 (5.8); 4.0581 (2.8); 3.9015 (8.9); 3.3226 (82.2); 3.2672 (0.4); 3.1741 (0.5); 3.1629 (0.5); 3.0475 (2.8); 3.0294 (5.8); 3.0112 (2.6); 2.7555 (0.3); 2.7311 (0.7); 2.7135 (1.0); 2.7002 (1.2); 2.6840 (1.3); 2.6746 (1.0); 2.6702 (1.3); 2.6420 (0.4); 2.5233 (2.0); 2.5098 (42.4); 2.5056 (82.9); 2.5012 (107.0); 2.4967 (77.9); 2.3323 (0.5); 2.3278 (0.6); 2.3235 (0.5); 1.9381 (0.6); 1.9295 (0.9); 1.9156 (1.6); 1.9035 (1.5); 1.8885 (0.8); 1.8695 (0.5); 1.8017 (0.4); 1.7968 (0.4); 1.7743 (1.0); 1.7548 (0.8); 1.7504 (0.8); 1.7316 (0.6); 1.7179 (0.5); 1.7005 (0.6); 1.6807 (0.7); 1.6611 (0.4); −0.0002 (1.8)

1H-NMR

I-282: ¹H-NMR(400.0 MHz, d₆-DMSO):

δ=7.4788 (1.5); 7.4723 (1.1); 7.4689 (1.2); 7.4610 (1.1); 7.4558 (2.0); 7.3208 (0.5); 7.3054 (2.3); 7.2956 (3.0); 7.2878 (2.2); 7.2845 (2.6); 7.2698 (0.6); 7.2565 (1.6); 7.2380 (2.1); 7.2014 (1.3); 7.1948 (1.8); 7.1831 (3.4); 7.1707 (2.0); 7.1516 (2.0); 7.1337 (1.0); 7.1145 (1.3); 7.0974 (1.6); 7.0792 (0.6); 6.3729 (1.9); 6.3514 (2.0); 4.9857 (4.3); 4.9820 (4.4); 4.9634 (1.4); 4.9425 (1.4); 4.9236 (0.5); 4.1847 (2.3); 4.1666 (5.2); 4.1486 (2.4); 3.9017 (16.0); 3.6151 (0.4); 3.3212 (46.1); 3.1742 (0.4); 3.1622 (0.4); 3.1371 (2.5); 3.1191 (5.3); 3.1010 (2.3); 2.9204 (0.4); 2.9127 (0.4); 2.8988 (0.5); 2.8909 (0.5); 2.8817 (0.7); 2.8736 (0.7); 2.8599 (0.8); 2.8519 (0.7); 2.7670 (0.5); 2.7465 (1.1); 2.7264 (0.9); 2.7075 (0.7); 2.6863 (0.4); 2.6742 (0.4); 2.6700 (0.6); 2.6657 (0.4); 2.5231 (1.7); 2.5054 (76.6); 2.5010 (99.3); 2.4965 (72.8); 2.4548 (0.5); 2.4463 (0.5); 2.4354 (0.8); 2.4268 (0.8); 2.4159 (0.8); 2.4047 (0.9); 2.3963 (0.7); 2.3855 (0.4); 2.3772 (0.4); 2.3322 (0.4); 2.3278 (0.6); 2.3234 (0.5); 1.8069 (0.4); 1.7853 (0.9); 1.7755 (0.4); 1.7649 (0.9); 1.7544 (0.9); 1.7434 (0.4); 1.7338 (0.8); −0.0002 (1.3)

I-283: ¹H-NMR(400.0 MHz, d₆-DMSO):

δ=7.1164 (4.2); 7.1008 (2.4); 7.0816 (3.3); 6.9939 (2.6); 6.9749 (1.8); 6.2468 (2.3); 6.2252 (2.4); 5.0028 (0.6); 4.9829 (1.7); 4.9628 (1.6); 4.9424 (0.6); 4.8813 (0.5); 4.8640 (1.4); 4.8466 (1.9); 4.8293 (1.4); 4.8121 (0.5); 4.0851 (0.3); 4.0302 (3.1); 4.0120 (6.8); 3.9940 (3.3); 3.9016 (5.9); 3.6003 (0.5); 3.3230 (49.2); 3.1748 (1.6); 3.1622 (1.6); 2.9181 (3.4); 2.9000 (7.0); 2.8820 (3.4); 2.8725 (0.8); 2.8642 (0.7); 2.8548 (0.9); 2.8476 (0.9); 2.8325 (0.9); 2.8260 (0.9); 2.7357 (0.6); 2.7150 (1.2); 2.6945 (1.0); 2.6752 (1.2); 2.6553 (0.5); 2.5056 (85.1); 2.5014 (107.1); 2.4971 (78.6); 2.4679 (1.2); 2.4563 (1.4); 2.4484 (1.3); 2.4371 (1.3); 2.4255 (1.3); 2.4179 (1.1); 2.4067 (0.8); 2.3986 (0.6); 2.3282 (0.7); 2.3232 (0.7); 2.3187 (0.7); 2.2531 (16.0); 1.8520 (0.5); 1.8307 (1.2); 1.8214 (0.6); 1.8096 (1.2); 1.7997 (1.2); 1.7878 (0.6); 1.7788 (1.1); 1.7570 (0.4); 1.4039 (13.1); 1.3867 (13.3); 1.3479 (0.7); 1.3326 (0.6); 1.2354 (0.4); 1.1938 (0.3); 1.1760 (0.6); 1.1587 (0.8); 1.1544 (0.6); 1.1428 (0.6); 1.1384 (0.6); −0.0002 (1.8 )

I-284: ¹H-NMR(400.0 MHz, d₆-DMSO):

δ=7.1020 (5.8); 7.0810 (3.2); 6.9930 (2.6); 6.9743 (1.8); 6.3062 (2.4); 6.2844 (2.5); 5.0053 (0.6); 4.9850 (1.7); 4.9649 (1.7); 4.9452 (0.6); 4.0544 (3.0); 4.0361 (6.6); 4.0179 (3.2); 3.9018 (6.5); 3.6376 (5.0); 3.6200 (5.1); 3.6035 (0.6); 3.3228 (97.4); 3.1748 (0.7); 3.1620 (0.6); 2.9789 (3.3); 2.9607 (6.9); 2.9425 (3.0); 2.8888 (0.5); 2.8819 (0.6); 2.8671 (0.6); 2.8596 (0.6); 2.8494 (0.9); 2.8428 (1.0); 2.8275 (0.9); 2.8214 (0.9); 2.7345 (0.6); 2.7135 (1.3); 2.6933 (1.0); 2.6746 (1.3); 2.6536 (0.5); 2.5057 (88.2); 2.5014 (110.4); 2.4970 (81.4); 2.4656 (0.8); 2.4539 (1.0); 2.4465 (1.0); 2.4351 (1.1); 2.4232 (1.0); 2.4159 (0.9); 2.4045 (0.6); 2.3968 (0.4); 2.3326 (0.6); 2.3282 (0.7); 2.3236 (0.5); 2.2515 (16.0); 1.8438 (0.4); 1.8221 (1.2); 1.8128 (0.5); 1.8009 (1.2); 1.7913 (1.2); 1.7792 (0.5); 1.7700 (1.1); 1.7485 (0.4); 1.2214 (0.4); 1.2104 (0.8); 1.2026 (0.8); 1.1910 (1.2); 1.1789 (0.9); 1.1723 (0.9); 1.1592 (0.5); 0.4559 (0.8); 0.4402 (3.2); 0.4306 (1.9); 0.4243 (2.6); 0.4203 (3.0); 0.4109 (1.3); 0.3760 (0.4); 0.3528 (1.3); 0.3405 (3.7); 0.3315 (3.3); 0.3281 (3.2); 0.3159 (0.8); −0.0001 (1.4)

I-285: ¹H-NMR(400.0 MHz, d₆-DMSO):

δ=7.6471 (0.4); 7.6413 (0.4); 7.3975 (0.5); 7.3736 (0.4); 7.3564 (0.6); 7.3317 (1.1); 7.3180 (12.2); 7.3071 (15.2); 7.2884 (0.9); 7.2778 (0.7); 7.2682 (1.5); 7.2582 (2.0); 7.2471 (1.8); 7.2372 (1.0); 7.2253 (0.5); 7.1988 (0.5); 7.1819 (0.4); 7.0883 (2.4); 7.0691 (7.6); 6.9828 (2.5); 6.9632 (1.8); 6.3115 (2.4); 6.2897 (2.5); 5.7337 (0.5); 5.7278 (0.5); 5.1455 (1.1); 5.0880 (1.1); 4.9888 (0.7); 4.9695 (1.8); 4.9473 (10.2); 4.9298 (0.9); 4.1052 (3.0); 4.0870 (6.8); 4.0690 (3.2); 3.9015 (8.1); 3.5991 (0.5); 3.5899 (0.4); 3.3243 (135.6); 3.2672 (0.6); 3.1750 (1.6); 3.1620 (1.5); 3.0527 (3.2); 3.0346 (6.8); 3.0165 (3.0); 2.8772 (0.5); 2.8699 (0.6); 2.8554 (0.6); 2.8473 (0.7); 2.8383 (0.9); 2.8311 (1.0); 2.8165 (0.9); 2.8095 (0.9); 2.7211 (0.6); 2.6999 (1.2); 2.6796 (1.2); 2.6701 (1.0); 2.6652 (1.1); 2.6406 (0.5); 2.5055 (103.3); 2.5011 (130.1); 2.4966 (96.0); 2.4567 (0.9); 2.4492 (0.9); 2.4377 (1.1); 2.4299 (1.2); 2.4186 (1.2); 2.4071 (1.2); 2.3992 (1.0); 2.3880 (0.7); 2.3800 (0.6); 2.3320 (0.9); 2.3281 (1.0); 2.2328 (16.0); 1.8249 (0.5); 1.8033 (1.2); 1.7939 (0.6); 1.7824 (1.2); 1.7726 (1.2); 1.7607 (0.6); 1.7515 (1.1); 1.7299 (0.4); −0.0002 (1.5)

1H-NMR

I-286: ¹H-NMR(400.0 MHz, d₆-DMSO):

δ=11.1441 (2.8); 7.6503 (0.5); 7.6305 (0.4); 7.2974 (1.5); 7.2800 (1.9); 7.2269 (1.2); 7.2092 (2.2); 7.1946 (0.9); 7.1915 (1.0); 7.1765 (1.9); 7.1590 (2.4); 7.1545 (2.1); 7.1400 (1.4); 7.1224 (0.5); 6.1936 (1.1); 6.1712 (1.2); 5.0348 (0.4); 5.0150 (I.I); 4.9950(I.I); 4.9749 (0.4); 4.0188 (2.4); 4.0007 (5.3); 3.9826 (2.6); 3.9262 (0.4); 3.9018 (16.0); 3.3280 (95.1); 3.2672 (0.3); 3.1684 (2.5); 3.1093 (0.4); 2.9490 (0.4); 2.9406 (0.5); 2.9275 (0.5); 2.9193 (0.6); 2.9097 (0.7); 2.9019 (0.8); 2.8874 (0.8); 2.8801 (0.8); 2.8414 (2.9); 2.8233 (6.2); 2.8051 (2.8); 2.7897 (0.7); 2.7794 (0.7); 2.7689 (1.2); 2.7486 (0.9); 2.7293 (0.7); 2.7089 (0.4); 2.6751 (0.4); 2.6707 (0.6); 2.6663 (0.5); 2.6463 (0.4); 2.6313 (0.4); 2.6169 (0.4); 2.5238 (1.6); 2.5104 (40.5); 2.5062 (81.4); 2.5017 (107.0); 2.4972 (79.6); 2.4929 (41.2); 2.4721 (1.2); 2.4635 (0.9); 2.4525 (1.0); 2.4441 (1.0); 2.4330 (1.0); 2.4212 (1.0); 2.4136 (0.9); 2.4023 (0.5); 2.3942 (0.5); 2.3330 (0.5); 2.3284 (0.7); 2.3239 (0.5); 1.8591 (0.4); 1.8375 (1.0); 1.8282 (0.4); 1.8169 (1.0); 1.8065 (1.0); 1.7953 (0.4): 1.7860 (0.9); 1.7644 (0.3); −0.0002 (0.5)

I-287: ¹H-NMR(400.0 MHz, d₆-DMSO):

δ=11.1322 (4.6); 7.3336 (1.9); 7.3186 (2.1); 7.3116 (2.2); 7.1380 (0.5); 7.1250 (2.1); 7.1206 (3.4); 7.1117 (5.8); 7.1031 (4.3); 7.0977 (2.7); 7.0851 (1.0); 7.0797 (0.8); 7.0697 (2.6); 7.0621 (2.0); 7.0475 (1.4); 6.1338 (2.5); 6.1108 (2.6); 4.6731 (0.6); 4.6626 (0.8); 4.6548 (1.0); 4.6429 (1.2); 4.6218 (0.6); 4.0077 (3.5); 3.9895 (7.7); 3.9714 (3.7); 3.9019 (16.0); 3.6003 (2.4); 3.3211 (49.7); 2.8363 (3.9); 2.8182 (8.1); 2.8000 (3.6); 2.7863 (0.4); 2.7701 (0.5); 2.7435 (1.0); 2.7260 (1.5); 2.7117 (1.6); 2.6939 (1.9); 2.6800 (1.5); 2.6703 (1.2); 2.6658 (1.1); 2.6519 (0.6); 2.5402 (0.4); 2.5231 (2.2); 2.5056 (86.8); 2.5012 (112.7); 2.4968 (83.3); 2.3322 (0.5); 2.3279 (0.6); 2.3236 (0.5); 1.9295 (1.5); 1.9161 (2.0); 1.9066 (2.4); 1.8930 (1.4); 1.8075 (0.5); 1.8038 (0.5); 1.7804 (1.2); 1.7616 (1.2); 1.7385 (0.8); 1.7220 (0.7); 1.7033 (0.8); 1.6976 (0.8); 1.6844 (1.0); 1.6648 (0.7);-0.0001 (1.4)

I-290: ¹H-NMR(400.0 MHz, d₆-DMSO):

δ=11.1576 (2.6); 7.2822 (1.4); 7.2651 (1.5); 7.1387 (0.6); 7.1349 (0.6); 7.1175 (1.4); 7.0998 (0.9); 7.0964 (0.9); 6.8589 (0.9); 6.8566 (1.0); 6.8400 (1.6); 6.8385 (1.6); 6.8217 (0.8); 6.8193 (0.8); 6.7557 (1.8); 6.7351 (1.8); 6.3576 (1.4); 6.3358 (1.5); 4.7009 (0.4); 4.6875 (0.8); 4.6763 (0.6); 4.6660 (0.8); 4.6528 (0.4); 4.2573 (0.4); 4.2468 (0.4); 4.2385 (0.9); 4.2292 (0.8); 4.2201 (0.7); 4.2104 (0.7); 4.1966 (0.6); 4.1825 (0.9); 4.1712 (0.8); 4.1541 (0.4); 4.0214 (1.9); 4.0033 (4.2); 3.9852 (2.0); 3.9020 (16.0); 3.6027 (1.2); 3.3227 (47.5); 2.8405 (2.0); 2.8224 (4.3); 2.8043 (1.9); 2.6748 (0.4); 2.6704 (0.5); 2.5405 (0.3); 2.5058 (55.7); 2.5014 (71.4); 2.4969 (52.0); 2.3282 (0.4); 2.0462 (0.5); 2.0333 (0.9); 2.0171 (1.4); 2.0071 (1.2); 1.9937 (0.5); 1.9826 (0.3); −0.0002 (0.9)

I-291: ¹H-NMR(400.0 MHz, d₆-DMSO):

δ=7.3072 (1.5); 7.2898 (1.8); 7.2171 (1.1); 7.2000 (2.2); 7.1817 (1.0); 7.1668 (1.9); 7.1493 (2.3); 7.1456 (2.1); 7.1301 (1.4); 7.1118 (0.5); 6.2892 (1.7); 6.2675 (1.7); 5.0487 (0.5); 5.0287 (1.3); 5.0086 (1.3); 4.9881 (0.4); 3.9549 (1.9); 3.9395 (4.1); 3.9241 (2.1); 3.9019 (16.0); 3.8753 (1.0); 3.8617 (2.0); 3.8421 (2.0); 3.8281 (1.0); 3.8083 (0.5); 3.5880 (0.4); 3.5765 (0.8); 3.5695 (0.8); 3.5586 (1.2); 3.5456 (0.8); 3.5384 (0.7); 3.5260 (0.4); 3.3215 (50.9); 3.1703 (0.4); 2.9520 (0.4); 2.9447 (0.5); 2.9304 (0.5); 2.9221 (0.5); 2.9125 (0.7); 2.9049 (0.8); 2.8907 (0.8); 2.8835 (0.7); 2.7937 (0.5); 2.7729 (1.1); 2.7524 (0.9); 2.7337 (0.7); 2.7129 (0.4); 2.6706 (0.6); 2.5396 (0.5); 2.5057 (87.1); 2.5015 (110.1); 2.4975 (82.1); 2.4688 (0.8); 2.4571 (0.8); 2.4489 (0.9); 2.4376 (0.9); 2.4262 (0.9); 2.4183 (0.8); 2.4068 (0.4); 2.3990 (0.4); 2.3284 (0.7); 2.3240 (0.6); 2.1049 (0.5); 2.0911 (1.8); 2.0763 (2.6); 2.0614 (1.8); 2.0470 (0.6); 1.8893 (0.4); 1.8677 (0.9); 1.8587 (0.4); 1.8470 (1.0); 1.8369 (0.9); 1.8257 (0.4); 1.8159 (0.9); 0.9064 (0.5); 0.8943 (1.8); 0.8866 (3.1); 0.8830 (3.0); 0.8758 (2.8); 0.8701 (1.9); 0.8530 (2.9); 0.8458 (1.6); 0.8392 (1.9); 0.8332 (2.7); 0.8265 (1.5); 0.8143 (0.8); −0.0002 (0.9)

1H-NMR

I-292: ¹H-NMR(400.0 MHz, d₆-DMSO):

δ=7.8290 (1.6); 7.8030 (1.4); 7.7816 (0.9); 7.6254 (0.6); 7.6060 (1.0); 7.5870 (0.4); 7.0841 (1.2); 7.0667 (0.7); 7.0372 (0.8); 7.0183 (1.2); 6.9807 (0.6); 6.9607 (0.4); 6.9461 (1.0); 6.9260 (0.6); 6.8993 (1.5); 5.9259 (0.9); 5.9042 (0.9); 4.9171 (0.3); 4.6224 (0.6); 4.6029 (0.6); 3.9974 (0.4); 3.9872 (0.4); 3.9737 (0.9); 3.9595 (1.3); 3.9459 (0.7); 3.9067 (2.9); 3.9020 (16.0); 3.8859 (0.5); 3.7744 (0.6); 3.7598 (1.1); 3.7451 (0.6); 3.3220 (34.8); 3.1871 (0.6); 3.1738 (0.6); 2.7297 (0.3); 2.7143 (0.3); 2.7074 (0.4); 2.6793 (0.4); 2.6746 (0.5); 2.6705 (0.6); 2.6654 (0.6); 2.5589 (0.5); 2.5400 (0.7); 2.5057 (74.2); 2.5013 (97.0); 2.4970 (72.8); 2.4084 (0.3); 2.3852 (0.3); 2.3764 (0.4); 2.3670 (0.5); 2.3278 (1.0); 2.3231 (0.9); 2.3116 (0.7); 2.2955 (0.8); 2.2861 (0.5); 2.2531 (3.6); 2.2220 (5.9); 2.1494 (0.3); 2.1421 (0.4); 2.1303 (0.4); 2.1184 (0.4); 2.1114 (0.4); 1.9743 (0.4); 1.9593 (0.6); 1.9461 (0.4); 1.6110 (0.4); 1.5898 (0.4); 1.5798 (0.4); 1.5592 (0.4); −0.0002 (0.8)

I-293: ¹H-NMR(400.0 MHz, d₆-DMSO):

δ=7.8338 (2.8); 7.8231 (1.7); 7.8038 (3.0); 7.7854 (1.6); 7.6245 (1.3); 7.6052 (2.0); 7.5858 (0.9); 7.1624 (0.7); 7.1432 (3.3); 7.1352 (1.4); 7.1294 (1.1); 7.1223 (1.5); 7.1118 (0.5); 7.1037 (0.7); 7.0913 (0.4); 7.0717 (2.0); 7.0668 (3.2); 7.0590 (3.4); 7.0564 (3.2); 5.9819 (1.8); 5.9603 (1.8); 4.6768 (0.5); 4.6571 (1.4); 4.6371 (1.3); 4.6171 (0.5); 3.9912 (0.6); 3.9773 (2.2); 3.9623 (4.4); 3.9464 (2.4); 3.9255 (1.2); 3.9019 (16.0); 3.8919 (0.7); 3.8769 (0.3); 3.3231 (69.1); 2.8412 (0.4); 2.8333 (0.5); 2.8195 (0.5); 2.8114 (0.5); 2.8022 (0.7); 2.7938 (0.7); 2.7805 (0.7); 2.7723 (0.7); 2.6747 (0.5); 2.6704 (0.7); 2.6659 (0.5); 2.6452 (0.5); 2.6240 (1.1); 2.6042 (0.9); 2.5848 (0.8); 2.5644 (0.4); 2.5403 (0.3); 2.5236 (2.1); 2.5101 (49.3); 2.5059 (97.9); 2.5015 (127.4); 2.4970 (93.1); 2.3329 (1.0); 2.3279 (1.3); 2.3227 (1.7); 2.3059 (1.9); 2.2912 (1.3); 2.1887 (0.4); 2.1807 (0.4); 2.1695 (0.6); 2.1612 (0.7); 2.1502 (0.8); 2.1387 (0.8); 2.1306 (0.7); 2.1198 (0.4); 2.1114 (0.4); 1.6661 (0.4); 1.6449 (0.9); 1.6350 (0.4); 1.6243 (0.9); 1.6138 (0.9); 1.6027 (0.4); 1.5933 (0.8); −0.0002 (1.5)

I-295: ¹H-NMR(400.0 MHz, d₆-DMSO):

δ=7.4836 (0.4); 7.4778 (0.4); 7.1005 (2.7); 7.0913 (1.7); 7.0721 (2.2); 6.9851 (1.6); 6.9664 (1.2); 6.2413 (1.5); 6.2196 (1.5); 5.0094 (0.4); 4.9896 (1.1); 4.9693 (1.0); 4.9496 (0.4); 3.9537 (1.7); 3.9383 (3.7); 3.9228 (1.9); 3.9018 (16.0); 3.8749 (1.0); 3.8610 (2.2); 3.8449 (2.2); 3.8308 (1.0); 3.8114 (0.5); 3.5898 (0.6); 3.5821 (0.7); 3.5707 (1.2); 3.5588 (0.8); 3.5510 (0.7); 3.5388 (0.4); 3.3216 (49.2); 3.1683 (1.4); 2.8798 (0.4); 2.8652 (0.4); 2.8575 (0.4); 2.8482 (0.5); 2.8407 (0.6); 2.8262 (0.6); 2.8198 (0.5); 2.7299 (0.4); 2.7092 (0.8); 2.6894 (0.6); 2.6703 (1.1); 2.5405 (0.3); 2.5233 (1.9); 2.5057 (86.4); 2.5013 (112.7); 2.4968 (82.9); 2.4655 (0.8); 2.4578 (0.7); 2.4460 (0.8); 2.4386 (0.8); 2.4272 (0.9); 2.4156 (0.8); 2.4078 (0.8); 2.3962 (0.8); 2.3880 (0.5); 2.3670 (1.4); 2.3324 (0.6); 2.3280 (0.8); 2.3236 (0.6); 2.2699 (0.4); 2.2491 (10.6); 2.1058 (0.5); 2.0911 (1.6); 2.0766 (2.3); 2.0620 (1.7); 2.0474 (0.6); 1.8385 (0.8); 1.8292 (0.4); 1.8177 (0.8); 1.8078 (0.8); 1.7961 (0.4); 1.7867 (0.7); 0.9109 (0.6); 0.8987 (1.8); 0.8914 (2.8); 0.8873 (2.8); 0.8801 (2.5); 0.8740 (1.9); 0.8569 (2.6); 0.8497 (1.6); 0.8436 (1.9); 0.8371 (2.6); 0.8298 (1.4); 0.8184 (0.7); −0.0002 (1.2)

I-297: ¹H-NMR(400.0 MHz, d₆-DMSO):

δ=7.8238 (2.5); 7.8203 (2.4); 7.7978 (2.3); 7.6326 (1.0); 7.6136 (1.5); 7.5941 (0.7); 7.0889 (0.6); 7.0853 (0.7); 7.0673 (1.3); 7.0470 (2.0); 7.0292 (1.5); 6.7862 (0.9); 6.7836 (1.0); 6.7654 (1.6); 6.7491 (0.7); 6.7465 (0.8); 6.6907 (1.7); 6.6702 (1.6); 6.1290 (1.5); 6.1075 (1.6); 4.2945 (0.4); 4.2812 (0.8); 4.2681 (0.6); 4.2602 (0.8); 4.2458 (0.4); 4.1405 (0.4); 4.1295 (0.5); 4.1241 (0.4); 4.1129 (0.9); 4.0997 (0.9); 4.0850 (0.5); 4.0540 (0.3); 4.0381 (0.5); 4.0121 (0.8); 4.0048 (1.7); 3.9918 (1.0); 3.9744 (1.5); 3.9595 (2.0); 3.9447 (0.9); 3.9394 (0.9); 3.9020 (16.0); 3.8914 (0.6); 3.8817 (0.6); 3.8718 (0.5); 3.8610 (0.7); 3.8487 (0.4); 3.8408 (0.4); 3.8301 (0.3); 3.7579 (0.4); 3.3207 (40.3); 3.1745 (0.4); 2.6750 (0.4); 2.6704 (0.6); 2.6657 (0.4); 2.5236 (1.8); 2.5101 (38.4); 2.5058 (77.3); 2.5013 (101.1); 2.4968 (73.5); 2.4926 (36.4); 2.3279 (1.1); 2.3236 (1.1); 2.3103 (1.0); 2.2960 (1.0); 2.2846 (0.7); 1.7300 (0.7); 1.7157 (1.7); 1.7027 (1.7); 1.6901 (0.7); −0.0002 (1.7)

1H-NMR

I-298: ¹H-NMR(400.0 MHz, d₆-DMSO):

δ=7.2930 (2.1); 7.2738 (2.2); 7.1260 (1.0); 7.1224 (1.1); 7.1048 (2.1); 7.0875 (1.3); 7.0839 (1.3); 6.8464 (1.5); 6.8440 (1.6); 6.8256 (2.8); 6.8094 (1.3); 6.8068 (1.3); 6.7427 (2.9); 6.7409 (2.9); 6.7224 (2.7); 6.4224 (2.5); 6.4008 (2.6); 4.7278 (0.6); 4.7140 (1.3); 4.7007 (0.9); 4.6926 (1.3); 4.6790 (0.6); 4.2766 (0.5); 4.2667 (0.7); 4.2583 (0.6); 4.2487 (1.4); 4.2379 (1.3); 4.2318 (1.2); 4.2206 (1.0); 4.2004 (0.9); 4.1874 (1.5); 4.1749 (1.3); 4.1630 (0.6); 4.1577 (0.7); 4.1479 (0.5); 3.9571 (2.4); 3.9417 (5.2); 3.9262 (2.4); 3.9019 (16.0); 3.8762 (1.2); 3.8624 (2.2); 3.8495 (1.6); 3.8373 (2.2); 3.8231 (1.2); 3.8041 (0.7); 3.7904 (0.4); 3.5800 (0.4); 3.5678 (0.9); 3.5605 (1.0); 3.5493 (1.6); 3.5365 (1.0); 3.5292 (1.0); 3.5171 (0.4); 3.3216 (68.8); 3.1746 (1.2); 3.1621 (1.1); 2.6745 (0.5); 2.6703 (0.7); 2.6659 (0.5); 2.5402 (0.3); 2.5235 (2.1); 2.5099 (46.5); 2.5058 (91.4); 2.5013 (118.4); 2.4969 (87.1); 2.3327 (0.5); 2.3280 (0.7); 2.3237 (0.5); 2.1061 (0.7); 2.0916 (2.3); 2.0770 (3.4); 2.0620 (2.8); 2.0433 (2.8); 2.0331 (2.2); 2.0188 (0.9); 2.0086 (0.4); 1.2117 (0.4); 1.1956 (0.3); 0.9102 (0.5); 0.8976 (2.0); 0.8903 (4.0); 0.8860 (3.6); 0.8787 (3.6); 0.8671 (2.2); 0.8620 (3.9); 0.8543 (1.9); 0.8482 (2.0); 0.8422 (3.7); 0.8346 (1.8); 0.8224 (0.6): -0.0002 (1.7)

I-301: ¹H-NMR(400.0 MHz, d₆-DMSO):

δ=7.4830 (0.6); 7.4775 (0.7); 7.4381 (0.5); 7.1112 (1.9); 7.0960 (1.1); 7.0770 (1.6); 6.9882 (1.2); 6.9697 (0.8); 6.2939 (0.5); 6.2726 (0.5); 4.9758 (0.6); 4.9562 (0.6); 3.9104 (1.3); 3.9019 (16.0); 3.8957 (3.0); 3.8803 (1.4); 3.8371 (0.4); 3.8218 (0.7); 3.8103 (1.4); 3.7968 (1.6); 3.7831 (1.4); 3.7607 (0.4); 3.7469 (0.3); 3.4203 (0.6); 3.4093 (11.9); 3.3908 (0.8); 3.3785 (2.8); 3.3307 (14.0); 3.1682 (3.9); 3.0616 (0.4); 3.0482 (0.4); 3.0336 (0.4); 2.8518 (0.4); 2.8446 (0.4); 2.8300 (0.4); 2.8235 (0.4); 2.7086 (0.6); 2.6882 (0.4); 2.6704 (0.8); 2.6316 (0.6); 2.6170 (0.5); 2.6026 (0.5); 2.5236 (1.2); 2.5101 (33.2); 2.5059 (66.8); 2.5014 (87.1); 2.4969 (63.8); 2.4655 (0.6); 2.4573 (0.5); 2.4460 (0.6); 2.4379 (0.6); 2.4266 (0.6); 2.4153 (0.6); 2.4073 (0.6); 2.3966 (0.4); 2.3884 (0.4); 2.3672 (2.4); 2.3326 (0.5); 2.3281 (0.6); 2.3236 (0.5); 2.2503 (7.6); 2.1857 (0.4); 2.1716 (1.0); 2.1574 (1.3); 2.1438 (1.1); 2.1308 (0.6); 2.1175 (0.4); 1.8178 (0.6); 1.7969 (0.6); 1.7868 (0.6); 1.7660 (0.5); −0.0002 (1.0)

I-302: ¹H-NMR(400.0 MHz, d₆-DMSO):

δ=7.3123 (1.9); 7.2943 (2.4); 7.2227 (1.3); 7.2050 (2.8); 7.1855 (1.2); 7.1702 (2.4); 7.1488 (2.6); 7.1299 (1.9); 7.1120 (0.7); 6.3415 (2.4); 6.3198 (2.5); 5.0350 (0.6); 5.0149 (1.7); 4.9948 (1.7); 4.9748 (0.6); 3.9108 (2.5); 3.9014 (16.0); 3.8964 (5.7); 3.8806 (2.4); 3.8086 (2.4); 3.7951 (3.0); 3.7815 (2.5); 3.4018 (21.8); 3.3224 (82.1); 3.1737 (0.6); 3.1610 (0.6); 2.9542 (0.5); 2.9466 (0.6); 2.9326 (0.6); 2.9245 (0.6); 2.9153 (0.9); 2.9071 (0.9); 2.8935 (0.9); 2.8857 (0.9); 2.7924 (0.7); 2.7721 (1.4); 2.7519 (1.1); 2.7324 (0.9); 2.7114 (0.5); 2.6697 (0.6); 2.6652 (0.5); 2.5394 (0.4); 2.5048 (81.2); 2.5007 (103.0); 2.4965 (76.6); 2.4697 (0.8); 2.4587 (1.0); 2.4502 (1.0); 2.4391 (1.0); 2.4278 (1.0); 2.4197 (0.9); 2.4086 (0.5); 2.4006 (0.4); 2.3315 (0.5); 2.3274 (0.6); 2.1852 (0.6); 2.1707 (1.9); 2.1570 (2.4); 2.1432 (1.8); 2.1282 (0.6); 1.8632 (0.4); 1.8417 (1.2); 1.8320 (0.5); 1.8207 (1.2); 1.8108 (1.2); 1.7994 (0.5): I 7899 (1.1); 1.7684 (0.4); −0.0003 (0.9)

I-304: ¹H-NMR(400.0 MHz, d₆-DMSO):

δ=7.2897 (1.7); 7.2708 (1.8); 7.1307 (0.8); 7.1271 (0.8); 7.1095 (1.7); 7.0923 (1.1); 7.0886 (1.0); 6.8468 (1.2); 6.8446 (1.2); 6.8262 (2.1); 6.8098 (1.0); 6.8074 (1.0); 6.7472 (2.4); 6.7269 (2.2); 6.4740 (1.8); 6.4523 (1.9); 4.7143 (0.5); 4.7003 (1.0); 4.6885 (0.8); 4.6791 (1.0); 4.6656 (0.5); 4.2763 (0.4); 4.2678 (0.5); 4.2563 (0.5); 4.2485 (1.2); 4.2400 (1.0); 4.2291 (0.9); 4.2202 (0.8); 4.2017 (0.7); 4.1925 (0.9); 4.1866 (1.0); 4.1764 (1.0); 4.1653 (0.4); 4.1586 (0.5); 4.1498 (0.4); 3.9148 (1.9); 3.9017 (16.0); 3.8846 (2.0); 3.8087 (1.9); 3.7952 (2.5); 3.7814 (2.0); 3.4068 (18.7); 3.3220 (54.6); 3.1684 (2.5); 2.6745 (0.4); 2.6700 (0.6); 2.5398 (0.4); 2.5231 (2.0); 2.5055 (83.7); 2.5011 (107.9); 2.4967 (78.8); 2.3324 (0.5); 2.3278 (0.6); 2.1858 (0.5); 2.1712 (1.5); 2.1573 (1.9); 2.1434 (1.4); 2.1283 (0.5); 2.0606 (0.6); 2.0510 (0.8); 2.0389 (1.1); 2.0278 (1.3); 2.0206 (1.3); 2.0125 (1.1); 1.9975 (0.6); 1.9865 (0.4); −0.0002 (1.1)

1H-NMR

I-305: ¹H-NMR(400.0 MHz, d₆-DMSO):

δ=7.8705 (0.5); 7.8506 (0.5); 7.5420 (0.5); 7.5264 (0.3); 7.5230 (0.3); 7.3628 (1.0); 7.3439 (1.1); 7.3358 (0.9); 7.3200 (1.0); 7.3137 (1.0); 7.1185 (0.9); 7.1128 (1.2); 7.1038 (2.6); 7.0961 (1.5); 7.0885 (1.1); 7.0757 (0.5); 7.0641 (1.2); 7.0573 (0.9); 7.0421 (0.6); 6.2637 (1.0); 6.2408 (1.0); 4.6580 (0.5); 4.6467 (0.6); 3.9020 (16.0); 3.8853 (2.8); 3.8702 (1.3); 3.8368 (0.4); 3.8215 (0.8); 3.8054 (1.6); 3.7916 (1.8); 3.7777 (1.5); 3.7602 (0.5); 3.7459 (0.4); 3.4205 (0.5); 3.4045 (12.5); 3.3782 (3.2); 3.3216 (28.7); 3.1683 (3.1); 2.9612 (0.5); 2.9461 (0.9); 2.9308 (0.6); 2.7431 (0.4); 2.7249 (0.6); 2.7108 (0.7); 2.6929 (0.8); 2.6790 (0.6); 2.6752 (0.6); 2.6702 (0.7); 2.6658 (0.6); 2.6156 (0.7); 2.6001 (0.9); 2.5831 (0.8); 2.5404 (0.3); 2.5234 (1.6); 2.5099 (38.0); 2.5056 (76.7); 2.5012 (100.7); 2.4967 (74.1); 2.4925 (37.4); 2.3323 (0.5); 2.3278 (0.6); 2.3236 (0.5); 2.1804 (0.4); 2.1657 (1.1); 2.1518 (1.4); 2.1381 (1.1); 2.1225 (0.6); 2.0573 (0.6); 2.0421 (0.8); 2.0263 (0.5); 1.9435 (0.6); 1.9291 (1.0); 1.9181 (1.0); 1.9042 (0.5); 1.7864 (0.6); 1.7671 (0.5); 1.7437 (0.4); 1.7294 (0.3); 1.7108 (0.4); 1.7061 (0.4); 1.6956 (0.4); −0.0002 (0.9)

I-306: ¹H-NMR(400.0 MHz, d₆-DMSO):

δ=7.3072 (1.5); 7.2881 (1.6); 7.1439 (0.7); 7.1401 (0.7); 7.1226 (1.5); 7.1055 (0.9); 7.1017 (0.9); 6.8611 (1.0); 6.8586 (1.1); 6.8422 (1.8); 6.8403 (1.9); 6.8240 (0.9); 6.8213 (0.9); 6.7639 (2.1); 6.7435 (1.9); 6.4038 (1.7); 6.3822 (1.8); 4.7566 (0.4); 4.7432 (0.9); 4.7328 (0.7); 4.7217 (0.9); 4.7087 (0.4); 4.2935 (0.3); 4.2818 (0.5); 4.2761 (0.4); 4.2653 (1.0); 4.2523 (1.1); 4.2371 (0.6); 4.2013 (0.6); 4.1895 (1.2); 4.1762 (0.8); 4.1607 (0.6); 4.1500 (0.3); 3.9221 (1.7); 3.9020 (16.0); 3.8916 (1.9); 3.8793 (0.6); 3.8721 (0.5); 3.8517 (1.0); 3.8441 (0.8); 3.8255 (1.6); 3.8174 (2.0); 3.8113 (1.7); 3.8027 (1.3); 3.3220 (57.9); 2.6750 (0.4); 2.6705 (0.5); 2.6659 (0.4); 2.5237 (1.5); 2.5100 (37.1); 2.5059 (73.6); 2.5015 (95.7); 2.4970 (70.0); 2.3328 (0.4); 2.3283 (0.6); 2.3238 (0.4); 2.0787 (0.6); 2.0538 (3.2); 2.0415 (3.1); 1.8331 (1.2); 1.8059 (1.6); 1.7021 (1.3); 1.6742 (1.3); 1.6252 (0.8); 1.5984 (0.6); 1.3399 (0.6); 1.3121 (1.0); 1.2862 (1.5); 1.2606 (1.4); 1.2308 (1.1); 1.2247 (1.0); 1.1958 (1.0); 1.1710 (0.6); 1.1411 (0.4); −0.0002 (1.3)

I-307: ¹H-NMR(400.0 MHz, d₆-DMSO):

δ=7.3225 (2.2); 7.3043 (2.7); 7.2340 (1.5); 7.2161 (3.2); 7.1960 (1.4); 7.1801 (2.7); 7.1584 (2.6); 7.1388 (2.2); 7.1210 (0.8); 6.2287 (2.4); 6.2068 (2.5); 5.0887 (0.6); 5.0687 (1.8); 5.0479 (1.8); 5.0285 (0.6); 3.9245 (2.7); 3.9090 (5.9); 3.9019 (16.0); 3.8938 (3.5); 3.8716 (1.1); 3.8649 (1.6); 3.8575 (1.4); 3.8378 (2.1); 3.8255 (3.6); 3.8108 (3.4); 3.7982 (1.4); 3.7783 (0.5); 3.3213 (41.1); 2.9759 (0.6); 2.9674 (0.7); 2.9543 (0.7); 2.9458 (0.7); 2.9364 (1.0); 2.9278 (1.0); 2.9151 (1.0); 2.9065 (1.0); 2.7968 (0.7); 2.7765 (1.5); 2.7560 (1.3); 2.7367 (1.0); 2.7165 (0.6); 2.6706 (0.7); 2.5057 (101.3); 2.5016 (125.7); 2.4979 (91.8); 2.4647 (1.2); 2.4551 (1.3); 2.4444 (1.1); 2.4338 (1.2); 2.4250 (1.0); 2.4141 (0.6); 2.4054 (0.5); 2.3322 (0.6); 2.3281 (0.8); 2.0811 (0.7); 2.0667 (2.3); 2.0522 (3.3); 2.0377 (2.2); 2.0234 (0.7); 1.9083 (0.5); 1.8869 (1.3); 1.8771 (0.6); 1.8667 (1.4); 1.8559 (1.4); 1.8353 (2.9); 1.8134 (1.7); 1.8040 (2.2); 1.6699 (1.8); 1.6078 (1.2); 1.5844 (1.0); 1.3540 (0.6); 1.3261 (1.7); 1.2970 (2.0); 1.2664 (1.8); 1.2356 (1.9); 1.2036 (2.1); 1.1809 (2.0); 1.1631 (1.0); 1.1321 (0.5); −0.0002 (1.9)

I-308: ¹H-NMR(400.0 MHz, d₆-DMSO):

δ=7.1168 (3.1); 7.1069 (1.9); 7.0876 (2.5); 6.9995 (1.9); 6.9804 (1.3); 6.1856 (1.9); 6.1639 (2.0); 5.0502 (0.5); 5.0305 (1.3); 5.0104 (1.3); 4.9908 (0.4); 3.9230 (1.9); 3.9076 (4.7); 3.9020 (16.0); 3.8925 (2.4); 3.8809 (0.8); 3.8731 (1.3); 3.8650 (0.8); 3.8605 (0.7); 3.8538 (0.5); 3.8409 (1.2); 3.8270 (2.2); 3.8099 (2.2); 3.7958 (1.0); 3.7765 (0.4); 3.3218 (70.2); 2.9106 (0.4); 2.9033 (0.4); 2.8899 (0.4); 2.8817 (0.5); 2.8722 (0.6); 2.8643 (0.6); 2.8508 (0.6); 2.8433 (0.6); 2.7327 (0.4); 2.7122 (0.9); 2.6921 (0.7); 2.6742 (1.0); 2.6708 (I.I); 2.6528 (0.4); 2.5237 (1.6); 2.5102 (38.6); 2.5059 (78.2); 2.5014 (102.8); 2.4969 (75.2); 2.4926 (37.5); 2.4724 (0.9); 2.4637 (0.7); 2.4528 (0.9); 2.4441 (0.8); 2.4330 (0.9); 2.4218 (0.9); 2.4135 (0.8); 2.4028 (0.5); 2.3944 (0.4); 2.3326 (0.5); 2.3282 (0.6); 2.3236 (0.5); 2.2543 (12.5); 2.0815 (0.5); 2.0671 (1.7); 2.0525 (2.5); 2.0377 (1.7); 2.0228 (0.5); 1.8790 (0.4); 1.8575 (1.2); 1.8471 (1.1); 1.8373 (2.0); 1.8267 (1.9); 1.8157 (1.5); 1.8065 (2.1); 1.7855 (0.5); 1.6700 (1.3); 1.6069 (0.8); 1.5838 (0.7); 1.3606 (0.4); 1.3527 (0.4); 1.3301 (1.2); 1.3010 (1.3); 1.2689 (1.2); 1.2360 (1.3); 1.2051 (1.4); 1.1830 (1.4); 1.1655 (0.7); 1.1357 (0.4); −0.0002 (1.5)

1H-NMR

I-310: ¹H-NMR(400.0 MHz, d₆-DMSO):

δ=7.7103 (2.0); 7.6369 (1.2); 7.6184 (2.4); 7.5798 (1.3); 7.5606 (1.1); 7.5419 (0.5); 7.1044 (2.0); 6.9308 (4.7); 6.2396 (1.2); 6.2169 (1.3); 5.2761 (0.6); 5.0655 (0.4); 5.0272 (2.2); 5.0154 (2.1); 4.9765 (0.4); 4.6249 (0.5); 4.6131 (0.6); 4.1000 (1.6); 4.0819 (3.3); 4.0639 (1.5); 3.9018 (16.0); 3.3245 (89.5); 3.0635 (1.6); 3.0455 (3.4); 3.0274 (1.5); 2.6755 (0.7); 2.6708 (0.8); 2.6661 (0.8); 2.6617 (0.8); 2.6460 (0.7); 2.6308 (0.7); 2.5239 (1.5); 2.5062 (71.3); 2.5018 (92.2); 2.4974 (67.8); 2.3329 (0.4); 2.3283 (0.6); 2.3190 (1.1); 2.1909 (8.3); 1.9223 (0.3); 1.9043 (0.5); 1.8893 (0.9); 1.8613 (0.5); 1.7453 (0.6); 1.7205 (0.5); 1.7028 (0.4); 1.6683 (0.4); 1.6474 (0.4); −0.0002 (0.9)

I-311: ¹H-NMR(400.0 MHz, d₆-DMSO):

δ=7.4809 (1.3); 7.4744 (1.0); 7.4712 (1.0); 7.4631 (1.0); 7.4580 (1.7); 7.3216 (0.4); 7.3063 (2.1); 7.2965 (2.6); 7.2856 (2.3); 7.2706 (0.5); 7.2005 (1.4); 7.1869 (1.0); 7.1771 (0.9); 7.0839 (2.8); 6.9161 (6.9); 6.2291 (1.8); 6.2064 (1.8); 4.9808 (5.7); 4.5970 (0.4); 4.5800 (0.6); 4.5660 (0.7); 4.5462 (0.4); 4.1759 (2.0); 4.1577 (4.4); 4.1396 (2.1); 3.9017 (16.0); 3.3259 (142.5); 3.1750 (0.4); 3.1620 (0.3); 3.1322 (2.1); 3.1142 (4.4); 3.0961 (1.9); 2.6791 (0.4); 2.6748 (0.6); 2.6702 (0.8); 2.6658 (0.7); 2.6602 (0.6); 2.6397 (1.0); 2.6297 (0.9); 2.6145 (1.0); 2.6012 (0.6); 2.5234 (1.8); 2.5058 (78.5); 2.5014 (100.9); 2.4969 (73.3); 2.3324 (0.5); 2.3281 (0.6); 2.3236 (0.5); 2.1816 (11.5); 1.9057 (0.5); 1.8947 (0.6); 1.8805 (0.9); 1.8691 (1.0); 1.8542 (0.6); 1.8392 (0.6); 1.8213 (0.4); 1.7157 (0.6); 1.6940 (0.7); 1.6872 (0.6); 1.6699 (0.8); 1.6567 (0.6); 1.6466 (0.5); 1.6302 (0.5); −0.0002 (1.5)

I-312: ¹H-NMR(400.0 MHz, d₆-DMSO):

δ=7.4812 (1.8); 7.4751 (1.4); 7.4721 (1.4); 7.4688 (1.2); 7.4633 (1.4); 7.4583 (2.5); 7.3284 (0.4); 7.3222 (0.6); 7.3092 (2.3); 7.3057 (3.0); 7.2964 (3.7); 7.2864 (3.2); 7.2835 (3.0); 7.2701 (0.7); 7.2648 (0.4); 7.1990 (1.9); 7.1939 (1.3); 7.1852 (1.4); 7.1757 (1.3); 7.0747 (2.3); 7.0555 (3.4); 7.0461 (4.0); 6.9703 (2.3); 6.9512 (1.7); 6.3220 (2.3); 6.3005 (2.4); 5.0296 (0.4); 4.9870 (4.9); 4.9821 (4.9); 4.9514 (0.6); 4.9308 (1.6); 4.9108 (1.6); 4.8908 (0.6); 4.1836 (2.8); 4.1655 (6.3); 4.1474 (3.0); 3.9019 (16.0); 3.6110 (0.8); 3.3237 (104.1); 3.1657 (0.4); 3.1364 (3.0); 3.1184 (6.2); 3.1002 (2.7); 2.8546 (0.5); 2.8479 (0.5); 2.8331 (0.6); 2.8261 (0.6); 2.8158 (0.8); 2.8090 (0.9); 2.7941 (0.9); 2.7872 (0.8); 2.7052 (0.6); 2.6837 (1.2); 2.6747 (0.8); 2.6653 (1.4); 2.6451 (0.8); 2.6242 (0.4); 2.5056 (98.2); 2.5012 (127.3); 2.4967 (94.7); 2.4432 (0.6); 2.4349 (0.6); 2.4238 (0.9); 2.4158 (1.0); 2.4047 (1.0); 2.3931 (1.0); 2.3855 (0.9); 2.3744 (0.6); 2.3667 (0.6); 2.3323 (0.6); 2.3279 (0.8); 2.3232 (0.6); 2.2212 (15.6); 1.7824 (0.4); 1.7608 (1.2); 1.7514 (0.5); 1.7402 (1.1); 1.7300 (1.1); 1.7181 (0.5); 1.7091 (1.1); 1.6874 (0.4); −0.0002 (1.4)

I-313: ¹H-NMR(400.0 MHz, d₆-DMSO):

δ=7.1367 (3.4); 6.9547 (0.4); 6.9347 (8.1); 6.9113 (0.4); 6.2318 (2.0); 6.2091 (2.1); 4.6127 (0.8); 4.6004 (0.9); 4.5784 (0.5); 4.0427 (2.5); 4.0245 (5.5); 4.0064 (2.6); 3.9017 (16.0); 3.6075 (3.5); 3.5890 (3.6); 3.3231 (96.2); 2.9648 (2.7); 2.9467 (5.6); 2.9285 (2.5); 2.6853 (0.7); 2.6701 (1.6); 2.6527 (1.0); 2.6354 (1.2); 2.5943 (0.4); 2.5058 (89.7); 2.5014 (114.4); 2.4969 (83.8); 2.3324 (0.6); 2.3282 (0.7); 2.3239 (0.6); 2.3189 (0.4); 2.2097 (13.9); 1.9061 (1.5); 1.8798 (0.7); 1.8605 (0.5); 1.8165 (0.5); 1.8071 (0.6); 1.7877 (1.0); 1.7815 (1.0); 1.7593 (1.4); 1.7399 (1.0); 1.7348 (1.0); 1.7158 (0.6); 1.6593 (2.3); 1.6159 (2.1); 1.5879 (2.6); 1.1337 (3.0); 1.1160 (1.8); 0.9618 (0.6); 0.9344 (1.3); 0.9068 (1.1); −0.0003 (1.6 )

I-314: ¹H-NMR(400.0 MHz, d₆-DMSO):

δ=7.7033 (2.8); 7.6385 (1.6); 7.6186 (3.1); 7.5793 (1.9); 7.5600 (1.7); 7.5410 (0.7); 7.0894 (1.8); 7.0702 (2.7); 7.0596 (3.4); 6.9829 (1.9); 6.9641 (1.4); 6.3279 (2.0); 6.3060 (2.0); 5.0244 (6.4); 4.9991 (0.6); 4.9788 (1.3); 4.9586 (1.3); 4.9378 (0.5); 4.1103 (2.3); 4.0921 (5.2); 4.0740 (2.5); 4.0629 (0.4); 3.9020 (16.0); 3.5955 (0.5); 3.3239 (106.3); 3.2673 (0.3); 3.1748 (0.3); 3.1632 (0.3); 3.0692 (2.4); 3.0511 (5.2); 3.0328 (2.3); 2.8759 (0.4); 2.8693 (0.4); 2.8543 (0.5); 2.8476 (0.5); 2.8380 (0.7); 2.8308 (0.7); 2.8159 (0.7); 2.8093 (0.7); 2.7204 (0.5); 2.6983 (1.0); 2.6755 (1.1); 2.6706 (0.9); 2.6656 (0.8); 2.6610 (0.8); 2.6386 (0.4); 2.5236 (1.9); 2.5059 (91.0); 2.5015 (117.8); 2.4970 (85.9); 2.4538 (0.7); 2.4458 (0.7); 2.4347 (0.8); 2.4268 (0.9); 2.4154 (0.9); 2.4036 (0.9); 2.3959 (0.8); 2.3848 (0.6); 2.3769 (0.5); 2.3328 (0.7); 2.3283 (0.8); 2.3237 (0.6); 2.2290 (12.6); 1.8224 (0.4); 1.8011 (1.0); 1.7919 (0.4); 1.7801 (1.0); 1.7702 (0.9); 1.7584 (0.4): 1.7493 (0.9); 1.7275 (0.3); 1.1562 (0.6); −0.0002 (1.8)

1H-NMR

I-315: ¹H-NMR(400.0 MHz, d₆-DMSO):

δ=7.3367 (0.3); 7.3165 (9.7); 7.3056 (15.3); 7.2840 (0.4); 7.2767 (0.4); 7.2669 (1.2); 7.2568 (1.5); 7.2454 (1.5); 7.2339 (0.8); 7.2239 (0.3); 7.1118 (3.5); 6.9487 (0.4); 6.9290 (8.0); 6.9058 (0.3); 6.2202 (2.0); 6.1974 (2.1); 4.9443 (6.8); 4.6316 (0.4); 4.6146 (0.8); 4.6022 (0.9); 4.5803 (0.5); 4.0980 (2.5); 4.0799 (5.5); 4.0618 (2.6); 3.9018 (16.0); 3.3234 (85.2); 3.1741 (0.4); 3.1618 (0.3); 3.0488 (2.7); 3.0307 (5.7); 3.0125 (2.5); 2.6749 (1.1); 2.6703 (1.1); 2.6652 (1.2); 2.6613 (1.2); 2.6446 (1.1); 2.6287 (1.2); 2.6141 (0.6); 2.5867 (0.4); 2.5055 (86.7); 2.5012 (112.5); 2.4969 (83.9); 2.3321 (0.5); 2.3276 (0.7); 2.3237 (0.7); 2.1939 (14.1); 1.9269 (0.5); 1.8878 (1.5); 1.8785 (1.0); 1.8620 (0.7); 1.8433 (0.5); 1.7732 (0.4); 1.7457 (0.9); 1.7264 (0.9); 1.7213 (0.8); 1.7026 (0.6); 1.6868 (0.4); 1.6712 (0.7); 1.6553 (0.6); 1 6471 (0.7); 1.6310 (0.4); −0.0002 (1.4)

I-316: ¹H-NMR(400.0 MHz, d₆-DMSO):

δ=7.1554 (3.9); 6.9603 (0.6); 6.9403 (8.9); 6.9170 (0.4); 6.1475 (2.3); 6.1248 (2.4); 4.8639 (0.5); 4.8469 (1.3); 4.8295 (1.8); 4.8121 (1.4); 4.7949 (0.5); 4.6400 (0.5); 4.6227 (1.0); 4.6100 (1.1); 4.5884 (0.6); 4.0210 (3.0); 4.0028 (6.5); 3.9848 (3.1); 3.9015 (12.2); 3.3237 (119.1); 3.1741 (0.8); 3.1622 (0.8); 2.9109 (3.3); 2.8928 (6.9); 2.8747 (3.1); 2.7183 (0.4); 2.6909 (0.8); 2.6742 (1.6); 2.6707 (1.6); 2.6604 (1.2); 2.6403 (1.4); 2.6260 (0.7); 2.5978 (0.5); 2.5054 (82.8); 2.5011 (107.2); 2.4967 (81.3); 2.3321 (0.5); 2.3278 (0.7); 2.3236 (0.5); 2.2138 (16.0); 1.9440 (0.6); 1.9233 (1.0); 1.9073 (1.8); 1.8811 (0.8); 1.8610 (0.6); 1.7979 (0.5); 1.7745 (1.1); 1.7554 (0.9); 1.7502 (1.0); 1.7316 (0.5); 1.6981 (0.4); 1.6799 (0.8); 1.6583 (0.8); 1.6372 (0.5); 1.4039 (11.0); 1.3994 (11.5); 1.3867 (11.2); 1.3821 (11.2); −0.0005 (0.9)

I-317: ¹H-NMR(400.0 MHz, d₆-DMSO):

δ=7.8166 (3.1); 7.7993 (1.9); 7.7823 (1.1); 7.6226 (0.7); 7.6038 (1.2); 7.5842 (0.5); 7.1418 (0.7); 6.9317 (2.4); 6.8829 (5.0); 5.8257 (1.2); 5.8032 (1.2); 4.2011 (0.5); 4.1819 (0.5); 4.0738 (0.4); 4.0668 (0.4); 4.0510 (0.4); 4.0416 (0.4); 4.0342 (0.5); 4.0250 (0.4); 3.9653 (0.8); 3.9593 (0.7); 3.9509 (1.3); 3.9447 (1.2); 3.9274 (0.7); 3.9069 (2.2); 3.9020 (16.0); 3.8596 (0.4); 3.8512 (0.5); 3.8391 (0.4); 3.8288 (0.6); 3.8184 (0.4); 3.8066 (0.3); 3.7694 (0.4); 3.7537 (0.8); 3.7392 (0.4); 3.3244 (73.0); 3.1883 (0.5); 3.1740 (0.6); 2.6754 (0.5); 2.6706 (0.7); 2.6662 (0.6); 2.5746 (0.7); 2.5603 (0.7); 2.5482 (0.7); 2.5235 (1.9); 2.5060 (77.8); 2.5016 (100.4); 2.4972 (74.6); 2.3281 (1.0); 2.3235 (0.9); 2.3184 (1.0); 2.3072 (0.7); 2.3025 (0.7); 2.2926 (0.7); 2.2775 (0.5); 2.2137 (2.9); 2.1749 (8.2); 1.9726 (0.4); 1.9596 (0.4); 1.9466 (0.3); 1.8721 (0.4); 1.8611 (0.3); 1.7440 (0.5); 1.7320 (0.5); 1.7069 (0.4); 1.6152 (0.4); 1.5905 (0.5); 1.5578 (0.4); 1.4758 (0.8); 1.4590 (1.0); −0.0002 (1.3)

I-318: ¹H-NMR(400.0 MHz, d₆-DMSO):

δ=7.1478 (2.6); 6.9324 (5.7); 6.1434 (1.5); 6.1208 (1.5); 4.6469 (0.3); 4.6283 (0.6); 4.6164 (0.7); 4.5945 (0.4); 3.9458 (1.5); 3.9306 (3.3); 3.9151 (1.6); 3.9018 (16.0); 3.8902 (0.4); 3.8696 (0.8); 3.8569 (2.0); 3.8423 (2.1); 3.8295 (0.8); 3.5927 (0.6); 3.5853 (0.6); 3.5741 (1.0); 3.5616 (0.7); 3.5540 (0.6); 3.3244 (100.6); 3.1747 (0.4); 3.1623 (0.4); 2.6702 (1.2); 2.6662 (1.1); 2.6526 (0.9); 2.6367 (0.9); 2.6228 (0.5); 2.5058 (68.6); 2.5015 (88.8); 2.4971 (66.0); 2.3282 (0.6); 2.3234 (0.5); 2.3187 (0.5); 2.2092 (10.7); 2.1016 (0.5); 2.0879 (1.4); 2.0735 (1.9); 2.0592 (1.4); 2.0446 (0.5); 1.9447 (0.4); 1.9309 (0.5); 1.9253 (0.5); 1.9153 (0.9); 1.9035 (1.2); 1.8928 (0.7); 1.8735 (0.6); 1.8589 (0.4); 1.7781 (0.7); 1.7580 (0.6); 1.7347 (0.4); 1.6837 (0.5); 1.6612 (0.5); 1.6377 (0.4); 1.2355 (0.4); 0.9109 (0.4); 0.8984 (1.4); 0.8912 (2.5); 0.8879 (2.4); 0.8797 (2.2); 0.8596 (2.3); 0.8521 (1.3); 0.8398 (2.2); 0.8326 (1.2); 0.8201 (0.5); −0.0002 (1.2)

I-320: ¹H-NMR(400.0 MHz, d₆-DMSO):

δ=7.1014 (3.9); 7.0793 (2.7); 6.9909 (2.0); 6.9720 (1.4); 6.1947 (2.0); 6.1730 (2.0); 5.0210 (0.5); 5.0006 (1.3); 4.9807 (1.3); 4.9603 (0.5); 3.9315 (2.1); 3.9161 (4.5); 3.9019 (16.0); 3.8476 (2.1); 3.8336 (2.4); 3.8195 (2.2); 3.3215 (57.2); 2.8887 (0.4); 2.8822 (0.4); 2.8675 (0.5); 2.8599 (0.5); 2.8503 (0.7); 2.8431 (0.7); 2.8286 (0.7); 2.8215 (0.7); 2.7328 (0.5); 2.7119 (1.0); 2.6916 (0.8); 2.6707 (1.0); 2.6523 (0.4); 2.5181 (24.5); 2.5100 (35.6); 2.5057 (69.7); 2.5013 (90.5); 2.4968 (65.8); 2.4704 (0.8); 2.4626 (0.7); 2.4513 (0.9); 2.4436 (0.9); 2.4319 (0.9); 2.4203 (0.9); 2.4126 (0.9); 2.4012 (0.6); 2.3935 (0.4); 2.3324 (0.4); 2.3278 (0.6); 2.3234 (0.4); 2.2538 (13.7); 2.0980 (0.6); 2.0833 (1.6); 2.0688 (2.1); 2.0545 (1.6); 2.0397 (0.6); 1.8534 (0.4); 1.8317 (1.0); 1.8227 (0.4); 1.8108 (1.0); 1.8009 (1.0); 1.7889 (0.4); 1.7797 (1.0); 1.7578 (0.3); −0.0002 (1.4)

1H-NMR

I-321: ¹H-NMR(400.0 MHz, d₆-DMSO):

δ=7.1474 (2.6); 6.9586 (0.3); 6.9386 (5.6); 6.0994 (1.4); 6.0768 (1.5); 4.6545 (0.3); 4.6367 (0.6); 4.6255 (0.7); 4.6042 (0.4); 3.9224 (1.6); 3.9067 (3.7); 3.9019 (16.0); 3.8920 (1.8); 3.8410 (1.2); 3.8329 (1.6); 3.8252 (1.6); 3.8165 (1.2); 3.7508 (0.4); 3.3235 (67.2); 3.1740 (0.7); 3.1619 (0.6); 2.6901 (0.5); 2.6750 (1.1); 2.6706 (1.2); 2.6591 (0.9); 2.6402 (1.0); 2.6254 (0.6); 2.5979 (0.3); 2.5183 (17.6); 2.5057 (61.7); 2.5013 (80.3); 2.4970 (59.9); 2.3323 (0.4); 2.3281 (0.5); 2.3232 (0.4); 2.3181 (0.4); 2.2134 (11.7); 2.0936 (0.4); 2.0786 (1.2); 2.0642 (1.6); 2.0501 (1.2); 2.0354 (0.4); 1.9561 (0.4); 1.9404 (0.5); 1.9205 (0.7); 1.9080 (1.2); 1.8791 (0.6); 1.8599 (0.5); 1.7730 (0.7); 1.7539 (0.6); 1.7493 (0.7); 1.7306 (0.4); 1.6799 (0.5); 1.6605 (0.6); 1.6390 (0.4); −0.0002 (0.8)

I-322: ¹H-NMR(400.0 MHz, d₆-DMSO):

δ=7.3082 (1.8); 7.2907 (2.4); 7.2243 (1.3); 7.2069 (2.7); 7.1882 (1.3); 7.1724 (2.3); 7.1545 (2.6); 7.1349 (1.8); 7.1171 (0.6); 6.2411 (2.2); 6.2192 (2.3); 5.0606 (0.6); 5.0406 (1.6); 5.0204 (1.6); 5.0004 (0.6); 3.9323 (2.4); 3.9170 (5.0); 3.9018 (16.0); 3.8468 (2.3); 3.8329 (2.7); 3.8187 (2.4); 3.7581 (0.3); 3.3267 (136.8); 3.1749 (0.4); 3.1620 (0.4); 2.9548 (0.5); 2.9469 (0.6); 2.9328 (0.6); 2.9251 (0.6); 2.9156 (0.9); 2.9077 (0.9); 2.8937 (0.9); 2.8859 (0.9); 2.7968 (0.6); 2.7759 (1.3); 2.7557 (1.1); 2.7365 (0.8); 2.7157 (0.5); 2.6705 (0.6); 2.5060 (79.7); 2.5017 (101.7); 2.4973 (75.5); 2.4619 (1.2); 2.4535 (1.2); 2.4424 (1.2); 2.4309 (1.2); 2.4230 (1.0); 2.4120 (0.6); 2.4037 (0.5); 2.3281 (0.6); 2.3241 (0.5); 2.0983 (0.6); 2.0831 (1.8); 2.0688 (2.4); 2.0545 (1.8); 2.0392 (0.6); 1.9086 (0.4); 1.8816 (0.5); 1.8601 (1.2); 1.8509 (0.5); 1.8392 (1.2); 1.8290 (1.1); 1.8175 (0.5); 1.8083 (1.1); 1.7864 (0.4); −0.0002 (0.7)

I-324: ¹H-NMR(400.0 MHz, d₆-DMSO):

δ=7.2914 (2.0); 7.2725 (2.2); 7.1330 (1.0); 7.1292 (1.0); 7.1119 (2.0); 7.0945 (1.3); 7.0906 (1.2); 6.8525 (1.4); 6.8501 (1.6); 6.8317 (2.6); 6.8154 (1.2); 6.8129 (1.2); 6.7502 (2.8); 6.7485 (2.8); 6.7298 (2.5); 6.3856 (2.3); 6.3639 (2.4); 4.7339 (0.6); 4.7202 (1.3); 4.7077 (0.9); 4.6993 (1.3); 4.6853 (0.6); 4.2799 (0.5); 4.2706 (0.6); 4.2605 (0.5); 4.2518 (1.4); 4.2422 (1.2); 4.2336 (1.1); 4.2237 (1.0); 4.2021 (0.9); 4.1919 (1.2); 4.1877 (1.3); 4.1766 (1.2); 4.1647 (0.6); 4.1590 (0.6); 4.1497 (0.5); 3.9338 (2.4); 3.9184 (5.3); 3.9022 (16.0); 3.8849 (0.3); 3.8714 (0.5); 3.8518 (1.2); 3.8382 (3.2); 3.8229 (3.2); 3.8092 (1.2); 3.7898 (0.5); 3.3244 (95.2); 3.1720 (0.5); 2.6754 (0.5); 2.6707 (0.6); 2.6664 (0.4); 2.5169 (28.4); 2.5102 (41.1); 2.5061 (80.3); 2.5017 (104.6); 2.4973 (77.0); 2.3330 (0.5); 2.3284 (0.6); 2.3238 (0.5); 2.0970 (0.8); 2.0822 (2.3); 2.0673 (3.6); 2.0527 (3.6); 2.0368 (2.8); 2.0268 (1.8); 2.0123 (0.8); 2.0025 (0.5); 1.2117 (0.4); 1.1956 (0.4); 0.0000 (0.8)

I-325: ¹H-NMR(400.0 MHz, d₆-DMSO):

δ=7.6694 (0.4); 7.2500 (0.3); 7.1373 (2.0); 6.9324 (4.7); 6.1919 (1.2); 6.1694 (1.2); 4.5992 (0.5); 4.5865 (0.6); 4.2175 (0.7); 4.2004 (1.0); 4.1834 (0.8); 3.9247 (1.3); 3.9096 (2.7); 3.9019 (16.0); 3.8351 (1.2); 3.8217 (1.6); 3.8083 (1.3); 3.3243 (82.8); 3.1719 (0.6); 3.1647 (0.6); 2.8904 (0.4); 2.6707 (1.0); 2.6668 (0.9); 2.6507 (0.6); 2.6324 (0.7); 2.5879 (0.5); 2.5717 (0.5); 2.5548 (0.4); 2.5060 (70.1); 2.5018 (89.9); 2.4980 (68.2); 2.3430 (0.5); 2.3281 (0.6); 2.3179 (2.0); 2.2100 (8.3); 2.1448 (1.1); 2.1315 (1.4); 2.1177 (1.1); 2.0303 (0.3); 2.0144 (0.5); 1.9175 (0.6); 1.9043 (1.0); 1.8931 (0.8); 1.8769 (0.5); 1.7569 (0.6); 1.7331 (0.6); 1.7138 (0.4); 1.6762 (0.4); 1.6574 (0.4); 1.3009 (6.0); 1.2949 (6.2); 1.2838 (6.2); 1.2779 (5.9); −0.0002 (0.7)

I-326: ¹H-NMR(400.0 MHz, d₆-DMSO):

δ=7.3109 (1.0); 7.2935 (1.2); 7.2218 (0.7); 7.2042 (1.5); 7.1843 (0.7); 7.1691 (1.2); 7.1485 (1.5); 7.1305 (1.0); 7.1111 (0.4); 6.3017 (1.2); 6.2800 (1.3); 5.0229 (0.8); 5.0023 (0.8); 4.9827 (0.3); 3.9255 (1.2); 3.9104 (2.7); 3.9018 (16.0); 3.8134 (1.2); 3.8001 (1.6); 3.7862 (1.4); 3.3227 (55.7); 3.2190 (0.5); 3.2111 (0.6); 3.1994 (1.0); 3.1871 (0.6); 3.1791 (0.7); 3.1668 (0.9); 2.9349 (0.3); 2.9265 (0.3); 2.9164 (0.5); 2.9091 (0.5); 2.8955 (0.5); 2.8876 (0.5); 2.7936 (0.3); 2.7721 (0.7); 2.7520 (0.6); 2.7325 (0.4); 2.6703 (0.4); 2.5235 (0.9); 2.5099 (27.2); 2.5057 (55.8); 2.5012 (73.9); 2.4967 (55.0); 2.4687 (0.8); 2.4579 (0.8); 2.4495 (0.7); 2.4382 (0.7); 2.4270 (0.7); 2.4188 (0.6); 2.4077 (0.4); 2.3996 (0.4); 2.3329 (0.4); 2.3279 (0.5); 2.3235 (0.4); 2.1765 (0.4); 2.1614 (1.0); 2.1478 (1.2); 2.1339 (1.0); 1.8488 (0.6); 1.8280 (0.6); 1.8176 (0.6); 1.7973 (0.6); 1.1789 (0.4); 1.1605 (1.8); 1.1493 (1.8); 1.1390 (0.7); 1.0919 (0.5); 1.0787 (1.6); 1.0726 (1.2); 1.0585 (1.5); −0.0002 (1.1)

1H-NMR

I-327: ¹H-NMR(400.0 MHz, d₆-DMSO):

δ=7.1477 (1.3); 6.9359 (2.8); 6.1449 (0.8); 6.1221 (0.8); 4.6146 (0.4); 3.9170 (0.9); 3.9017 (16.0); 3.8867 (0.9); 3.8119 (0.8); 3.7982 (1.0); 3.7847 (0.9); 3.3240 (61.7); 3.2240 (0.4); 3.2160 (0.4); 3.2042 (0.7); 3.1920 (0.4); 3.1842 (0.4); 3.1729 (0.5); 3.1629 (0.3); 2.6748 (0.6); 2.6705 (0.7); 2.6609 (0.4); 2.6391 (0.4); 2.5236 (0.9); 2.5102 (22.1); 2.5059 (44.8); 2.5014 (58.8); 2.4968 (43.1); 2.4925 (21.8); 2.3281 (0.4); 2.3175 (1.1); 2.2103 (5.5); 2.1587 (0.7); 2.1447 (0.9); 2.1305 (0.7); 1.9218 (0.4); 1.9084 (0.6); 1.8964 (0.5); 1.8808 (0.3); 1.7612 (0.4); 1.7429 (0.3); 1.7379 (0.3); 1.1651 (1.1); 1.1564 (1.1); 1.1449 (0.6); 1.0976 (0.4); 1.0843 (1.1); 1.0782 (0.9); 1.0641 (1.0); 1.0596 (0.8); −0.0002 (0.6)

I-328: ¹H-NMR(400.0 MHz, d₆-DMSO):

δ=7.4773 (0.3); 7.1070 (1.8); 7.0915 (1.1); 7.0725 (1.5); 6.9850 (1.2); 6.9658 (0.8); 6.2948 (0.9); 6.2736 (1.0); 4.9538 (0.7); 4.9337 (0.7); 4.2221 (0.6); 4.2051 (0.9); 4.1879 (0.7); 3.9312 (1.1); 3.9162 (2.3); 3.9017 (16.0); 3.8377 (1.2); 3.8247 (1.5); 3.8109 (1.2); 3.3240 (55.5); 3.1684 (1.0); 2.8490 (0.4); 2.8427 (0.4); 2.8285 (0.4); 2.8212 (0.4); 2.7042 (0.6); 2.6835 (0.5); 2.6661 (0.7); 2.5054 (64.0); 2.5013 (81.4); 2.4971 (61.1); 2.4566 (0.5); 2.4483 (0.5); 2.4370 (0.6); 2.4295 (0.6); 2.4181 (0.6); 2.4064 (0.6); 2.3988 (0.6); 2.3872 (0.4); 2.3791 (0.4); 2.3672 (1.2); 2.3276 (0.6); 2.2769 (0.3); 2.2485 (7.1); 2.1478 (0.9); 2.1342 (1.2); 2.1214 (1.0); 2.0921 (0.4); 2.0110 (0.4); 1.8176 (0.5); 1.7967 (0.6); 1.7874 (0.5); 1.7658 (0.5); 1.2973 (6.3); 1.2821 (6.3); 1.2489 (0.3); −0.0002 (0.7)

I-329: ¹H-NMR(400.0 MHz, d₆-DMSO):

δ=7.1442 (2.6); 6.9363 (6.1); 6.1898 (1.5); 6.1669 (1.6); 4.6363 (0.3); 4.6184 (0.6); 4.6060 (0.7); 4.5851 (0.4); 3.9019 (16.0); 3.8883 (3.3); 3.8731 (1.6); 3.8070 (1.6); 3.7936 (2.0); 3.7800 (1.7); 3.4124 (15.2); 3.3221 (43.8); 3.2670 (0.4); 3.1685 (2.0); 2.6877 (0.5); 2.6701 (1.2); 2.6550 (0.8); 2.6374 (0.9); 2.6228 (0.5); 2.5232 (1.5); 2.5097 (33.5); 2.5056 (66.1); 2.5012 (85.8); 2.4967 (63.4); 2.4926 (32.5); 2.3323 (0.4); 2.3279 (0.6); 2.3231 (0.5); 2.3181 (0.6); 2.2115 (10.9); 2.1807 (0.5); 2.1665 (1.3); 2.1530 (1.6); 2.1388 (1.2); 1.9385 (0.4); 1.9327 (0.5); 1.9203 (0.7); 1.9070 (1.2); 1.8951 (1.0); 1.8792 (0.6); 1.8596 (0.4); 1.7590 (0.7); 1.7400 (0.6); 1.7344 (0.7); 1.7155 (0.4); 1.6789 (0.5); 1.6590 (0.5); 1.6393 (0.4); −0.0002 (1.1)

I-330: ¹H-NMR(400.0 MHz, d₆-DMSO):

δ=7.1646 (2.5); 6.9690 (0.4); 6.9485 (5.3); 6.1103 (1.5); 6.0877 (1.6); 4.7058 (0.3); 4.6765 (0.6); 4.6566 (0.3); 3.9116 (1.5); 3.9018 (16.0); 3.8970 (3.8); 3.8814 (1.8); 3.8708 (0.6); 3.8635 (0.9); 3.8550 (0.7); 3.8341 (1.1); 3.8215 (1.8); 3.8068 (1.7); 3.7951 (0.8); 3.3246 (96.3); 2.6983 (0.4); 2.6810 (0.8); 2.6750 (0.8); 2.6707 (1.0); 2.6663 (1.0); 2.6478 (0.8); 2.6334 (0.4); 2.5239 (1.3); 2.5103 (31.8); 2.5062 (63.2); 2.5017 (82.1); 2.4972 (60.0); 2.3329 (0.4); 2.3285 (0.5); 2.3238 (0.4); 2.2176 (10.4); 2.0767 (0.4); 2.0622 (1.3); 2.0478 (1.8); 2.0332 (1.3); 2.0180 (0.4); 1.9270 (0.8); 1.9044 (1.2); 1.8404 (1.0); 1.8141 (1.4); 1.7755 (0.7); 1.7522 (0.4); 1.6984 (1.2); 1.6937 (1.2); 1.6761 (1.6); 1.6167 (0.8); 1.5931 (0.6); 1.3358 (0.7); 1.3110 (1.0); 1.2822 (1.2); 1.2493 (1.2); 1.2131 (1.0); 1.1903 (1.1); 1.1697 (0.6); 1.1386 (0.3); −0.0002 (0.6)

I-331: ¹H-NMR(400.0 MHz, d₆-DMSO):

δ=7.3418 (1.1); 7.3269 (1.2); 7.3198 (1.2); 7.1204 (1.3); 7.1157 (2.0); 7.1068 (3.4); 7.0983 (2.6); 7.0927 (1.7); 7.0800 (0.6); 7.0664 (1.5); 7.0588 (1.2); 7.0441 (0.8); 6.1677 (1.5); 6.1450 (1.6); 4.6987 (0.4); 4.6787 (0.6); 4.6679 (0.7); 4.6455 (0.4); 3.9205 (1.7); 3.9017 (16.0); 3.8898 (1.8); 3.8467 (0.9); 3.8343 (1.9); 3.8215 (1.9); 3.8092 (0.9); 3.3260 (95.7); 3.1751 (0.6); 3.1624 (0.6); 2.7459 (0.6); 2.7279 (0.8); 2.7135 (1.0); 2.6963 (1.0); 2.6806 (0.7); 2.6710 (0.7); 2.5112 (42.5); 2.5061 (61.2); 2.5018 (76.9); 2.4974 (56.2); 2.3327 (0.4); 2.3286 (0.5); 2.0923 (0.5); 2.0775 (1.4); 2.0633 (1.9); 2.0488 (1.4); 2.0337 (0.5); 1.9965 (0.4); 1.9807 (0.3); 1.9469 (0.9); 1.9311 (2.1); 1.9201 (1.5); 1.9074 (0.8); 1.8882 (0.5); 1.8315 (0.4); 1.8265 (0.4); 1.8035 (0.8); 1.7836 (0.7); 1.7612 (0.5); 1.7305 (0.4); 1.7118 (0.6); 1.6955 (0.6); 1.6741 (0.4); −0.0001 (0.3)

1H-NMR

I-332: ¹H-NMR(400.0 MHz, d₆-DMSO):

δ=7.3629 (0.4); 7.3442 (0.5); 7.3273 (0.7); 7.3119 (0.7); 7.3064 (0.7); 7.1140 (0.6); 7.1071 (0.8); 7.0989 (1.8); 7.0919 (1.0); 7.0830 (0.8); 7.0598 (0.9); 7.0534 (0.7); 7.0377 (0.5); 6.2708 (0.7); 6.2482 (0.7); 4.6354 (0.4); 4.6241 (0.4); 4.2059 (0.6); 4.1887 (0.9); 4.1716 (0.6); 3.9203 (1.0); 3.9018 (16.0); 3.8907 (1.1); 3.8318 (0.9); 3.8188 (1.3); 3.8049 (1.0); 3.3271 (58.8); 3.1684 (1.0); 2.9460 (0.4); 2.7189 (0.4); 2.7053 (0.5); 2.6896 (0.6); 2.6750 (0.6); 2.6709 (0.5); 2.6660 (0.4); 2.6000 (0.3); 2.5238 (1.0); 2.5103 (24.7); 2.5060 (50.3); 2.5015 (66.3); 2.4970 (49.0); 2.4929 (25.1); 2.3283 (0.4); 2.3242 (0.3); 2.1427 (0.8); 2.1291 (1.0); 2.1156 (0.8); 2.0419 (0.4); 1.9392 (0.5); 1.9250 (0.7); 1.9149 (0.8); 1.9017 (0.4); 1.7862 (0.4); 1.7667 (0.4); 1.6905 (0.3); 1.2969 (4.6); 1.2866 (4.9); 1.2798 (4.9); 1.2695 (4.4); −0.0002 (0.5)

I-333: ¹H-NMR(400.0 MHz, d₆-DMSO):

δ=7.3385 (0.7); 7.3230 (0.8); 7.3164 (0.8); 7.1172 (0.7); 7.1121 (1.3); 7.1033 (2.2); 7.0949 (1.5); 7.0887 (1.0); 7.0759 (0.4); 7.0641 (1.0); 7.0567 (0.8); 7.0476 (0.4); 7.0418 (0.5); 6.2176 (1.1); 6.1950 (1.1); 4.6641 (0.4); 4.6529 (0.5); 3.9138 (1.1); 3.9017 (16.0); 3.8837 (1.1); 3.8094 (1.0); 3.7963 (1.3); 3.7827 (1.1); 3.3296 (102.4); 3.2119 (0.4); 3.2040 (0.5); 3.1922 (0.9); 3.1800 (0.6); 3.1747 (0.9); 3.1617 (0.8); 2.7439 (0.4); 2.7259 (0.5); 2.7124 (0.6); 2.6954 (0.7); 2.6799 (0.5); 2.6756 (0.5); 2.6707 (0.5); 2.6667 (0.4); 2.5238 (1.2); 2.5103 (25.7); 2.5061 (51.1); 2.5016 (66.6); 2.4971 (48.8); 2.3284 (0.4); 2.3236 (0.3); 2.1568 (0.8); 2.1432 (1.0); 2.1293 (0.8); 2.1140 (0.3); 1.9446 (0.6); 1.9307 (0.9); 1.9196 (0.9); 1.9057 (0.5); 1.7914 (0.5); 1.7712 (0.5); 1.7486 (0.3); 1.7116 (0.3); 1.6957 (0.4); 1.1600 (1.3); 1.1513 (1.3); 1.1398 (0.5): 1.0914 (0.4); 1.0777 (1.4); 1.0723 (1.1); 1.0577 (1.3): -0.0002 (0.6)

I-334: ¹H-NMR(400.0 MHz, d₆-DMSO):

δ=7.6506 (0.4); 7.3098 (0.8); 7.2913 (1.0); 7.2196 (0.6); 7.2020 (1.2); 7.1835 (0.6); 7.1678 (1.0); 7.1459 (1.0); 7.1268 (0.8); 6.3455 (0.7); 6.3241 (0.7); 4.9925 (0.6); 4.9730 (0.6); 4.2113 (0.7); 4.1940 (0.9); 4.1768 (0.7); 3.9328 (1.1); 3.9176 (2.3); 3.9018 (16.0); 3.8370 (1.1); 3.8237 (1.4); 3.8095 (1.2); 3.7903 (0.4); 3.3269 (61.6); 3.1685 (2.3); 2.9138 (0.4); 2.9057 (0.4); 2.8917 (0.4); 2.8844 (0.4); 2.7679 (0.6); 2.7478 (0.4); 2.7284 (0.4); 2.6749 (0.3); 2.6707 (0.5); 2.6465 (0.3); 2.5236 (1.3); 2.5060 (66.8); 2.5016 (86.3); 2.4972 (63.0); 2.4625 (0.6); 2.4511 (0.6); 2.4427 (0.6); 2.4318 (0.6); 2.4206 (0.6); 2.4122 (0.5); 2.3326 (0.4); 2.3283 (0.6); 2.3237 (0.4); 2.1479 (0.9); 2.1345 (1.1): 2.1209 (0.9); 1.8408 (0.5); 1.8204 (0.5); 1.8097 (0.5); 1.7895 (0.5); 1.2970 (5.4); 1.2912 (6.5); 1.2799 (5.4); 1.2740 (6.1); 1.2536 (0.5); 1.2363 (0.6); −0.0002 (0.5)

I-336: ¹H-NMR(400.0 MHz, d₆-DMSO):

δ=7.1098 (2.0); 7.0961 (1.2); 7.0770 (1.6); 6.9882 (1.2); 6.9686 (0.9); 6.2517 (1.2); 6.2299 (1.3); 4.9845 (0.8); 4.9644 (0.8); 3.9246 (1.2); 3.9094 (2.8); 3.9017 (16.0); 3.8150 (1.2); 3.8014 (1.5); 3.7878 (1.3); 3.3295 (131.4); 3.2280 (0.5); 3.2200 (0.6); 3.2084 (1.0); 3.1963 (0.6); 3.1883 (0.5); 3.1751 (0.6); 3.1621 (0.5); 2.8532 (0.4); 2.8456 (0.4); 2.8315 (0.4); 2.8240 (0.4); 2.7094 (0.6); 2.6894 (0.5); 2.6706 (0.8); 2.5238 (1.3); 2.5061 (53.7); 2.5017 (69.9); 2.4973 (51.4); 2.4641 (0.4); 2.4562 (0.3); 2.4449 (0.5); 2.4371 (0.5); 2.4256 (0.5); 2.4141 (0.6); 2.4062 (0.5); 2.3955 (0.3); 2.3673 (0.4); 2.3328 (0.4); 2.3283 (0.4); 2.3241 (0.3); 2.2493 (8.1); 2.1769 (0.4); 2.1621 (1.0); 2.1483 (1.2); 2.1349 (1.0); 2.1195 (0.4); 1.8236 (0.6); 1.8028 (0.6); 1.7928 (0.6); 1.7719 (0.6); 1.1841 (0.4); 1.1712 (1.1); 1.1653 (1.7); 1.1544 (1.7); 1.1443 (0.7); 1.0960 (0.5); 1.0829 (1.5); 1.0771 (1.2); 1.0628 (1.5); −0.0001 (0.7)

I-337: ¹H-NMR(400.0 MHz, d₆-DMSO):

δ=7.2856 (1.0); 7.2666 (1.1); 7.1264 (0.5); 7.1226 (0.5); 7.1053 (1.0); 7.0880 (0.7); 7.0844 (0.6); 6.8383 (0.8); 6.8199 (1.3); 6.8037 (0.6); 6.8011 (0.6); 6.7403 (1.4); 6.7216 (1.2); 6.7198 (1.2); 6.4755 (1.2); 6.4539 (1.2); 4.6809 (0.6); 4.6718 (0.5); 4.6596 (0.6); 4.2442 (0.7); 4.2360 (0.6); 4.2250 (0.8); 4.2159 (0.6); 4.2075 (0.8); 4.1901 (1.4); 4.1826 (0.7); 4.1729 (1.3); 4.1553 (0.6); 3.9366 (1.1); 3.9216 (2.3); 3.9017 (16.0); 3.8366 (1.0); 3.8233 (1.4); 3.8099 (1.1); 3.3262 (91.9); 3.1747 (0.8); 3.1616 (0.8); 2.6706 (0.3); 2.5099 (22.7); 2.5058 (45.1); 2.5014 (59.1); 2.4969 (44.0); 2.3277 (0.4); 2.1490 (0.9); 2.1351 (1.1); 2.1212 (0.8); 2.1069 (0.3); 2.0558 (0.4); 2.0453 (0.5); 2.0344 (0.8); 2.0236 (0.9); 2.0106 (0.8); 1.9950 (0.4); 1.2996 (5.1); 1.2929 (5.4); 1.2824 (5.2); 1.2758 (5.2)

1H-NMR

I-338: ¹H-NMR(400.0 MHz, d₆-DMSO):

δ=7.2921 (1.0); 7.2742 (1.0); 7.1301 (0.5); 7.1259 (0.5); 7.1086 (1.0); 7.0912 (0.6); 7.0874 (0.6); 6.8478 (0.7); 6.8453 (0.7); 6.8269 (1.2); 6.8107 (0.6); 6.8082 (0.6); 6.7473 (1.4); 6.7270 (1.2); 6.4326 (1.2); 6.4108 (1.2); 4.7073 (0.6); 4.6964 (0.4); 4.6854 (0.6); 4.2518 (0.7); 4.2426 (0.6); 4.2327 (0.5); 4.2237 (0.4); 4.2023 (0.4); 4.1925 (0.5); 4.1878 (0.6); 4.1768 (0.6); 3.9285 (1.1); 3.9133 (2.4); 3.9017 (16.0); 3.8145 (1.1); 3.8011 (1.4); 3.7871 (1.1); 3.3282 (110.3); 3.2157 (0.5); 3.2074 (0.5); 3.1960 (0.9); 3.1839 (0.5); 3.1752 (1.0); 3.1620 (0.7); 2.6706 (0.4); 2.5238 (1.1); 2.5103 (23.6); 2.5061 (46.8); 2.5016 (60.8); 2.4971 (44.4); 2.3283 (0.4); 2.1771 (0.3); 2.1616 (0.8); 2.1480 (1.1); 2.1346 (0.8); 2.0613 (0.4); 2.0520 (0.5); 2.0422 (0.8); 2.0284 (0.9); 2.0186 (0.8); 2.0029 (0.4); 1.1645 (1.3); 1.1566 (1.4); 1.1455 (0.7); 1.1395 (0.4); 1.0978 (0.4); 1.0844 (1.4); 1.0783 (1.1); 1.0644 (1.3); 1.0593 (1.0); −0.0002 (0.4)

I-340: ¹H-NMR(400.0 MHz, CDCl3):

δ=7.5184 (1.0); 7.3108 (0.5); 7.2595 (182.0); 7.2103 (0.6); 7.0852 (1.6); 7.0671 (4.8); 7.0223 (2.3); 7.0025 (1.2); 6.9955 (1.3); 4.9028 (3.8); 4.8700 (2.8); 4.7597 (1.7); 4.7390 (1.7); 4.2035 (1.1); 4.1897 (2.0); 4.1756 (2.3); 4.1642 (2.7); 4.1147 (1.6); 4.1016 (1.6); 3.4911 (4.0); 3.0615 (0.9); 3.0417 (1.0); 3.0231 (1.1); 3.0042 (1.2); 2.5209 (1.3); 2.4980 (1.6); 2.4833 (1.3); 2.4602 (1.5); 2.3011 (16.0); 2.2684 (1.6); 2.2510 (1.1); 2.2290 (0.6); 1.2872 (10.2); 1.2705 (10.2); 0.0079 (1.8); −0.0002 (64.6); −0.0085 (2.8)

I-341: ¹H-NMR(400.0 MHz, d₆-DMSO):

δ=9.4632 (2.3); 8.3152 (0.6); 7.6870 (0.9); 7.6694 (1.2); 7.6500 (1.6); 7.6302 (1.1); 7.6008 (0.9); 7.5823 (0.8); 7.5025 (0.4); 7.4387 (0.7); 7.4206 (0.9); 7.4016 (0.5); 7.3535 (0.3); 7.3376 (0.4); 7.2988 (0.4); 7.2761 (2.4); 7.2584 (6.3); 7.2406 (7.8); 7.2233 (6.6); 7.2032 (3.1); 7.1870 (5.0); 7.1788 (3.3); 7.1695 (2.9); 7.1555 (5.2); 7.1355 (5.4); 7.1175 (1.9); 7.0967 (0.4); 6.4547 (1.5); 6.4328 (1.6); 6.3726 (0.5); 6.3484 (0.6); 6.3267 (0.5); 5.0703 (2.6); 5.0544 (2.9); 4.7961 (2.4); 4.7481 (5.3); 4.6964 (0.6); 4.3834 (14.6); 4.3273 (1.6); 4.2122 (0.4); 4.1950 (0.5); 4.1810 (0.5); 4.1563 (3.9); 4.1419 (7.5); 4.1281 (4.9); 4.1087 (2.0); 4.0984 (2.0); 4.0708 (12.6); 4.0052 (0.7); 4.0005 (0.7); 3.9637 (0.6); 3.9318 (0.4); 3.9019 (15.0); 3.8365 (0.5); 3.7679 (0.7); 3.6798 (5.1); 3.6659 (8.3); 3.6515 (5.5); 3.6232 (3.4); 3.5848 (4.7); 3.5028 (16.0); 3.3232 (1.9); 3.2257 (0.6); 3.2119 (0.6); 3.1681 (15.5); 3.1244 (1.0); 3.1103 (1.0); 3.0955 (1.0); 3.0764 (0.4); 3.0574 (0.4); 2.9543 (1.6); 2.9416 (1.5); 2.9332 (1.7); 2.9234 (2.2); 2.9152 (2.6); 2.9023 (2.2); 2.8929 (2.4); 2.8033 (1.3); 2.7831 (2.8); 2.7628 (2.2); 2.7432 (1.6); 2.7231 (0.8); 2.6746 (2.4); 2.6705 (3.4); 2.6663 (2.4); 2.6461 (0.8); 2.6314 (0.6); 2.6169 (0.6); 2.5408 (2.0); 2.5235 (7.1); 2.5058 (517.4); 2.5015 (681.6); 2.4972 (503.8); 2.4617 (5.6); 2.4520 (5.2); 2.4416 (4.3); 2.4305 (4.2); 2.4220 (3.6); 2.4109 (2.4); 2.4026 (2.1); 2.3663 (0.9); 2.3595 (0.9); 2.3325 (3.3); 2.3281 (4.4); 2.3240 (3.4); 2.2830 (0.6); 2.2226 (0.5); 2.2192 (0.5); 2.2049 (0.5); 2.1788 (0.4); 2.1553 (0.4); 2.1047 (0.4); 2.0897 (0.4); 1.9083 (0.4); 1.8689 (1.0); 1.8605 (0.8); 1.8472 (2.4); 1.8379 (2.2); 1.8268 (2.6); 1.8165 (3.3); 1.8060 (2.0); 1.7958 (2.6); 1.7869 (1.5); 1.7738 (0.9); 1.7657 (0.6); 1.3359 (0.3); 1.2585 (0.4); 1.2496 (0.6); 1.2357 (0.8); −0.0003 (5.4)

I-342: ¹H-NMR(400.0() MHz, CDCl3):

δ=7.5187 (1.9); 7.3249 (7.3); 7.3058 (7.9); 7.2598 (322.4); 7.2057 (3.4); 7.1852 (7.1); 7.1671 (4.4); 6.9958 (1.9); 6.9161 (5.4); 6.8973 (9.1); 6.8787 (4.3); 6.8518 (9.8); 6.8313 (8.6); 4.8524 (16.0); 4.8232 (14.1); 4.8084 (9.6); 4.7964 (4.7); 4.3012 (1.4); 4.2904 (2.6); 4.2734 (5.6); 4.2594 (8.4); 4.2488 (8.9); 4.2346 (5.0); 4.2266 (5.8); 4.2068 (2.3); 4.1982 (2.9); 4.1799 (4.5); 4.1684 (6.8); 4.1525 (9.1); 4.1418 (11.2); 4.0935 (6.1); 4.0800 (7.0); 4.0677 (3.3); 3.4907 (2.8); 2.2736 (1.0); 2.2635 (1.5); 2.2527 (1.6); 2.2394 (3.2); 2.2287 (4.6); 2.2175 (5.0); 2.2062 (6.4); 2.1958 (5.8); 2.1840 (3.9); 2.1709 (2.2); 2.1570 (1.4); 0.0079 (4.9); −0.0002 (120.2); −0.0085 (3.6)

I-343: ¹H-NMR(400.0 MHz, CDCl3):

δ=7.5183 (0.8); 7.2595 (154.0); 7.0678 (1.9); 7.0499 (6.7); 7.0089 (2.7); 6.9953 (1.2); 6.9904 (1.3); 4.7230 (2.5); 4.7020 (2.4); 4.1375 (1.6); 4.1260 (2.9); 4.1137 (1.6); 3.8988 (5.7); 3.1409 (1.6); 3.1294 (2.7); 3.1183 (1.5); 3.1130 (1.4); 3.0684 (1.2); 3.0492 (1.3); 3.0304 (1.4); 3.0118 (1.4); 2.8254 (1.4); 2.8073 (4.2); 2.7893 (4.3); 2.7713 (1.4); 2.5113 (0.9); 2.4887 (1.3); 2.4750 (0.7); 2.4519 (1.2); 2.4065 (0.6); 2.3876 (0.8); 2.3673 (1.1); 2.3500 (0.8); 2.2956 (16.0); 1.2770 (12.4); 1.2604 (12.6); 1.2514 (6.1); 1.2334 (12.1); 1.2154 (5.4); 0.0078 (2.0); −0.0002 (57.0); −0.0085 (2.0)

1H-NMR

I-344: ¹H-NMR(400.0 MHz, CDCl3):

δ=7.5184 (0.8); 7.2595 (151.7); 7.0681 (5.5); 7.0481 (3.3); 7.0055 (2.4); 6.9955 (1.0); 6.9857 (1.3); 4.7411 (2.2); 4.7199 (2.2); 4.0985 (1.8); 4.0858 (3.4); 4.0728 (2.0); 3.8067 (5.9); 3.0590 (2.3); 3.0499 (2.9); 3.0385 (2.7); 3.0197 (1.6); 3.0010 (1.5); 2.6387 (2.6); 2.6203 (3.4); 2.6013 (2.9); 2.5092 (0.9); 2.4862 (1.2); 2.4706 (0.7); 2.4483 (1.1); 2.3487 (0.8); 2.3298 (1.1); 2.3122 (0.9); 2.2940 (16.0); 1.6391 (1.6); 1.6204 (2.8); 1.6018 (2.9); 1.5832 (1.7); 1.2815 (12.2); 1.2649 (12.1); 0.9877 (6.2); 0.9694 (12.8); 0.9509 (5.7); 0.0079 (1.4); −0.0002 (55.8); −0.0083 (2.3)

I-345: ¹H-NMR(400.0 MHz, CDCl3):

δ=7.8361 (1.7); 7.5186 (2.0); 7.3920 (4.5); 7.3751 (5.5); 7.3716 (4.6); 7.2597 (336.0); 7.2474 (2.4); 7.2387 (6.6); 7.2351 (9.7); 7.2308 (7.9); 7.2293 (7.9); 7.2235 (11.4); 7.2201 (8.6); 7.2133 (5.9); 7.2070 (4.9); 7.2022 (3.4); 7.1961 (4.1); 7.1892 (2.7); 7.1803 (1.9); 7.1735 (1.2); 6.9957 (2.0); 5.2283 (1.4); 5.2095 (3.4); 5.1890 (3.5); 5.1700 (1.4); 4.2913 (1.9); 4.2763 (3.4); 4.2613 (3.0); 4.2476 (2.0); 4.1245 (10.4); 4.1095 (16.0); 4.0944 (11.3); 3.2213 (1.7); 3.2065 (1.2); 3.1930 (1.9); 3.1796 (11.9); 3.1714 (4.9); 3.1652 (8.9); 3.1594 (4.8); 3.1513 (12.7); 3.0131 (1.5); 3.0041 (1.7); 2.9910 (1.7); 2.9823 (1.8); 2.9733 (2.9); 2.9645 (3.0); 2.9517 (3.0); 2.9430 (3.0); 2.8858 (2.2); 2.8647 (4.6); 2.8449 (3.7); 2.8252 (2.5); 2.8053 (1.6); 2.7111 (2.2); 2.7022 (2.1); 2.6932 (2.5); 2.6842 (2.5); 2.6795 (2.7); 2.6736 (2.1); 2.6706 (2.5); 2.6646 (2.0); 2.6614 (2.8); 2.6525 (2.7); 2.6420 (1.9); 2.6330 (1.7); 2.4693 (0.6); 2.4544 (1.0); 2.4420 (3.8); 2.4342 (2.3); 2.4271 (6.4); 2.4195 (3.8); 2.4128 (8.6); 2.4069 (3.6); 2.4042 (3.6); 2.3987 (5.4); 2.3837 (2.8); 1.8898 (2.1); 1.8685 (4.4); 1.8581 (2.1); 1.8496 (4.3); 1.8472 (2.6); 1.8392 (2.5); 1.8368 (4.0); 1.8282 (2.0); 1.8179 (4.0); 1.7965 (1.8); 1.5646 (17.8); 1.3331 (1.0); 1.2845 (1.6); 1.2563 (2.1); 0.0079 (4.2); −0.0002 (129.4); −0.0085 (3.9)

I-346: ¹H-NMR(400.0 MHz, CDCl3):

δ=7.5193 (1.4); 7.3662 (12.4); 7.3487 (15.5); 7.3116 (0.5); 7.2937 (0.8); 7.2603 (239.3); 7.2536 (26.1); 7.2507 (27.3); 7.2447 (20.7); 7.2393 (25.8); 7.2358 (21.0); 7.2216 (12.5); 7.2175 (9.9); 7.2060 (7.1); 7.2026 (7.1); 7.1870 (3.4); 6.9963 (1.3); 5.2691 (5.1); 5.2609 (5.2); 5.2510 (10.3); 5.2429 (9.8); 5.2330 (5.3); 5.2249 (4.8); 4.3891 (5.4); 4.3831 (6.6); 4.3773 (5.6); 4.3709 (2.9); 4.3610 (3.4); 4.3548 (7.0); 4.3490 (7.7); 4.3429 (6.1); 4.3362 (4.0); 4.1299 (3.1); 4.1193 (4.5); 4.1109 (3.2); 4.1004 (5.2); 4.0937 (5.3); 4.0892 (6.2); 4.0706 (2.8); 4.0663 (2.7); 4.0595 (3.2); 3.4885 (6.9); 3.4196 (1.1); 3.3950 (6.0); 3.3801 (10.3); 3.3652 (7.2); 3.3497 (8.9); 3.3225 (0.9); 3.1355 (1.8); 3.1232 (1.8); 3.1022 (4.7); 3.0905 (5.8); 3.0744 (15.1); 3.0695 (16.0); 3.0574 (5.5); 3.0453 (15.6); 3.0384 (10.1); 3.0302 (4.6); 3.0220 (4.0); 3.0154 (5.5); 3.0092 (5.0); 2.9991 (5.7); 2.9902 (6.6); 2.9824 (5.2); 2.9765 (4.9); 2.9691 (6.9); 2.9611 (4.6); 2.9057 (4.7); 2.8854 (9.5); 2.8654 (7.6); 2.8455 (5.6); 2.8254 (3.5); 2.7130 (2.5); 2.7030 (4.1); 2.6936 (5.2); 2.6838 (6.3); 2.6748 (5.7); 2.6714 (5.6); 2.6619 (6.1); 2.6527 (6.5); 2.6430 (5.3); 2.6334 (3.6); 2.6236 (2.1); 2.3971 (3.3); 2.3896 (4.2); 2.3849 (4.1); 2.3775 (3.9); 2.3742 (3.9); 2.3624 (5.5); 2.3546 (8.4); 2.3414 (2.8); 2.3372 (2.9); 2.3304 (2.0); 2.3196 (1.3); 1.9076 (3.5); 1.8869 (7.7); 1.8756 (3.8); 1.8674 (7.5); 1.8551 (7.3); 1.8468 (3.4); 1.8356 (6.8); 1.8150 (2.7); 0.0071 (3.4); −0.0002 (87.7); −0.0084 (3.3)

I-347: ¹H-NMR(400.0 MHz, CDCl3):

δ=7.5182 (0.5); 7.2592 (86.6); 7.1914 (3.5); 7.1291 (2.0); 7.1101 (3.1); 7.0445 (2.3); 7.0252 (1.4); 5.2978 (3.8); 5.1896 (0.5); 5.1705 (1.3); 5.1494 (1.4); 5.1303 (0.6); 4.2520 (1.1); 4.2289 (1.0); 4.1246 (3.8); 4.1096 (5.8); 4.0944 (4.1); 3.1801 (4.2); 3.1718 (1.8); 3.1657 (3.2); 3.1600 (1.7); 3.1518 (4.6); 2.9492 (0.5); 2.9283 (0.6); 2.9188 (0.8); 2.9103 (0.9); 2.8973 (0.9); 2.8891 (0.9); 2.8306 (0.6); 2.8111 (1.3); 2.7911 (1.0); 2.7722 (0.7); 2.6985 (0.8); 2.6900 (0.8); 2.6806 (0.9); 2.6722 (0.9); 2.6672 (0.9); 2.6613 (0.7); 2.6586 (0.9); 2.6527 (0.7); 2.6490 (1.0); 2.6405 (0.9); 2.6299 (0.6); 2.6213 (0.6); 2.4422 (1.2); 2.4273 (2.2); 2.4195 (1.3); 2.4129 (3.2); 2.4069 (1.3); 2.3987 (1.9); 2.3837 (1.0); 2.3248 (16.0); 1.8712 (0.7); 1.8502 (1.6); 1.8399 (0.7); 1.8311 (1.5); 1.8187 (1.4); 1.8098 (0.7); 1.7996 (1.4); 1.7782 (0.6); 1.5764 (1.2); 0.0080 (1.0); −0.0002 (32.0); −0.0085 (0.9)

1H-NMR

I-348: ¹H-NMR(400.0 MHz, CDCl3):

δ=7.2587 (23.4); 7.1071 (1.6); 7.0653 (0.9); 7.0463 (1.6); 6.9996 (1.2); 6.9806 (0.7); 6.9518 (2.6); 6.8284 (1.0); 6.7868 (1.1); 6.7645 (0.6); 5.2949 (5.8); 4.7791 (0.5); 4.7565 (1.0); 4.7341 (0.6); 4.2015 (0.6); 4.1773 (0.6); 4.1121 (1.9); 4.0971 (2.9); 4.0820 (2.0); 3.8124 (5.1); 3.7013 (16.0); 3.5535 (4.9); 3.1822 (1.8); 3.1776 (0.7); 3.1733 (0.8); 3.1691 (1.2); 3.1671 (1.3); 3.1623 (0.8); 3.1583 (0.7); 3.1539 (2.0); 3.0293 (0.6); 3.0103 (0.6); 2.9910 (0.7); 2.9719 (0.7); 2.4777 (0.5); 2.4409 (1.0); 2.4327 (0.9); 2.4259 (1.1); 2.4208 (0.8); 2.4121 (1.7); 2.4057 (0.8); 2.4029 (0.8); 2.3981 (1.4); 2.3824 (0.6); 2.2948 (7.5); 2.2139 (0.7); 2.1970 (0.6); 2.1933 (0.5); 2.1910 (0.5); 1.5957 (2.4); 1.3097 (5.8); 1.2922 (6.7); 1.2895 (7.8); 1.2727 (7.0); 1.1962 (3.8); 1.1792 (3.7); 1.1500 (0.6); 1.1316 (0.5); 0.9258 (4.1); 0.9175 (0.6); 0.9090 (3.8): -0.0002 (8.6)

I-349: ¹H-NMR(400.0 MHz, CDCl3):

δ=7.5183 (0.9); 7.4466 (3.7); 7.4322 (3.8); 7.4238 (4.2); 7.2593 (150.8); 7.1784 (1.1); 7.1659 (5.5); 7.1615 (7.2); 7.1526 (14.4); 7.1437 (9.5); 7.1391 (5.6); 7.1268 (1.2); 7.1047 (0.8); 7.0950 (4.8); 7.0864 (3.9); 7.0726 (2.6); 6.9954 (0.8); 5.2973 (16.0); 4.8531 (1.5); 4.8393 (2.4); 4.8312 (2.0); 4.8183 (2.6); 4.8038 (1.4); 4.2693 (3.0); 4.2481 (2.7); 4.1299 (0.9); 4.1134 (10.4); 4.0985 (15.4); 4.0833 (10.4); 3.1740 (10.2); 3.1699 (3.3); 3.1655 (4.2); 3.1598 (8.1); 3.1539 (4.2); 3.1457 (10.8); 2.8610 (0.7); 2.8460 (1.4); 2.8308 (0.9); 2.8199 (2.0); 2.8044 (3.7); 2.7890 (2.2); 2.7793 (2.2); 2.7628 (3.2); 2.7467 (2.4); 2.7201 (1.0); 2.7049 (0.8); 2.4365 (2.9); 2.4323 (1.2); 2.4216 (5.4); 2.4162 (3.5); 2.4073 (8.1); 2.4012 (3.0); 2.3987 (3.2); 2.3929 (4.7); 2.3781 (2.4); 2.1239 (0.9); 2.1165 (I.I); 2.1113 (0.8); 2.1015 (1.4); 2.0921 (2.2); 2.0809 (2.3); 2.0703 (2.2); 2.0608 (2.0); 2.0483 (1.6); 2.0430 (3.3); 1.9563 (1.1); 1.9487 (1.4); 1.9405 (1.6); 1.9348 (2.6); 1.9285 (2.4); 1.9159 (2.6); 1.9032 (3.0); 1.8935 (2.2); 1.8864 (3.2); 1.8815 (2.4); 1.8712 (1.5); 1.8668 (1.9); 1.8589 (1.6); 1.8521 (1.7); 1.8434 (2.0); 1.8347 (2.0); 1.8270 (1.6); 1.8186 (1.8); 1.8066 (1.3); 1.7983 (1.3); 1.7854 (0.7); 1.5851 (2.9); 1.2762 (0.9); 1.2582 (2.1); 1.2403 (0.9); 0.0079 (2.0); −0.0002 (53.4); −0.0084 (1.4)

I-350: ¹H-NMR(400.0 MHz, CDCl3):

δ=7.4373 (0.6); 7.4193 (0.8); 7.4098 (1.7); 7.4035 (1.0); 7.4011 (0.9); 7.3957 (1.8); 7.3870 (1.9); 7.2595 (68.2); 7.1493 (1.0); 7.1443 (0.9); 7.1374 (2.7); 7.1326 (3.8); 7.1249 (7.1); 7.1155 (4.4); 7.1114 (3.3); 7.0994 (0.6); 7.0843 (0.9); 7.0799 (0.9); 7.0681 (2.6); 7.0612 (1.8); 7.0590 (1.9); 7.0514 (0.8); 7.0457 (1.2); 5.2969 (16.0); 4.5130 (1.1); 4.4910 (2.0); 4.4697 (1.3); 4.2004 (1.6); 4.1770 (1.4); 4.1662 (0.7); 4.1298 (0.5); 4.1120 (0.6); 4.0988 (5.1); 4.0839 (8.0); 4.0687 (5.2); 3.1749 (4.9); 3.1664 (2.4); 3.1601 (4.4); 3.1549 (2.4); 3.1509 (2.1); 3.1467 (5.6); 2.8403 (0.8); 2.8316 (1.2); 2.8179 (2.3); 2.8037 (2.5); 2.7964 (1.6); 2.7899 (1.8); 2.7755 (0.5); 2.4356 (1.6); 2.4312 (0.7); 2.4274 (1.0); 2.4206 (3.1); 2.4152 (2.1); 2.4063 (4.8); 2.4001 (1.9); 2.3977 (2.0); 2.3920 (2.9); 2.3818 (0.7); 2.3772 (1.5); 2.0428 (2.0); 1.9959 (0.5); 1.9881 (0.7); 1.9821 (1.1); 1.9743 (1.3); 1.9683 (0.6); 1.9604 (1.0); 1.9554 (1.0); 1.9494 (0.9); 1.9419 (2.1); 1.9292 (1.4); 1.9160 (0.7); 1.9099 (0.8); 1.9070 (0.9); 1.9032 (1.0); 1.8993 (0.7); 1.8956 (0.6); 1.8866 (0.8); 1.8789 (0.6); 1.6776 (0.7); 1.6619 (0.8); 1.6547 (1.2); 1.6444 (1.0); 1.6387 (1.3); 1.6327 (1.2); 1.6290 (1.3); 1.6221 (1.6); 1.6168 (1.9); 1.6071 (1.6); 1.6004 (0.9); 1.2759 (0.7); 1.2581 (1.5); 1.2402 (0.6); 1.1200 (14.5); 1.1033 (14.5); 1.0383 (5.1); 1.0212 (5.0); 0.0080 (0.7); −0.0002 (25.5); −0.0085 (0.8)

I-352: ¹H-NMR(400.0 MHz, CDCl3):

δ=7.5184 (1.0); 7.3871 (5.4); 7.3696 (6.6); 7.3662 (5.3); 7.2596 (175.0); 7.2484 (2.2); 7.2392 (8.2); 7.2358 (11.3); 7.2317 (9.5); 7.2242 (12.1); 7.2206 (9.8); 7.2127 (6.5); 7.2073 (5.3); 7.2027 (4.3); 7.1966 (4.6); 7.1889 (3.1); 7.1797 (2.0); 7.1730 (1.2); 6.9956 (1.0); 5.2976 (13.7); 5.2384 (1.8); 5.2194 (4.3); 5.1984 (4.4); 5.1795 (1.9); 4.2720 (14.9); 4.2598 (8.5); 4.2582 (8.3); 4.2460 (16.0); 4.2156 (3.1); 3.0143 (1.8); 3.0053 (2.0); 2.9926 (1.9); 2.9836 (2.1); 2.9747 (3.3); 2.9656 (3.5); 2.9531 (3.4); 2.9443 (3.5); 2.8853 (2.6); 2.8646 (6.1); 2.8579 (14.2); 2.8516 (6.3); 2.8445 (13.5); 2.8361 (6.2); 2.8301 (15.4); 2.8051 (1.9); 2.7107 (2.4); 2.7016 (2.3); 2.6926 (3.0); 2.6836 (3.0); 2.6790 (3.0); 2.6731 (2.4); 2.6699 (2.8); 2.6640 (2.4); 2.6609 (3.4); 2.6519 (3.2); 2.6414 (2.2); 2.6324 (1.9); 2.2150 (3.0); 2.2090 (2.6); 2.2003 (5.6); 2.1932 (4.6); 2.1862 (8.6); 2.1797 (4.8); 2.1720 (6.0); 2.1641 (2.9); 2.1578 (3.3); 2.0432 (1.8); 1.8886 (5.8); 1.8809 (2.3); 1.8739 (6.6); 1.8704 (6.9); 1.8674 (8.9); 1.8618 (7.9); 1.8487 (9.7); 1.8359 (6.6); 1.8276 (2.6); 1.8172 (4.5); 1.7958 (2.0); 1.6003 (1.9); 1.2762 (0.7); 1.2583 (1.6); 1.2404 (0.6): 0.8818 (0.8): 0.0079 (2.2): −0.0002 (65.6): -00085 (L9)

1H-NMR

I-353: ¹H-NMR(400.0 MHz, CDCl3):

δ=7.2592 (57.8); 7.1859 (3.5); 7.1307 (2.0); 7.1116 (3.0); 7.0451 (2.3); 7.0261 (1.4); 5.2974 (2.5); 5.1990 (0.6); 5.1799 (1.3); 5.1589 (1.4); 5.1400 (0.6); 4.2733 (4.5); 4.2612 (2.5); 4.2595 (2.6); 4.2474 (4.7); 4.2144 (1.2); 4.1918 (1.2); 2.9514 (0.5); 2.9294 (0.6); 2.9210 (0.8); 2.9123 (0.9); 2.8995 (0.9); 2.8910 (0.9); 2.8582 (4.2); 2.8519 (1.9); 2.8444 (2.8); 2.8363 (2.1); 2.8304 (5.2); 2.8111 (1.3); 2.7911 (1.0); 2.7719 (0.7); 2.6987 (0.8); 2.6901 (0.8); 2.6809 (0.9); 2.6722 (0.9); 2.6674 (1.0); 2.6615 (0.7); 2.6586 (0.9); 2.6527 (0.7); 2.6494 (1.0); 2.6405 (1.0); 2.6300 (0.6); 2.6212 (0.6); 2.3233 (16.0); 2.2148 (0.9); 2.2088 (0.8); 2.2001 (1.7); 2.1930 (1.5); 2.1859 (2.6); 2.1796 (1.5); 2.1718 (1.9); 2.1639 (1.0); 2.1577 (1.0); 1.8879 (1.1); 1.8843 (0.9); 1.8714 (2.2); 1.8658 0.6); 1.8614 (2.3); 1.8500 (2.8); 1.8399 (1.3); 1.8365 (0.9); 1.8315 (2.1); 1.8187 (1.4); 1.8101 (0.7); 1.7998 (1.4); 1.7784 (0.6); 1.5793 (1.3); 0.0079 (0.7); −0.0002 (21.9); −0.0085 (0.7)

I-354: ¹H-NMR(400.0 MHz, CDCl3):

δ=7.2593 (60.4); 7.0978 (3.6); 7.0696 (1.8); 7.0505 (3.3); 7.0036 (2.4); 6.9953 (0.6); 6.9847 (1.4); 5.2974 (3.4); 4.7863 (1.1); 4.7642 (1.9); 4.7418 (1.2); 4.2651 (4.3); 4.2532 (2.4); 4.2510 (2.5); 4.2391 (4.4); 4.1587 (1.3); 4.1350 (1.2); 3.0371 (1.2); 3.0180 (1.3); 2.9986 (1.4); 2.9796 (1.5); 2.8663 (4.1); 2.8599 (1.9); 2.8532 (2.6); 2.8446 (1.9); 2.8385 (4.6); 2.4999 (1.0); 2.4766 (1.1); 2.4611 (0.8); 2.4382 (1.0); 2.2938 (16.0); 2.2592 (0.6); 2.2394 (1.0); 2.2198 (1.4); 2.2167 (1.7); 2.2101 (1.0); 2.2008 (2.5); 2.1872 (2.8); 2.1808 (2.0); 2.1738 (1.8); 2.1653 (1.0); 2.1591 (1.0); 1.8896 (1.1); 1.8857 (0.9); 1.8751 (1.9); 1.8676 (1.6); 1.8629 (2.3); 1.8564 (1.6); 1.8488 (1.7); 1.8380 (0.8); 1.8341 (0.9); 1.5933 (0.7); 1.2834 (14.6); 1.2666 (14.3); 0.0080 (0.7); −0.0002 (21.5); −0.0085 (0.6)

I-355: ¹H-NMR(400.0 MHz, CDCl3):

δ=7.5184 (0.6); 7.4512 (2.8); 7.4455 (1.8); 7.4401 (1.4); 7.4361 (3.0); 7.4285 (3.2); 7.2595 (106.3); 7.1787 (0.9); 7.1663 (3.7); 7.1618 (6.0); 7.1528 (11.4); 7.1441 (7.5); 7.1388 (4.3); 7.1264 (0.9); 7.1065 (0.5); 7.0964 (3.6); 7.0879 (3.1); 7.0794 (1.5); 7.0739 (2.0); 6.9954 (0.6); 5.2972 (16.0); 4.8612 (1.1); 4.8479 (1.8); 4.8390 (1.5); 4.8264 (1.9); 4.8122 (1.1); 4.2619 (8.6); 4.2499 (4.8); 4.2480 (4.9); 4.2360 (10.8); 4.2134 (2.2); 2.8568 (8.1); 2.8501 (3.8); 2.8434 (5.8); 2.8348 (3.6); 2.8290 (9.1); 2.8204 (1.8); 2.8048 (2.8); 2.7896 (1.6); 2.7801 (1.7); 2.7629 (2.4); 2.7466 (1.8); 2.7204 (0.8); 2.7049 (0.6); 2.2112 (1.6); 2.2051 (1.5); 2.1961 (3.0); 2.1894 (2.6); 2.1822 (4.8); 2.1759 (2.8); 2.1683 (3.2); 2.1603 (1.7); 2.1540 (1.9); 2.1205 (0.7); 2.1127 (0.8); 2.1080 (0.6); 2.0979 (1.0); 2.0888 (1.6); 2.0769 (1.8); 2.0666 (1.7); 2.0574 (1.4); 2.0432 (2.1); 1.9539 (0.8); 1.9470 (1.0); 1.9332 (1.9); 1.9271 (1.8); 1.9161 (2.2); 1.9101 (1.7); 1.9003 (2.4); 1.8930 (2.2); 1.8857 (4.4); 1.8780 (2.9); 1.8715 (4.5); 1.8641 (3.7); 1.8593 (5.0); 1.8525 (3.6); 1.8450 (4.2); 1.8308 (3.0); 1.8165 (1.4); 1.8043 (1.1); 1.7963 (1.0); 1.7827 (0.6); 1.5933 (1.8); 1.2760 (0.6); 1.2582 (1.2); 0.8818 (0.8); 0.0080 (1.2); −0.0002 (38.7); −0.0085 (1.1)

I-356: ¹H-NMR(400.0 MHz, CDCl3):

δ=7.4385 (0.8); 7.4216 (0.9); 7.4169 (1.0); 7.4064 (2.1); 7.3913 (2.3); 7.3839 (2.4); 7.2596 (65.7); 7.1529 (0.8); 7.1482 (0.9); 7.1403 (2.9); 7.1353 (4.7); 7.1313 (2.7); 7.1264 (9.9); 7.1180 (5.6); 7.1123 (3.7); 7.0999 (0.8); 7.0944 (0.6); 7.0852 (1.1); 7.0810 (1.3); 7.0711 (3.0); 7.0629 (2.7); 7.0540 (1.2); 7.0488 (1.5); 5.2968 (15.0); 4.9284 (0.6); 4.9168 (0.6); 4.9035 (0.6); 4.8921 (0.6); 4.5156 (1.4); 4.4948 (2.4); 4.4739 (1.6); 4.2460 (7.5); 4.2339 (4.4); 4.2323 (4.4); 4.2202 (7.6); 4.1721 (2.0); 4.1492 (1.8); 4.1332 (0.8); 4.1085 (0.6); 2.8610 (6.1); 2.8575 (4.0); 2.8465 (5.0); 2.8333 (7.1); 2.8299 (3.9); 2.8186 (2.0); 2.8046 (4.4); 2.7898 (3.1); 2.7747 (0.6); 2.2080 (1.7); 2.2019 (1.7); 2.1924 (3.2); 2.1863 (2.9); 2.1790 (4.7); 2.1723 (2.8); 2.1661 (3.0); 2.1572 (1.7); 2.1507 (1.7); 2.0429 (0.7); 2.0004 (0.6); 1.9926 (0.9); 1.9860 (1.4); 1.9780 (1.9); 1.9687 (1.6); 1.9641 (1.6); 1.9605 (1.4); 1.9515 (3.0); 1.9455 (1.7); 1.9361 (2.3); 1.9284 (1.2); 1.9165 (0.9); 1.9088 (0.7); 1.8843 (2.0); 1.8805 (1.7); 1.8701 (3.5); 1.8624 (2.9); 1.8578 (4.5); 1.8512 (3.0); 1.8436 (3.5); 1.8331 (1.6); 1.8290 (1.8); 1.8091 (0.6); 1.8018 (0.5); 1.6738 (1.0); 1.6573 (1.2); 1.6516 (1.3); 1.6403 (1.6); 1.6355 (1.5); 1.6313 (1.3); 1.6243 (1.8); 1.6198 (2.1); 1.6154 (2.3); 1.6041 (1.5); 1.2758 (0.5); 1.2627 (0.8); 1.2581 (0.9); 1.1076 (16.0); 1.0909 (16.0); 1.0301 (6.1): 1.0129 (5.9); 0.8818 (1.2); 0.8642 (0.5); 0.0079 (0.8); −0.0002 (24.2); −0.0085 (0.7)

1H-NMR

I-358: ¹H-NMR(400.0 MHz, CDCl3):

δ=7.5182 (1.4); 7.3714 (1.2); 7.3536 (1.5); 7.3418 (2.5); 7.3248 (3.1); 7.3192 (2.8); 7.2593 (248.7); 7.1872 (0.5); 7.1790 (0.6); 7.1735 (1.8); 7.1688 (1.5); 7.1610 (2.7); 7.1560 (3.9); 7.1499 (3.8); 7.1450 (6.6); 7.1389 (4.3); 7.1314 (2.6); 7.1265 (3.7); 7.1046 (2.3); 7.0895 (3.4); 7.0844 (3.7); 7.0683 (1.8); 6.9953 (1.4); 4.9680 (0.6); 4.9580 (0.7); 4.9443 (0.7); 4.9340 (0.6); 4.5448 (1.8); 4.5251 (1.5); 4.4978 (1.4); 4.4402 (0.8); 4.4164 (0.7); 4.2442 (2.7); 4.2368 (6.0); 4.2290 (5.0); 4.2220 (9.0); 4.2144 (3.7); 4.2069 (5.8); 3.5370 (6.4); 3.5295 (3.2); 3.5212 (5.6); 3.5069 (7.2); 2.8959 (0.5); 2.8662 (0.8); 2.8524 (1.4); 2.8307 (2.8); 2.8179 (3.5); 2.8090 (2.6); 2.7739 (2.6); 2.7594 (4.2); 2.7520 (3.0); 2.7441 (6.0); 2.7303 (3.9); 2.7139 (1.4); 2.2064 (0.5); 2.1994 (0.6); 2.1886 (0.7); 2.1819 (0.6); 2.0029 (0.6); 1.9949 (1.0); 1.9891 (1.4); 1.9813 (1.8); 1.9677 (1.8); 1.9626 (1.4); 1.9537 (3.2); 1.9397 (2.0); 1.9180 (0.8); 1.9104 (0.6); 1.8133 (0.6); 1.8002 (0.7); 1.7923 (0.6); 1.6824 (0.6); 1.6597 (1.3); 1.6495 (1.3); 1.6432 (1.5); 1.6377 (1.5); 1.6278 (1.7); 1.6216 (1.2); 1.6120 (1.2); 1.5862 (0.6); 1.5425 (9.8); 1.1068 (16.0); 1.0902 (15.9); 1.0376 (8.5); 1.0204 (8.2); 0.0079 (3.2); −0.0002 (92.1); −0.0085 (3.0)

I-359: ¹H-NMR(400.0 MHz, CDCl3):

δ=7.5188 (0.5); 7.2599 (93.8); 7.1651 (5.2); 7.1433 (2.7); 7.1242 (4.0); 7.0614 (3.7); 7.0422 (2.4); 6.9958 (0.6); 5.2297 (1.1); 5.2198 (1.0); 5.2114 (2.3); 5.2022 (2.0); 5.1934 (1.3); 5.1843 (1.0); 4.3923 (1.2); 4.3868 (1.5); 4.3828 (1.2); 4.3756 (0.8); 4.3640 (0.8); 4.3534 (2.1); 4.3456 (1.3); 4.3407 (1.2); 4.1374 (0.5); 4.1258 (0.9); 4.1216 (1.0); 4.1153 (0.8); 4.1079 (1.0); 4.1034 (1.3); 4.0975 (1.3); 4.0919 (1.6); 4.0737 (0.8); 4.0692 (0.7); 4.0628 (0.9); 3.4892 (1.0); 3.4118 (0.6); 3.3960 (1.3); 3.3812 (2.1); 3.3734 (1.1); 3.3664 (1.7); 3.3512 (2.0); 3.3414 (0.7); 3.1065 (0.9); 3.0931 (1.0); 3.0777 (3.4); 3.0725 (3.5); 3.0592 (1.4); 3.0477 (3.2); 3.0415 (2.3); 3.0151 (0.8); 2.9751 (0.9); 2.9669 (0.7); 2.9534 (0.9); 2.9446 (1.2); 2.9357 (1.4); 2.9278 (1.1); 2.9143 (1.5); 2.9071 (1.0); 2.8509 (0.9); 2.8308 (1.8); 2.8110 (1.5); 2.7914 (1.1); 2.7715 (0.8); 2.7002 (0.6); 2.6911 (0.6); 2.6874 (0.8); 2.6804 (0.9); 2.6786 (0.9); 2.6731 (0.9); 2.6685 (1.5); 2.6594 (1.5); 2.6557 (1.1); 2.6500 (1.4); 2.6469 (1.1); 2.6411 (1.4); 2.6379 (1.2); 2.6287 (1.1); 2.6218 (0.7); 2.6183 (0.8); 2.6092 (0.6); 2.3983 (0.7); 2.3910 (1.0); 2.3872 (1.0); 2.3802 (0.9); 2.3757 (1.0); 2.3717 (0.9); 2.3629 (1.2); 2.3561 (2.5); 2.3333 (16.0); 2.3027 (0.8); 1.8881 (0.7); 1.8691 (1.7); 1.8565 (0.8); 1.8481 (1.9); 1.8375 (1.7); 1.8295 (0.8); 1.8269 (0.8); 1.8164 (1.7); 1.7976 (0.6); 1.7952 (0.6); 0.0079 (1.0); −0.0002 (35.4); −0.0085 (1.4)

I-360: ¹H-NMR(400.0 MHz, CDCl3):

δ=7.5192 (0.5); 7.2603 (96.1); 7.0844 (2.9); 7.0654 (7.8); 7.0225 (4.3); 7.0028 (2.4); 6.9964 (1.2); 4.8195 (2.5); 4.7994 (2.6); 4.3815 (1.2); 4.3758 (1.4); 4.3697 (1.4); 4.3541 (0.8); 4.3469 (1.9); 4.3414 (1.7); 4.3356 (1.6); 4.3291 (1.1); 4.1275 (0.6); 4.1162 (1.3); 4.1052 (1.1); 4.0971 (1.1); 4.0919 (1.3); 4.0864 (1.4); 4.0674 (0.8); 4.0566 (0.8); 3.4898 (7.0); 3.4043 (0.8); 3.3983 (1.2); 3.3899 (1.4); 3.3828 (1.5); 3.3744 (1.3); 3.3685 (1.4); 3.3594 (1.5); 3.3529 (1.7); 3.1149 (0.6); 3.1051 (1.3); 3.0895 (2.0); 3.0830 (3.3); 3.0801 (3.7); 3.0669 (1.5); 3.0574 (4.1); 3.0534 (3.8); 3.0435 (2.0); 3.0338 (1.6); 3.0220 (2.1); 3.0141 (1.6); 3.0025 (1.7); 2.9951 (1.5); 2.5201 (1.8); 2.4975 (2.2); 2.4816 (1.7); 2.4590 (2.0); 2.3972 (1.0); 2.3894 (1.2); 2.3859 (1.0); 2.3819 (1.1); 2.3787 (1.1); 2.3746 (1.0); 2.3577 (2.2); 2.3457 (1.0); 2.3420 (1.1); 2.3310 (0.8); 2.3071 (12.7); 2.2966 (16.0); 2.2815 (2.0); 2.2644 (1.9); 2.2614 (1.9); 2.2482 (1.6); 2.2443 (1.7); 2.2278 (1.1); 2.2087 (0.6); 1.2843 (13.6); 1.2809 (12.3); 1.2675 (13.9); 1.2641 (12.3); 0.0080 (0.9); −0.0002 (35.1); −0.0084 (1.7)

I-361: ¹H-NMR(400.0 MHz, CDCl3):

δ=7.5182 (15.3); 7.4013 (10.0); 7.3847 (11.4); 7.3791 (11.2); 7.3208 (4.7); 7.2900 (8.5); 7.2593 (2808.0); 7.2328 (16.6); 7.2241 (10.8); 7.1789 (16.0); 7.1707 (25.9); 7.1629 (18.5); 7.1547 (12.8); 7.1128 (14.1); 7.0901 (8.3); 6.9953 (16.0); 4.8895 (8.0); 4.3790 (4.9); 4.3493 (7.6); 4.1185 (5.3); 4.0900 (6.6); 4.0558 (4.2); 3.3923 (5.4); 3.3782 (8.2); 3.3621 (5.9); 3.3476 (7.0); 3.0930 (4.9); 3.0715 (12.2); 3.0677 (12.5); 3.0401 (9.5); 2.8351 (4.7); 2.8188 (8.3); 2.8037 (6.2); 2.7944 (5.8); 2.7788 (7.9); 2.7619 (6.4); 2.3520 (7.1); 2.0784 (5.5); 2.0680 (5.2); 2.0046 (3.5); 1.9115 (9.2); 1.8921 (9.1); 1.8560 (8.0); 0.1459 (3.4); 0.0080 (32.5); −0.0002 (976.5); −0.0085 (36.2); −0.1494 (4.5)

1H-NMR

I-362: ¹H-NMR(400.0 MHz, CDCl3):

δ=7.5187 (1.2); 7.3782 (1.3); 7.3585 (1.5); 7.3501 (1.6); 7.3418 (2.7); 7.3348 (2.5); 7.3252 (3.0); 7.3200 (2.4); 7.2597 (200.4); 7.1715 (1.4); 7.1669 (2.1); 7.1590 (1.9); 7.1534 (4.1); 7.1489 (5.1); 7.1433 (5.6); 7.1361 (7.2); 7.1304 (7.2); 7.1125 (3.6); 7.1047 (2.1); 7.0880 (5.1); 7.0830 (4.7); 7.0661 (2.5); 6.9957 (1.1); 4.9619 (1.2); 4.9517 (1.2); 4.5605 (4.1); 4.5404 (4.1); 4.3626 (2.1); 4.3281 (3.2); 4.3171 (2.1); 4.1168 (1.1); 4.1049 (2.0); 4.0942 (1.7); 4.0754 (2.0); 4.0556 (1.1); 4.0449 (1.0); 3.4910 (5.1); 3.3935 (2.1); 3.3784 (2.8); 3.3640 (2.5); 3.3485 (3.1); 3.1017 (1.7); 3.0949 (1.5); 3.0790 (5.5); 3.0741 (5.9); 3.0526 (4.7); 3.0429 (2.8); 3.0201 (1.1); 2.9008 (0.7); 2.8889 (0.7); 2.8726 (1.0); 2.8584 (1.5); 2.8471 (2.4); 2.8329 (3.9); 2.8265 (3.6); 2.8182 (4.8); 2.8044 (3.0); 2.7914 (1.2); 2.7750 (1.3); 2.7314 (0.5); 2.3935 (1.4); 2.3817 (1.5); 2.3746 (1.5); 2.3541 (3.1); 2.3396 (1.3); 2.3274 (0.9); 2.2053 (0.6); 2.1936 (0.6); 2.0078 (0.9); 1.9999 (1.3); 1.9942 (1.9); 1.9864 (2.7); 1.9808 (1.6); 1.9663 (2.6); 1.9548 (3.2); 1.9453 (2.7); 1.9380 (2.0); 1.9284 (1.3); 1.9152 (0.7); 1.8175 (0.5); 1.8039 (0.6); 1.7970 (0.6); 1.6843 (0.8); 1.6625 (1.5); 1.6476 (1.9); 1.6379 (1.7); 1.6313 (1.6); 1.6133 (1.3); 1.5912 (0.5); 1.1095 (15.5); 1.0930 (16.0); 1.0418 (5.3); 1.0350 (3.6); 1.0246 (5.2); 1.0179 (3.3); 0.0079 (2.1); −0.0002 (73.2); −0.0084 (2.8)

I-364: ¹H-NMR(400.0 MHz, CDCl3):

δ=7.5186 (1.3); 7.3559 (6.4); 7.3383 (8.5); 7.2596 (231.2); 7.2531 (17.5); 7.2462 (12.2); 7.2422 (15.4); 7.2385 (11.9); 7.2257 (7.6); 7.2196 (5.0); 7.2091 (4.8); 7.2033 (3.1); 7.1946 (2.7); 7.1871 (1.6); 6.9956 (1.3); 5.2429 (4.9); 5.2247 (9.9); 5.2066 (5.0); 4.4746 (14.5); 4.4617 (9.0); 4.4487 (14.9); 3.4915 (16.0); 3.3597 (13.6); 3.3529 (6.4); 3.3450 (10.5); 3.3368 (6.6); 3.3302 (14.5); 3.0328 (2.1); 3.0236 (2.4); 3.0110 (2.4); 3.0020 (2.5); 2.9930 (3.9); 2.9839 (4.2); 2.9712 (4.1); 2.9624 (4.1); 2.9025 (3.0); 2.8820 (6.4); 2.8619 (5.1); 2.8424 (3.7); 2.8219 (2.3); 2.7124 (2.5); 2.7033 (2.5); 2.6934 (3.4); 2.6847 (3.4); 2.6806 (3.3); 2.6747 (2.7); 2.6716 (3.1); 2.6617 (3.8); 2.6530 (3.5); 2.6429 (2.4); 2.6338 (2.1); 2.3992 (3.3); 2.3927 (3.5); 2.3836 (6.2); 2.3762 (5.5); 2.3698 (10.2); 2.3635 (5.7); 2.3553 (6.7); 2.3469 (3.8); 2.3403 (3.8); 2.0314 (3.9); 2.0177 (6.5); 2.0049 (8.2); 1.9906 (6.0); 1.9762 (3.3); 1.8997 (2.4); 1.8785 (5.0); 1.8679 (2.5); 1.8598 (4.9); 1.8467 (4.8); 1.8386 (2.5); 1.8280 (4.6); 1.8067 (2.0); 0.0077 (2.8); −0.0002 (84.2); −0.0084 (2.8)

I-365: ¹H-NMR(400.0 MHz, CDCl3):

δ=7.2599 (44.5); 7.1511 (3.7); 7.1250 (3.1); 7.0637 (2.3); 7.0448 (1.4); 5.2051 (1.2); 5.1869 (2.3); 5.1687 (1.2); 4.4740 (3.8); 4.4608 (2.4); 4.4481 (3.9); 3.4901 (5.6); 3.3591 (3.5); 3.3523 (1.7); 3.3443 (2.7); 3.3362 (1.8); 3.3296 (3.8); 2.9462 (0.5); 2.9372 (0.8); 2.9286 (0.9); 2.9156 (0.8); 2.9072 (0.9); 2.8475 (0.6); 2.8270 (1.3); 2.8071 (1.0); 2.7878 (0.7); 2.6995 (0.6); 2.6908 (0.6); 2.6809 (0.8); 2.6724 (0.9); 2.6681 (0.8); 2.6620 (0.7); 2.6592 (0.8); 2.6532 (0.6); 2.6493 (1.0); 2.6407 (0.9); 2.6305 (0.6); 2.6215 (0.5); 2.3986 (0.8); 2.3921 (0.8); 2.3829 (1.5); 2.3755 (1.4); 2.3691 (2.7); 2.3629 (1.5); 2.3547 (1.8); 2.3463 (1.1); 2.3319 (16.0); 2.0293 (1.0); 2.0157 (1.7); 2.0026 (2.2); 1.9885 (1.7); 1.9744 (1.0); 1.8798 (0.6); 1.8588 (1.4); 1.8482 (0.7); 1.8398 (1.3); 1.8272 (1.3); 1.8186 (0.7); 1.8082 (1.2); 1.7868 (0.6); −0.0002 (16.0); −0.0082 (0.8)

I-366: ¹H-NMR(400.0 MHz, CDCl3):

δ=7.2596 (56.2); 7.0861 (1.8); 7.0655 (5.7); 7.0279 (2.5); 7.0073 (1.4); 4.7896 (1.8); 4.7692 (1.9); 4.4627 (3.9); 4.4511 (2.5); 4.4483 (2.5); 4.4367 (4.2); 3.4894 (6.5); 3.3680 (3.6); 3.3612 (1.8); 3.3528 (2.7); 3.3452 (1.9); 3.3385 (4.0); 3.0553 (1.2); 3.0362 (1.3); 3.0166 (1.5); 2.9974 (1.5); 2.5158 (1.0); 2.4936 (1.2); 2.4774 (0.9); 2.4557 (1.1); 2.4003 (0.8); 2.3936 (0.9); 2.3841 (1.6); 2.3773 (1.5); 2.3708 (2.7); 2.3644 (1.6); 2.3562 (1.8); 2.3480 (1.2); 2.3413 (1.1); 2.3070 (16.0); 2.2854 (0.9); 2.2656 (1.0); 2.2461 (1.3); 2.2267 (0.9); 2.0303 (1.0); 2.0162 (1.6); 2.0039 (2.2); 1.9907 (1.6); 1.9754 (0.9); 1.2823 (13.8); 1.2655 (13.7); 0.0080 (0.6); −0.0002 (20.7); −0.0085 (1.0)

1H-NMR

I-367: ¹H-NMR(400.0 MHz, CDCl3):

δ=7.5188 (0.6); 7.4015 (2.3); 7.3848 (3.0); 7.3790 (2.8); 7.2599 (105.8); 7.2501 (1.3); 7.2485 (1.1); 7.2341 (0.6); 7.2023 (0.8); 7.1974 (1.0); 7.1838 (2.9); 7.1793 (3.2); 7.1745 (3.0); 7.1682 (7.6); 7.1613 (4.2); 7.1563 (3.1); 7.1512 (3.3); 7.1381 (1.2); 7.1332 (0.9); 7.1243 (0.7); 7.1112 (3.1); 7.1051 (3.0); 7.0959 (1.6); 7.0930 (1.8); 7.0894 (2.0); 6.9959 (0.7); 4.8739 (1.8); 4.8609 (2.8); 4.8466 (1.8); 4.4652 (6.0); 4.4514 (3.7); 4.4392 (6.4); 3.4907 (16.0); 3.3599 (5.7); 3.3531 (2.8); 3.3452 (4.2); 3.3369 (3.0); 3.3303 (6.4); 2.8605 (0.8); 2.8455 (0.6); 2.8340 (1.2); 2.8181 (2.3); 2.8033 (1.4); 2.7923 (1.5); 2.7756 (2.2); 2.7591 (1.6); 2.7343 (0.8); 2.7178 (0.7); 2.3960 (1.3); 2.3896 (1.4); 2.3802 (2.4); 2.3729 (2.3); 2.3665 (4.2); 2.3602 (2.6); 2.3524 (2.9); 2.3436 (1.9); 2.3370 (1.8); 2.1239 (0.6); 2.1161 (0.7); 2.1112 (0.6); 2.1022 (0.9); 2.0924 (1.4); 2.0818 (1.6); 2.0704 (1.6); 2.0606 (1.3); 2.0485 (1.0); 2.0299 (1.6); 2.0162 (2.8); 2.0034 (3.5); 1.9887 (2.8); 1.9747 (1.7); 1.9537 (0.9); 1.9462 (1.0); 1.9329 (1.7); 1.9261 (1.7); 1.9161 (2.0); 1.9094 (1.7); 1.9058 (1.7); 1.9003 (2.2); 1.8924 (2.0); 1.8849 (2.4); 1.8774 (1.9); 1.8703 (1.6); 1.8664 (2.0); 1.8586 (1.6); 1.8518 (2.1); 1.8435 (2.0); 1.8355 (1.4); 1.8271 (1.5); 1.8154 (1.1); 1.8069 (1.1); 1.7934 (0.7); 0.0079 (1.0); −0.0002 (39.3); −0.0085 (1.7)

I-368: ¹H-NMR(400.0 MHz, CDCl3):

δ=7.5186 (0.5); 7.3891 (1.0); 7.3688 (1.2); 7.3549 (2.5); 7.3383 (3.0); 7.3326 (2.9); 7.2596 (87.5); 7.1778 (0.6); 7.1710 (1.3); 7.1664 (1.3); 7.1598 (2.7); 7.1550 (3.2); 7.1489 (3.7); 7.1436 (7.7); 7.1377 (4.0); 7.1313 (2.8); 7.1267 (3.6); 7.1130 (1.1); 7.1033 (1.8); 7.0889 (3.4); 7.0834 (3.6); 7.0673 (1.9); 6.9956 (0.5); 4.9316 (1.2); 4.9207 (1.2); 4.5210 (3.1); 4.5016 (3.1); 4.4528 (2.5); 4.4459 (6.5); 4.4322 (4.8); 4.4272 (4.4); 4.4201 (6.2); 3.4901 (3.0); 3.3656 (5.2); 3.3593 (4.5); 3.3512 (5.1); 3.3434 (4.0); 3.3362 (6.6); 3.3303 (2.8); 2.8692 (0.6); 2.8542 (1.0); 2.8410 (1.8); 2.8299 (2.9); 2.8171 (3.9); 2.8066 (2.8); 2.7900 (0.9); 2.7686 (0.7); 2.3935 (1.6); 2.3871 (1.8); 2.3780 (3.3); 2.3705 (3.0); 2.3642 (5.5); 2.3580 (3.2); 2.3499 (3.8); 2.3412 (2.1); 2.3348 (2.0); 2.2142 (0.6); 2.2033 (0.6); 2.1965 (0.6); 2.0257 (1.9); 2.0123 (3.5); 1.9998 (4.9); 1.9877 (4.6); 1.9805 (4.4); 1.9741 (2.9); 1.9639 (3.3); 1.9528 (2.6); 1.9467 (2.8); 1.9391 (2.1); 1.9315 (1.4); 1.9192 (0.8); 1.8446 (0.5); 1.8367 (0.5); 1.8245 (0.5); 1.8103 (0.6); 1.8028 (0.6); 1.6837 (0.8); 1.6686 (1.1); 1.6606 (1.5); 1.6484 (1.6); 1.6446 (1.7); 1.6390 (1.3); 1.6343 (1.4); 1.6284 (1.5); 1.6236 (1.5); 1.6126 (1.2); 1.6039 (0.8); 1.5883 (0.7); 1.2838 (0.8); 1.2668 (0.7); 1.1022 (15.9); 1.0857 (16.0); 1.0298 (6.6); 1.0126 (6.5); 0.0076 (0.8);-0.0002 (32.2); −0.0083 (1.6)

I-369: ¹H-NMR(400.0 MHz, CDCl3):

δ=7.5186 (3.2); 7.3671 (8.9); 7.3498 (11.6); 7.2916 (1.0); 7.2710 (3.4); 7.2702 (3.6); 7.2598 (570.7); 7.2547 (30.3); 7.2532 (30.6); 7.2515 (29.2); 7.2445 (24.4); 7.2394 (27.7); 7.2358 (23.3); 7.2241 (15.2); 7.2172 (11.3); 7.2068 (9.5); 7.2015 (6.9); 7.1921 (5.1); 7.1847 (3.4); 6.9957 (3.2); 5.2264 (5.5); 5.2084 (11.4); 5.1904 (6.1); 4.5918 (5.0); 4.5620 (8.4); 4.5584 (7.9); 4.5286 (7.8); 4.3351 (5.3); 4.3230 (5.2); 4.2989 (4.2); 4.2913 (4.1); 4.2868 (3.9); 3.5122 (5.9); 3.5006 (5.7); 3.4901 (9.0); 3.4807 (5.8); 3.4657 (5.7); 3.4617 (5.0); 3.0336 (3.1); 3.0248 (3.5); 3.0121 (3.5); 3.0032 (3.9); 2.9940 (5.7); 2.9849 (6.2); 2.9726 (6.1); 2.9633 (6.3); 2.9044 (5.8); 2.8834 (16.0); 2.8641 (10.4); 2.8516 (9.2); 2.8457 (9.8); 2.8219 (5.9); 2.7950 (11.9); 2.7632 (13.3); 2.7285 (5.4); 2.7040 (4.0); 2.6947 (3.9); 2.6845 (5.9); 2.6751 (6.0); 2.6725 (6.1); 2.6664 (4.5); 2.6630 (5.1); 2.6526 (6.8); 2.6432 (5.9); 2.6344 (3.9); 2.6250 (3.4); 2.1707 (8.2); 2.1617 (9.0); 2.1540 (8.2); 2.1397 (9.3); 2.1288 (9.9); 2.1236 (9.8); 1.9068 (2.8); 1.9009 (2.0); 1.8857 (5.7); 1.8799 (4.0); 1.8749 (3.5); 1.8673 (7.3); 1.8645 (6.2); 1.8618 (6.7); 1.8568 (6.8); 1.8540 (6.7); 1.8465 (5.4); 1.8403 (4.3); 1.8353 (8.0); 1.8325 (8.3); 1.8268 (9.0); 1.8142 (4.6); 1.8085 (3.2); 1.7971 (6.1); 1.7712 (2.1); 0.1461 (0.7); 0.0079 (6.4); −0.0002 (215.5); −0.0085 (8.7);- 0.1496 (0.8)

I-370: ¹H-NMR(400.0 MHz, CDCl3):

δ=7.2597 (88.3); 7.1669 (3.2); 7.1427 (1.8); 7.1237 (2.8); 7.0607 (2.3); 7.0410 (1.5); 6.9957 (0.5); 5.1862 (1.0); 5.1681 (2.2); 5.1504 (I. I); 4.5952 (0.9); 4.5625 (1.4); 4.5591 (1.4); 4.5294 (1.4); 4.3371 (0.9); 4.3247 (0.9); 4.3012 (0.7); 4.2887 (0.7); 3.5103 (1.0); 3.4986 (1.0); 3.4941 (0.9); 3.4896 (1.5); 3.4790 (0.9); 3.4635 (1.0); 3.4596 (0.8); 2.9391 (0.8); 2.9300 (0.9); 2.9168 (1.0); 2.9086 (1.0); 2.8831 (1.0); 2.8517 (1.8); 2.8294 (1.6); 2.8097 (1.4); 2.7930 (2.3); 2.7593 (2.0); 2.7276 (0.8); 2.6905 (0.6); 2.6817 (0.6); 2.6715 (1.0); 2.6622 (0.9); 2.6595 (0.9); 2.6502 (0.8); 2.6398 (1.1); 2.6305 (1.0); 2.6215 (0.6); 2.6123 (0.5); 2.3316 (16.0); 2.1694 (1.4); 2.1601 (1.5); 2.1520 (1.4); 2.1381 (1.6); 2.1277 (1.7); 2.1218 (1.6); 1.8657 (1.1); 1.8607 (1.0); 1.8553 (0.9); 1.8473 (1.0); 1.8425 (1.0); 1.8342 (1.3); 1.8289 (1.5); 1.8261 (1.5); 1.8156 (1.1); 1.8111 (1.0); 1.7944 (1.3); 0.0079 (0.9); −0.0002 (32.5); −0.0085 (1.5)

1H-NMR

I-371: ¹H-NMR(400.0 MHz, CDCl3):

δ=7.2595 (73.1); 7.0850 (1.6); 7.0653 (5.0); 7.0246 (2.7); 7.0054 (1.3); 4.7683 (2.2); 4.7482 (2.2); 4.5954 (0.7); 4.5604 (1.4); 4.5265 (1.0); 4.3303 (0.9); 4.3215 (0.9); 4.2944 (0.7); 4.2855 (0.7); 3.5234 (1.0); 3.5117 (0.9); 3.4920 (2.0); 3.4766 (0.9); 3.0592 (0.8); 3.0397 (0.9); 3.0203 (1.0); 3.0016 (1.0); 2.8860 (0.8); 2.8555 (1.3); 2.8240 (2.5); 2.7913 (2.0); 2.7582 (0.7); 2.5159 (0.9); 2.4937 (1.1); 2.4774 (0.8); 2.4555 (1.0); 2.3068 (16.0); 2.2677 (0.6); 2.2514 (0.8); 2.2365 (0.6); 2.1776 (1.6); 2.1615 (1.3); 2.1462 (1.7); 2.1314 (1.9); 1.8416 (1.0); 1.8122 (0.8); 1.2845 (6.4); 1.2730 (6.6); 1.2677 (6.8); 1.2562 (6.0); 0.0079 (0.8); −0.0002 (26.6); −0.0080 (1.0)

I-372: ¹H-NMR(400.0 MHz, CDCl3):

δ=7.5186 (2.3); 7.4167 (3.5); 7.4071 (5.8); 7.4002 (6.6); 7.3917 (6.9); 7.3844 (5.7); 7.3725 (0.8); 7.2597 (408.8); 7.2014 (1.2); 7.1964 (2.6); 7.1833 (8.2); 7.1781 (11.9); 7.1689 (20.2); 7.1608 (13.8); 7.1543 (8.9); 7.1412 (1.8); 7.1215 (1.1); 7.1107 (9.5); 7.1043 (7.5); 7.0889 (5.6); 6.9956 (2.3); 4.8543 (5.6); 4.8407 (9.4); 4.8269 (5.4); 4.5822 (3.8); 4.5524 (6.5); 4.5184 (5.3); 4.3275 (4.2); 4.3161 (3.9); 4.2914 (3.1); 4.2795 (2.9); 3.5118 (4.8); 3.5005 (4.6); 3.4908 (10.3); 3.4813 (4.9); 3.4771 (4.2); 3.4660 (4.1); 3.4623 (3.8); 2.9103 (1.7); 2.8754 (5.4); 2.8601 (4.2); 2.8480 (7.9); 2.8330 (6.2); 2.8171 (12.1); 2.8056 (14.9); 2.7927 (5.3); 2.7742 (16.0); 2.7593 (5.4); 2.7398 (4.7); 2.7174 (2.0); 2.1699 (7.2); 2.1612 (6.1); 2.1526 (6.3); 2.1382 (7.6); 2.1231 (8.7); 2.1111 (4.1); 2.0980 (2.4); 2.0879 (3.7); 2.0776 (4.5); 2.0662 (4.3); 2.0562 (4.1); 2.0443 (3.1); 2.0356 (1.4); 1.9589 (2.2); 1.9397 (4.7); 1.9243 (5.4); 1.9120 (5.4); 1.9072 (5.4); 1.8914 (6.2); 1.8700 (5.4); 1.8544 (4.9); 1.8354 (7.2); 1.8303 (7.1); 1.8055 (5.1); 1.7734 (1.5); 0.1461 (0.6); 0.0079 (4.9); −0.0002 (150.2); −0.0085 (4.7); −0.1494 (0.6)

I-373: ¹H-NMR(400.0 MHz, CDCl3):

δ=7.5185 (2.4); 7.3952 (0.8); 7.3770 (1.4); 7.3632 (2.8); 7.3468 (3.8); 7.3413 (3.6); 7.3344 (2.8); 7.3276 (2.9); 7.3092 (0.6); 7.2934 (0.7); 7.2596 (447.6); 7.2196 (1.6); 7.1831 (1.2); 7.1696 (2.2); 7.1649 (2.2); 7.1589 (4.5); 7.1526 (6.4); 7.1444 (10.7); 7.1365 (7.2); 7.1292 (5.6); 7.1026 (2.5); 7.0882 (5.6); 7.0827 (5.2); 7.0662 (3.2); 6.9956 (2.5); 4.9090 (1.6); 4.8983 (1.7); 4.5715 (3.0); 4.5414 (3.9); 4.5357 (4.2); 4.5072 (9.2); 4.4880 (5.4); 4.3126 (2.8); 4.3032 (2.8); 4.2767 (2.3); 4.2680 (2.3); 3.5125 (2.8); 3.5024 (2.9); 3.4912 (4.1); 3.4825 (2.9); 3.4680 (2.7); 2.9095 (0.9); 2.8779 (3.0); 2.8556 (2.1); 2.8442 (5.2); 2.8177 (12.1); 2.8021 (6.0); 2.7900 (7.0); 2.7563 (2.9); 2.4579 (0.7); 2.1688 (5.5); 2.1529 (4.5); 2.1378 (5.4); 2.1228 (6.3); 1.9932 (2.4); 1.9856 (3.8); 1.9707 (6.0); 1.9546 (5.2); 1.9438 (3.3); 1.9351 (2.8); 1.9271 (1.7); 1.8701 (1.2); 1.8396 (2.9); 1.8099 (2.9); 1.6968 (0.5); 1.6825 (1.2); 1.6605 (2.2); 1.6480 (2.4); 1.6238 (2.2); 1.6106 (1.6); 1.5873 (0.9); 1.1048 (14.0); 1.0954 (14.7); 1.0883 (16.0); 1.0789 (14.7); 1.0358 (5.0); 1.0289 (5.4); 1.0186 (5.0); 1.0118 (5.3); 0.0079 (3.8); −0.0002 (159.9); −0.0085 (7.6); −0.1498 (0.6)

I-376: ¹H-NMR(400.0 MHz, d₆-DMSO):

δ=7.0494 (2.9); 6.9321 (1.5); 6.9270 (1.4); 6.9115 (1.7); 6.9064 (1.6); 6.6436 (3.7); 6.6229 (3.2); 6.2863 (2.0); 6.2645 (2.0); 4.6573 (0.5); 4.6442 (1.1); 4.6324 (0.8); 4.6225 (1.1); 4.6095 (0.5); 4.2103 (0.5); 4.2020 (0.6); 4.1890 (0.5); 4.1819 (1.3); 4.1743 (1.1); 4.1619 (1.1); 4.1536 (1.1); 4.1494 (1.0); 4.1392 (1.1); 4.1330 (I. I); 4.1232 (1.1); 4.1117 (0.4); 4.1050 (0.5); 4.0966 (0.4); 4.0133 (2.2); 3.9987 (3.9); 3.9838 (2.3); 3.9019 (8.1); 3.3256 (156.7); 3.2892 (0.4); 3.2669 (0.3); 3.2283 (2.4); 3.2147 (2.4); 3.2009 (2.5); 2.6748 (0.5); 2.6704 (0.6); 2.6660 (0.5); 2.5407 (0.4); 2.5236 (1.8); 2.5099 (46.2); 2.5059 (91.5); 2.5015 (118.5); 2.4970 (85.6); 2.3328 (0.6); 2.3280 (0.7); 2.3239 (0.6); 2.2859 (0.7); 2.2656 (1.8); 2.2582 (2.2); 2.2452 (1.5); 2.2299 (0.6); 2.1791 (16.0); 2.0368 (0.4); 2.0245 (0.5); 2.0114 (0.7); 2.0021 (0.9); 1.9904 (1.0); 1.9792 (0.8); 1.9688 (0.9); 1.9599 (0.7); 1.9531 (1.0); 1.9451 (1.0); 1.9382 (0.7); 1.9305 (0.6); 1.9183 (0.5); 1.9103 (0.4);-0.0002 (0.5)

I-377: ¹H-NMR(400.0 MHz, d₆-DMSO):

δ=7.0683 (2.9); 7.0646 (2.9); 6.9419 (1.5); 6.9371 (1.4); 6.9212 (1.7); 6.9163 (1.6); 6.6534 (3.7); 6.6327 (3.2); 6.2863 (2.0); 6.2646 (2.0); 4.6590 (0.5); 4.6461 (1.1); 4.6351 (0.9); 4.6245 (1.1); 4.6113 (0.5); 4.2169 (0.4); 4.2094 (0.5); 4.1890 (1.3); 4.1815 (1.2); 4.1656 (3.3); 4.1549 (2.9); 4.1407 (3.7); 4.1316 (1.4); 4.1193 (0.5); 4.1129 (0.6); 4.1041 (0.4); 3.9101 (8.8); 3.3341 (154.4); 3.2752 (0.4); 2.8992 (2.3); 2.8930 (1.8); 2.8858 (2.2); 2.8777 (1.9); 2.8722 (2.4); 2.6829 (0.5); 2.6787 (0.6); 2.5489 (0.5); 2.5140 (89.8); 2.5096 (115.1); 2.5053 (83.0); 2.3409 (0.6); 2.3364 (0.7); 2.3320 (0.5); 2.2737 (0.4); 2.1864 (16.0); 2.0676 (2.1); 2.0590 (1.8); 2.0383 (1.0); 2.0243 (0.8); 2.0144 (0.9); 2.0029 (1.0); 1.9920 (0.8); 1.9817 (0.6); 1.9759 (0.6); 1.9668 (0.7); 1.9603 (1.0); 1.9523 (1.0); 1.9458 (0.7); 1.9385 (0.7): 1.9255 (0.5); 1.9174 (0.4): 1.9111 (0.3); 1.7901 (0.9); 1.7788 (1.6); 1.7671 (2.0); 1.7557 (1.5)

1H-NMR

I-378: ¹H-NMR(400.0 MHz, CDCl3):

δ=7.2596 (47.0); 7.0884 (2.5); 7.0042 (1.3); 6.9988 (1.2); 6.9833 (1.5); 6.9781 (1.4); 6.7482 (3.6); 6.7274 (3.0); 4.8084 (1.0); 4.2507 (3.4); 4.2358 (5.0); 4.2207 (3.9); 4.2091 (1.0); 4.2020 (1.2); 4.1945 (1.5); 4.1792 (1.2); 4.1720 (1.4); 4.1663 (0.5); 4.1514 (0.6); 3.5419 (2.9); 3.5341 (1.3); 3.5267 (2.2); 3.5192 (1.3); 3.5117 (3.2); 3.4894 (7.0); 2.7786 (0.8); 2.7708 (0.5); 2.7633 (1.6); 2.7568 (1.1); 2.7486 (2.6); 2.7406 (1.1); 2.7334 (1.4); 2.7264 (0.5); 2.7184 (0.7); 2.2498 (16.0); 2.2354 (0.5); 2.2221 (0.8); 2.2127 (0.9); 2.1998 (1.0); 2.1878 (0.8); 2.1782 (0.6); 2.1689 (0.6); 2.1612 (0.8); 2.1556 (1.0); 2.1483 (1.1); 2.1423 (0.7); 2.1345 (0.8); 0.0079 (0.6); −0.0002 (16.6); −0.0083 (0.6)

I-379: ¹H-NMR(400.0 MHz, CDCl3):

δ=7.5182 (1.4); 7.2594 (246.2); 7.0985 (3.7); 7.0048 (2.1); 6.9997 (1.9); 6.9956 (1.9); 6.9838 (2.2); 6.9787 (2.1); 6.7506 (4.3); 6.7298 (3.6); 4.8270 (1.4); 4.8150 (2.9); 4.8027 (1.4); 4.3904 (1.1); 4.3850 (1.1); 4.3551 (1.6); 4.3440 (1.2); 4.2535 (0.8); 4.2336 (1.4); 4.2260 (1.9); 4.2207 (1.6); 4.2120 (2.4); 4.2050 (2.1); 4.1904 (1.4); 4.1833 (2.0); 4.1624 (0.6); 4.1549 (0.6); 4.1363 (0.7); 4.1248 (1.0); 4.1067 (1.0); 4.0955 (1.3); 4.0725 (0.7); 4.0621 (0.7); 3.4456 (0.5); 3.4082 (1.1); 3.3932 (1.8); 3.3783 (1.5); 3.3624 (1.6); 3.1047 (0.9); 3.0927 (1.2); 3.0784 (2.7); 3.0741 (2.3); 3.0552 (2.0); 3.0432 (1.3); 2.4083 (0.8); 2.4008 (0.8); 2.3677 (1.6); 2.2788 (2.1); 2.2528 (16.0); 2.2475 (11.2); 2.2258 (1.0); 2.2166 (1.2); 2.2036 (1.3); 2.1927 (1.1); 2.1818 (0.7); 2.1768 (0.8); 2.1683 (1.1); 2.1627 (1.5); 2.1555 (1.6); 2.1421 (1.1); 2.1282 (0.7); 2.1206 (0.7); 0.0080 (3.0); −0.0002 (91.6); −0.0084 (2.7)

I-380: ¹H-NMR(400.0 MHz, CDCl3):

δ=7.5189 (0.5); 7.2601 (95.3); 7.1165 (3.2); 7.0024 (2.0); 6.9963 (2.3); 6.9814 (2.2); 6.9765 (2.0); 6.7489 (4.2); 6.7281 (3.6); 4.7778 (1.4); 4.7662 (2.8); 4.7551 (1.5); 4.5915 (0.7); 4.5842 (0.8); 4.5564 (1.6); 4.5499 (1.1); 4.5259 (1.0); 4.5207 (1.1); 4.3287 (1.0); 4.3209 (1.0); 4.3165 (1.0); 4.2978 (0.8); 4.2924 (0.8); 4.2848 (0.8); 4.2803 (0.8); 4.2648 (0.6); 4.2531 (0.9); 4.2362 (2.0); 4.2269 (2.3); 4.2130 (2.2); 4.2084 (1.8); 4.2043 (1.3); 4.1936 (1.2); 4.1861 (1.5); 4.1820 (1.2); 4.1659 (0.5); 4.1614 (0.5); 4.1580 (0.5); 3.5153 (1.1); 3.5041 (1.1); 3.4904 (7.5); 3.4697 (1.1); 3.4448 (0.7); 3.4014 (0.7); 2.8819 (1.1); 2.8510 (1.5); 2.8343 (0.6); 2.8174 (0.8); 2.7955 (1.8); 2.7607 (2.0); 2.7292 (1.0); 2.2774 (1.9); 2.2502 (16.0); 2.2223 (0.8); 2.2127 (1.1); 2.2005 (1.2); 2.1890 (1.4); 2.1670 (2.8); 2.1597 (3.0); 2.1461 (2.7); 2.1336 (2.7); 2.1123 (1.1); 1.8279 (0.8); 1.8193 (0.8); 1.7897 (0.8); 0.0079 (1.2); −0.0002 (35.7); −0.0085 (1.3)

I-381: ¹H-NMR(400.0 MHz, CDCl3):

δ=7.2605 (33.2); 7.1050 (2.3); 7.1004 (2.5); 7.0019 (1.3); 6.9966 (1.4); 6.9811 (1.5); 6.9759 (1.4); 6.7479 (3.5); 6.7270 (3.0); 4.8029 (0.9); 4.7908 (1.9); 4.7788 (0.9); 4.4694 (3.0); 4.4566 (1.9); 4.4435 (3.0); 4.2346 (1.1); 4.2253 (1.1); 4.2205 (1.3); 4.2078 (1.4); 4.2000 (1.5); 4.1849 (1.2); 4.1777 (1.4); 4.1718 (0.5); 4.1570 (0.6); 3.4917 (6.5); 3.3611 (2.7); 3.3543 (1.4); 3.3464 (2.1); 3.3383 (1.4); 3.3316 (2.9); 2.4014 (0.7); 2.3948 (0.7); 2.3856 (1.3); 2.3783 (1.2); 2.3719 (2.1); 2.3656 (1.2); 2.3575 (1.4); 2.3489 (0.8); 2.3425 (0.8); 2.2478 (16.0); 2.2261 (0.8); 2.2164 (0.9); 2.2037 (1.0); 2.1920 (0.8); 2.1822 (0.5); 2.1693 (0.6); 2.1617 (0.7); 2.1557 (0.9); 2.1486 (1.1); 2.1426 (0.7); 2.1348 (0.8); 2.0317 (0.8); 2.0180 (1.4); 2.0047 (1.8); 1.9906 (1.3); 1.9767 (0.7); −0.0002 (12.3)

I-402: ¹H-NMR(400.0 MHz, CDCl3):

δ=7.5181 (0.6); 7.2592 (123.9); 6.9952 (0.8); 6.9833 (10.5); 6.9805 (9.4); 5.2979 (7.4); 4.8148 (0.6); 4.8024 (0.9); 4.7816 (1.0); 4.7664 (0.6); 4.2678 (4.2); 4.2539 (2.4); 4.2419 (4.3); 4.2190 (1.4); 4.1974 (1.2); 2.8583 (4.0); 2.8522 (1.7); 2.8446 (2.7); 2.8367 (1.7); 2.8305 (4.3); 2.7985 (0.5); 2.7714 (0.7); 2.7562 (1.3); 2.7418 (0.8); 2.7305 (0.8); 2.7137 (1.1); 2.6974 (0.8); 2.3434 (0.6); 2.2744 (16.0); 2.2130 (0.8); 2.2069 (0.8); 2.1977 (1.5); 2.1914 (1.4); 2.1841 (2.4); 2.1777 (1.4); 2.1703 (1.6); 2.1622 (0.9); 2.1557 (1.0); 2.0730 (0.8); 2.0613 (0.9); 2.0511 (0.8); 2.0431 (1.0); 2.0289 (0.6); 1.9289 (0.6); 1.9145 (1.0); 1.9089 (0.9); 1.8948 (1.2); 1.8837 (2.1); 1.8736 (2.4); 1.8665 (2.4); 1.8615 (3.0); 1.8472 (2.2); 1.8323 (1.3); 1.8240 (0.7); 1.8172 (0.7); 1.8095 (0.8); 1.7933 (0.7); 1.7734 (0.6); 1.5695 (0.6); 1.2584 (0.6); 0.0079 (1.2); −0.0002 (42.5); −0.0085 (1.4)

I-402: ¹H-NMR(400.0 MHz, d₆-DMSO):

δ=7.1232 (3.3); 6.9393 (8.6); 6.0237 (1.8); 6.0008 (1.9); 5.7526 (16.0); 4.6085 (0.6); 4.5965 (0.9); 4.1460 (2.5); 4.1333 (1.8); 4.1205 (2.6); 3.3075 (36.4); 2.8888 (2.3); 2.8825 (1.6); 2.8752 (2.0); 2.8674 (1.6); 2.8613 (2.5); 2.6849 (0.6); 2.6734 (1.0); 2.6689 (1.4); 2.6645 (1.2); 2.6531 (1.0); 2.6349 (1.1); 2.6202 (0.6); 2.5225 (1.7); 2.5178 (2.3); 2.5091 (28.3); 2.5045 (59.8); 2.4999 (82.8); 2.4953 (57.1); 2.4908 (26.0); 2.3266 (0.6); 2.2099 (14.8); 2.0857 (0.6); 2.0717 (1.3); 2.0647 (1.3); 2.0574 (1.8); 2.0507 (1.4); 2.0440 (1.3); 2.0362 (0.8); 2.0296 (0.6); 1.9074 (0.6); 1.8985 (0.9); 1.8839 (1.6); 1.8724 (1.4); 1.8582 (0.8); 1.7816 (0.8); 1.7697 (1.4); 1.7560 (1.9); 1.7275 (1.3); 1.7085 (0.8); 1.7026 (1.0); 1.6844 (0.8); 1.6703 (0.8); 1.6496 (0.6); 0.0080 (0.6); −0.0002 (19.0); −0.0085 (0.6)

### B. FORMULATION EXAMPLES

a) A dusting product is obtained by mixing 10 parts by weight of a compound of the formula (I) and/or salts thereof and 90 parts by weight of talc as an inert substance and comminuting the mixture in a hammer mill.
b) A readily water-dispersible, wettable powder is obtained by mixing 25 parts by weight of a compound of the formula (I) and/or salts thereof, 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill.
c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277° C.), and grinding the mixture in a friction ball mill to a fineness of below 5 microns.
d) An emulsifiable concentrate is obtained from 15 parts by weight of a compound of the formula (I) and/or salts thereof, 75 parts by weight of cyclohexanone as a solvent and 10 parts by weight of ethoxylated nonylphenol as an emulsifier.
e) Water-dispersible granules are obtained by mixing
   75 parts by weight of a compound of the formula (I) and/or salts thereof,
   10 parts by weight of calcium lignosulfonate,
   5 parts by weight of sodium lauryl sulfate,
   3 parts by weight of polyvinyl alcohol and
   7 parts by weight of kaolin,
grinding the mixture in a pinned-disk mill, and granulating the powder in a fluidized bed by spray application of water as a granulating liquid.
f) Water-dispersible granules are also obtained by homogenizing and precomminuting, in a colloid mill,
   25 parts by weight of a compound of the formula (I) and/or salts thereof,
   5 parts by weight of sodium 2,2′-dinaphthylmethane-6,6′-disulfonate
   2 parts by weight of sodium oleoylmethyltaurate,
   1 part by weight of polyvinyl alcohol,
   17 parts by weight of calcium carbonate and
   50 parts by weight of water,
then grinding the mixture in a bead mill and atomizing and drying the resulting suspension in a spray tower by means of a one-phase nozzle.

### C. BIOLOGICAL EXAMPLES

C.1 Pre-Emergence Herbicidal Action and Crop Plant Compatibility

Seeds of monocotyledonous and dicotyledonous weed plants and crop plants are placed in plastic or organic planting pots and covered with soil. The compounds of the invention, formulated in the form of wettable powders (WP) or as emulsion concentrates (EC), are then applied onto the surface of the covering soil as aqueous suspension or emulsion with addition of 0.5% additive at a water application rate of 6001/ha (converted). After the treatment, the pots are placed in a greenhouse and kept under good growth conditions for the trial plants. After about 3 weeks, the effect of the preparations is scored visually in comparison with untreated controls as percentages. For example, 100% activity=the plants have died, 0% activity=like control plants.

In the tables below, the following abbreviations are used:

Undesired plants/weeds:

ABUTH: Abutilon theophrasti ALOMY: Alopecurus myosuroides
AMARE: Amaranthus retroflexus AVEFA: Avena fatua
CYPES: Cyperus esculentus ECHCG: Echinochloa crus-galli
LOLMU: Lolium multiflorum MATIN: Matricaria inodora
PHBPU: Ipomoea purpurea POLCO: Polygonum convolvulus
SETVI: Setaria viridis STEME: Stellaria media
VERPE: Veronica persica VTOTR: Viola tricolor

TABLE B1
Pre-emergence herbicidal efficacy

| | | Herbicidal efficacy against [%] | | | | | | | | | |
|---|---|---|---|---|---|---|---|---|---|---|---|
| Example | Application | | | | | | | | | | |
| No.: | rate [g/ha] | ECHCG | LOLRI | SETVI | ABUTH | AMARE | MATIN | POLCO | STEME | VIOTR | VERPE |
| I-152 | 320 | 100 | 100 | 100 | 100 | 100 | 100 | 100 | 100 | 100 | 100 |
| I-163 | 320 | 100 | 80 | 100 | 100 | 100 | 100 | 100 | 100 | 100 | 100 |
| I-165 | 320 | 100 | 100 | 100 | 100 | 100 | 100 | 100 | 100 | 100 | 100 |
| I-167 | 320 | 100 | 100 | 100 | 100 | 100 | 100 | 100 | 100 | 100 | 100 |
| I-171 | 320 | 90 | 100 | 100 | 100 | 100 | 100 | 100 | 100 | 100 | 100 |
| I-173 | 320 | 100 | 100 | 100 | 100 | 100 | 100 | 100 | 100 | 100 | 100 |
| I-175 | 320 | 100 | 100 | 100 | 100 | 100 | 100 | 100 | 100 | 100 | 100 |
| I-180 | 320 | 100 | 100 | 100 | 100 | 100 | 100 | 100 | 100 | 100 | 100 |
| I-182 | 320 | 90 | 100 | 100 | 100 | 100 | 100 | 100 | 100 | 100 | 100 |
| I-190 | 320 | 100 | 100 | 100 | 100 | 100 | 100 | 100 | 100 | 100 | 100 |
| I-191 | 320 | 100 | 100 | 100 | 100 | 100 | 100 | 100 | 100 | 100 | 100 |
| I-196 | 320 | 100 | 100 | 100 | 100 | 100 | 100 | 100 | 100 | 100 | 100 |
| I-197 | 320 | 90 | 80 | 100 | 100 | 100 | 100 | 80 | 100 | 100 | 100 |
| I-198 | 320 | 90 | 100 | 90 | 100 | 100 | 100 | 100 | 100 | 100 | 100 |
| I-199 | 320 | 100 | 100 | 100 | 100 | 100 | 100 | 100 | 100 | 100 | 100 |
| I-200 | 320 | 100 | 100 | 100 | 100 | 100 | 100 | 100 | 100 | 100 | 100 |
| I-213 | 320 | 100 | 100 | 100 | 100 | 100 | 100 | 100 | 100 | 100 | 100 |
| I-214 | 320 | 100 | 100 | 100 | 100 | 100 | 100 | 100 | 100 | 100 | 100 |
| I-215 | 320 | 100 | 100 | 100 | 100 | 100 | 100 | 100 | 100 | 100 | 100 |
| I-227 | 320 | 100 | 100 | 100 | 100 | 100 | 100 | 100 | 100 | 100 | 100 |
| I-231 | 320 | 100 | 100 | 100 | 100 | 100 | 100 | 100 | 100 | 100 | 100 |
| I-232 | 320 | 100 | 100 | 100 | 100 | 100 | 100 | 100 | 100 | 100 | 100 |
| I-234 | 320 | 100 | 100 | 100 | 100 | 100 | 100 | 100 | 100 | 100 | 100 |
| I-238 | 320 | 80 | 90 | 100 | 100 | 100 | 100 | 100 | 100 | 100 | 100 |
| I-245 | 320 | 100 | 100 | 100 | 100 | 100 | 100 | 100 | 100 | 100 | 100 |
| I-246 | 320 | 100 | 100 | 100 | 80 | 100 | 100 | 100 | 100 | 100 | 100 |
| I-248 | 320 | 100 | 100 | 100 | 100 | 100 | 100 | 100 | 100 | 100 | 100 |
| I-249 | 320 | 100 | | 100 | 100 | 100 | 100 | 100 | 100 | 100 | 100 |
| I-254 | 320 | 80 | 100 | 100 | 100 | 100 | 100 | 100 | 100 | 100 | 100 |
| I-256 | 320 | 90 | 80 | 100 | 100 | 100 | 100 | 100 | 100 | 100 | 100 |
| I-321 | 320 | | 100 | | 80 | 100 | 100 | 90 | 100 | 100 | 100 |
| I-347 | 320 | 100 | | 100 | 100 | 100 | 100 | 100 | 100 | 100 | 100 |
| I-348 | 320 | 100 | 80 | 100 | 100 | 100 | 100 | 100 | 100 | 100 | 100 |
| I-353 | 320 | 100 | 100 | 100 | 100 | 100 | 100 | 100 | 100 | 100 | 100 |
| I-354 | 320 | 100 | 100 | 100 | 100 | 100 | 100 | 100 | 100 | 100 | 100 |
| I-355 | 320 | 100 | 80 | 100 | 100 | 100 | 100 | 100 | 100 | 100 | 100 |
| I-356 | 320 | 100 | 100 | 100 | | 100 | 100 | 100 | 100 | 100 | 100 |
| I-360 | 320 | 90 | 100 | 100 | 100 | 100 | 100 | 100 | 100 | 100 | 100 |
| I-360 | 320 | | 80 | 100 | 100 | 100 | 100 | 100 | 100 | 100 | 100 |
| I-365 | 320 | 90 | 100 | 100 | 100 | 100 | 100 | 100 | 100 | 100 | 100 |
| I-366 | 320 | 100 | 100 | 100 | 100 | 100 | 100 | 100 | 100 | 100 | 100 |
| I-370 | 320 | 100 | 100 | 100 | 100 | 100 | 100 | 100 | 100 | 100 | 100 |
| I-371 | 320 | 100 | 100 | 100 | 100 | 100 | 100 | 100 | 100 | 100 | 100 |
| I-376 | 320 | 100 | 100 | 100 | 100 | 100 | 100 | 100 | 100 | 100 | 100 |
| I-377 | 320 | 100 | 100 | 100 | 100 | 100 | 100 | 100 | 100 | 100 | 100 |
| I-380 | 320 | 100 | 80 | 100 | 100 | 100 | 100 | 100 | 100 | 100 | 100 |
| I-381 | 320 | | 100 | 100 | 100 | 100 | 100 | 100 | 100 | 100 | 100 |
| I-402 | 320 | 100 | 100 | 100 | 100 | 100 | 100 | 100 | 100 | 100 | 100 |

TABLE B2
Pre-emergence herbicidal efficacy

| | | Herbicidal efficacy against [%] | | | | | |
|---|---|---|---|---|---|---|---|
| Example | Application | | | | | | |
| No.: | rate [g/ha] | AMARE | MATIN | POLCO | STEME | VIOTR | VERPE |
| I-151 | 320 | 90 | 100 | | 100 | 100 | 90 |
| I-187 | 320 | 100 | 90 | 100 | 100 | 100 | 100 |
| I-192 | 320 | 100 | 100 | 100 | 100 | 100 | 100 |
| I-193 | 320 | 100 | 90 | 100 | 100 | 100 | 100 |
| I-201 | 320 | 100 | 90 | 100 | 100 | 100 | 100 |
| I-203 | 320 | 100 | 90 | 100 | 100 | 100 | 100 |
| I-207 | 320 | 100 | 100 | 100 | 100 | 100 | 100 |
| I-239 | 320 | 100 | 100 | 100 | 100 | 100 | 100 |
| I-250 | 320 | 100 | 100 | 100 | 100 | 100 | 90 |
| I-253 | 320 | 100 | 100 | 100 | 100 | 100 | 100 |
| I-278 | 320 | 100 | 100 | 100 | 100 | 100 | 100 |
| I-301 | 320 | 100 | 100 | 80 | 100 | 100 | 90 |
| I-315 | 320 | 80 | 80 | | 90 | 100 | 100 |
| I-316 | 320 | | 100 | 100 | 100 | 100 | 100 |
| I-320 | 320 | 90 | 100 | | 100 | 100 | 100 |
| I-327 | 320 | 100 | 100 | | 100 | 100 | 100 |
| I-329 | 320 | 100 | 100 | 100 | 100 | 100 | 100 |
| I-333 | 320 | 100 | | 100 | 100 | 100 | 100 |
| I-336 | 320 | 100 | 100 | | 100 | 100 | 100 |
| I-343 | 320 | 100 | 100 | | 100 | 100 | 100 |
| I-349 | 320 | 100 | 100 | 100 | 100 | 100 | 100 |
| I-361 | 320 | 100 | 100 | 100 | 100 | 100 | 100 |
| I-362 | 320 | 100 | 100 | 100 | 100 | 100 | 100 |
| I-364 | 320 | 100 | 100 | 100 | 100 | 100 | 100 |
| I-368 | 320 | 100 | 100 | | 100 | 100 | 100 |
| I-373 | 320 | 100 | 100 | 100 | 100 | 100 | 100 |
| I-378 | 320 | 100 | 100 | 100 | 100 | 100 | 100 |

TABLE B3
Pre-emergence herbicidal efficacy

| | | Herbicidal efficacy against [%] | | |
|---|---|---|---|---|
| Example | Application | | | |
| No.: | rate [g/ha] | MATIN | STEME | VIOTR |
| I-060 | 320 | 100 | 100 | 90 |
| I-061 | 320 | 100 | 100 | 100 |
| I-068 | 320 | 100 | 100 | 100 |
| I-076 | 320 | | 90 | 100 |
| I-078 | 320 | 70 | 90 | 100 |
| I-087 | 320 | 100 | 100 | 100 |
| I-094 | 320 | 100 | 100 | 100 |
| I-097 | 320 | 100 | 100 | 100 |
| I-157 | 320 | 80 | 100 | 100 |
| I-158 | 320 | 90 | 100 | 100 |
| I-160 | 320 | 90 | 100 | 100 |
| I-164 | 320 | 90 | 100 | 100 |
| I-166 | 320 | 90 | 100 | 100 |
| I-168 | 320 | 80 | 90 | 100 |
| I-169 | 320 | 90 | 100 | 100 |
| I-170 | 320 | 90 | 100 | 100 |
| I-172 | 320 | 90 | 100 | 100 |
| I-174 | 320 | 90 | 100 | 100 |
| I-176 | 320 | 80 | 80 | 90 |
| I-177 | 320 | 90 | 100 | 100 |
| I-178 | 320 | 70 | 100 | 100 |
| I-179 | 320 | 90 | 100 | 70 |
| I-181 | 320 | 100 | 100 | 100 |
| I-183 | 320 | 90 | 100 | 90 |
| I-184 | 320 | 90 | 100 | 100 |
| I-185 | 320 | 90 | 100 | 100 |
| I-186 | 320 | 80 | 100 | 100 |
| I-194 | 320 | 90 | 100 | 100 |
| I-195 | 320 | 90 | 100 | 100 |
| I-202 | 320 | 90 | 100 | 100 |
| I-204 | 320 | 100 | 100 | |
| I-209 | 320 | 100 | 100 | 100 |
| I-211 | 320 | 100 | 100 | 100 |
| I-212 | 320 | 100 | 100 | 100 |
| I-216 | 320 | 100 | 100 | 100 |
| I-219 | 320 | 100 | 100 | 100 |
| I-223 | 320 | 90 | 100 | 90 |
| I-228 | 320 | 100 | 100 | 100 |
| I-236 | 320 | 100 | 100 | 100 |
| I-242 | 320 | 100 | 80 | |
| I-247 | 320 | 100 | 100 | 100 |
| I-250 | 320 | 100 | 100 | 100 |
| I-252 | 320 | 100 | 100 | |
| I-253 | 320 | 100 | 100 | 100 |
| I-255 | 320 | | 90 | 100 |
| I-262 | 320 | | 100 | 100 |
| I-271 | 320 | 90 | 90 | 90 |
| I-273 | 320 | 80 | 100 | 100 |
| I-278 | 320 | 80 | 80 | 90 |
| I-305 | 320 | 100 | 100 | 100 |
| I-316 | 320 | 100 | 100 | 100 |
| I-321 | 320 | 100 | 100 | 100 |
| I-325 | 320 | | 100 | |
| I-327 | 320 | 100 | 100 | 100 |

As the results show, compounds according to the invention, for example compound Nos. I-231, I-199 and other compounds from table B1, when applied pre-emergence, have very good herbicidal efficacy against harmful plants. Here, for example, compound Nos. I-214 and I-196, when applied pre-emergence, have very good activity (80% to 100% herbicidal activity) against harmful plants such as Abutilon theophrasti, Amaranthus retroflexus, Echinochloa crus-galli, Lolium rigidum, Matricaria inodora, Polygonum convolvulus, Setaria viridis, Stellaria media, Veronica persica and Viola tricolor at an application rate of 0.32 kg of active substance or less per hectare. At the same time, some of the compounds according to the invention leave Gramineae crops such as barley, wheat, rye, millet/sorghum, corn, rice or sugar cane virtually undamaged when applied pre-emergence, even at high active compound dosages. In addition, some substances are also harmless to dicotyledonous crops such as soya, cotton, oilseed rape or sugar beet.

Some of the compounds according to the invention exhibit high selectivity and are therefore suitable for controlling unwanted vegetation in agricultural crops by the pre-emergence method.

C.2 Post-Emergence Herbicidal Action and Crop Plant Compatibility

Seeds of monocotyledonous and dicotyledonous weeds and crop plants are placed in sandy loam in plastic or organic planting pots, covered with soil and cultivated in a greenhouse under controlled growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants as aqueous suspension or emulsion with addition of 0.5% additive at a water application rate of 6001/ha (converted). After the test plants had been kept in the greenhouse under optimum growth conditions for about 3 weeks, the activity of the preparations is rated visually in comparison to untreated controls. For example, 100% activity=the plants have died, 0% activity=like control plants.

TABLE B4
Post-emergence herbicidal efficacy

Herbicidal efficacy against [%]

| Example | Application | | |
|---|---|---|---|
| No.: | rate [g/ha] | VIOTR | VERPE |
| I-068 | 1280 | 80 | 90 |
| I-087 | 1280 | 80 | 90 |
| I-195 | 1280 | 90 | 90 |
| I-219 | 1280 | 80 | 90 |
| I-247 | 1280 | 80 | 80 |
| I-249 | 1280 | 80 | 80 |
| I-273 | 1280 | 90 | 100 |

TABLE B5
Post-emergence herbicidal efficacy

| | | Herbicidal efficacy against [%] | | | | | |
|---|---|---|---|---|---|---|---|
| Example | Application | | | | | | |
| No.: | rate [g/ha] | ECHCG | LOLRI | AMARE | STEME | VIOTR | VERPE |
| I-231 | 320 | 100 | 80 | 80 | 80 | 90 | 90 |
| I-232 | 320 | 80 | 90 | 100 | 100 | 90 | 90 |
| I-248 | 320 | 100 | 100 | 90 | 100 | 90 | 90 |
| I-353 | 320 | 100 | 100 | 90 | 100 | 80 | 90 |
| I-354 | 320 | 100 | 100 | 80 | 100 | 80 | 100 |
| I-355 | 320 | 100 | | 80 | 100 | 80 | 80 |
| I-366 | 320 | 100 | 90 | 80 | 90 | | |
| I-402 | 320 | 100 | | 90 | 90 | 90 | 90 |

TABLE B6
Post-emergence herbicidal efficacy

| | | Herbicidal efficacy against [%] | | | |
|---|---|---|---|---|---|
| Example | Application | | | | |
| No.: | rate [g/ha] | SETVI | ABUTH | AMARE | VERPE |
| I-180 | 320 | 80 | 80 | 80 | 80 |
| I-214 | 320 | 100 | 80 | 80 | 100 |
| I-377 | 320 | 90 | 80 | 80 | 90 |

TABLE B7
Post-emergence herbicidal efficacy

Herbicidal efficacy against [%]

| Example | Application | | |
|---|---|---|---|
| No.: | rate [g/ha] | VIOTR | VERPE |
| I-087 | 320 | 80 | 80 |
| I-163 | 320 | 80 | 90 |
| I-165 | 320 | 80 | 90 |
| I-170 | 320 | 80 | 90 |
| I-173 | 320 | 80 | 90 |
| I-190 | 320 | 80 | 80 |
| I-192 | 320 | 80 | 80 |
| I-194 | 320 | 80 | 100 |
| I-195 | 320 | 80 | 80 |
| I-198 | 320 | 80 | 90 |
| I-200 | 320 | 80 | 100 |
| I-202 | 320 | 80 | 80 |
| I-213 | 320 | 90 | 90 |
| I-227 | 320 | 90 | 100 |
| I-234 | 320 | 90 | 80 |
| I-245 | 320 | 80 | 90 |
| I-327 | 320 | 80 | 90 |

As the results show, compounds according to the invention, for example compound Nos. I-248, I-353 and I-231 and other compounds from table B5, when applied post-emergence, have very good herbicidal efficacy against harmful plants. Here, for example, compound Nos. I-377 and I-214, when applied post-emergence, have very good herbicidal activity (80% to 100% herbicidal activity) against harmful plants such as Abutilon theophrasti, Amaranthus retroflexus, Setaria viridis, and Veronica persica at an application rate of 0.32 kg of active substance or less per hectare. At the same time, some of the compounds according to the invention leave Gramineae crops such as barley, wheat, rye, millet/sorghum, corn, rice or sugar cane virtually undamaged when applied post-emergence, even at high active compound dosages. In addition, some substances are also harmless to dicotyledonous crops such as soya, cotton, oilseed rape or sugar beet. Some of the compounds according to the invention have high selectivity and are therefore suitable for controlling unwanted vegetation in agricultural crops by the post-emergence method.

1. A compound of formula (I) 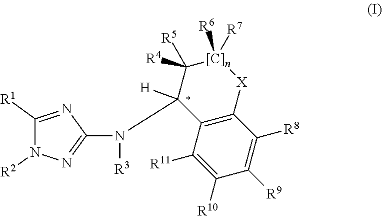
or an agrochemically compatible salt thereof, in which
R¹ is selected from the group consisting of
hydrogen, halogen, hydroxyl, cyano, nitro, amino, C(O)OH, C(O)NH₂;
(C₁-C₆)-alkyl, (C₁-C₆)-haloalkyl, (C₁-C₆)-alkylcarbonyl, (C₁-C₆)-haloalkylcarbonyl, (C₁-C₆)-alkylcarbonyloxy, (C₁-C₆)-haloalkylcarbonyloxy, (C₁-C₆)-alkylcarbonyl-(C₁-C₄)-alkyl;
(C₁-C₆)-alkoxy, (C₁-C₄)-alkoxyalkyl, (C₁-C₆)-haloalkoxy, (C₁-C₆)-alkoxycarbonyl, (C₁-C₆)-haloalkoxycarbonyl, (C₁-C₆)-alkoxycarbonyl-(C₁-C₆)-alkyl, (C₁-C₆)-haloalkoxycarbonyl-(C₁-C₆)-alkyl, (C₁-C₆)-alkoxycarbonyl-(C₁-C₆)-haloalkyl, (C₁-C₆)-haloalkoxycarbonyl-(C₁-C₆)-haloalkyl;
(C₂-C₆)-alkenyl, (C₂-C₆)-haloalkenyl, (C₂-C₆)-alkenylcarbonyl, (C₂-C₆)-haloalkenylcarbonyl, (C₂-C₆)-alkenyloxy, (C₂-C₆)-haloalkenyloxy, (C₂-C₆)-alkenyloxycarbonyl, (C₂-C₆)-haloalkenyloxycarbonyl;
(C₂-C₆)-alkynyl, (C₂-C₆)-haloalkynyl, (C₂-C₆)-alkynylcarbonyl, (C₂-C₆)-haloalkynylcarbonyl, (C₂-C₆)-alkynyloxy, (C₂-C₆)-haloalkynyloxy, (C₂-C₆)-alkynyloxycarbonyl, (C₂-C₆)-haloalkynyloxycarbonyl;
tri-(C₁-C₆)-alkylsilyl-(C₂-C₆)-alkynyl, di-(C₁-C₆)-alkylsilyl-(C₂-C₆)-alkynyl, mono-(C₁-C₆)-alkylsilyl-(C₂-C₆)-alkynyl, phenylsilyl-(C₂-C₆)-alkynyl;
(C₆-C₁₄)-aryl, (C₆-C₁₄)-aryloxy, (C₆-C₁₄)-arylcarbonyl and (C₆-C₁₄)-aryloxycarbonyl, each of which may be substituted in the aryl moiety by halogen, (C₁-C₆)-alkyl and/or (C₁-C₆)-haloalkyl;
(C₆-C₁₄)-aryl-(C₁-C₆)-alkyl, (C₆-C₁₄)-aryl-(C₁-C₆)-alkoxy, (C₆-C₁₄)-aryl-(C₁-C₆)-alkylcarbonyl, (C₆-C₁₄)-aryl-(C₁-C₆)-alkylcarbonyloxy, (C₆-C₁₄)-aryl-(C₁-C₆)-alkoxycarbonyl, (C₆-C₄)-aryl-(C₁-C₆)-alkoxycarbonyloxy;
mono-((C₁-C₆)-alkyl)amino, mono-((C₁-C₆)-haloalkyl)amino, di-((C₁-C₆)-alkyl)amino, di-((C₁-C₆)-haloalkyl)amino, ((C₁-C₆)-alkyl-(C₁-C₆)-haloalkyl)amino, N—((C₁-C₆)-alkanoyl)amino, N—((C₁-C₆)-haloalkanoyl)amino, aminocarbonyl-(C₁-C₆)-alkyl, di-(C₁-C₆)-alkylaminocarbonyl-(C₁-C₆)-alkyl;
mono-((C₁-C₆)-alkyl)aminocarbonyl, mono-((C₁-C₆)-haloalkyl)aminocarbonyl, di-((C₁-C₆)-alkyl)aminocarbonyl, di-((C₁-C₆)-haloalkyl)aminocarbonyl, ((C₁-C₆)-alkyl-(C₁-C₆)-haloalkyl)aminocarbonyl, N—((C₁-C₆)-alkanoyl)aminocarbonyl, N—((C₁-C₆)-haloalkanoyl)aminocarbonyl, mono-((C₆-C₁₄)-aryl)aminocarbonyl, di-((C₆-C₁₄)-aryl)aminocarbonyl,
di-((C₁-C₆)-alkyl)amino, with the proviso that the two (C₁-C₆)-alkyl radicals form a cycle that may optionally be interrupted by NH, O or S;
(C₁-C₆)-alkoxy-(C₁-C₆)-alkyl, (C₁-C₆)-alkoxy-(C₁-C₆)-alkoxy, (C₁-C₆)-alkoxycarbonyl-(C₁-C₆)-alkoxy;
(C₃-C₈)-cycloalkyl which may optionally be substituted on the cycloalkyl radical by (C₁-C₆)-alkyl and/or halogen; (C₃-C₈)-cycloalkoxy, (C₃-C₈)-cycloalkyl-(C₁-C₆)-alkyl, (C₃-C₈)-cycloalkyl-(C₁-C₆)-haloalkyl, (C₃-C₈)-cycloalkyl-(C₁-C₆)-alkoxy, (C₃-C₈)-cycloalkyl-(C₁-C₆)-haloalkoxy, (C₃-C₈)-cycloalkylcarbonyl, (C₃-C₈)-cycloalkoxycarbonyl, (C₃-C₈)-cycloalkyl-(C₁-C₆)-alkylcarbonyl, (C₃-C₈)-cycloalkyl-(C₁-C₆)-haloalkylcarbonyl, (C₃-C₈)-cycloalkyl-(C₁-C₆)-alkoxycarbonyl, (C₃-C₈)-cycloalkyl-(C₁-C₆)-haloalkoxycarbonyl, (C₃-C₈)-cycloalkylcarbonyloxy, (C₃-C₈)-cycloalkoxycarbonyloxy, (C₃-C₈)-cycloalkyl-(C₁-C₆)-alkylcarbonyloxy, (C₃-C₈)-cycloalkyl-(C₁-C₆)-halogenalkylcarbonyloxy, (C₃-C₈)-cycloalkyl-(C₁-C₆)-alkoxycarbonyloxy, (C₃-C₈)-cycloalkyl-(C₁-C₆)-haloalkoxycarbonyloxy; (C₅-C₆)-cycloheteroalkyl which may optionally be interrupted independently of one another once or twice by NH, O or S;
(C₃-C₈)-cycloalkenyl, (C₃-C₈)-cycloalkenyloxy, (C₃-C₈)-cycloalkenyl-(C₁-C₆)-alkyl, (C₃-C₈)-cycloalkenyl-(C₁-C₆)-haloalkyl, (C₃-C₈)-cycloalkenyl-(C₁-C₆)-alkoxy, (C₃-C₈)-cycloalkenyl-(C₁-C₆)-haloalkoxy, (C₃-C₈)-cycloalkenylcarbonyl, (C₃-C₈)-cycloalkenyloxycarbonyl, (C₃-C₈)-cycloalkenyl-(C₁-C₆)-alkylcarbonyl, (C₃-C₈)-cycloalkenyl-(C₁-C₆)-haloalkylcarbonyl, (C₃-C₈)-cycloalkenyl-(C₁-C₆)-alkoxycarbonyl, (C₃-C₈)-cycloalkenyl-(C₁-C₆)-haloalkoxycarbonyl, (C₃-C₈)-cycloalkenylcarbonyloxy, (C₃-C₈)-cycloalkenyloxycarbonyloxy, (C₃-C₈)-cycloalkenyl-(C₁-C₆)-alkylcarbonyloxy, (C₃-C₈)-cycloalkenyl-(C₁-C₆)-haloalkylcarbonyloxy, (C₃-C₈)-cycloalkenyl-(C₁-C₆)-alkoxycarbonyloxy, (C₃-C₈)-cycloalkenyl-(C₁-C₆)-haloalkoxycarbonyloxy;
hydroxy-(C₁-C₆)-alkyl, hydroxy-(C₁-C₆)-alkoxy, cyano-(C₁-C₆)-alkoxy, cyano-(C₁-C₆)-alkyl;
(C₁-C₆)-alkylsulfonyl, (C₁-C₆)-alkylthio, (C₁-C₆)-alkylsulfinyl, (C₁-C₆)-haloalkylsulfonyl, (C₁-C₆)-haloalkylthio, (C₁-C₆)-haloalkylsulfinyl, (C₁-C₆)-alkylsulfonyl-(C₁-C₆)-alkyl, (C₁-C₆)-alkylthio-(C₁-C₆)-alkyl, (C₁-C₆)-alkylsulfinyl-(C₁-C₆)-alkyl, (C₁-C₆)-haloalkylsulfonyl-(C₁-C₆)-alkyl, (C₁-C₆)-haloalkylthio-(C₁-C₆)-alkyl, (C₁-C₆)-haloalkylsulfinyl-(C₁-C₆)-alkyl, (C₁-C₆)-alkylsulfonyl-(C₁-C₆)-haloalkyl, (C₁-C₆)-alkylthio-(C₁-C₆)-haloalkyl, (C₁-C₆)-alkylsulfinyl-(C₁-C₆)-haloalkyl, (C₁-C₆)-haloalkylsulfonyl-(C₁-C₆)-haloalkyl, (C₁-C₆)-haloalkylthio-(C₁-C₆)-haloalkyl, (C₁-C₆)-haloalkylsulfinyl-(C₁-C₆)-haloalkyl, (C₁-C₆)-alkylsulfonyloxy, (C₁-C₆)-haloalkylsulfonyloxy, (C₁-C₆)-alkylthiocarbonyl, (C₁-C₆)-haloalkylthiocarbonyl, (C₁-C₆)-alkylthiocarbonyloxy, (C₁-C₆)-haloalkylthiocarbonyloxy, (C₁-C₆)-alkylthio-(C₁-C₆)-alkyl, (C₁-C₆)-alkylthio-(C₁-C₆)-alkoxy, (C₁-C₆)-alkylthio-(C₁-C₆)-alkylcarbonyl, (C₁-C₆)-alkylthio-(C₁-C₆)-alkylcarbonyloxy; (C₄-C₁₄)-arylsulfonyl, (C₆-C₁₄)-arylthio, (C₆-C₁₄)-arylsulfinyl, (C₃-C₈)-cycloalkylthio, (C₃-C₈)-alkenylthio, (C₃-C₈)-cycloalkenylthio, (C₃-C₆)-alkynylthio;
R² is selected from the group consisting of
(C₁-C₆)-alkyl, (C₁-C₆)-haloalkyl, (C₁-C₆)-alkylcarbonyl, (C₁-C₆)-haloalkylcarbonyl, (C₁-C₆)-alkylcarbonyloxy, (C₁-C₆)-haloalkylcarbonyloxy, (C₁-C₆)-alkylcarbonyl-(C₁-C₄)-alkyl;
(C₁-C₆)-alkoxy, (C₁-C₆)-haloalkoxy, (C₁-C₆)-alkoxycarbonyl, (C₁-C₆)-haloalkoxycarbonyl, (C₁-C₆)-alkoxycarbonyl-(C₁-C₆)-alkyl, (C₁-C₆)-haloalkoxycarbonyl-(C₁-C₆)-alkyl, (C₁-C₆)-alkoxycarbonyl-(C₁-C₆)-haloalkyl, (C₁-C₆)-haloalkoxycarbonyl-(C₁-C₆)-haloalkyl;
(C₂-C₆)-alkenyl, (C₂-C₆)-haloalkenyl, (C₂-C₆)-alkenylcarbonyl, (C₂-C₆)-haloalkenylcarbonyl, (C₂-C₆)-alkenyloxy, (C₂-C₆)-haloalkenyloxy, (C₂-C₆)-alkenyloxycarbonyl, (C₂-C₆)-haloalkenyloxycarbonyl;
(C₂-C₆)-alkynyl, (C₂-C₆)-haloalkynyl, (C₂-C₆)-alkynylcarbonyl, (C₂-C₆)-haloalkynylcarbonyl, (C₂-C₆)-alkynyloxy, (C₂-C₆)-haloalkynyloxy, (C₂-C₆)-alkynyloxycarbonyl, (C₂-C₆)-haloalkynyloxycarbonyl;
tri-(C₁-C₆)-alkylsilyl-(C₂-C₆)-alkynyl, di-(C₁-C₆)-alkylsilyl-(C₂-C₆)-alkynyl, mono-(C₁-C₆)-alkylsilyl-(C₂-C₆)-alkynyl, phenylsilyl-(C₂-C₆)-alkynyl;
(C₆-C₁₄)-aryl, (C₆-C₁₄)-aryloxy, (C₆-C₁₄)-arylcarbonyl and (C₆-C₁₄)-aryloxycarbonyl, each of which may be substituted in the aryl moiety by halogen, (C₁-C₆)-alkyl and/or (C₁-C₆)-haloalkyl;
(C₆-C₁₄)-aryl-(C₁-C₆)-alkyl, (C₆-C₁₄)-aryl-(C₁-C₆)-alkoxy, (C₆-C₁₄)-aryl-(C₁-C₆)-alkylcarbonyl, (C₆-C₁₄)-aryl-(C₁-C₆)-alkylcarbonyloxy, (C₆-C₁₄)-aryl-(C₁-C₆)-alkoxycarbonyl, (C₆-C₁₄)-aryl-(C₁-C₆)-alkoxycarbonyloxy;
mono-((C₁-C₆)-alkyl)amino, mono-((C₁-C₆)-haloalkyl)amino, di-((C₁-C₆)-alkyl)amino, di-((C₁-C₆)-haloalkyl)amino, ((C₁-C₆)-alkyl-(C₁-C₆)-haloalkyl)amino, N—((C₁-C₆)-alkanoyl)amino, N—((C₁-C₆)-haloalkanoyl)amino, aminocarbonyl-(C₁-C₆)-alkyl, di-(C₁-C₆)-alkylaminocarbonyl-(C₁-C₆)-alkyl;
mono-((C₁-C₆)-alkyl)aminocarbonyl, mono-((C₁-C₆)-haloalkyl)aminocarbonyl, di-((C₁-C₆)-alkyl)aminocarbonyl, di-((C₁-C₆)-haloalkyl)aminocarbonyl, ((C₁-C₆)-alkyl-(C₁-C₆)-haloalkyl)aminocarbonyl, N—((C₁-C₆)-alkanoyl)aminocarbonyl, N—((C₁-C₆)-haloalkanoyl)aminocarbonyl, mono-((C₆-C₁₄)-aryl)aminocarbonyl, di-((C₆-C₁₄)-aryl)aminocarbonyl,
(C₁-C₆)-alkoxy-(C₁-C₆)-alkyl, (C₁-C₆)-alkoxy-(C₁-C₆)-alkoxy, (C₁-C₆)-alkoxycarbonyl-(C₁-C₆)-alkoxy;
(C₃-C₈)-cycloalkyl which may optionally be substituted in the cycloalkyl radical by (C₁-C₆)-alkyl and/or halogen; (C₃-C₈)-cycloalkoxy, (C₃-C₈)-cycloalkyl-(C₁-C₆)-alkyl, (C₃-C₈)-cycloalkyl-(C₁-C₆)-haloalkyl, (C₃-C₈)-cycloalkyl-(C₁-C₆)-alkoxy, (C₃-C₈)-cycloalkyl-(C₁-C₆)-haloalkoxy, (C₃-C₈)-cycloalkylcarbonyl, (C₃-C₈)-cycloalkoxycarbonyl, (C₃-C₈)-cycloalkyl-(C₁-C₆)-alkylcarbonyl, (C₃-C₈)-cycloalkyl-(C₁-C₆)-haloalkylcarbonyl, (C₃-C₈)-cycloalkyl-(C₁-C₆)-alkoxycarbonyl, (C₃-C₈)-cycloalkyl-(C₁-C₆)-haloalkoxycarbonyl, (C₃-C₈)-cycloalkylcarbonyloxy, (C₃-C₈)-cycloalkoxycarbonyloxy, (C₃-C₈)-cycloalkyl-(C₁-C₆)-alkylcarbonyloxy, (C₃-C₈)-cycloalkyl-(C₁-C₆)-haloalkylcarbonyloxy, (C₃-C₈)-cycloalkyl-(C₁-C₆)-alkoxycarbonyloxy, (C₃-C₈)-cycloalkyl-(C₁-C₆)-haloalkoxycarbonyloxy;
(C₃-C₈)-cycloalkenyl, (C₃-C₈)-cycloalkenyloxy, (C₃-C₈)-cycloalkenyl-(C₁-C₆)-alkyl, (C₃-C₈)-cycloalkenyl-(C₁-C₆)-haloalkyl, (C₃-C₈)-cycloalkenyl-(C₁-C₆)-alkoxy, (C₃-C₈)-cycloalkenyl-(C₁-C₆)-haloalkoxy, (C₃-C₈)-cycloalkenylcarbonyl, (C₃-C₈)-cycloalkenyloxycarbonyl, (C₃-C₈)-cycloalkenyl-(C₁-C₆)-alkylcarbonyl, (C₃-C₈)-cycloalkenyl-(C₁-C₆)-haloalkylcarbonyl, (C₃-C₈)-cycloalkenyl-(C₁-C₆)-alkoxycarbonyl, (C₃-C₈)-cycloalkenyl-(C₁-C₆)-haloalkoxycarbonyl, (C₃-C₈)-cycloalkenylcarbonyloxy, (C₃-C₈)-cycloalkenyloxycarbonyloxy, (C₃-C₈)-cycloalkenyl-(C₁-C₆)-alkylcarbonyloxy, (C₃-C₈)-cycloalkenyl-(C₁-C₆)-haloalkylcarbonyloxy, (C₃-C₈)-cycloalkenyl-(C₁-C₆)-alkoxycarbonyloxy, (C₃-C₈)-cycloalkenyl-(C₁-C₆)-haloalkoxycarbonyloxy;
hydroxy-(C₁-C₆)-alkyl, hydroxy-(C₁-C₆)-alkoxy, cyano-(C₁-C₆)-alkoxy, cyano-(C₁-C₆)-alkyl;
(C₁-C₆)-alkylsulfonyl, (C₁-C₆)-alkylthio, (C₁-C₆)-alkylsulfinyl, (C₁-C₆)-haloalkylsulfonyl, (C₁-C₆)-haloalkylthio, (C₁-C₆)-haloalkylsulfinyl, (C₁-C₆)-alkylsulfonyl-(C₁-C₆)-alkyl, (C₁-C₆)-alkylthio-(C₁-C₆)-alkyl, (C₁-C₆)-alkylsulfinyl-(C₁-C₆)-alkyl, (C₁-C₆)-haloalkylsulfonyl-(C₁-C₆)-alkyl, (C₁-C₆)-haloalkylthio-(C₁-C₆)-alkyl, (C₁-C₆)-haloalkylsulfinyl-(C₁-C₆)-alkyl, (C₁-C₆)-alkylsulfonyl-(C₁-C₆)-haloalkyl, (C₁-C₆)-alkylthio-(C₁-C₆)-haloalkyl, (C₁-C₆)-alkylsulfinyl-(C₁-C₆)-haloalkyl, (C₁-C₆)-haloalkylsulfonyl-(C₁-C₆)-haloalkyl, (C₁-C₆)-haloalkylthio-(C₁-C₆)-haloalkyl, (C₁-C₆)-haloalkylsulfinyl-(C₁-C₆)-haloalkyl, (C₁-C₆)-alkylsulfonyloxy, (C₁-C₆)-haloalkylsulfonyloxy, (C₁-C₆)-alkylthiocarbonyl, (C₁-C₆)-haloalkylthiocarbonyl, (C₁-C₆)-alkylthiocarbonyloxy, (C₁-C₆)-haloalkylthiocarbonyloxy, (C₁-C₆)-alkylthio-(C₁-C₆)-alkyl, (C₁-C₆)-alkylthio-(C₁-C₆)-alkoxy, (C₁-C₆)-alkylthio-(C₁-C₆)-alkylcarbonyl, (C₁-C₆)-alkylthio-(C₁-C₆)-alkylcarbonyloxy; (C₄-C₁₄)-arylsulfonyl, (C₆-C₁₄)-arylthio, (C₆-C₁₄)-arylsulfinyl, (C₃-C₈)-cycloalkylthio, (C₃-C₈)-alkenylthio, (C₃-C₈)-cycloalkenylthio, (C₃-C₆)-alkynylthio;
or
R¹ and R² together with the carbon atom or nitrogen atom to which they are bonded form a saturated five- to seven-membered ring that may be interrupted by the Y group, where Y is selected from C(O), O, S, S(O), S(O)₂, NR^{1a}, C(O)—NR^{1a}, where the saturated five- to seven-membered ring may be substituted by m substituents selected from halogen, acetyl, (C₁-C₆)-alkyl, (C₃-C₆)-cycloalkyl, trifluoromethyl, COOMe, COOEt, CONH₂, nitrile, (C₁-C₆)-alkylsulfonyl, (C₃-C₆)-cycloalkylsulfonyl, phenylsulfonyl or phenyl-(C₁-C₆)-alkylsulfonyl, and where R^{1a} is selected from hydrogen, acetyl, trifluoroacetyl, (C₁-C₆)-alkyl, (C₃-C₆)-cycloalkyl, (C₃-C₆)-cycloalkylcarbonyl, (C₃-C₆)-cycloalkyl-(C₁-C₆)-alkyl, (C₁-C₄)-alkylsulfonyl, phenyl, phenyl-(C₁-C₆)-alkyl, phenylcarbonyl, 2-pyridinyl, 3-pyridinyl, 4-pyridinyl, 2-pyridinylcarbonyl, 3-pyridinylcarbonyl, 4-pyridinylcarbonyl, where the 14 latter radicals may be substituted by halogen, (C₁-C₆)-alkyl, (C₁-C₆)-alkyloxy, nitrile or trifluoromethyl;
R³ is hydrogen, (C₁-C₆)-alkyl, (C₁-C₆)-haloalkyl, (C₁-C₆)-alkylcarbonyl, (C₁-C₆)-haloalkylcarbonyl or (C₁-C₆)-alkylcarbonyloxy;
R⁴ and R⁵ are each independently of one another hydrogen, (C₁-C₆)-alkyl, (C₁-C₆)-haloalkyl, hydroxyl, (C₁-C₆)-alkoxy or (C₁-C₆)-haloalkoxy;
or
R⁴ and R⁵ together with the carbon atom to which they are attached form a saturated three- to seven-membered ring which may contain one or more heteroatoms selected from the group consisting of oxygen or sulfur or NH or NR¹a;
R⁶ and R⁷ are each independently of one another hydrogen, (C₁-C₆)-alkyl, (C₁-C₆)-haloalkyl, (C₁-C₆)-alkoxy, (C₁-C₆)-haloalkoxy, (C₆-C₁₄)-aryl, (C₆-C₁₄)-aryloxy, (C₆-C₁₄)-arylcarbonyl or (C₆-C₁₄)-aryloxycarbonyl;
or
R⁶ and R⁷ together with the carbon to which they are attached form a (C₃-C₇)-alkylene group which may contain one or more oxygen and/or sulfur atoms, where the (C₃-C₇)-alkylene group may be mono- or polysubstituted by halogen and the respective halogen substituents may be identical or different;
R⁸, R⁹, R¹⁰ and R¹¹ are each independently of one another hydrogen, halogen, cyano, C(O)OH, C(O)NH₂, (C₁-C₆)-alkyl, (C₁-C₆)-alkylcarbonyl, (C₁-C₆)-alkyloxycarbonyl, (C₁-C₆)-alkylaminocarbonyl, (C₁-C₆)-dialkylaminocarbonyl, (C₁-C₆)-haloalkyl, (C₁-C₆)-alkoxy, (C₁-C₆)-haloalkoxy, (C₂-C₆)-alkenyl, (C₂-C₆)-alkynyl, (C₂-C₆)-haloalkynyl, (C₂-C₆)-alkynylcarbonyl, (C₂-C₆)-haloalkynylcarbonyl, (C₂-C₆)-alkynyloxy, (C₂-C₆)-haloalkynyloxy, (C₂-C₆)-alkynyloxycarbonyl, (C₂-C₆)-haloalkynyloxycarbonyl or nitro, where the R⁹ and R¹⁰ radicals may be joined by an —O—CH₂—O-group to form a ring;
X represents a bond, CH₂, O, S, carbonyl, NH, CR¹²R¹³, NR¹⁴, CH₂O or CH₂S, where in the two latter groups the carbon atom is attached to the aromatic moiety and the heteroatom O or S is attached to the partially hydrogenated moiety of the amine; where, when n=0, X cannot be a bond;
R¹² and R¹³ are each independently of one another hydrogen, (C₁-C₆)-alkyl or (C₁-C₆)-haloalkyl;
R¹⁴ is hydrogen, (C₁-C₆)alkyl or (C₁-C₆)haloalkyl;
n is the serial number 0, 1 or 2; and
m is the serial number 0, 1, 2, 3, 4 or 5.

2. A compound of formula (I) as claimed in claim 1, where R¹ is hydrogen, cyano, fluorine, chlorine, bromine, iodine, nitro, trimethylsilylethynyl, methyl, ethyl, propyl, isopropyl, butyl, tert-butyl, n-pentyl, n-heptyl, cyclopropyl, cyclobutyl, acetyl, ethynyl, amino, dimethylamino, trifluoromethyl, difluoromethyl, monofluoromethyl, methoxy, ethoxy or methoxymethyl.

3. A compound of formula (I) as claimed in claim 1, where R¹ is hydrogen, chlorine, phenyl, 2-methylphenyl, 3-trifluoromethylphenyl, methyl, ethyl, isopropyl, butyl, tert-butyl, n-pentyl, n-heptyl, trifluoromethyl, 1-methylcyclopropyl, 1-(p-xylyl)-cyclopropyl, 1-(2,4-dichlorophenyl)cyclopropyl, amino, dimethylamino, trifluoromethyl, difluoromethyl, monofluoromethyl, CHFCH₃, CF(CH₃)₂, CHF(CH₂CH₃), 1-fluorocyclopropyl, cyclopentyl, methoxy, ethoxy, methoxymethyl, ethoxymethyl, thiomethyl, methylthio or methoxy.

4. A compound of formula (i) as claimed in claim 1, where R¹ and R² together with the carbon or nitrogen atom to which they are attached form a saturated six- or seven-membered ring selected from the following rings (a) to (k): 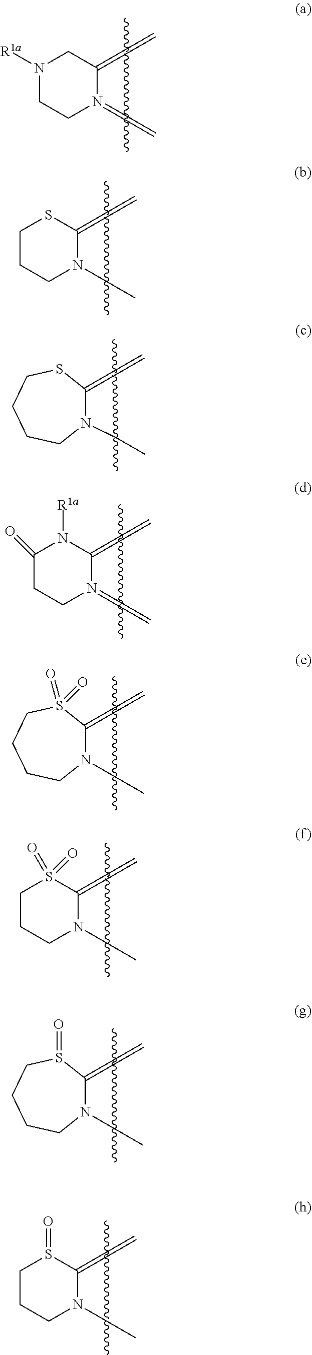 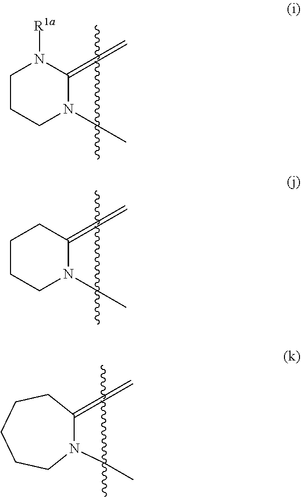
where the saturated six- or seven-membered ring may be substituted by m substituents selected from halogen, acetyl, (C₁-C₆)-alkyl, (C₃-C₆)-cycloalkyl, trifluoromethyl, (C₁-C₆)-alkylsulfonyl, (C₃-C₆)-cycloalkylsulfonyl or phenylsulfonyl,
and where R^{1a} is hydrogen, acetyl, (C₁-C₄)-alkyl, (C₁-C₄)-alkylsulfonyl or phenylcarbonyl, and where the phenyl may be substituted by a halogen.

5. A compound of formula (I) as claimed in claim 1, where R³ is hydrogen.

6. A compound of formula (I) as claimed in claim 1, where R⁴ and R⁵ each independently of one another are hydrogen, methyl, ethyl, propyl, cyclopropyl, hydroxyl or methoxy.

7. A compound of formula (I) as claimed in claim 1, where R⁶ and R⁷ independently of one another are hydrogen, methyl or phenyl.

8. A compound of formula (I) as claimed in claim 1, where R⁸ is hydrogen, methyl or fluorine.

9. A compound of formula (I) as claimed in claim 1, where R⁹ is hydrogen or methyl.

10. A compound of formula (I) as claimed in claim 1, where R¹⁰ is hydrogen, methyl, propyl, isopropyl, butyl, tert-butyl, dimethylamino, fluorine, chlorine, bromine, iodine, ethenyl, ethynyl, methylethynyl, ethylethynyl, MeOCH₂C≡C—, cyano, COOMe or CONH₂.

11. A compound of formula (I) as claimed in claim 1, where R¹¹ is hydrogen or methyl.

12. A compound of formula (I) as claimed in claim 1, where X represents CH₂, O or a bond, where, when n=0, X cannot be a bond.

13. A compound of formula (I) 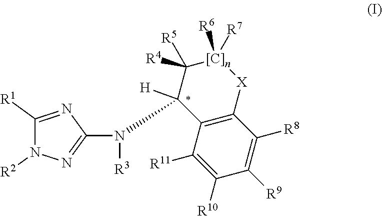
as claimed in claim 1, where the chiral carbon atom labeled (*) has an (R) configuration.

14. A compound of formula (I) 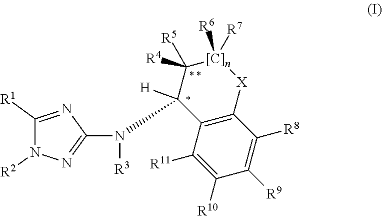
as claimed in claim 1, where the chiral carbon atom labeled (*) has an (R) configuration and the chiral carbon atom labeled (**) has an (S) configuration.

15. A process for preparing a compound of formula (I) or agrochemically compatible salt thereof: 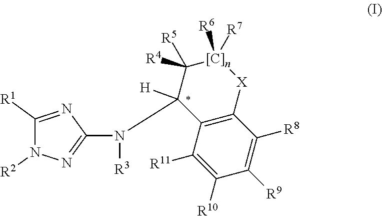
comprising reacting
a compound of formula (II) 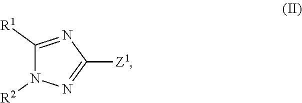
where —Z¹ represents an exchangeable radical or a leaving group with an amine of formula (III) 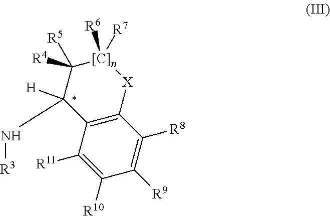
or with an acid addition salt of the amine of formula (III).

16. The process as claimed in claim 15, where Z¹ is fluorine, chlorine, bromine, iodine, (C₁₋₄)-alkylsulfanyl, (C₁₋₄)-alkylsulfinyl, (C₁₋₄)-alkylsulfonyl, an unsubstituted or a mono- or poly-fluorine-, -chlorine-, -bromine- or —(C₁₋₄)-alkyl- or —(C₁₋₄)-alkoxy-substituted phenyl-(C₁₋₄)-alkylsulfonyl or (C₁₋₄)-alkylphenylsulfonyl.

17. The process as claimed in claim 15, wherein a compound of formula (II) is reacted with an amine of the formula (III), wherein the reaction is catalyzed by the reagents potassium phosphate, copper(I) iodide or N,N-diethyl-2-hydroxybenzamide, or in the manner of Buchwald-Hartwig coupling by transition metal catalyst systems and bases.

18. A herbicidal composition or plant growth-regulating composition comprising one or more compounds of formula (I) or salts thereof as claimed in claim 1.

19. A method of controlling harmful plants or regulating the growth of a plant, comprising applying an effective amount of one or more compounds of formula (I) or salts thereof as claimed in claim 1 to one or more plants, plant parts, plant seeds and/or an area under cultivation.

20. A product comprising one or more compounds of formula (I) or salts thereof as claimed in claim 1 as a herbicide or as a plant growth regulator.

21. The product as claimed in claim 20, wherein the one or more compounds of formula (I) or salts thereof are used to control harmful plants and/or to regulate the growth of plants in one or more crops of useful plants or ornamental plants.

22. The product as claimed in claim 20, wherein the crop plants are transgenic crop plants.
